(19) United States
(12) Patent Application Publication
(10) Pub. No.: US 2020/0370042 A1
(43) Pub. Date: Nov. 26, 2020

(54) COMPOSITIONS AND METHODS FOR CORRECTING DYSTROPHIN MUTATIONS IN HUMAN CARDIOMYOCYTES

(71) Applicant: The Board of Regents of the University of Texas System, Austin, TX (US)

(72) Inventors: Eric N. OLSON, Dallas, TX (US); Chengzu LONG, New York, NY (US)

(73) Assignee: The Board of Regents of the University of Texas System, Austin, TX (US)

(21) Appl. No.: 16/966,274

(22) PCT Filed: Jan. 31, 2019
(86) PCT No.: PCT/US2019/015988

§ 371 Date: Jul. 30, 2020

Related U.S. Application Data
(60) Provisional application No. 62/624,748, filed on Jan. 31, 2018.

(51) Int. Cl. C12N 15/11; C12N 9/22; C12N 7/00; C12N 15/86; C12N 5/10; C12N 5/077; A61P 21/00; A61K 35/34; C12N 5/074

(52) U.S. Cl. CPC C12N 15/11; C12N 2330/51; C12N 7/00; C12N 15/86; C12N 5/10; C12N 5/0657; A61P 21/00; A61K 35/34; C12N 5/0696; C12N 2310/20; C12N 2800/80; C12N 2750/14143; C12N 2510/00; C12N 2506/45; C12N 9/22

(57) ABSTRACT

The disclosure provides a method for treating or preventing Duchene Muscular Dystrophy (DMD) in a subject in need thereof, the method comprising administering to the subject a Cas9 nuclease or a sequence encoding a Cas9 nuclease, and a gRNA or a sequence encoding a gRNA, wherein the gRNA targets a splice donor or splice acceptor site of the dystrophin gene. The administering restores dystrophin expression in at least a subset of the subjects cardiomyocytes, and may at least partially or fully restore cardiac contractility.

### CROSS-REFERENCE TO RELATED APPLICATIONS

This application claims priority to U.S. Provisional Application Ser. No. 62/624,748, filed Jan. 31, 2018, which is incorporated by reference herein in its entirety for all purposes.

### FEDERAL FUNDING SUPPORT CLAUSE

This invention was made with government support under grants no. HL-130253, HL-077439, DK-099653, and AR-067294 awarded by the National Institutes of Health (NIH). The government has certain rights in the invention.

### SEQUENCE LISTING

The instant application contains a Sequence Listing which has been submitted electronically in ASCII format and is hereby incorporated by reference in its entirety. Said ASCII copy, created on Jan. 31, 2019, is named UTFDP0002WO.txt and is 1,722,119 bytes in size.

### FIELD OF THE DISCLOSURE

The present disclosure relates to the fields of molecular biology, medicine and genetics. More particularly, the disclosure relates to compositions and uses thereof for genome editing to correct mutations in vivo using an exon-skipping approach.

### BACKGROUND

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable and lethal features of DMD. The disease is caused by mutations in the gene encoding dystrophin (DMD), a large intracellular protein that links the dystroglycan complex at the cell surface with the underlying cytoskeleton, thereby maintaining integrity of the muscle cell membrane during contraction. Mutations in the dystrophin gene result in loss of expression of dystrophin, causing muscle membrane fragility and progressive muscle wasting.

### SUMMARY

Genomic editing with CRISPR/Cas9 is a promising new approach for correcting or mitigating disease-causing mutations. Duchenne muscular dystrophy (DMD) is associated with lethal degeneration of cardiac and skeletal muscle caused by more than 3000 different mutations in the X-linked dystrophin gene (DMD). Most of these mutations are clustered in “hotspots.” As described in the Examples herein, a screen was performed for optimal guide RNAs capable of introducing insertion/deletion (indel) mutations by nonhomologous end joining that abolish conserved RNA splice sites in 12 exons that potentially allow skipping of the most common mutant or out-of-frame DMD exons within or nearby mutational hotspots. The correction of DMD mutations by exon skipping is referred to herein as “myoediting.” In proof-of-concept studies, myoediting was performed in representative induced pluripotent stem cells from multiple patients with large deletions, point mutations, or duplications within the DMD gene and efficiently restored dystrophin protein expression in derivative cardiomyocytes. In three-dimensional engineered heart muscle (EHM), myoediting of DMD mutations restored dystrophin expression and the corresponding mechanical force of contraction. Correcting only a subset of cardiomyocytes (30 to 50%) was sufficient to rescue the mutant EHM phenotype to near-normal control levels. Thus, it is shown that abolishing conserved RNA splicing acceptor/donor sites and directing the splicing machinery to skip mutant or out-of-frame exons through myoediting allows correction of the cardiac abnormalities associated with DMD by eliminating the underlying genetic basis of the disease.

Thus, in some embodiments, the disclosure provides a method for editing a mutant dystrophin gene in a cardiomyocyte, the method comprising contacting the cardiomyocyte with a Cas9 nuclease or a sequence encoding a Cas9 nuclease, and a gRNA or a sequence encoding a gRNA, wherein the gRNA targets a splice donor or splice acceptor site of the dystrophin gene.

The disclosure also provides a method for treating or preventing Duchene Muscular Dystrophy (DMD) in a subject in need thereof, the method comprising administering to the subject a Cas9 nuclease or a sequence encoding a Cas9 nuclease, and a gRNA or a sequence encoding a gRNA, wherein the gRNA targets a splice donor or splice acceptor site of the dystrophin gene; wherein the administering restores dystrophin expression in at least 10% of the subject's cardiomyocytes.

The disclosure also provides a method for treating or preventing Duchene Muscular Dystrophy (DMD) in a subject in need thereof, the method comprising contacting an induced pluripotent stem cell (iPSC) with a Cas9 nuclease or a sequence encoding a Cas9 nuclease, and a gRNA, or a sequence encoding a gRNA, wherein the gRNA targets a splice donor or splice acceptor site of the dystrophin gene; differentiating the iPSC into a cardiomyocyte; and administering the cardiomyocyte to a the subject.

Also provided is a cell (such as an induced pluripotent stem cell (iPSC) or cardiomyocyte) produced according to the methods of the disclosure, and compositions thereof. In some embodiments, the cell expresses a dystrophin protein.

Also provided is an induced pluripotent stem cell (iPSC) comprising a Cas9 nuclease, or a sequence encoding a Cas9 nuclease, and a gRNA, or a sequence encoding a gRNA, wherein the gRNA targets a splice donor or splice acceptor site of the dystrophin gene.

As used in the specification, “a” or “an” may mean one or more. As used in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

Throughout this application, nucleotide sequences are listed in the 5′ to 3′ direction, and amino acid sequences are listed in the N-terminal to C-terminal direction, unless indicated otherwise.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A-1C. Myoediting strategy and identification of optimal guide RNAs to target the top 12 exons in DMD. (FIG. 1A) Conserved splice sites contain multiple NAG and NGG sequences, which enable cleavage by SpCas9. The numbers indicate the frequency of occurrence (%). (FIG. 1B) Human DMD exon structure. Shapes of intron-exon junctions indicate complementarity that maintains the open reading frame upon splicing. Red arrowheads indicate the top 12 targeted exons. The numbers indicate the order of the exons. (FIG. 1C) T7E1 assays in human 293 cells transfected with plasmids expressing the corresponding guide RNA (gRNA), SpCas9, and GFP for the top 12 exons. The PCR products from GFP+ and GFP− cells were cut with T7 endonuclease I (T7E1), which is specific to heteroduplex DNA caused by CRISPR/Cas9-mediated genome editing. Red arrowhead indicates cleavage bands of T7E1. M denotes size marker lane. bp indicates the base pair length of the marker bands.

FIG. 2A-2J. Rescue of dystrophin mRNA expression in iPSC-derived cardiomyocytes with diverse mutations by myoediting. (FIG. 2A) Schematic of the myoediting of DMD iPSCs and 3D-EHMs-based functional assay. (FIG. 2B) Myoediting targets the exon 51 splice acceptor site in Del DMD iPSCs. A deletion (exons 48 to 50) in a DMD patient creates a frameshift mutation in exon 51. The red box indicates out-of-frame exon 51 with a stop codon. Destruction of the exon 51 splice acceptor in DMD iPSCs allows splicing from exons 47 to 52 and restoration of the dystrophin open reading frame. (FIG. 2C) Using the guide RNA library, three guide RNAs (Ex51-g1, Ex51-g2, and Ex51-g3) that target sequences 5′ of exon 51 were selected. FIG. 2C discloses SEQ ID NO: 2481. (FIG. 2D) RT-PCR of cardiomyocytes differentiated from uncorrected DMD (Del), corrected DMD iPSCs (Del-Cor.), and WT. Skipping of exon 51 allows splicing from exons 47 to 52 (lower band) and restoration of the DMD open reading frame. (FIG. 2E) Myoediting strategy for pseudo-exon 47A (pEx). DMD exons are represented as blue boxes. Pseudo-exon 47A (red) with stop codon is marked by a stop sign. The black box indicates myoediting-mediated indel. (FIG. 2F) Sequence of guide RNAs for pseudo-exon 47A of pEx. DMD exons are represented as blue boxes, and pseudo-exons are represented as red boxes (47A). sgRNA, single-guide RNA. FIG. 2F discloses SEQ ID NOS 2482-2484, respectively, in order of appearance. (FIG. 2G) RT-PCR of human cardiomyocytes differentiated from WT, uncorrected DMD (pEx), and corrected DMD iPSCs (pEx-Cor.) by guide RNAs In47A-g1 and In47A-g2. Skipping of pseudo-exon 47A allows splicing from exons 47 to 48 (lower band) and restoration of the DMD open reading frame. (FIG. 2H) Myoediting strategy for the duplication (Dup) of exons 55 to 59. DMD exons are represented as blue boxes. Duplicated exons are represented as red boxes. The black box indicates myoediting-mediated indel. (FIG. 2I) Sequence of guide RNAs for intron 54 of Dup (In54-g1, In54-g2, and In54-g3). FIG. 2I discloses SEQ ID NOS 2485-2487, respectively, in order of appearance. (FIG. 2J) RT-PCR of human cardiomyocytes differentiated from WT, uncorrected DMD (Dup), and corrected DMD iPSCs (Dup-Cor.). Skipping of duplicated exons 55 to 59 allows splicing from exons 54 to 55 and restoration of the DMD open reading frame. RT-PCR of RNA was performed with the indicated sets of primers (F and R) (Table 4).

FIG. 3A-3F. Immunocytochemistry and Western blot analysis show dystrophin protein expression rescued by myoediting. (FIG. 3A to 3C) Immunocytochemistry of dystrophin expression (green) shows DMD iPSC cardiomyocytes lacking dystrophin expression. Following successful myoediting, the corrected DMD iPSC cardiomyocytes express dystrophin. Immunofluorescence (red) detects cardiac marker troponin-I. Nuclei are labeled by Hoechst dye (blue). (FIG. 3D to 3F) Western blot analysis of WT (100 and 50%), uncorrected (Del, pEx, and Dup) and corrected DMD (Del-Cor #27, pEx-Cor #19, and Dup-Cor #6.) iCM. Red arrowhead (above 250 kD) indicates the immunoreactive bands of dystrophin. Blue arrowhead (above 150 kD) indicates the immunoreactive bands of MyHC loading controls. kD indicates protein molecular weight. Scale bar, 100 mm.

FIG. 4A-4F. Rescued DMD cardiomyocyte-derived EHM showed enhanced FOC (force of contraction). (FIG. 4A) Experimental setup for EHM preparation, culture, and analysis of contractile function. (FIG. 4B to 4D) Contractile dysfunction in DMD EHM can be rescued by myoediting. FOC normalized to muscle content of each individual EHM in response to increasing extracellular calcium concentrations; n=8/8/6/4/6/6/4/4; *P<0.05 by two-way analysis of variance (ANOVA) and Tukey's multiple comparison test. WT EHM data are pooled from parallel experiments with indicated DMD lines and applied to FIG. 4 (B to D). (FIG. 4E) Maximal cardiomyocyte FOC normalized to WT. n=8/8/6/4/6/6/4/4; *P<0.05 by one-way ANOVA and Tukey's multiple comparison test. (FIG. 4F) Titration of corrected cardiomyocytes revealed that 30% of cardio-myocytes needed to be repaired to partially rescue the phenotype, and 50% of cardiomyocytes needed to be repaired to fully rescue the phenotype (100% Del-Cor.) in EHMs. WT, Del, and 100% Del-Cor. are pooled data, as displayed in FIG. 4.

FIG. 5A-5B. Genome editing of DMD top 12 exons by CRISPR/Cas9. (FIG. 5A) DNA sequences of DMD top 12 exons (51, 45, 53, 44, 46, 52, 50, 43, 6, 7, 8 and 55) from GPF+ human 293 cells edited by SpCas9 using the corresponding guide RNAs (Table 5). PCR products from genomic DNA of each sample were subcloned into pCRII-TOPO vector and individual clones were picked and sequenced. Unedited wild type (WT) sequences are on the top and representative edited sequences are on the bottom. Deleted sequences are replaced by black dashes. Red lower case letters (ag) indicate the splice acceptor sites (SA, 3′ end of the intron). Blue lower case letters (gt) indicate the splice donor sites (SD, 5′ end of the intron). FIG. 5A discloses SEQ ID NOS 2488-2526 in the left column and SEQ ID NOS 2427-2546 in the right column, all respectively, in order of appearance. (FIG. 5B) RT-PCR of RNA from edited 293 cells indicate deletion of targeted DMD Dp140 isoform exons (51, 53, 46, 52, 50 and 55). Black arrows indicate the RT-PCR products with exon deletions. M denotes size marker lane. bp indicates the length of the marker bands. Sequence of the RT-PCR products of exon deletion bands contained the two flanking exons, but skipped the targeted exon. For example, sequence of the RT-PCR products of ΔEx51 band confirmed that exon 50 spliced directly to exon 52, excluding exon 51. FIG. 5B discloses “GAGCCTGCAACA” as SEQ ID NO: 2547, “ATCGAACAGTTG” as SEQ ID NO: 2548, “AAAGAGTTACTG” as SEQ ID NO: 2549, “CAGAAGTTGAAA” as SEQ ID NO: 2550, “GTGAAGCTCCTA” as SEQ ID NO: 2551 and “TAAAAGGACCTC” as SEQ ID NO: 2552.

FIG. 6A-6D. Correction of a large deletion mutation (Del. Ex47-50) in DMD iPSCs and iPSC-derived cardiomyocytes. (FIG. 6A) T7E1 assay using human 293 cells transfected with plasmid expressing SpCas9, gRNAs (Ex51-g1, g2 and g3), and GFP show genome cleavage at DMD exon 51. Red arrowheads point to cleavage products. M, marker; bp, base pair. (FIG. 6B) DNA sequences of DMD exon 51 from GPF+ DMD Del iPSCs edited by SpCas9 and the guide RNA Ex51 g3. PCR products from genomic DNA of a mixture of myoedited DMD iPSCs were subcloned into pCRII-TOPO vector and sequenced as described above. Uncorrected exon51 sequence is on the top and representative edited sequences are on the bottom. Deleted sequences are replaced by black dashes. Red lower-case letters (ag) indicate the splice acceptor sites. The number of deleted nucleotides is indicated by (-). FIG. 6B discloses SEQ ID NOS 2553-2561, respectively, in order of appearance. (FIG. 6C) Sequence of the lower RT-PCR band from FIG. 2D (Del-Cor. lane) confirms skipping of exon 51, which reframed the DMD ORF (dystrophin transcript from exons 47 to 52). FIG. 6C discloses SEQ ID NO: 2562. (FIG. 6D) Immunocytochemistry shows dystrophin expression in iPSC-derived cardiomyocyte mixtures (Del-Cor.) and single colony (Del-Cor-SC) following SpCas9-mediated exon skipping with guide RNA Ex51-g3 compared to WT and uncorrected cardiomyocyte (Del). Green, dystrophin staining; red, troponin I staining; blue, nuclei staining. Scale bar=100 μm.

FIG. 7A-7D. Correction of a pseudo-exon mutation (pEx47A) in DMD iPSCs and iPSC-derived cardiomyocytes. (FIG. 7A) T7E1 assay using DMD pEx47A iPSCs nucleofected with vector expressing SpCas9, gRNAs (pEx47A-g1 and g2), and GFP show genome cleavage at DMD pseudo-exon 47A. Red arrowheads point to cleavage products. M, marker; bp, base pair. (FIG. 7B) DNA sequences of DMD pseudo-exon 47A from GPF+ DMD Del iPSCs edited by SpCas9 and the guide RNA pEx47A-g1 and g2. PCR products from genomic DNA of a mixture of myoedited DMD iPSCs were subcloned and sequenced as described above. Uncorrected pseudo-exon 47A sequence is on the top and representative edited sequences are on the bottom. Deleted sequences are replaced by black dashes. Red lower case letter (g) indicate point mutation in the cryptic splice acceptor site. The number of deleted nucleotides is indicated by (-). FIG. 7B discloses SEQ ID NOS 2563-2567, respectively, in order of appearance. (FIG. 7C) Sequence of the lower RT-PCR bands from FIG. 2G (pEx and pEx-Cor. lanes) confirms skipping of pseudo-exon 47A, which reframed the DMD ORF (dystrophin transcript from exons 47 to 48). FIG. 7C discloses SEQ ID NOS 2568-2569, respectively, in order of appearance. (FIG. 7D) Immunocytochemistry shows dystrophin expression in iPSC-derived cardiomyocyte mixtures (pEx-Cor.) and single colony (pEx-Cor-SC) following SpCas9-mediated exon skipping with guide RNA pEx47A-g2 compared to WT and uncorrected cardiomyocyte (pEx). Green, dystrophin staining; red, troponin I staining; blue, nuclei staining. Scale bar=100 μm.

FIG. 8A-8E. Correction of a large duplication mutation (Dup. Ex55-59) in DMD iPSCs and iPSC-derived cardiomyocytes. (FIG. 8A) This insertion site (In59-In54 junction) was confirmed by PCR using a forward primer targeting intron 59 (F2) and a reverse primer targeting intron 54 (F1) (FIG. 2H and Table 4). The duplication-specific PCR band was absent in WT cells and was presented in Dup cells. (FIG. 8B) T7E1 assays using 293 cells with vector expressing SpCas9, gRNAs (In54-g1, g2 and g3), and GFP show genome cleavage at DMD intron 54. Red arrowheads point to cleavage products. M, marker; bp, base pair. (FIG. 8C) mRNA with duplicated exons was semi-quantified by RT-PCR using the primers flanking the duplication borders exon 53 and exon 55 (Ex53F, a forward primer in exon 53 and Ex59R, a reverse primer in exon 59). Similarly, duplicated exons was semi-quantified by RT-PCR using the primers flanking the duplication borders exon 59 and exon 60 (Ex59F, a forward primer in exon 59 and Ex60R, a reverse primer in exon 60). The duplication-specific RT-PCR upper bands (red arrowhead) were absent in WT cells and were decreased dramatically in Dup-Cor. cells. (FIG. 8D) PCR results of three representative corrected single colonies (Dup-Cor-SC #4, 6 and 26) and the uncorrected control (Dup). The absence of a duplication-specific PCR band (F2-R1) in colonies 4, 6 and 26 confirmed the deletion of the duplicated DNA region. M denotes size marker lane. bp indicates the length of the marker bands. (FIG. 8E) Immunocytochemistry shows dystrophin expression in iPSC-derived cardiomyocyte mixtures (Dup-Cor.) and single colony (Dup-Cor-SC #6) following SpCas9-mediated exon skipping with guide RNA In54-g1 compared to WT and uncorrected cardiomyocyte (Dup). Green, dystrophin staining; red, troponin I staining; blue, nuclei staining. Scale bar=100 μm.

### DETAILED DESCRIPTION

DMD is a new mutation syndrome with more than 4,000 independent mutations that have been identified in humans (world-wide web at dmd.nl). The majority of patient mutations include deletions that cluster in a hotspot, and thus a therapeutic approach for skipping certain exon applies to large group of patients. The rationale of the exon skipping approach is based on the genetic difference between DMD and Becker muscular dystrophy (BMD) patients. In DMD patients, the reading frame of dystrophin mRNA is disrupted resulting in prematurely truncated, non-functional dystrophin proteins. BMD patients have mutations in the DMD gene that maintain the reading frame allowing the production of internally deleted, but partially functional dystrophins leading to much milder disease symptoms compared to DMD patients.

Duchenne muscular dystrophy (DMD) afflicts ˜1 in 5000 males and is caused by mutations in the X-linked dystrophin gene (DMD). These mutations include large deletions, large duplications, point mutations, and other small mutations. The rod-shaped dystrophin protein links the cytoskeleton and the extracellular matrix of muscle cells and maintains the integrity of the plasma membrane. In its absence, muscle cells degenerate. Although DMD causes many severe symptoms, dilated cardiomyopathy is a leading cause of death of DMD patients.

CRISPR (clustered regularly interspaced short palindromic repeats)/Cas9 (CRISPR-associated protein 9)-mediated genome editing is emerging as a promising tool for correction of genetic disorders. Briefly, an engineered RNA-guided nuclease, such as Cas9 or Cpf1, generates a double-strand break (DSB) at the targeted genomic locus adjacent to a short protospacer adjacent motif (PAM) sequence. There are three primary pathways to repair the DSB: (i) Nonhomologous end joining (NHEJ) directly ligates two DNA ends and leads to imprecise insertion/deletion (indel) mutations. (ii) Homology-directed repair (HDR) uses sister chromatid or exogenous DNA as a repair template and generates a precise modification at the target sites. (iii) Microhomology-mediated end joining (MMEJ) uses short sequences of nucleotide homology (5 to 25 base pairs) flanking the original DSB to ligate the broken ends and deletes the region between the microhomologies. Although NHEJ can effectively generate indel mutations in most cell types, HDR- or MMEJ-mediated editing is generally thought to be restricted to proliferating cells.

Internal in-frame deletions of dystrophin are associated with Becker muscular dystrophy (BMD), a relatively mild form of muscular dystrophy. Inspired by the attenuated clinical severity of BMD versus DMD, exon skipping has been advanced as a therapeutic strategy to bypass mutations that disrupt the dystrophin open reading frame by modulating splicing patterns of the DMD gene. Several recent studies used CRISPR/Cas9-mediated genome editing to correct various types of DMD mutations in human cells and mice. Some have deployed pairs of guide RNAs to correct the mutation, which requires simultaneous cutting of DNA and excision of large intervening genomic sequences (23 to 725 kb). Fortuitously, the PAM sequence for Streptococcus pyogenes Cas9 (SpCas9), the first and most widely used form of Cas9, contains NAG or NGG, corresponding to the universal splice acceptor sequence (AG) and most of the donor sequences (GG). Thus, in principle, directing Cas9 to splice junctions and the elimination of these consensus sequences by indels can allow for efficient exon skipping. In addition, only a single cleavage of DNA, which disrupts the splice site, can enable skipping of an entire exon.

Given the thousands of individual DMD mutations that have been identified in humans, an obvious question is how such a large number of mutations might be corrected by CRISPR/Cas9-mediated genome editing. Human DMD mutations are clustered in specific “hotspot” areas of the gene (exons 45 to 55 and exons 2 to 10) such that skipping 1 or 2 of 12 targeted exons within or nearby the hotspots (termed “top 12 exons”) can, in principle, rescue dystrophin function in a majority (˜60%) of DMD patients. Here, CRISPR/Cas9 is used with single-guide RNAs to destroy the conserved splice acceptor or donor sites preceding DMD mutations or to bypass mutant or out-of-frame exons, thereby allowing splicing between surrounding exons to recreate in-frame dystrophin proteins lacking the mutations. This approach was first tested by screening for optimal guide RNAs capable of inducing skipping of the DMD 12 exons that would potentially allow skipping of the most commonly mutated or out-of-frame exons within nearby mutational hotspots. As examples of this approach, the restoration of dystrophin expression is demonstrated in induced pluripotent stem cell (iPSC)-derived cardiomyocytes harboring exon deletions and a pseudo-exon point mutation. Finally, human iPSC-derived three-dimensional (3D) engineered heart muscle (EHM) was used to test the efficacy of gene editing to overcome abnormalities in cardiac contractility associated with DMD. Contractile dysfunction was observed in DMD EHM, recapitulating the dilated cardiomyopathy (DCM) clinical phenotype of DMD patients, and contractile function was effectively restored in corrected DMD EHM. Thus, genome editing represents a powerful means of eliminating the genetic cause and correcting the muscle and cardiac abnormalities associated with DMD.

These and other aspects of the disclosure are described in further detail below.

### CRISPR Systems

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

Guide RNA (gRNA).

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6. Synthetic gRNAs are slightly over 100 bp at the minimum length and contain a portion which is targets the 20 protospacer nucleotides immediately preceding the PAM sequence NGG; gRNAs do not contain a PAM sequence.

In some embodiments, the gRNA targets a site within a wildtype dystrophin gene. An exemplary wildtype dystrophin gene includes the human sequence (see GenBank Accession NO. NC_000023.11), located on the human X chromosome, which codes for the protein dystrophin (GenBank Accession No. AAA53189; SEQ ID NO: 5), the sequence of which is reproduced below:

| | |
|---|---|
| 1 | MLWWEEVEDC YEREDVQKKT FTKWVNAQFS KFGKQHIENL FSDLQDGRRL LDLLEGLTGQ |
| 61 | KLPKEKGSTR VHALNNVNKA LRVLQNNNVD LVNIGSTDIV DGNHKLTLGL IWNIILHWQV |
| 121 | KNVMKNIMAG LQQTNSEKIL LSWVRQSTRN YPQVNVINFT TSWSDGLALN ALIHSHRPDL |
| 181 | FDWNSVVCQQ SATQRLEHAF NIARYQLGIE KLLDPEDVDT TYPDKKSILM YITSLFQVLP |
| 241 | QQVSIEAIQE VEMLPRPPKV TKEEHFQLHH QMHYSQQITV SLAQGYERTS SPKPRFKSYA |
| 301 | YTQAAYVTTS DPTRSPFPSQ HLEAPEDKSF GSSLMESEVN LDRYQTALEE VLSWLLSAED |
| 361 | TLQAQGEISN DVEVVKDQFH THEGYMMDLT AHQGRVGNIL QLGSKLIGTG KLSEDEETEV |
| 421 | QEQMNLLNSR WECLRVASME KQSNLHRVLM DLQNQKLKEL NDWLTKTEER TRKMEEEPLG |
| 481 | PDLEDLKRQV QQHKVLQEDL EQEQVRVNSL THMVVVVDES SGDHATAALE EQLKVLGDRW |
| 541 | ANICRWTEDR WVLLQDILLK WQRLTEEQCL FSAWLSEKED AVNKIHTTGF KDQNEMLSSL |
| 601 | QKLAVLKADL EKKKQSMGKL YSLKQDLLST LKNKSVTQKT EAWLDNFARC WDNLVQKLEK |
| 661 | STAQISQAVT TTQPSLTQTT VMETVTTVTT REQILVKHAQ EELPPPPPQK KRQITVDSEI |
| 721 | RKRLDVDITE LHSWITRSEA VLQSPEFAIF RKEGNFSDLK EKVNAIEREK AEKFRKLQDA |
| 781 | SRSAQALVEQ MVNEGVNADS IKQASEQLNS RWIEFCQLLS ERLNWLEYQN NIIAFYNQLQ |
| 841 | QLEQMTTTAE NWLKIQPTTP SEPTAIKSQL KICKDEVNRL SGLQPQIERL KIQSIALKEK |
| 901 | GQGPMFLDAD FVAFTNHFKQ VFSDVQAREK ELQTIFDTLP PMRYQETMSA IRTWVQQSET |
| 961 | KLSIPQLSVT DYEIMEQRLG ELQALQSSLQ EQQSGLYYLS TTVKEMSKKA PSEISRKYQS |
| 1021 | EFEEIEGRWK KLSSQLVEHC QKLEEQMNKL RKIQNHIQTL KKWMAEVDVF LKEEWPALGD |
| 1081 | SEILKKQLKQ CRLLVSDIQT IQPSLNSVNE GGQKIKNEAE PEFASRLETE LKELNTQWDH |
| 1141 | MCQQVYARKE ALKGGLEKTV SLQKDLSEMH EWMTQAEEEY LERDFEYKTP DELQKAVEEM |
| 1201 | KRAKEEAQQK EAKVKLLTES VNSVIAQAPP VAQEALKKEL ETLTTNYQWL CTRLNGKCKT |
| 1261 | LEEVWACWHE LLSYLEKANK WLNEVEFKLK TTENIPGGAE EISEVLDSLE NLMRHSEDNP |
| 1321 | NQIRILAQTL TDGGVMDELI NEELETFNSR WRELHEEAVR RQKLLEQSIQ SAQETEKSLH |
| 1381 | LIQESLTFID KQLAAYIADK VDAAQMPQEA QKIQSDLTSH EISLEEMKKH NQGKEAAQRV |
| 1441 | LSQIDVAQKK LQDVSMKFRL FQKPANFELR LQESKMILDE VKMHLPALET KSVEQEVVQS |
| 1501 | QLNHCVNLYK SLSEVKSEVE MVIKTGRQIV QKKQTENPKE LDERVTALKL HYNELGAKVT |
| 1561 | ERKQQLEKCL KLSRKMRKEM NVLTEWLAAT DMELTKRSAV EGMPSNLDSE VAWGKATQKE |
| 1621 | IEKQKVHLKS ITEVGEALKT VLGKKETLVE DKLSLLNSNW IAVTSRAEEW LNLLLEYQKH |
| 1681 | METFDQNVDH ITKWIIQADT LLDESEKKKP QQKEDVLKRL KAELNDIRPK VDSTRDQAAN |
| 1741 | LMANRGDHCR KLVEPQISEL NHRFAAISHR IKTGKASIPL KELEQFNSDI QKLLEPLEAE |
| 1801 | IQQGVNLKEE DFNKDMNEDN EGTVKELLQR GDNLQQRITD ERKREEIKIK QQLLQTKHNA |
| 1861 | LKDLRSQRRK KALEISHQWY QYKRQADDLL KCLDDIEKKL ASLPEPRDER KIKEIDRELQ |
| 1921 | KKKEELNAVR RQAEGLSEDG AAMAVEPTQI QLSKRWREIE SKFAQFRRLN FAQIHTVREE |
| 1981 | TMMVMTEDMP LEISYVPSTY LTEITHVSQA LLEVEQLLNA PDLCAKDFED LFKQEESLKN |
| 2041 | IKDSLQQSSG RIDIIHSKKT AALQSATPVE RVKLQEALSQ LDFQWEKVNK MYKDRQGRFD |
| 2101 | RSVEKWRRFH YDIKIFNQWL TEAEQFLRKT QIPENWEHAK YKWYLKELQD GIGQRQTVVR |
| 2161 | TLNATGEEII QQSSKTDASI LQEKLGSLNL RWQEVCKQLS DRKKRLEEQK NILSEFQRDL |
| 2221 | NEFVLWLEEA DNIASIPLEP GKEQQLKEKL EQVKLLVEEL PLRQGILKQL NETGGPVLVS |
| 2281 | APISPEEQDK LENKLKQTNL QWIKVSRALP EKQGEIEAQI KDLGQLEKKL EDLEEQLNHL |
| 2341 | LLWLSPIRNQ LEIYNQPNQE GPFDVQETEI AVQAKQPDVE EILSKGQHLY KEKPATQPVK |
| 2401 | RKLEDLSSEW KAVNRLLQEL RAKQPDLAPG LTTIGASPTQ TVTLVTQPVV TKETAISKLE |
| 2461 | MPSSLMLEVP ALADFNRAWT ELTDWLSLLD QVIKSQRVMV GDLEDINEMI IKQKATMQDL |
| 2521 | EQRRPQLEEL ITAAQNLKNK TSNQEARTII TDRIERIQNQ WDEVQEHLQN RRQQLNEMLK |
| 2581 | DSTQWLEAKE EAEQVLGQAR AKLESWKEGP YTVDAIQKKI TETKQLAKDL RQWQTNVDVA |
| 2641 | NDLALKLLRD YSADDTRKVH MITENINASW RSIHKRVSER EAALEETHRL LQQFPLDLEK |
| 2701 | FLAWLTEAET TANVLQDATR KERLLEDSKG VKELMKQWQD LQGEIEAHTD VYHNLDENSQ |
| 2761 | KILRSLEGSD DAVLLQRRLD NMNFKWSELR KKSLNIRSHL EASSDQWKRL HLSLQELLVW |
| 2821 | LQLKDDELSR QAPIGGDFPA VQKQNDVHRA FKRELKTKEP VIMSTLETVR IFLTEQPLEG |
| 2881 | LEKLYQEPRE LPPEERAQNV TRLLRKQAEE VNTEWEKLNL HSADWQRKID ETLERLQELQ |
| 2941 | EATDELDLKL RQAEVIKGSW QPVGDLLIDS LQDHLEKVKA LRGEIAPLKE NVSHVNDLAR |
| 3001 | QLTTLGIQLS PYNLSTLEDL NTRWKLLQVA VEDRVRQLHE AHRDFGPASQ HFLSTSVQGP |
| 3061 | WERAISPNKV PYYINHETQT TCWDHPKMTE LYQSLADLNN VRFSAYRTAM KLRRLQKALC |
| 3121 | LDLLSLSAAC DALDQHNLKQ NDQPMDILQI INCLTTIYDR LEQEHNNLVN VPLCVDMCLN |
| 3181 | WLLNVYDTGR TGRIRVLSFK TGIISLCKAH LEDKYRYLFK QVASSTGFCD QRRLGLLLHD |
| 3241 | SIQIPRQLGE VASFGGSNIE PSVRSCFQFA NNKPEIEAAL FLDWMRLEPQ SMVWLPVLHR |
| 3301 | VAAAETAKHQ AKCNICKECP IIGFRYRSLK HFNYDICQSC FFSGRVAKGH KMHYPMVEYC |
| 3361 | TPTTSGEDVR DFAKVLKNKF RTKRYFAKHP RMGYLPVQTV LEGDNMETPV TLINFWPVDS |
| 3421 | APASSPQLSH DDTHSRIEHY ASRLAEMENS NGSYLNDSIS PNESIDDEHL LIQHYCQSLN |
| 3481 | QDSPLSQPRS PAQILISLES EERGELERIL ADLEEENRNL QAEYDRLKQQ HEHKGLSPLP |
| 3541 | SPPEMMPTSP QSPRDAELIA EAKLLRQHKG RLEARMQILE DHNKQLESQL HRLRQLLEQP |
| 3601 | QAEAKVNGTT VSSPSTSLQR SDSSQPMLLR VVGSQTSDSM GEEDLLSPPQ DTSTGLEEVM |
| 3661 | EQLNNSFPSS RGRNTPGKPM REDTM. |

In some embodiments, the gRNA targets a site within a mutant dystrophin gene. In some embodiments, the gRNA targets a dystrophin intron. In some embodiments, the gRNA targets a dystrophin exon. In some embodiments, the gRNA targets a site in a dystrophin exon that is expressed and is present in one or more of the dystrophin isoforms shown in Table 1. In embodiments, the gRNA targets a dystrophin splice site. In some embodiments, the gRNA targets a splice donor site on the dystrophin gene. In embodiments, the gRNA targets a splice acceptor site on the dystrophin gene.

TABLE 1
Dystrophin isoforms

| | | | | Pro- | |
|---|---|---|---|---|---|
| | | Nucleic | | tein | |
| | | Acid | Protein | SEQ | |
| Sequence | Nucleic Acid | SEQ ID | Accession | ID | |
| Name | Accession No. | NO: | No. | NO: | Description |
| DMD | NC_000023.11 | None | None | None | Sequence from Human X Chromosome (at |
| Genomic | (positions | | | | positions Xp21.2 to p21.1) from Assembly |
| Sequence | 31119219 to | | | | GRCh38.p7 (GCF_000001405.33) |
| | 33339609) | | | | |
| Dystrophin | NM_000109.3 | 6 | NP_000100.2 | 7 | Transcript Variant: transcript Dp427c is |
| Dp427c | | | | | expressed predominantly in neurons of the |
| isoform | | | | | cortex and the CA regions of the hippocampus. |
| | | | | | It uses a unique promoter/exon 1 located about |
| | | | | | 130 kb upstream of the Dp427m transcript |
| | | | | | promoter. The transcript includes the common |
| | | | | | exon 2 of transcript Dp427m and has a similar |
| | | | | | length of 14 kb. The Dp427c isoform contains a |
| | | | | | unique N-terminal MED sequence, instead of the |
| | | | | | MLWWEEVEDCY sequence (SEQ ID NO: 2476) of |
| | | | | | isoform Dp427m. The remainder of isoform |
| | | | | | Dp427c is identical to isoform Dp427m. |
| Dystrophin | NM_004006.2 | 8 | NP_003997.1 | 9 | Transcript Variant: transcript Dp427m encodes |
| Dp427m | | | | | the main dystrophin protein found in muscle. |
| isoform | | | | | As a result of alternative promoter use, exon |
| | | | | | 1 encodes a unique N-terminal MLWWEEVEDCY |
| | | | | | (SEQ ID NO: 2476) aa sequence. |
| Dystrophin | NM_004009.3 | 10 | NP_004000.1 | 11 | Transcript Variant: transcript Dp427p1 |
| Dp427p1 | | | | | initiates from a unique promoter/exon 1 |
| isoform | | | | | located in what corresponds to the first |
| | | | | | intron of transcript Dp427m. The transcript |
| | | | | | adds the common exon 2 of Dp427m and has a |
| | | | | | similar length (14 kb). The Dp427p1 isoform |
| | | | | | replaces the MLWWEEVEDCY (SEQ ID NO: 2476)- |
| | | | | | start of Dp427m with a unique N-terminal |
| | | | | | MSEVSSD (SEQ ID NO: 2477) aa sequence. |
| Dystrophin | NM_004011.3 | 12 | NP_004002.2 | 13 | Transcript Variant: transcript Dp260-1 uses |
| Dp260-1 | | | | | exons 30-79, and originates from a |
| isoform | | | | | promoter/exon 1 sequence located in intron 29 |
| | | | | | of the dystrophin gene. As a result, Dp260-1 |
| | | | | | contains a 95 bp exon 1 encoding a unique N- |
| | | | | | terminal 16 aa MTEIILLIFFPAYFLN-sequence (SEQ |
| | | | | | ID NO: 2478) that replaces amino acids 1-1357 |
| | | | | | of the full-length dystrophin product (Dp427m |
| | | | | | isoform). |
| Dystrophin | NM_004012.3 | 14 | NP_004003.1 | 15 | Transcript Variant: transcript Dp260-2 uses |
| Dp260-2 | | | | | exons 30-79, starting from a promoter/exon 1 |
| isoform | | | | | sequence located in intron 29 of the |
| | | | | | dystrophin gene that is alternatively spliced |
| | | | | | and lacks N-terminal amino acids 1-1357 of the |
| | | | | | full length dystrophin (Dp427m isoform). The |
| | | | | | Dp260-2 transcript encodes a unique N-terminal |
| | | | | | MSARKLRNLSYKK sequence (SEQ ID NO: 2479). |
| Dystrophin | NM_004013.2 | 16 | NP_004004.1 | 17 | Transcript Variant: Dp140 transcripts use exons |
| Dp140 | | | | | 45-79, starting at a promoter/exon 1 located in |
| isoform | | | | | intron 44. Dp140 transcripts have a long (1 kb) |
| | | | | | 5′ UTR since translation is initiated in exon |
| | | | | | 51 (corresponding to aa 2461 of dystrophin). In |
| | | | | | addition to the alternative promoter and exon |
| | | | | | 1, differential splicing of exons 71-74 and 78 |
| | | | | | produces at least five Dp140 isoforms. Of |
| | | | | | these, this transcript (Dp140) contains all of |
| | | | | | the exons. |
| Dystrophin | NM_004014.2 | 18 | NP_004005.1 | 19 | Transcript Variant: transcript Dp116 uses exons |
| Dp116 | | | | | 56-79, starting from a promoter/exon 1 within |
| isoform | | | | | intron 55. As a result, the Dp116 isoform |
| | | | | | contains a unique N-terminal MLHRKTYHVK aa |
| | | | | | sequence (SEQ ID NO: 2480), instead of aa 1- |
| | | | | | 2739 of dystrophin. Differential splicing |
| | | | | | produces several Dp116-subtypes. The Dp116 |
| | | | | | isoform is also known as S-dystrophin or apo- |
| | | | | | dystrophin-2. |
| Dystrophin | NM_004015.2 | 20 | NP_004006.1 | 21 | Transcript Variant: Dp71 transcripts use exons |
| Dp71 | | | | | 63-79 with a novel 80- to 100-nt exon |
| isoform | | | | | containing an ATG start site for a new coding |
| | | | | | sequence of 17 nt. The short coding sequence is |
| | | | | | in-frame with the consecutive dystrophin |
| | | | | | sequence from exon 63. Differential splicing of |
| | | | | | exons 71 and 78 produces at least four Dp71 |
| | | | | | isoforms. Of these, this transcript (Dp71) |
| | | | | | includes both exons 71 and 78. |
| Dystrophin | NM_004016.2 | 22 | NP_004007.1 | 23 | Transcript Variant: Dp71 transcripts use exons |
| Dp71b | | | | | 63-79 with a novel 80- to 100-nt exon |
| isoform | | | | | containing an ATG start site for a new coding |
| | | | | | sequence of 17 nt. The short coding sequence is |
| | | | | | in-frame with the consecutive dystrophin |
| | | | | | sequence from exon 63. Differential splicing of |
| | | | | | exons 71 and 78 produces at least four Dp71 |
| | | | | | isoforms. Of these, this transcript (Dp71b) |
| | | | | | lacks exon 78 and encodes a protein with a |
| | | | | | different C-terminus than Dp71 and Dp71a |
| | | | | | isoforms. |
| Dystrophin | NM_004017.2 | 24 | NP_004008.1 | 25 | Transcript Variant: Dp71 transcripts use exons |
| Dp71a | | | | | 63-79 with a novel 80- to 100-nt exon |
| isoform | | | | | containing an ATG start site for a new coding |
| | | | | | sequence of 17 nt. The short coding sequence is |
| | | | | | in-frame with the consecutive dystrophin |
| | | | | | sequence from exon 63. Differential splicing of |
| | | | | | exons 71 and 78 produces at least four Dp71 |
| | | | | | isoforms. Of these, this transcript (Dp71a) |
| | | | | | lacks exon 71. |
| Dystrophin | NM_004018.2 | 26 | NP_004009.1 | 27 | Transcript Variant: Dp71 transcripts use exons |
| Dp71ab | | | | | 63-79 with a novel 80- to 100-nt exon |
| isoform | | | | | containing an ATG start site for a new coding |
| | | | | | sequence of 17 nt. The short coding sequence is |
| | | | | | in-frame with the consecutive dystrophin |
| | | | | | sequence from exon 63. Differential splicing of |
| | | | | | exons 71 and 78 produces at least four Dp71 |
| | | | | | isoforms. Of these, this transcript (Dp71ab) |
| | | | | | lacks both exons 71 and 78 and encodes a |
| | | | | | protein with a C-terminus like isoform Dp71b. |
| Dystrophin | NM_004019.2 | 28 | NP_004010.1 | 29 | Transcript Variant: transcript Dp40 uses exons |
| Dp40 | | | | | 63-70. The 5′ UTR and encoded first 7 aa are |
| isoform | | | | | identical to that in transcript Dp71, but the |
| | | | | | stop codon lies at the splice junction of the |
| | | | | | exon/intron 70. The 3′ UTR includes nt from |
| | | | | | intron 70 which includes an alternative |
| | | | | | polyadenylation site. The Dp40 isoform lacks |
| | | | | | the normal C-terminal end of full-length |
| | | | | | dystrophin (aa 3409-3685). |
| Dystrophin | NM_004020.3 | 30 | NP_004011.2 | 31 | Transcript Variant: Dp140 transcripts use exons |
| Dp140c | | | | | 45-79, starting at a promoter/exon 1 located in |
| isoform | | | | | intron 44. Dp140 transcripts have a long (1 kb) |
| | | | | | 5′ UTR since translation is initiated in exon |
| | | | | | 51 (corresponding to aa 2461 of dystrophin). In |
| | | | | | addition to the alternative promoter and exon |
| | | | | | 1, differential splicing of exons 71-74 and 78 |
| | | | | | produces at least five Dp140 isoforms. Of |
| | | | | | these, this transcript (Dp140c) lacks exons |
| | | | | | 71-74. |
| Dystrophin | NM_004021.2 | 32 | NP_004012.1 | 33 | Transcript Variant: Dp140 transcripts use exons |
| Dp140b | | | | | 45-79, starting at a promoter/exon 1 located in |
| isoform | | | | | intron 44. Dp140 transcripts have a long (1 kb) |
| | | | | | 5′ UTR since translation is initiated in exon |
| | | | | | 51 (corresponding to aa 2461 of dystrophin). In |
| | | | | | addition to the alternative promoter and exon |
| | | | | | 1, differential splicing of exons 71-74 and 78 |
| | | | | | produces at least five Dp140 isoforms. Of |
| | | | | | these, this transcript (Dp140b) lacks exon 78 |
| | | | | | and encodes a protein with a unique C-terminus. |
| Dystrophin | NM_004022.2 | 34 | NP_004013.1 | 35 | Transcript Variant: Dp140 transcripts use exons |
| Dp140ab | | | | | 45-79, starting at a promoter/exon 1 located in |
| isoform | | | | | intron 44. Dp140 transcripts have a long (1 kb) |
| | | | | | 5′ UTR since translation is initiated in exon |
| | | | | | 51 (corresponding to aa 2461 of dystrophin). In |
| | | | | | addition to the alternative promoter and exon |
| | | | | | 1, differential splicing of exons 71-74 and 78 |
| | | | | | produces at least five Dp140 isoforms. Of |
| | | | | | these, this transcript (Dp140ab) lacks exons 71 |
| | | | | | and 78 and encodes a protein with a unique |
| | | | | | C-terminus. |
| Dystrophin | NM_004023.2 | 36 | NP_004014.1 | 37 | Transcript Variant: Dp140 transcripts use exons |
| Dp140bc | | | | | 45-79, starting at a promoter/exon 1 located in |
| isoform | | | | | intron 44. Dp140 transcripts have a long (1 kb) |
| | | | | | 5′ UTR since translation is initiated in exon |
| | | | | | 51 (corresponding to aa 2461 of dystrophin). In |
| | | | | | addition to the alternative promoter and exon |
| | | | | | 1, differential splicing of exons 71-74 and 78 |
| | | | | | produces at least five Dp140 isoforms. Of |
| | | | | | these, this transcript (Dp140bc) lacks exons |
| | | | | | 71-74 and 78 and encodes a protein with a |
| | | | | | unique C-terminus. |
| Dystrophin | XM_006724469.3 | 38 | XP_006724532.1 | 39 | |
| isoform X2 | | | | | |
| Dystrophin | XM_011545467.1 | 40 | XP_011543769.1 | 41 | |
| isoform X5 | | | | | |
| Dystrophin | XM_006724473.2 | 42 | XP_006724536.1 | 43 | |
| isoform X6 | | | | | |
| Dystrophin | XM_006724475.2 | 44 | XP_006724538.1 | 45 | |
| isoform X8 | | | | | |
| Dystrophin | XM_017029328.1 | 46 | XP_016884817.1 | 47 | |
| isoform X4 | | | | | |
| Dystrophin | XM_006724468.2 | 48 | XP_006724531.1 | 49 | |
| isoform X1 | | | | | |
| Dystrophin | XM_017029331.1 | 50 | XP_016884820.1 | 51 | |
| isoform | | | | | |
| X13 | | | | | |
| Dystrophin | XM_006724470.3 | 52 | XP_006724533.1 | 53 | |
| isoform X3 | | | | | |
| Dystrophin | XM_006724474.3 | 54 | XP_006724537.1 | 55 | |
| isoform X7 | | | | | |
| Dystrophin | XM_011545468.2 | 56 | XP_011543770.1 | 57 | |
| isoform X9 | | | | | |
| Dystrophin | XM_017029330.1 | 58 | XP_016884819.1 | 59 | |
| isoform | | | | | |
| X11 | | | | | |
| Dystrophin | XM_017029329.1 | 865 | XP_016884818.1 | 866 | |
| isoform | | | | | |
| X10 | | | | | |
| Dystrophin | XM_011545469.1 | 867 | XP_011543771.1 | 868 | |
| isoform | | | | | |
| X12 | | | | | |

In embodiments, the guide RNA targets a mutant DMD exon. In some embodiments, the mutant exon is exon 23 or 51. In some embodiments, the guide RNA targets at least one of exons 1, 23, 41, 44, 46, 47, 48, 49, 50, 51, 52, 53, 54, or 55 of the dystrophin gene. In embodiments, the guide RNA targets at least one of introns 44, 45, 50, 51, 52, 53, 54, or 55 of the dystrophin gene. In preferred embodiments, the guide RNAs are designed to induce skipping of exon 51 or exon 23. In embodiments, the gRNA is targeted to a splice acceptor site of exon 51 or exon 23.

Suitable gRNAs and genomic target sequences for use in various compositions and methods disclosed herein are provided as SEQ ID NOs: 60-705, 712-862, and 947-2377.

In some embodiments, the gRNA or gRNA target site has a sequence of any one of the gRNAs or gRNA target sites shown in Tables 5-19.

In some embodiments, gRNAs of the disclosure comprise a sequence that is complementary to a target sequence within a coding sequence or a non-coding sequence corresponding to the DMD gene, and, therefore, hybridize to the target sequence. In some embodiments, gRNAs for Cpf1 comprise a single crRNA containing a direct repeat scaffold sequence followed by 24 nucleotides of guide sequence. In some embodiments, a “guide” sequence of the crRNA comprises a sequence of the gRNA that is complementary to a target sequence. In some embodiments, crRNA of the disclosure comprises a sequence of the gRNA that is not complementary to a target sequence. “Scaffold” sequences of the disclosure link the gRNA to the Cpf1 polypeptide. “Scaffold” sequences of the disclosure are not equivalent to a tracrRNA sequence of a gRNA-Cas9 construct.

In some embodiments, a nucleic acid may comprise one or more sequences encoding a gRNA. In some embodiments, a nucleic acid may comprise 1, 2, 3, 4, 5, 6, 7, 8, 9, 10, 11, 12, 13, 14, 15, 16, 17, 18, 19, or 20 sequences encoding a gRNA. In some embodiments, all of the sequences encode the same gRNA. In some embodiments, all of the sequences encode different gRNAs. In some embodiments, at least 2 of the sequences encode the same gRNA, for example at least 3, 4, 5, 6, 7, 8, 9, 10, 11, 12, 13, 14, 15, 16, 17, 18, 19, or 20 of the sequences encode the same gRNA.

### Nucleases

Cas Nucleases.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. One or both sites may be deactivated while preserving Cas9's ability to locate its target DNA. Jinek et al. (2012) combined tracrRNA and spacer RNA into a “single-guide RNA” molecule that, mixed with Cas9, can find and cut the correct DNA targets and such synthetic guide RNAs are used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. It has been shown that coinjection of Cas9 mRNA and sgRNAs into the germline (zygotes) can be used to generate mice with mutations. Delivery of Cas9 DNA sequences also is contemplated.

The CRISPR/Cas systems are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas system containing a 1,300 amino acid protein. See also U.S. Patent Publication 2014/0068797, which is incorporated by reference in its entirety.

In some embodiments, the compositions of the disclosure include a small version of a Cas9 from the bacterium Staphylococcus aureus (UniProt Accession No. J7RUA5). The small version of the Cas9 provides advantages over wildtype or full length Cas9. In some embodiments the Cas9 is a Streptococcus pyogenes (spCas9).

Cpf1 Nucleases.

Clustered Regularly Interspaced Short Palindromic Repeats from Prevotella and Francisella 1 or CRISPR/Cpf1 is a DNA-editing technology which shares some similarities with the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Cpf1 appears in many bacterial species. The ultimate Cpf1 endonuclease that was developed into a tool for genome editing was taken from one of the first 16 species known to harbor it.

In embodiments, the Cpf1 is a Cpf1 enzyme from Acidaminococcus (species BV3L6, UniProt Accession No. U2UMQ6; SEQ ID NO: 870), having the sequence set forth below:

| | |
|---|---|
| 1 | MTQFEGFTNL YQVSKTLRFE LIPQGKTLKH IQEQGFIEED KARNDHYKEL KPIIDRIYKT |
| 61 | YADQCLQLVQ LDWENLSAAI DSYRKEKTEE TRNALIEEQA TYRNAIHDYF IGRTDNLTDA |
| 121 | INKRHAEIYK GLFKAELFNG KVLKQLGTVT TTEHENALLR SFDKFTTYFS GFYENRKNVF |
| 181 | SAEDISTAIP HRIVQDNFPK FKENCHIFTR LITAVPSLRE HFENVKKAIG IFVSTSIEEV |
| 241 | FSFPFYNQLL TQTQIDLYNQ LLGGISREAG TEKIKGLNEV LNLAIQKNDE TAHIIASLPH |
| 301 | RFIPLFKQIL SDRNTLSFIL EEFKSDEEVI QSFCKYKTLL RNENVLETAE ALFNELNSID |
| 361 | LTHIFISHKK LETISSALCD HWDTLRNALY ERRISELTGK ITKSAKEKVQ RSLKHEDINL |
| 421 | QEIISAAGKE LSEAFKQKTS EILSHAHAAL DQPLPTTLKK QEEKEILKSQ LDSLLGLYHL |
| 481 | LDWFAVDESN EVDPEFSARL TGIKLEMEPS LSFYNKARNY ATKKPYSVEK FKLNFQMPTL |
| 541 | ASGWDVNKEK NNGAILFVKN GLYYLGIMPK QKGRYKALSF EPTEKTSEGF DKMYYDYFPD |
| 601 | AAKMIPKCST QLKAVTAHFQ THTTPILLSN NFIEPLEITK EIYDLNNPEK EPKKFQTAYA |
| 661 | KKTGDQKGYR EALCKWIDFT RDFLSKYTKT TSIDLSSLRP SSQYKDLGEY YAELNPLLYH |
| 721 | ISFQRIAEKE IMDAVETGKL YLFQIYNKDF AKGHHGKPNL HTLYWTGLFS PENLAKTSIK |
| 781 | LNGQAELFYR PKSRMKRMAH RLGEKMLNKK LKDQKTPIPD TLYQELYDYV NHRLSHDLSD |
| 841 | EARALLPNVI TKEVSHEIIK DRRFTSDKFF FHVPITLNYQ AANSPSKFNQ RVNAYLKEHP |
| 901 | ETPIIGIDRG ERNLIYITVI DSTGKILEQR SLNTIQQFDY QKKLDNREKE RVAARQAWSV |
| 961 | VGTIKDLKQG YLSQVIHEIV DLMIHYQAVV VLENLNFGFK SKRTGIAEKA VYQQFEKMLI |
| 1021 | DKLNCLVLKD YPAEKVGGVL NPYQLTDQFT SFAKMGTQSG FLFYVPAPYT SKIDPLTGFV |
| 1081 | DPFVWKTIKN HESRKHFLEG FDFLHYDVKT GDFILHFKMN RNLSFQRGLP GFMPAWDIVF |
| 1141 | EKNETQFDAK GTPFIAGKRI VPVIENHRFT GRYRDLYPAN ELIALLEEKG IVFRDGSNIL |
| 1201 | PKLLENDDSH AIDTMVALIR SVLQMRNSNA ATGEDYINSP VRDLNGVCFD SRFQNPEWPM |
| 1261 | DADANGAYHI ALKGQLLLNH LKESKDLKLQ NGISNQDWLA YIQELRN |

In some embodiments, the Cpf1 is a Cpf1 enzyme from Lachnospiraceae (species ND2006, UniProt Accession No. A0A182DWE3; SEQ ID NO: 871), having the sequence set forth below:

| | |
|---|---|
| 1 | AASKLEKFTN CYSLSKTLRF KAIPVGKTQE NIDNKRLLVE DEKRAEDYKG VKKLLDRYYL |
| 61 | SFINDVLHSI KLKNLNNYIS LFRKKTRTEK ENKELENLEI NLRKEIAKAF KGAAGYKSLF |
| 121 | KKDIIETILP EAADDKDEIA LVNSFNGFTT AFTGFFDNRE NMFSEEAKST SIAFRCINEN |
| 181 | LTRYISNMDI FEKVDAIFDK HEVQEIKEKI LNSDYDVEDF FEGEFFNFVL TQEGIDVYNA |
| 241 | IIGGFVTESG EKIKGLNEYI NLYNAKTKQA LPKFKPLYKQ VLSDRESLSF YGEGYTSDEE |
| 301 | VLEVFRNTLN KNSEIFSSIK KLEKLFKNFD EYSSAGIFVK NGPAISTISK DIFGEWNLIR |
| 361 | DKWNAEYDDI HLKKKAVVTE KYEDDRRKSF KKIGSFSLEQ LQEYADADLS VVEKLKEIII |
| 421 | QKVDEIYKVY GSSEKLFDAD FVLEKSLKKN DAVVAIMKDL LDSVKSFENY IKAFFGEGKE |
| 481 | TNRDESFYGD FVLAYDILLK VDHIYDAIRN YVTQKPYSKD KFKLYFQNPQ FMGGWDKDKE |
| 541 | TDYRATILRY GSKYYLAIMD KKYAKCLQKI DKDDVNGNYE KINYKLLPGP NKMLPKVFFS |
| 601 | KKWMAYYNPS EDIQKIYKNG TFKKGDMFNL NDCHKLIDFF KDSISRYPKW SNAYDFNFSE |
| 661 | TEKYKDIAGF YREVEEQGYK VSFESASKKE VDKLVEEGKL YMFQIYNKDF SDKSHGTPNL |
| 721 | HTMYFKLLFD ENNHGQIRLS GGAELFMRRA SLKKEELVVH PANSPIANKN PDNPKKTTTL |
| 781 | SYDVYKDKRF SEDQYELHIP IAINKCPKNI FKINTEVRVL LKHDDNPYVI GIDRGERNLL |
| 841 | YIVVVDGKGN IVEQYSLNEI INNFNGIRIK TDYHSLLDKK EKERFEARQN WTSIENIKEL |
| 901 | KAGYISQVVH KICELVEKYD AVIALEDLNS GFKNSRVKVE KQVYQKFEKM LIDKLNYMVD |
| 961 | KKSNPCATGG ALKGYQITNK FESFKSMSTQ NGFIFYIPAW LTSKIDPSTG FVNLLKTKYT |
| 1021 | SIADSKKFIS SFDRIMYVPE EDLFEFALDY KNFSRTDADY IKKWKLYSYG NRIRIFAAAK |
| 1081 | KNNVFAWEEV CLTSAYKELF NKYGINYQQG DIRALLCEQS DKAFYSSFMA LMSLMLQMRN |
| 1141 | SITGRTDVDF LISPVKNSDG IFYDSRNYEA QENAILPKNA DANGAYNIAR KVLWAIGQFK |
| 1201 | KAEDEKLDKV KIAISNKEWL EYAQTSVK |

In some embodiments, the Cpf1 is codon optimized for expression in mammalian cells. In some embodiments, the Cpf1 is codon optimized for expression in human cells or mouse cells.

The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 does not require a tracrRNA, therefore, only crRNA is required. This benefits genome editing because Cpf1 is not only smaller than Cas9, but also it has a smaller sgRNA molecule (approximately half as many nucleotides as Cas9).

The Cpf1-crRNA complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

The CRISPR/Cpf1 system consist of a Cpf1 enzyme and a guide RNA that finds and positions the complex at the correct spot on the double helix to cleave target DNA. CRISPR/Cpf1 systems activity has three stages:
Adaptation, during which Cas1 and Cas2 proteins facilitate the adaptation of small fragments of DNA into the CRISPR array;
Formation of crRNAs: processing of pre-cr-RNAs producing of mature crRNAs to guide the Cas protein; and
Interference, in which the Cpf1 is bound to a crRNA to form a binary complex to identify and cleave a target DNA sequence.

Cas9 Versus Cpf1.

Cas9 requires two RNA molecules to cut DNA while Cpf1 needs one. The proteins also cut DNA at different places, offering researchers more options when selecting an editing site. Cas9 cuts both strands in a DNA molecule at the same position, leaving behind ‘blunt’ ends. Cpf1 leaves one strand longer than the other, creating ‘sticky’ ends that are easier to work with. Cpf1 appears to be more able to insert new sequences at the cut site, compared to Cas9. Although the CRISPR/Cas9 system can efficiently disable genes, it is challenging to insert genes or generate a knock-in. Cpf1 lacks tracrRNA, utilizes a T-rich PAM and cleaves DNA via a staggered DNA DSB.

In summary, important differences between Cpf1 and Cas9 systems are that Cpf1 recognizes different PAMs, enabling new targeting possibilities, creates 4-5 nt long sticky ends, instead of blunt ends produced by Cas9, enhancing the efficiency of genetic insertions and specificity during NHEJ or HDR, and cuts target DNA further away from PAM, further away from the Cas9 cutting site, enabling new possibilities for cleaving the DNA.

TABLE 2
Differences between Cas9 and Cpf1

| Feature | Cas9 | Cpf1 |
|---|---|---|
| Structure | Two RNA required | One RNA required |
| | (Or 1 fusion transcript | |
| | (crRNA + tracrRNA = gRNA)) | |
| Cutting | Blunt end cuts | Staggered end cuts |
| mechanism | | |
| Cutting site | Proximal to recognition site | Distal from |
| | | recognition site |
| Target sites | G-rich PAM | T-rich PAM |

Other Nucleases.

In some embodiments, the nuclease is a Cas9 or a Cpf1 nuclease. In addition to Cas9 nucleases and Cpf1 nucleases, other nucleases may be used in the compositions and methods of the disclosure. For example, in some embodiments, the nuclease is a Type II, Type V-A, Type V-B, Type V-C, Type V-U, Type VI-B nuclease. In some embodiments, the nuclease is a Cas9, Cas12a, Cas12b, Cas12c, Tnp-B like, Cas13a (C2c2), or Cas13b nuclease. In some embodiments, the nuclease is a TAL nuclease, a meganuclease, or a zinc-finger nuclease.

CRISPR-Mediated Gene Editing.

The first step in editing the DMD gene using CRISPR/Cpf1 or CRISPR/Cas9 (or another nuclease) is to identify the genomic target sequence. The genomic target for the gRNAs of the disclosure can be any approximately 24 nucleotide DNA sequence, provided that the sequence is unique compared to the rest of the genome. In some embodiments, the genomic target sequence corresponds to a sequence within exon 51, exon 45, exon 44, exon 53, exon 46, exon 52, exon 50, exon 43, exon 6, exon 7, exon 8, and/or exon 55 of the human dystrophin gene. In some embodiments, the genomic target sequence is a 5′ or 3′ splice site of exon 51, exon 45, exon 44, exon 53, exon 46, exon 52, exon 50, exon 43, exon 6, exon 7, exon 8, and/or exon 55 of the human dystrophin gene. In some embodiments, the genomic target sequence corresponds to a sequence within an intron immediately upstream or downstream of exon 51, exon 45, exon 44, exon 53, exon 46, exon 52, exon 50, exon 43, exon 6, exon 7, exon 8, and/or exon 55 of the human dystrophin gene. Exemplary genomic target sequences can be found in Tables 2, 6, 8, 10, 12, 14 and 19.

The next step in editing the DMD gene is to identify all Protospacer Adjacent Motif (PAM) sequences within the genetic region to be targeted. The target sequence must be immediately upstream of a PAM. Once all possible PAM sequences and putative target sites have been identified, the next step is to choose which site is likely to result in the most efficient on-target cleavage. The gRNA targeting sequence needs to match the target sequence, and the gRNA targeting sequence must not match additional sites within the genome. In preferred embodiments, the gRNA targeting sequence has perfect homology to the target with no homology elsewhere in the genome. In some embodiments, a given gRNA targeting sequence will have additional sites throughout the genome where partial homology exists. These sites are called “off-targets” and should be considered when designing a gRNA. In general, off-target sites are not cleaved as efficiently when mismatches occur near the PAM sequence, so gRNAs with no homology or those with mismatches close to the PAM sequence will have the highest specificity. In addition to “off-target activity”, factors that maximize cleavage of the desired target sequence (“on-target activity”) must be considered. It is known to those of skill in the art that two gRNA targeting sequences, each having 100% homology to the target DNA may not result in equivalent cleavage efficiency. In fact, cleavage efficiency may increase or decrease depending upon the specific nucleotides within the selected target sequence. Close examination of predicted on-target and off-target activity of each potential gRNA targeting sequence is necessary to design the best gRNA. Several gRNA design programs have been developed that are capable of locating potential PAM and target sequences and ranking the associated gRNAs based on their predicted on-target and off-target activity (e.g. CRISPRdirect, available at www.crispr.dbcls.jp).

The next step is to synthesize and clone desired gRNAs. Targeting oligos can be synthesized, annealed, and inserted into plasmids containing the gRNA scaffold using standard restriction-ligation cloning. However, the exact cloning strategy will depend on the gRNA vector that is chosen. The gRNAs for Cpf1 are notably simpler than the gRNAs for Cas9, and only consist of a single crRNA containing direct repeat scaffold sequence followed by approximately 24 nucleotides of guide sequence.

Each gRNA should then be validated in one or more target cell lines. For example, after the Cas9 or Cpf1 and the gRNA are delivered to the cell, the genomic target region may be amplified using PCR and sequenced according to methods known to those of skill in the art.

In some embodiments, gene editing may be performed in vitro or ex vivo. In some embodiments, cells are contacted in vitro or ex vivo with a Cas9 or a Cpf1 and a gRNA that targets a dystrophin splice site. In some embodiments, the cells are contacted with one or more nucleic acids encoding the Cas9 or Cpf1 and the guide RNA. In some embodiments, the one or more nucleic acids are introduced into the cells using, for example, lipofection or electroporation. Gene editing may also be performed in zygotes. In embodiments, zygotes may be injected with one or more nucleic acids encoding Cas9 or Cpf1 and a gRNA that targets a dystrophin splice site. The zygotes may subsequently be injected into a host.

In embodiments, the Cas9 or Cpf1 is provided on a vector. In embodiments, the vector contains a Cas9 derived from S. pyogenes (SpCas9, SEQ ID NO. 872). In embodiments, the vector contains a Cas9 derived from S. aureus (SaCas9, SEQ ID NO. 873). In embodiments, the vector contains a Cpf1 sequence derived from a Lachnospiraceae bacterium. See, for example, Uniprot Accession No. A0A182DWE3; SEQ ID NO. 871. In embodiments, the vector contains a Cpf1 sequence derived from an Acidaminococcus bacterium. See, for example, Uniprot Accession No. U2UMQ6; SEQ ID NO. 870. In some embodiments, the Cas9 or Cpf1 sequence is codon optimized for expression in human cells or mouse cells. In some embodiments, the vector further contains a sequence encoding a fluorescent protein, such as GFP, which allows Cas9 or Cpf1-expressing cells to be sorted using fluorescence activated cell sorting (FACS). In some embodiments, the vector is a viral vector such as an adeno-associated viral vector.

In embodiments, the gRNA is provided on a vector. In some embodiments, the vector is a viral vector such as an adeno-associated viral vector. In embodiments, the Cas9 or Cpf1 and the guide RNA are provided on the same vector. In embodiments, the Cas9 or Cpf1 and the guide RNA are provided on different vectors.

In some embodiments, the cells are additionally contacted with a single-stranded DMD oligonucleotide to effect homology directed repair. In some embodiments, small INDELs restore the protein reading frame of dystrophin (“reframing” strategy). When the reframing strategy is used, the cells may be contacted with a single gRNA. In embodiments, a splice donor or splice acceptor site is disrupted, which results in exon skipping and restoration of the protein reading frame (“exon skipping” strategy). When the exon skipping strategy is used, the cells may be contacted with two or more gRNAs.

Efficiency of in vitro or ex vivo Cas9 or Cpf1-mediated DNA cleavage may be assessed using techniques known to those of skill in the art, such as the T7 E1 assay. Restoration of DMD expression may be confirmed using techniques known to those of skill in the art, such as RT-PCR, western blotting, and immunocytochemistry.

In some embodiments, in vitro or ex vivo gene editing is performed in a muscle or satellite cell. In some embodiments, gene editing is performed in iPSC or iCM cells. In embodiments, the iPSC cells are differentiated after gene editing. For example, the iPSC cells may be differentiated into a muscle cell or a satellite cell after editing. In embodiments, the iPSC cells are differentiated into cardiac muscle cells, skeletal muscle cells, or smooth muscle cells. In embodiments, the iPSC cells are differentiated into cardiomyocytes. iPSC cells may be induced to differentiate according to methods known to those of skill in the art.

In some embodiments, contacting the cell with the Cas9 or the Cpf1 and the gRNA restores dystrophin expression. In embodiments, cells which have been edited in vitro or ex vivo, or cells derived therefrom, show levels of dystrophin protein that is comparable to wildtype cells. In embodiments, the edited cells, or cells derived therefrom, express dystrophin at a level that is 50%, 60%, 70%, 80%, 90%, 95% or any percentage in between of wildtype dystrophin expression levels. In embodiments, the cells which have been edited in vitro or ex vivo, or cells derived therefrom, have a mitochondrial number that is comparable to that of wildtype cells. In embodiments the edited cells, or cells derived therefrom, have 50%, 60%, 70%, 80%, 90%, 95% or any percentage in between as many mitochondria as wildtype cells. In embodiments, the edited cells, or cells derived therefrom, show an increase in oxygen consumption rate (OCR) compared to non-edited cells at baseline.

Nucleic Acid Expression Vectors.

As discussed above, in certain embodiments, expression cassettes are employed to express a transcription factor product, either for subsequent purification and delivery to a cell/subject, or for use directly in a genetic-based delivery approach. Provided herein are expression vectors which contain one or more nucleic acids encoding Cas9 or Cpf1 and at least one DMD guide RNA that targets a dystrophin splice site. In some embodiments, a nucleic acid encoding Cas9 or Cpf1 and a nucleic acid encoding at least one guide RNA are provided on the same vector. In further embodiments, a nucleic acid encoding Cas9 or Cpf1 and a nucleic acid encoding least one guide RNA are provided on separate vectors.

Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

Regulatory Elements.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

RNA Polymerase and Pol III Promoters.

In eukaryotes, RNA polymerase III (also called Pol III) transcribes DNA to synthesize ribosomal 5S rRNA, tRNA and other small

RNAs. The genes transcribed by RNA Pol III fall in the category of “housekeeping” genes whose expression is required in all cell types and most environmental conditions. Therefore, the regulation of Pol III transcription is primarily tied to the regulation of cell growth and the cell cycle, thus requiring fewer regulatory proteins than RNA polymerase II. Under stress conditions however, the protein Maf1 represses Pol III activity.

In the process of transcription (by any polymerase) there are three main stages: (i) initiation, requiring construction of the RNA polymerase complex on the gene's promoter; (ii) elongation, the synthesis of the RNA transcript; and (iii) termination, the finishing of RNA transcription and disassembly of the RNA polymerase complex.

Promoters under the control of RNA Pol III include those for ribosomal 5S rRNA, tRNA and few other small RNAs such as U6 spliceosomal RNA, RNase P and RNase MRP RNA, 7SL RNA (the RNA component of the signal recognition particles), Vault RNAs, Y RNA, SINEs (short interspersed repetitive elements), 7SK RNA, two microRNAs, several small nucleolar RNAs and several few regulatory antisense RNAs.

### Additional Promoters and Elements

In some embodiments, the Cas9 or Cpf1 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promoters such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), Duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone a gene. In some embodiments, the inducer is phorbol ester (TFA), heavy metals, glucocorticoids, poly(rI)x, poly(rc), E1A, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are muscle specific promoters. These include the myosin light chain-2 promoter, the α-actin promoter, the troponin 1 promoter; the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter and the ANF promoter. In some embodiments, the muscle specific promoter is the CK8 promoter. The CK8 promoter has the following sequence (SEQ ID NO. 874):

| | |
|---|---|
| 1 | CTAGACTAGC ATGCTGCCCA TGTAAGGAGG CAAGGCCTGG GGACACCCGA GATGCCTGGT |
| 61 | TATAATTAAC CCAGACATGT GGCTGCCCCC CCCCCCCCAA CACCTGCTGC CTCTAAAAAT |
| 121 | AACCCTGCAT GCCATGTTCC CGGCGAAGGG CCAGCTGTCC CCCGCCAGCT AGACTCAGCA |
| 181 | CTTAGTTTAG GAACCAGTGA GCAAGTCAGC CCTTGGGGCA GCCCATACAA GGCCATGGGG |
| 241 | CTGGGCAAGC TGCACGCCTG GGTCCGGGGT GGGCACGGTG CCCGGGCAAC GAGCTGAAAG |
| 301 | CTCATCTGCT CTCAGGGGCC CCTCCCTGGG GACAGCCCCT CCTGGCTAGT CACACCCTGT |
| 361 | AGGCTCCTCT ATATAACCCA GGGGCACAGG GGCTGCCCTC ATTCTACCAC CACCTCCACA |
| 421 | GCACAGACAG ACACTCAGGA GCCAGCCAGC |

In some embodiments, the muscle-cell cell specific promoter is a variant of the CK8 promoter, called CK8e. The CK8e promoter has the following sequence (SEQ ID NO. 875):

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 301 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

### Therapeutic Compositions

### AAV-Cas9 Vectors

In some embodiments, a Cas9 may be packaged into an AAV vector. In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV or any combination thereof.

Exemplary AAV-Cas9 vectors contain two ITR (inverted terminal repeat) sequences which flank a central sequence region comprising the Cas9 sequence. In some embodiments, the ITRs are isolated or derived from an AAV vector of serotype AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype.

In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-Cas9 vector may contain one or more nuclear localization signals (NLS). In some embodiments, the AAV-Cas9 vector contains 1, 2, 3, 4, or 5 nuclear localization signals. Exemplary NLS include the c-myc NLS (SEQ ID NO: 884), the SV40 NLS (SEQ ID NO: 885), the hnRNPAI M9 NLS (SEQ ID NO: 886), the nucleoplasmin NLS (SEQ ID NO: 887), the sequence RMRKFKNKGKDTAELRRRRVEVSVELRKAKKDEQILKRRNV (SEQ ID NO: 888) of the IBB domain from importin-alpha, the sequences VSRKRPRP (SEQ ID NO: 889) and PPKKARED (SEQ ID NO: 890) of the myoma T protein, the sequence PQPKKKPL (SEQ ID NO: 891) of human p53, the sequence SALIKKKKMAP (SEQ ID NO: 892) of mouse c-abl IV, the sequences DRLRR (SEQ ID NO: 893) and KQKKRK (SEQ ID NO: 894) of the influenza virus NS1, the sequence RKLKKKIKKL (SEQ ID NO: 895) of the Hepatitis virus delta antigen and the sequence REKKKFLKRR (SEQ ID NO: 896) of the mouse Mx1 protein. Further acceptable nuclear localization signals include bipartite nuclear localization sequences such as the sequence KRKGDEVDGVDEVAKKKSKK (SEQ ID NO: 897) of the human poly(ADP-ribose) polymerase or the sequence RKCLQAGMNLEARKTKK (SEQ ID NO: 898) of the steroid hormone receptors (human) glucocorticoid.

In some embodiments, the AAV-Cas9 vector may comprise additional elements to facilitate packaging of the vector and expression of the Cas9. In some embodiments, the AAV-Cas9 vector may comprise a polyA sequence. In some embodiments, the polyA sequence may be a mini-polyA sequence. In some embodiments, the AAV-CAs9 vector may comprise a transposable element. In some embodiments, the AAV-Cas9 vector may comprise a regulator element. In some embodiments, the regulator element is an activator or a repressor.

In some embodiments, the AAV-Cas9 may contain one or more promoters. In some embodiments, the one or more promoters drive expression of the Cas9. In some embodiments, the one or more promoters are muscle-specific promoters. Exemplary muscle-specific promoters include myosin light chain-2 promoter, the a-actin promoter, the troponin 1 promoter, the Na+/Ca2+ exchanger promoter, the dystrophin promoter, the α7 integrin promoter, the brain natriuretic peptide promoter, the αB-crystallin/small heat shock protein promoter, α-myosin heavy chain promoter, the ANF promoter, the CK8 promoter and the CK8e promoter.

In some embodiments, the AAV-Cas9 vector may be optimized for production in yeast, bacteria, insect cells, or mammalian cells. In some embodiments, the AAV-Cas9 vector may be optimized for expression in human cells. In some embodiments, the AAV-Cas9 vector may be optimized for expression in a baculovirus expression system.

### AAV-sgRNA Vectors

In some embodiments, at least a first sequence encoding a gRNA and a second sequence encoding a gRNA may be packaged into an AAV vector. In some embodiments, at least a first sequence encoding a gRNA, a second sequence encoding a gRNA, and a third sequence encoding a gRNA may be packaged into an AAV vector. In some embodiments, at least a first sequence encoding a gRNA, a second sequence encoding a gRNA, a third sequence encoding a gRNA, and a fourth sequence encoding a gRNA may be packaged into an AAV vector. In some embodiments, at least a first sequence encoding a gRNA, a second sequence encoding a gRNA, a third sequence encoding a gRNA, a fourth sequence encoding a gRNA, and a fifth sequence encoding a gRNA may be packaged into an AAV vector. In some embodiments, a plurality of sequences encoding a gRNA are packaged into an AAV vector. For example, 1, 2, 3, 4, 5, 6, 7, 8, 9, 10, 11, 12, 13, 14, 15, 16, 17, 18, 19, or 20 sequences encoding a gRNA may be packaged into an AAV vector. In some embodiments, each sequence encoding a gRNA is different. In some embodiments, at least 1, 2, 3, 4, 5, 6, 7, 8, 9, 10, 11, 12, 13, 14, 15, 16, 17, 18, 19, or 20 of the sequences encoding a gRNA are the same. In some embodiments, all of the sequence encoding a gRNA are the same.

In some embodiments, the AAV vector is a wildtype AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV or any combination thereof.

Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV or any combination thereof. In some embodiments, the ITRs are isolated or derived from an AAV vector of a first serotype and a sequence encoding a capsid protein of the AAV-sgRNA vector is isolated or derived from an AAV vector of a second serotype. In some embodiments, the first serotype and the second serotype are the same. In some embodiments, the first serotype and the second serotype are not the same. In some embodiments, the first serotype is AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In some embodiments, the second serotype is AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In some embodiments, the first serotype is AAV2 and the second serotype is AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In some embodiments, the first serotype is AAV2 and the second serotype is AAV9.

In some embodiments, a first ITR is isolated or derived from an AAV vector of a first serotype, a second ITR is isolated or derived from an AAV vector of a second serotype and a sequence encoding a capsid protein of the AAV-sgRNA vector is isolated or derived from an AAV vector of a third serotype. In some embodiments, the first serotype and the second serotype are the same. In some embodiments, the first serotype and the second serotype are not the same. In some embodiments, the first serotype, the second serotype, and the third serotype are the same. In some embodiments, the first serotype, the second serotype, and the third serotype are not the same. In some embodiments, the first serotype is AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV. In some embodiments, the second serotype is AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV. In some embodiments, the third serotype is AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV. In some embodiments, the first serotype is AAV2, the second serotype is AAV4 and the third serotype is AAV1, AAV2, AAV3, AAV4, AAV5, AAV6, AAV7, AAV8, AAV9, AAV10, or AAV11. In some embodiments, the first serotype is AAV2, the second serotype is AAV4 and the third serotype is AAV9. Exemplary AAV-sgRNA vectors contain two ITR (inverted terminal repeat) sequences which flank a central sequence region comprising the sgRNA sequences. In some embodiments, the ITRs are isolated or derived from an AAV vector of serotype AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, ovine AAV or any combination thereof. In some embodiments, the ITRs comprise or consist of full-length and/or wildtype sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wildtype sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV-sgRNA vector may comprise additional elements to facilitate packaging of the vector and expression of the sgRNA. In some embodiments, the AAV-sgRNA vector may comprise a transposable element. In some embodiments, the AAV-sgRNA vector may comprise a regulatory element. In some embodiments, the regulatory element comprises an activator or a repressor. In some embodiments, the AAV-sgRNA sequence may comprise a non-functional or “stuffer” sequence. Exemplary stuffer sequences of the disclosure may have some (a non-zero percentage of) identity or homology to a genomic sequence of a mammal (including a human). Alternatively, exemplary stuffer sequences of the disclosure may have no identity or homology to a genomic sequence of a mammal (including a human). Exemplary stuffer sequences of the disclosure may comprise or consist of naturally occurring non-coding sequences or sequences that are neither transcribed nor translated following administration of the AAV vector to a subject.

In some embodiments, the AAV-sgRNA vector may be optimized for production in yeast, bacteria, insect cells, or mammalian cells. In some embodiments, the AAV-sgRNA vector may be optimized for expression in human cells. In some embodiments, the AAV-Cas9 vector may be optimized for expression in a baculovirus expression system.

In some embodiments, the AAV-sgRNA vector comprises at least one promoter. In some embodiments, the AAV-sgRNA vector comprises at least two promoters. In some embodiments, the AAV-sgRNA vector comprises at least three promoters. In some embodiments, the AAV-sgRNA vector comprises at least four promoters. In some embodiments, the AAV-sgRNA vector comprises at least five promoters. Exemplary promoters include, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus. Further exemplary promoters include the U6 promoter, the H1 promoter, and the 7SK promoter.

In some embodiments, the sequence encoding the gRNA or the genomic target sequence comprises a sequence selected from SEQ ID NOs. 60-705, 712-862, and 947-2377.

### Pharmaceutical Compositions and Delivery Methods

Also provided herein are compositions comprising one or more vectors and/or nucleic acids of the disclosure. In some embodiments, the composition further comprises a pharmaceutically acceptable carrier.

For clinical applications, pharmaceutical compositions are prepared in a form appropriate for the intended application. Generally, this entails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine) and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the Cas9 or Cpf1 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding Cas9 or Cpf1 and a guide RNA that targets a dystrophin splice site are provided to a cell ex vivo before the cell is introduced or reintroduced to a patient.

### Cells and Cell Compositions

Also provided is a cell comprising one or more nucleic acids of the disclosure. In some embodiments, the cell is a human cell. In some embodiments, the cell is a muscle cell or satellite cell. In some embodiments, the cell is an induced pluripotent stem (iPS) cell. In some embodiments, the cell is a cardiomyocyte. In some embodiments, the cell (e.g., a cardiomyocyte) is derived from an iPS cell.

Also provided is a cell comprising a composition comprising one or more vectors of the disclosure. In some embodiments, the cell is a human cell. In some embodiments, the cell is a muscle cell or satellite cell. In some embodiments, the cell is an induced pluripotent stem (iPS) cell. In some embodiments, the cell is a cardiomyocyte. In some embodiments, the cell (e.g., a cardiomyocyte) is derived from an iPS cell.

Also provided is a cell produced by one or more methods of the disclosure. In some embodiments, the cell is a human cell. In some embodiments, the cell is a muscle cell or satellite cell. In some embodiments, the cell is an induced pluripotent stem (iPS) cell. In some embodiments, the cell is a cardiomyocyte. In some embodiments, the cell (e.g., a cardiomyocyte) is derived from an iPS cell.

Also provided is a composition comprising a cell comprising one or more nucleic acids of the disclosure. In some embodiments, the composition further comprises a pharmaceutically acceptable carrier.

### Therapeutic Methods and Uses

The disclosure also provides methods for editing a dystrophin gene, such as a mutant dystrophin gene, in a cell. In some embodiments, the cell is a human cell. In some embodiments, the cell is a muscle cell or satellite cell. In some embodiments, the cell is an induced pluripotent stem (iPS) cell. In some embodiments, the cell is a cardiomyocyte. In some embodiments, the cell (e.g., a cardiomyocyte) is derived from an iPS cell.

In some embodiments, the disclosure provides a method for editing a mutant dystrophin gene in a cardiomyocyte, the method comprising contacting the cardiomyocyte with a Cas9 nuclease, or a sequence encoding a Cas9 nuclease, and a gRNA, or a sequence encoding a gRNA, wherein the gRNA targets a splice donor or splice acceptor site of the dystrophin gene. The mutant dystrophin gene may comprise one or more mutations, such as a point mutation (e.g., a pseudo-exon mutation), a deletion, and/or a duplication mutation. A deletion may be a deletion of at least 20, at least 50, at least 100, at least 500, at least 1000, at least 3000 nucleotides, at least 5000 nucleotides or at least 10,000 nucleotides. In some embodiments, the deletion comprises a deletion of one or more exons, one or more introns, or at least a portion of one intron and one exon.

In some embodiments, the disclosure provides a method for treating or preventing Duchenne Muscular Dystrophy (DMD) in a subject in need thereof, the method comprising administering to the subject a Cas9 nuclease, or a sequence encoding a Cas9 nuclease, and a gRNA, or a sequence encoding a gRNA, wherein the gRNA targets a splice donor or splice acceptor site of the dystrophin gene, wherein the administering restores dystrophin expression in at least 10% of the subject's cardiomyocytes. In some embodiments, the administering restores dystrophin expression in at least 20%, at least 30%, at least 40%, at least 50%, at least 60%, at least 70%, at least 80%, at least 90%, or at least 95% of the subject's cardiomyocytes. The average human heart has approximately 2 to 3 billion cardiomyocytes. Accordingly, in some embodiments, the administering restores dystrophin expression in at least 2×10⁸, at least 3×10⁸, at least 4×10⁸, at least 5×10⁸, at least 6×10⁸, at least 7×10⁸, at least 8×10⁸, at least 9×10⁸, at least 10×10⁸, at least 11×10⁸, at least 12×10⁸, at least 13×10⁸, at least 14×10⁸, at least 15×10⁸, at least 16×10⁸, at least 17×10⁸, at least 18×10⁸, at least 19×10⁸, at least 20×10⁸, at least 21×10⁸, at least 22×10⁸, at least 23×10⁸, at least 24×10⁸, at least 25×10⁸, at least 26×10⁸, at least 27×10⁸, at least 28×10⁸, at least 29×10⁸, at least 30×10⁸ of the subject's cardiomyocytes. In some embodiments, the subject suffers from dilated cardiomyopathy. In some embodiments, the administering at least partially rescues cardiac contractility, or completely rescues cardiac contractility.

In some embodiments, a method for treating or preventing Duchenne Muscular Dystrophy (DMD) in a subject in need thereof, is provided, the method comprising contacting an induced pluripotent stem cell (iPSC) with a Cas9 nuclease or a sequence encoding a Cas9 nuclease, and a gRNA or a sequence encoding a gRNA, wherein the gRNA targets a splice donor or splice acceptor site of the dystrophin gene; differentiating the iPSC into a cardiomyocyte; and administering the cardiomyocyte to the subject. In some embodiments, at least 1×10³, at least 1×10⁴, at least 1×10⁵, at least 1×10⁶, at least 1×10⁷ or at least 1×10⁸ cardiomyocytes are administered to the patient.

The gRNA may target, for example a splice donor or splice acceptor site of exon 51, 45, 53, 44, 46, 52, 50, 43, 6, 7, 8, or 55 of the cardiomyocyte dystrophin gene. In some embodiments, the gRNA or the genomic targeting sequence has a sequence of any one of SEQ ID NOs. 60-705, 712-862, 947-2377. The cas9 nuclease may be isolated or derived from, for example, a S. pyogenes (spCas9) or a S. aureus cas9 (saCas9).

In some embodiments, a vector comprising the gRNA, or a sequence encoding the gRNA, is contacted with the cardiomyocyte. The vector may be, for example, non-viral vector such as a plasmid or a nanoparticle. In some embodiments, the vector may be a viral vector, such as an adeno-associated viral (AAV) vector. In some embodiments, the AAV vector is selected from AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

In some embodiments, a single vector comprising the Cas9 nuclease, or a sequence encoding the Cas9 nuclease, and the gRNA, or a sequence encoding the gRNA, are contacted with the cardiomyocyte. In other embodiments, a first vector comprising the Cas9 nuclease, or a sequence encoding the Cas9 nuclease, and a second vector comprising the gRNA or a sequence encoding the gRNA, are contacted with the cardiomyocyte. The first and second vector may be the same or may be different. For example, the first vector and the second vector may both be AAVs, or the first vector may be an AAV and the second vector may be a plasmid.

Also provided is a method for correcting a dystrophin defect, the method comprising contacting a cell with one or more compositions of the disclosure under conditions suitable for expression of the guide RNA, the Cas9 protein or a nuclease domain thereof, wherein the guide RNA forms a complex with the Cas9 protein or the nuclease domain thereof to form at least one guide RNA-Cas9 complex, wherein the at least one guide RNA-Cas9 complex disrupts a dystrophin splice site and induces selective skipping of a DMD exon and/or reframing. In some embodiments, the at least one guide RNA-Cas9 complex disrupts a dystrophin splice site and induces a reframing of a dystrophin reading frame. In some embodiments, the at least one guide RNA-Cas9 complex disrupts a dystrophin splice site and produces an insertion which restores the dystrophin protein reading frame. In some embodiments, the insertion comprises an insertion of a single adenosine.

Also provided is a method for inducing selective skipping and/or reframing of a DMD exon, the method comprising contacting a cell with one or more compositions of the disclosure under conditions suitable for expression of the guide RNA and the Cas9 protein or a nuclease domain thereof, wherein the guide RNA and the second guide RNA form a complex with the Cas9 protein or the nuclease domain thereof to form at least one guide RNA-Cas9 complex, wherein the at least one guide RNA-Cas9 complex disrupts a dystrophin splice site and induces selective skipping and/or reframing of a DMD exon.

Also provided is a method for inducing a reframing event in the dystrophin reading frame, the method comprising contacting a cell with one or more compositions of the disclosure under conditions suitable for expression of the guide RNA and the Cas9 protein or a nuclease domain thereof, wherein the guide RNA forms a complex with the Cas9 protein or the nuclease domain thereof to form at least one guide RNA-Cas9 complex, wherein the guide RNA-Cas9 complex disrupts a dystrophin splice site and induces selective skipping and/or reframing of a DMD exon. In some embodiments, the at least one guide RNA-Cas9 complex disrupts a dystrophin splice site and induces selective skipping and/or reframing of exon 51 of a human DMD gene.

Also provided is a method of treating or preventing muscular dystrophy in a subject in need thereof comprising administering to the subject a therapeutically effective amount of one or more compositions of the disclosure. In some embodiments, the composition is administered locally. In some embodiments, the composition is administered directly to a muscle tissue. In some embodiments, the composition is administered by an intramuscular infusion or injection. In some embodiments, the muscle tissue comprises a tibialis anterior tissue, a quadriceps tissue, a soleus tissue, a diaphragm tissue, or a heart tissue. In some embodiments, the composition is administered by an intra-cardiac injection. In some embodiments, the composition is administered systemically. In some embodiments, the composition is administered by an intravenous infusion or injection. In some embodiments, following administration of the composition, the subject exhibits normal dystrophin-positive myofibers, and mosaic dystrophin-positive myofibers containing centralized nuclei, or a combination thereof. In some embodiments, following administration of the composition, the subject exhibits an emergence or an increase in a level of abundance of normal dystrophin-positive myofibers when compared to an absence or a level of abundance of normal dystrophin-positive myofibers prior to administration of the composition. In some embodiments, following administration of the composition, the subject exhibits an emergence or an increase in a level of abundance of mosaic dystrophin-positive myofibers containing centralized nuclei when compared to an absence or an level of abundance of mosaic dystrophin-positive myofibers containing centralized nuclei prior to administration of the composition. In some embodiments, following administration of the composition, the subject exhibits a decreased serum CK level when compared to a serum CK level prior to administration of the composition. In some embodiment, following administration of the composition, the subject exhibits improved grip strength when compared to a grip strength prior to administration of the composition. In some embodiments, the subject is a neonate, an infant, a child, a young adult, or an adult. In some embodiments, the subject has muscular dystrophy. In some embodiments, the subject is a genetic carrier for muscular dystrophy. In some embodiments, the subject is male. In some embodiments, the subject is female. In some embodiments, the subject appears to be asymptomatic and a genetic diagnosis reveals a mutation in one or both copies of a DMD gene that impairs function of the DMD gene product. In some embodiments, the subject presents an early sign or symptom of muscular dystrophy. In some embodiments, the early sign or symptom of muscular dystrophy comprises loss of muscle mass or proximal muscle weakness. In some embodiments, the loss of muscle mass or proximal muscle weakness occurs in one or both leg(s) and/or a pelvis, followed by one or more upper body muscle(s). In some embodiments, the early sign or symptom of muscular dystrophy further comprises pseudohypertrophy, low endurance, difficulty standing, difficulty walking, difficulty ascending a staircase or a combination thereof. In some embodiments, the subject presents a progressive sign or symptom of muscular dystrophy. In some embodiments, the progressive sign or symptom of muscular dystrophy comprises muscle tissue wasting, replacement of muscle tissue with fat, or replacement of muscle tissue with fibrotic tissue. In some embodiments, the subject presents a later sign or symptom of muscular dystrophy. In some embodiments, the later sign or symptom of muscular dystrophy comprises abnormal bone development, curvature of the spine, loss of movement, and paralysis. In some embodiments, the subject presents a neurological sign or symptom of muscular dystrophy. In some embodiments, the neurological sign or symptom of muscular dystrophy comprises intellectual impairment and paralysis. In some embodiments, administration of the composition occurs prior to the subject presenting one or more progressive, later or neurological signs or symptoms of muscular dystrophy. In some embodiments, the subject greater than 18 years old, greater than 25 years old, or greater than 30 years old. In some embodiments, the subject is less than 18 years old, less than 16 years old, less than 12 years old, less than 10 years old, less than 5 years old, or less than 2 years old. Also provided is the use of a therapeutically-effective amount of one or more compositions of the disclosure for treating muscular dystrophy in a subject in need thereof.

### Delivery Vectors

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation. In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Improved methods for culturing 293 cells and propagating adenovirus are known in the art. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenoviruses of the disclosure are replication defective, or at least conditionally replication defective. The adenovirus may be of any of the 42 different known serotypes or subgroups A-F. Adenovirus type 5 of subgroup C is the preferred starting material in order to obtain the conditional replication-defective adenovirus vector for use in the present disclosure.

As stated above, the typical vector according to the present disclosure is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors, or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors.

Adenovirus vectors have been used in eukaryotic gene expression and vaccine development. Animal studies suggested that recombinant adenovirus could be used for gene therapy. Studies in administering recombinant adenovirus to different tissues include trachea instillation, muscle injection, peripheral intravenous injections and stereotactic inoculation into the brain.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses may be used, in which biotinylated antibodies against a retroviral envelope protein and against a specific cell receptor are used. The antibodies are coupled via the biotin components by using streptavidin. Using antibodies against major histocompatibility complex class I and class II antigens, it has been demonstrated the infection of a variety of human cells that bore those surface antigens with an ecotropic virus in vitro.

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (see, for example, Markowitz et al., 1988; Hersdorffer et al., 1990).

Other viral vectors may be employed as expression constructs in the present disclosure. Vectors derived from viruses such as vaccinia virus adeno-associated virus (AAV) and herpesviruses may be employed. They offer several attractive features for various mammalian cells.

In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV or any combination thereof.

In some embodiments, a single viral vector is used to deliver a nucleic acid encoding a Cas9 or a Cpf1 and at least one gRNA to a cell. In some embodiments, Cas9 or Cpf1 is provided to a cell using a first viral vector and at least one gRNA is provided to the cell using a second viral vector.

In some embodiments, a single viral vector is used to deliver a nucleic acid encoding Cas9 or Cpf1 and at least one gRNA to a cell. In some embodiments, Cas9 or Cpf1 is provided to a cell using a first viral vector and at least one gRNA is provided to the cell using a second viral vector. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, diaphragm or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment.

Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000′ is widely used and commercially available.

In certain embodiments, the liposome may be complexed with a hemagglutinating virus (HVJ) to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### Duchenne Muscular Dystrophy

Duchenne muscular dystrophy (DMD) is a recessive X-linked form of muscular dystrophy, affecting around 1 in 5000 boys, which results in muscle degeneration and premature death. The disorder is caused by a mutation in the gene dystrophin (see GenBank Accession NO. NC_000023.11), located on the human X chromosome, which codes for the protein dystrophin (GenBank Accession No. AAA53189; SEQ ID NO: 5).

In humans, dystrophin mRNA contains 79 exons. Dystrophin mRNA is known to be alternatively spliced, resulting in various isoforms. Exemplary dystrophin isoforms are listed in Table 1.

The murine dystrophin protein has the following amino acid sequence (Uniprot Accession No. P11531, SEQ. ID. NO: 869):

| | |
|---|---|
| 1 | MWWVDCYRDV KKTTKWNASK GKHDNSDDGK RDGTGKKKGS TRVHANNVNK ARVKNNVDVN |
| 61 | GSTDVDGNHK TGWNHWVKNV MKTMAGTNSK SWVRSTRNYV NVNTSSWSDG ANAHSHRDDW |
| 121 | NSVVSHSATR HANAKCGKDD VATTYDKKSM YTSVVSAVMR TSSKVTRHHH MHYSTVSAGY |
| 181 | TSSSKRKSYA TAAYVATSDS TSYSHARDKS DSSMTVNDSY TAVSWSADTR AGSNDVVKHA |
| 241 | HGMMDTSHGV GNVGSVGKGK SDAVMNNSRW CRVASMKSKH KVMDNKKDDW TKTRTKKMGD |
| 301 | DKCVHKVDVR VNSTHMVVVV DSSGDHATAA KVGDRWANCR WTDRWVDKWH TCSTWSKDAM |
| 361 | KNTSGKDNMM SSHKSTKDKK KTMKSSNDSA KNKSVTKMWM NARWDNTKKS SASAVTTTST |
| 421 | TTVMTVTMVT TRMVKHAKKR TVDSRKRDVD THSWTRSAVS SAVYRKGNSD KVNAARKAKR |
| 481 | KDASRSAAVM ANGVNASRAS NSRWTCSRVN WYTNTYNMTT TANKTSTTST AKSKCKDVNR |
| 541 | SAKSKKGGMD ADVATNHNHD GVRAKKTDTM RYTMSSRTWS SKSVYSVTYM RGKASSKNGN |
| 601 | YSDTVKMAKK ASCKYSGHWK KSSVSCKHMN KRKNHKTKWM AVDVKWAGDA KKKCRVGDTS |
| 661 | NSVNGGKKSA ASRTRNTWDH CRVYTRKAKA GDKTVSKDSM HWMTAYRDYK TDTAVMKRAK |
| 721 | AKTKVKTTVN SVAHASAAKK TTTNYWCTRN GKCKTVWACW HSYKANKWNV KKTMNVAGTV |
| 781 | SNMHHSNNRA TTDGGVMDNT NSRWRHAVRK KSSAKSHSDK AAYTDKVDAA MAKSDTSHSM |
| 841 | KKHNGKDANR VSDVAKKDVS MKRKANRSKM DVKMHATKSV VSSHCVNYKS SVKSVMVKTG |
| 901 | RVKKTNKDRV TAKHYNGAKV TRKKCKSRKM RKMNVTWAAT DTTKRSAVGM SNDSVAWGKA |
| 961 | TKKKAHKSVT GSKMVGKKTV DKSNSNWAVT SRVWNYKHMT DNTKWHADDS KKKKDKRKAM |
| 1021 | NDMRKVDSTR DAAKMANRGD HCRKVVSNRR AASHRKTGKA SKNSDKAGVN KDNKDMSDNG |
| 1081 | TVNRGDNRTD RKRKKTKHNA KDRSRRKKAS HWYYKRADDK CDKKASRDRK KDRKKKNAVR |
| 1141 | RAGSNGAAMA VTSKRWRSNA RRNAHTHTMV VTTDMDVSYV STYTSHASVD HNTCAKDDKS |
| 1201 | KNKDNSGRDH KKKTAASATS MKVKVAVAMD GKHRMYKRGR DRSVKWRHHY DMKVNWNVKK |
| 1261 | TNNWHAKYKW YKDGGRAVVR TNATGSSKTD VNKGSSRWHD CKARRKRKNV SRDNVWADNA |
| 1321 | TGDKVKARGK NTGGAVVSAR DKKKKTNWKV SRAKGVHKDR DHWSRNYNSA GDKVTVHGKA |
| 1381 | DVRSKGHYKK STVKRKDRSW AVNHRRTKDR AGSTTGASAS TVTVTSVVTK TVSKMSSVAA |
| 1441 | DNRAWTTDWS DRVKSRVMVG DDNMKKATDR RTAANKNKTS NARTTDRRWD VNRRNMKDST |
| 1501 | WAKAVGVRGK DSWKGHTVDA KKTTKAKDRR SVDVANDAKR DYSADDTRKV HMTNNTSWGN |
| 1561 | HKRVSAATHR DKSWTATTAN VDASRKKDSR GVRMKWDGTH TDYHNDNGKR SGSDARRDNM |
| 1621 | NKWSKKSNRS HASSDWKRHS VWKDDSRAGG DAVKNDHRAK RKTKVMSTTV RTGKYRRANV |
| 1681 | TRRKAVNAWD KNRSADWRKD ARAADDKRAV KGSWVGDDSD HKVKARGAKN VNRVNDAHTT |
| 1741 | GSYNSTDNTR WRVAVDRVRH AHRDGASHST SVGWRASNKV YYNHTTTCWD HKMTYSADNN |
| 1801 | VRSAYRTAMK RRKACDSSAA CDADHNKNDM DNCTTYDRHN NVNVCVDMCN WNVYDTGRTG |
| 1861 | RRVSKTGSCK AHDKYRYKVA SSTGCDRRGH DSRGVASGGS NSVRSCANNK AADWMRSMVW |
| 1921 | VHRVAAATAK HAKCNCKCGR YRSKHNYDCS CSGRVAKGHK MHYMVYCTTT SGDVRDAKVK |
| 1981 | NKRTKRYAKH RMGYVTVGDN MTVTNWVDSA ASSSHDDTHS RHYASRAMNS NGSYNDSSNS |
| 2041 | DDHHYCSNDS SRSASSRGRA DNRNAYDRKH HKGSSMMTSS RDAAAKRHKG RARMDHNKSH |
| 2101 | RRAAKVNGTT VSSSTSRSDS SMRVVGSTSS MGDSDTSTGV MNNSSSRGRN AGKMRDTM |

Dystrophin is an important component within muscle tissue that provides structural stability to the dystroglycan complex (DGC) of the cell membrane. While both sexes can carry the mutation, females are rarely affected with the skeletal muscle form of the disease.

Mutations vary in nature and frequency. Large genetic deletions are found in about 60-70% of cases, large duplications are found in about 10% of cases, and point mutants or other small changes account for about 15-30% of cases. Bladen et al. (2015), who examined some 7000 mutations, catalogued a total of 5,682 large mutations (80% of total mutations), of which 4,894 (86%) were deletions (1 exon or larger) and 784 (14%) were duplications (1 exon or larger). There were 1,445 small mutations (smaller than 1 exon, 20% of all mutations), of which 358 (25%) were small deletions and 132 (9%) small insertions, while 199 (14%) affected the splice sites. Point mutations totaled 756 (52% of small mutations) with 726 (50%) nonsense mutations and 30 (2%) missense mutations. Finally, 22 (0.3%) mid-intronic mutations were observed. In addition, mutations were identified within the database that would potentially benefit from novel genetic therapies for DMD including stop codon read-through therapies (10% of total mutations) and exon skipping therapy (80% of deletions and 55% of total mutations).

DMD Subject Characteristics and Clinical Presentation.

Symptoms usually appear in boys between the ages of 2 and 3 and may be visible in early infancy. Even though symptoms do not appear until early infancy, laboratory testing can identify children who carry the active mutation at birth. Progressive proximal muscle weakness of the legs and pelvis associated with loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking but most patients are wheelchair dependent by age 12. Later symptoms may include abnormal bone development that lead to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for males afflicted with DMD is around 25.

The main symptom of Duchenne muscular dystrophy, a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age 6 and may appear in early infancy. Other physical symptoms are:
1. Awkward manner of walking, stepping, or running—(patients tend to walk on their forefeet, because of an increased calf muscle tone. Also, toe walking is a compensatory adaptation to knee extensor weakness.)
2. Frequent falls.
3. Fatigue.
4. Difficulty with motor skills (running, hopping, jumping).
5. Lumbar hyperlordosis, possibly leading to shortening of the hip-flexor muscles. This has an effect on overall posture and a manner of walking, stepping, or running.
6. Muscle contractures of Achilles tendon and hamstrings impair functionality because the muscle fibers shorten and fibrose in connective tissue.
7. Progressive difficulty walking.
8. Muscle fiber deformities.
9. Pseudohypertrophy (enlarging) of tongue and calf muscles. The muscle tissue is eventually replaced by fat and connective tissue, hence the term pseudohypertrophy.
10. Higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain.
11. Eventual loss of ability to walk (usually by the age of 12).
12. Skeletal deformities (including scoliosis in some cases).
13. Trouble getting up from lying or sitting position.

The condition can often be observed clinically from the moment the patient takes his first steps, and the ability to walk usually completely disintegrates between the time the boy is 9 to 12 years of age. Most men affected with DMD become essentially “paralyzed from the neck down” by the age of 21. Muscle wasting begins in the legs and pelvis, then progresses to the muscles of the shoulders and neck, followed by loss of arm muscles and respiratory muscles. Calf muscle enlargement (pseudohypertrophy) is quite obvious. Cardiomyopathy particularly (dilated cardiomyopathy) is common, but the development of congestive heart failure or arrhythmia (irregular heartbeat) is only occasional.

A positive Gowers' sign reflects the more severe impairment of the lower extremities muscles. The child helps himself to get up with upper extremities: first by rising to stand on his arms and knees, and then “walking” his hands up his legs to stand upright. Affected children usually tire more easily and have less overall strength than their peers. Creatine kinase (CPK-MM) levels in the bloodstream are extremely high. An electromyography (EMG) shows that weakness is caused by destruction of muscle tissue rather than by damage to nerves. Genetic testing can reveal genetic errors in the Xp21 gene. A muscle biopsy (immunohistochemistry or immunoblotting) or genetic test (blood test) confirms the absence of dystrophin, although improvements in genetic testing often make this unnecessary.

DMD patients may suffer from:
1. Abnormal heart muscle (cardiomyopathy).
2. Congestive heart failure or irregular heart rhythm (arrhythmia).
3. Deformities of the chest and back (scoliosis).
4. Enlarged muscles of the calves, buttocks, and shoulders (around age 4 or 5). These muscles are eventually replaced by fat and connective tissue (pseudohypertrophy).
5. Loss of muscle mass (atrophy).
6. Muscle contractures in the heels, legs.
7. Muscle deformities.
8. Respiratory disorders, including pneumonia and swallowing with food or fluid passing into the lungs (in late stages of the disease).

Duchenne muscular dystrophy (DMD) is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

DMD is inherited in an X-linked recessive pattern. Females will typically be carriers for the disease while males will be affected. Typically, a female carrier will be unaware they carry a mutation until they have an affected son. The son of a carrier mother has a 50% chance of inheriting the defective gene from his mother. The daughter of a carrier mother has a 50% chance of being a carrier and a 50% chance of having two normal copies of the gene. In all cases, an unaffected father will either pass a normal Y to his son or a normal X to his daughter. Female carriers of an X-linked recessive condition, such as DMD, can show symptoms depending on their pattern of X-inactivation.

Exon deletions preceding exon 51 of the human DMD gene, which disrupt the open reading frame (ORF) by juxtaposing out of frame exons, represent the most common type of human DMD mutation. Skipping of exon 51 can, in principle, restore the DMD ORF in 13% of DMD patients with exon deletions.

Duchenne muscular dystrophy has an incidence of 1 in 5000 male infants. Mutations within the dystrophin gene can either be inherited or occur spontaneously during germline transmission.

### Sequences

The following tables provide exemplary primer, gRNA and genomic targeting sequences for use in connection with the compositions and methods disclosed herein.

TABLE 4
Sequence of Primers for DMD iPSCs
PCR/T7E1 and RT-PCR primers

| | | SEQ | | SEQ |
|---|---|---|---|---|
| DMD | | ID | | ID |
| # | PCR/T7E1 | NO: | RT-PCR | NO: |
| Del. | F: TTCCCTGGCAAG | 2463 | F: CCCAGAAGAGC | 2469 |
| | GTCTGA | | AAGATAAACTTGAA | |
| | R: ATCCTCAAGGTC | 2464 | R: CTCTGTTCCAA | 2470 |
| | ACCCACC | | ATCCTGCATTGT | |
| pEx. | F: CACACCTGTTAT | 2465 | F: CATAAGCCCAG | 2471 |
| | ATTTTTCCGTGAAG | | AAGAGCAAGATAAA | |
| | R: CAAAGGAGAAGC | 2466 | R: ATAGGAGATAA | 2472 |
| | AAAAACACATTCTA | | CCACAGCAGCAGAT | |
| Dup. | F: GTAATGTATAAC | 2467 | E59F: GGGAAAAA | 2473 |
| | TGTATAACGTGGGGC | | TTGAACCTGCAC | |
| | ACTC | | | |
| | R: GGTGAGTTGTT | 2468 | E55R: CATCAGCT | 2474 |
| | GCTACAGCTCTTCC | | CTTTTACTCCCTT | |
| | | | E53F: GGAGGGTC | 2475 |
| | | | CCTATACAGTAG | |

TABLE 5
Genomic targeting sequences of top 12 exons.

| | Applica- | | SEQ | | SEQ |
|---|---|---|---|---|---|
| | bility | gRNA/PAM at | ID | gRNA/PAM at | ID |
| Exon | (30) | acceptor site | NO. | donor site | NO. |
| 51 | 13.0% | #1: | 2378 | | |
| | | TGCAAAAACCCAA | | | |
| | | AATATTTTAG | | | |
| | | #2: | 2379 | | |
| | | AAAATATTTTAGC | | | |
| | | TCCTACTCAG | | | |
| | | #3: | 2380 | | |
| | | CAGAGTAACAGTC | | | |
| | | TGAGTAGGAG* | | | |
| 45 | 8.1% | #1: | 2381 | | |
| | | TTGCCTTTTTGGT | | | |
| | | ATCTTACAGG | | | |
| | | #2: | 2382 | | |
| | | TTTGCCTTTTTGG | | | |
| | | TATCTTACAG | | | |
| | | #3: | 2383 | | |
| | | CGCTGCCCAATGC | | | |
| | | CATCCTGGAG | | | |
| 53 | 7.7% | #1: | 2384 | #4: | 2414 |
| | | ATTTATTTTTCCT | | AAAGAAAATCAC | |
| | | TTATTCTAG | | AGAAACCAAGG | |
| | | #2: | 2385 | #5: | 2415 |
| | | TTTCCTTTTATTC | | AAAATCACAGAA | |
| | | TAGTTGAAAG | | ACCAAGGTTAG | |
| | | #3: | 2386 | #6: | 2416 |
| | | TGATTCTGAATTC | | GGTATCTTTGAT | |
| | | TTTCAACTAG | | ACTAACCTTGG | |
| 44 | 6.2% | #1: | 2387 | #4: | 2417 |
| | | ATCCATATGCTTT | | GTAATACAAATG | |
| | | TACCTGCAGG | | GTATCTTAAGG | |
| | | #2: | 2388 | | |
| | | GATCCATATGCTT | | | |
| | | TTACCTGCAG | | | |
| | | 3: | 2389 | | |
| | | CAGATCTGTCAAA | | | |
| | | TCGCCTGCAG | | | |
| 46 | 4.3% | #1: | 2390 | | |
| | | TTATTCTTCTTTC | | | |
| | | TCCAGGCTAG | | | |
| | | #2: | 2391 | | |
| | | AATTTTATTCTTC | | | |
| | | TTTCTCCAGG | | | |
| | | #3: | 2392 | | |
| | | CAATTTTATTCTT | | | |
| | | CTTTCTCCAG | | | |
| 52 | 4.1% | #1: | 2393 | | |
| | | TAAGGGATATTTG | | | |
| | | TTCTTACAGG | | | |
| | | #2: | 2394 | | |
| | | CTAAGGGATATTT | | | |
| | | GTTCTTACAG | | | |
| | | #3: | 2395 | | |
| | | TGTTCTTACAGGC | | | |
| | | AACAATGCAG | | | |
| 50 | 4.0% | #1: | 2396 | | |
| | | TGTATGCTTTTCT | | | |
| | | GTTAAAGAGG | | | |
| | | #2: | 2397 | | |
| | | ATGTGTATGCTTT | | | |
| | | TCTGTTAAAG | | | |
| | | #3: | 2398 | | |
| | | GTGTATGCTTTTC | | | |
| | | TGTTAAAGAG | | | |
| 43 | 3.8% | #1: | 2399 | #4: | 2418 |
| | | GTTTTAAAATTTT | | TATGTGTTACCT | |
| | | TATATTACAG | | ACCCTTGTCGG | |
| | | #2: | 2400 | #5: | 2419 |
| | | TTTTATATTACAG | | AAATGTACAAGG | |
| | | AATATAAAAG | | ACCGACAAGGG | |
| | | #3: | 2401 | #6: | 2420 |
| | | ATATTACAGAATA | | GTACAAGGACCG | |
| | | TAAAAGATAG | | ACAAGGGTAGG | |
| 6 | 3.0%† | #1: | 2402 | #4: | 2421 |
| | | TGAAAATTTATTT | | ATGCTCTCATCC | |
| | | CCACATGTAG | | ATAGTCATAGG | |
| | | #2: | 2403 | #5: | 2422 |
| | | GAAAATTTATTTC | | TCTCATCCATAG | |
| | | CACATGTAGG | | TCATAGGTAAG | |
| | | #3: | 2404 | #6: | 2423 |
| | | TTACATTTTTGAC | | CATCCATAGTCA | |
| | | CTACATGTGG | | TAGGTAAGAAG | |
| 7 | 3.0%† | #1: | 2405 | | |
| | | TGTGTATGTGTAT | | | |
| | | GTGTTTTAGG | | | |
| | | #2: | 2406 | | |
| | | TATGTGTATGTGT | | | |
| | | TTTAGGCCAG | | | |
| | | #3: | 2407 | | |
| | | CTATTCCAGTCAA | | | |
| | | ATAGGTCTGG | | | |
| 8 | 2.3% | #1: | 2408 | #4: | 2424 |
| | | GTGTAGTGTTAAT | | TGCACTATTCTC | |
| | | GTGCTTACAG | | AACAGGTAAAG | |
| | | #2: | 2409 | #5: | 2425 |
| | | GGACTTCTTATCT | | TCAAATGCACTA | |
| | | GGATAGGTGG | | TTCTCAACAGG | |
| | | #3: | 2410 | #6: | 2426 |
| | | TAGGTGGTATCAA | | CTTTACACACTT | |
| | | CATCTGTAAG | | TACCTGTTGAG | |
| 55 | 2.09% | #1: | 2411 | | |
| | | TGAACATTTGGTC | | | |
| | | CTTTGCAGGG | | | |
| | | #2: | 2412 | | |
| | | TCTGAACATTTGG | | | |
| | | TCCTTTGCAG | | | |
| | | #3: | 2413 | | |
| | | TCTCGCTCACTCA | | | |
| | | CCCTGCAAAG | | | |

†Dual exon skipping (exons 6 and 7).

TABLE 6
Genomic Target Sequences

| Targeted | Guide | | Genomic | | SEQ |
|---|---|---|---|---|---|
| gRNA Exon | # | Strand | Target Sequence* | PAM | ID NO. |
| Human-Exon 51 | 4 | 1 | tctttttcttcttttttccttttt | tttt | 60 |
| Human-Exon 51 | 5 | 1 | ctttttcttcttttttcctttttG | tttt | 61 |
| Human-Exon 51 | 6 | 1 | tttttcttcttttttcctttttGC | tttc | 62 |
| Human-Exon 51 | 7 | 1 | tcttcttttttcctttttGCAAAA | tttt | 63 |
| Human-Exon 51 | 8 | 1 | cttcttttttcctttttGCAAAAA | tttt | 64 |
| Human-Exon 51 | 9 | 1 | ttcttttttcctttttGCAAAAAC | tttc | 65 |
| Human-Exon 51 | 10 | 1 | ttcctttttGCAAAAACCCAAAAT | tttt | 66 |
| Human-Exon 51 | 11 | 1 | tcctttttGCAAAAACCCAAAATA | tttt | 67 |
| Human-Exon 51 | 12 | 1 | cctttttGCAAAAACCCAAAATAT | tttt | 68 |
| Human-Exon 51 | 13 | 1 | ctttttGCAAAAACCCAAAATATT | tttc | 69 |
| Human-Exon 51 | 14 | 1 | tGCAAAAACCCAAAATATTTTAGC | tttt | 70 |
| Human-Exon 51 | 15 | 1 | GCAAAAACCCAAAATATTTTAGCT | tttt | 71 |
| Human-Exon 51 | 16 | 1 | CAAAAACCCAAAATATTTTAGCTC | tttG | 72 |
| Human-Exon 51 | 17 | 1 | AGCTCCTACTCAGACTGTTACTCT | TTTT | 73 |
| Human-Exon 51 | 18 | 1 | GCTCCTACTCAGACTGTTACTCTG | TTTA | 74 |
| Human-Exon 51 | 19 | -1 | CTTAGTAACCACAGGTTGTGTCAC | TTTC | 75 |
| Human-Exon 51 | 20 | -1 | GAGATGGCAGTTTCCTTAGTAACC | TTTG | 76 |
| Human-Exon 51 | 21 | -1 | TAGTTTGGAGATGGCAGTTTCCTT | TTTC | 77 |
| Human-Exon 51 | 22 | -1 | TTCTCATACCTTCTGCTTGATGAT | TTTT | 78 |
| Human-Exon 51 | 23 | -1 | TCATTTTTTCTCATACCTTCTGCT | TTTA | 79 |
| Human-Exon 51 | 24 | -1 | ATCATTTTTTCTCATACCTTCTGC | TTTT | 80 |
| Human-Exon 51 | 25 | -1 | AAGAAAAACTTCTGCCAACTTTTA | TTTA | 81 |
| Human-Exon 51 | 26 | -1 | AAAGAAAAACTTCTGCCAACTTTT | TTTT | 82 |
| Human-Exon 51 | 27 | 1 | TCTTTAAAATGAAGATTTTCCACC | TTTT | 83 |
| Human-Exon 51 | 28 | 1 | CTTTAAAATGAAGATTTTCCACCA | TTTT | 84 |
| Human-Exon 51 | 29 | 1 | TTTAAAATGAAGATTTTCCACCAA | TTTC | 85 |
| Human-Exon 51 | 30 | 1 | AAATGAAGATTTTCCACCAATCAC | TTTA | 86 |
| Human-Exon 51 | 31 | 1 | CCACCAATCACTTTACTCTCCTAG | TTTT | 87 |
| Human-Exon 51 | 32 | 1 | CACCAATCACTTTACTCTCCTAGA | TTTC | 88 |
| Human-Exon 51 | 33 | 1 | CTCTCCTAGACCATTTCCCACCAG | TTTA | 89 |
| Human-Exon 45 | 1 | -1 | agaaaagattaaacagtgtgctac | tttg | 90 |
| Human-Exon 45 | 2 | -1 | tttgagaaaagattaaacagtgtg | TTTa | 91 |
| Human-Exon 45 | 3 | -1 | atttgagaaaagattaaacagtgt | TTTT | 92 |
| Human-Exon 45 | 4 | -1 | Tatttgagaaaagattaaacagtg | TTTT | 93 |
| Human-Exon 45 | 5 | 1 | atcttttctcaaatAAAAAGACAT | ttta | 94 |
| Human-Exon 45 | 6 | 1 | ctcaaatAAAAAGACATGGGGCTT | tttt | 95 |
| Human-Exon 45 | 7 | 1 | tcaaatAAAAAGACATGGGGCTTC | tttc | 96 |
| Human-Exon 45 | 8 | 1 | TGTTTTGCCTTTTTGGTATCTTAC | TTTT | 97 |
| Human-Exon 45 | 9 | 1 | GTTTTGCCTTTTTGGTATCTTACA | TTTT | 98 |
| Human-Exon 45 | 10 | 1 | TTTTGCCTTTTTGGTATCTTACAG | TTTG | 99 |
| Human-Exon 45 | 11 | 1 | GCCTTTTTGGTATCTTACAGGAAC | TTTT | 100 |
| Human-Exon 45 | 12 | 1 | CCTTTTTGGTATCTTACAGGAACT | TTTG | 101 |
| Human-Exon 45 | 13 | 1 | TGGTATCTTACAGGAACTCCAGGA | TTTT | 102 |
| Human-Exon 45 | 14 | 1 | GGTATCTTACAGGAACTCCAGGAT | TTTT | 103 |
| Human-Exon 45 | 15 | -1 | AGGATTGCTGAATTATTTCTTCCC | TTTG | 104 |
| Human-Exon 45 | 16 | -1 | GAGGATTGCTGAATTATTTCTTCC | TTTT | 105 |
| Human-Exon 45 | 17 | -1 | TGAGGATTGCTGAATTATTTCTTC | TTTT | 106 |
| Human-Exon 45 | 18 | -1 | CTGTAGAATACTGGCATCTGTTTT | TTTC | 107 |
| Human-Exon 45 | 19 | -1 | CCTGTAGAATACTGGCATCTGTTT | TTTT | 108 |
| Human-Exon 45 | 20 | -1 | TCCTGTAGAATACTGGCATCTGTT | TTTT | 109 |
| Human-Exon 45 | 21 | -1 | CAGACCTCCTGCCACCGCAGATTC | TTTG | 110 |
| Human-Exon 45 | 22 | -1 | TGTCTGACAGCTGTTTGCAGACCT | TTTC | 111 |
| Human-Exon 45 | 23 | -1 | CTGTCTGACAGCTGTTTGCAGACC | TTTT | 112 |
| Human-Exon 45 | 24 | -1 | TCTGTCTGACAGCTGTTTGCAGAC | TTTT | 113 |
| Human-Exon 45 | 25 | -1 | TTCTGTCTGACAGCTGTTTGCAGA | TTTT | 114 |
| Human-Exon 45 | 26 | -1 | ATTCCTATTAGATCTGTCGCCCTA | TTTC | 115 |
| Human-Exon 45 | 27 | -1 | CATTCCTATTAGATCTGTCGCCCT | TTTT | 116 |
| Human-Exon 45 | 28 | 1 | AGCAGACTTTTTAAGCTTTCTTTA | TTTT | 117 |
| Human-Exon 45 | 29 | 1 | GCAGACTTTTTAAGCTTTCTTTAG | TTTA | 118 |
| Human-Exon 45 | 30 | 1 | TAAGCTTTCTTTAGAAGAATATTT | TTTT | 119 |
| Human-Exon 45 | 31 | 1 | AAGCTTTCTTTAGAAGAATATTTC | TTTT | 120 |
| Human-Exon 45 | 32 | 1 | AGCTTTCTTTAGAAGAATATTTCA | TTTA | 121 |
| Human-Exon 45 | 33 | 1 | TTTAGAAGAATATTTCATGAGAGA | TTTC | 122 |
| Human-Exon 45 | 34 | 1 | GAAGAATATTTCATGAGAGATTAT | TTTA | 123 |
| Human-Exon 44 | 1 | 1 | TCAGTATAACCAAAAAATATACGC | TTTG | 124 |
| Human-Exon 44 | 2 | 1 | acataatccatctatttttcttga | tttt | 125 |
| Human-Exon 44 | 3 | 1 | cataatccatctatttttcttgat | ttta | 126 |
| Human-Exon 44 | 4 | 1 | tcttgatccatatgcttttACCTG | tttt | 127 |
| Human-Exon 44 | 5 | 1 | cttgatccatatgcttttACCTGC | tttt | 128 |
| Human-Exon 44 | 6 | 1 | ttgatccatatgcttttACCTGCA | tttc | 129 |
| Human-Exon 44 | 7 | -1 | TCAACAGATCTGTCAAATCGCCTG | TTTC | 130 |
| Human-Exon 44 | 8 | 1 | ACCTGCAGGCGATTTGACAGATCT | tttt | 131 |
| Human-Exon 44 | 9 | 1 | CCTGCAGGCGATTTGACAGATCTG | tttA | 132 |
| Human-Exon 44 | 10 | 1 | ACAGATCTGTTGAGAAATGGCGGC | TTTG | 133 |
| Human-Exon 44 | 11 | -1 | TATCATAATGAAAACGCCGCCATT | TTTA | 134 |
| Human-Exon 44 | 12 | 1 | CATTATGATATAAAGATATTTAAT | TTTT | 135 |
| Human-Exon 44 | 13 | -1 | TATTTAGCATGTTCCCAATTCTCA | TTTG | 136 |
| Human-Exon 44 | 14 | -1 | GAAAAAACAAATCAAAGACTTACC | TTTC | 137 |
| Human-Exon 44 | 15 | 1 | ATTTGTTTTTTCGAAATTGTATTT | TTTG | 138 |
| Human-Exon 44 | 16 | 1 | TTTTTTCGAAATTGTATTTATCTT | TTTG | 139 |
| Human-Exon 44 | 17 | 1 | TTCGAAATTGTATTTATCTTCAGC | TTTT | 140 |
| Human-Exon 44 | 18 | 1 | TCGAAATTGTATTTATCTTCAGCA | TTTT | 141 |
| Human-Exon 44 | 19 | 1 | CGAAATTGTATTTATCTTCAGCAC | TTTT | 142 |
| Human-Exon 44 | 20 | 1 | GAAATTGTATTTATCTTCAGCACA | TTTC | 143 |
| Human-Exon 44 | 21 | -1 | AGAAGTTAAAGAGTCCAGATGTGC | TTTA | 144 |
| Human-Exon 44 | 22 | 1 | TCTTCAGCACATCTGGACTCTTTA | TTTA | 145 |
| Human-Exon 44 | 23 | -1 | CATCACCCTTCAGAACCTGATCTT | TTTC | 146 |
| Human-Exon 44 | 24 | 1 | ACTTCTTAAAGATCAGGTTCTGAA | TTTA | 147 |
| Human-Exon 44 | 25 | 1 | GACTGTTGTTGTCATCATTATATT | TTTT | 148 |
| Human-Exon 44 | 26 | 1 | ACTGTTGTTGTCATCATTATATTA | TTTG | 149 |
| Human-Exon 53 | 1 | -1 | AACTAGAATAAAAGGAAAAATAAA | TTTC | 150 |
| Human-Exon 53 | 2 | 1 | CTACTATATATTTATTTTTCCTTT | TTTA | 151 |
| Human-Exon 53 | 3 | 1 | TTTTTCCTTTTATTCTAGTTGAAA | TTTA | 152 |
| Human-Exon 53 | 4 | 1 | TCCTTTTATTCTAGTTGAAAGAAT | TTTT | 153 |
| Human-Exon 53 | 5 | 1 | CCTTTTATTCTAGTTGAAAGAATT | TTTT | 154 |
| Human-Exon 53 | 6 | 1 | CTTTTATTCTAGTTGAAAGAATTC | TTTC | 155 |
| Human-Exon 53 | 7 | 1 | ATTCTAGTTGAAAGAATTCAGAAT | TTTT | 156 |
| Human-Exon 53 | 8 | 1 | TTCTAGTTGAAAGAATTCAGAATC | TTTA | 157 |
| Human-Exon 53 | 9 | -1 | ATTCAACTGTTGCCTCCGGTTCTG | TTTC | 158 |
| Human-Exon 53 | 10 | -1 | ACATTTCATTCAACTGTTGCCTCC | TTTA | 159 |
| Human-Exon 53 | 11 | -1 | CTTTTGGATTGCATCTACTGTATA | TTTT | 160 |
| Human-Exon 53 | 12 | -1 | TGTGATTTTCTTTTGGATTGCATC | TTTC | 161 |
| Human-Exon 53 | 13 | -1 | ATACTAACCTTGGTTTCTGTGATT | TTTG | 162 |
| Human-Exon 53 | 14 | -1 | AAAAGGTATCTTTGATACTAACCT | TTTA | 163 |
| Human-Exon 53 | 15 | -1 | AAAAAGGTATCTTTGATACTAACC | TTTT | 164 |
| Human-Exon 53 | 16 | -1 | TTTTAAAAAGGTATCTTTGATACT | TTTA | 165 |
| Human-Exon 53 | 17 | -1 | ATTTTAAAAAGGTATCTTTGATAC | TTTT | 166 |
| Human-Exon 46 | 1 | -1 | TTAATGCAAACTGGGACACAAACA | TTTG | 167 |
| Human-Exon 46 | 2 | 1 | TAAATTGCCATGTTTGTGTCCCAG | TTTT | 168 |
| Human-Exon 46 | 3 | 1 | AAATTGCCATGTTTGTGTCCCAGT | TTTT | 169 |
| Human-Exon 46 | 4 | 1 | AATTGCCATGTTTGTGTCCCAGTT | TTTA | 170 |
| Human-Exon 46 | 5 | 1 | TGTCCCAGTTTGCATTAACAAATA | TTTG | 171 |
| Human-Exon 46 | 6 | -1 | CAACATAGTTCTCAAACTATTTGT | tttC | 172 |
| Human-Exon 46 | 7 | -1 | CCAACATAGTTCTCAAACTATTTG | tttt | 173 |
| Human-Exon 46 | 8 | -1 | tCCAACATAGTTCTCAAACTATTT | tttt | 174 |
| Human-Exon 46 | 9 | -1 | tttCCAACATAGTTCTCAAACTAT | tttt | 175 |
| Human-Exon 46 | 10 | -1 | ttttCCAACATAGTTCTCAAACTA | tttt | 176 |
| Human-Exon 46 | 11 | -1 | tttttCCAACATAGTTCTCAAACT | tttt | 177 |
| Human-Exon 46 | 12 | 1 | CATTAACAAATAGTTTGAGAACTA | TTTG | 178 |
| Human-Exon 46 | 13 | 1 | AGAACTATGTTGGaaaaaaaaaTA | TTTG | 179 |
| Human-Exon 46 | 14 | -1 | GTTCTTCTAGCCTGGAGAAAGAAG | TTTT | 180 |
| Human-Exon 46 | 15 | 1 | ATTCTTCTTTCTCCAGGCTAGAAG | TTTT | 181 |
| Human-Exon 46 | 16 | 1 | TTCTTCTTTCTCCAGGCTAGAAGA | TTTA | 182 |
| Human-Exon 46 | 17 | 1 | TCCAGGCTAGAAGAACAAAAGAAT | TTTC | 183 |
| Human-Exon 46 | 18 | -1 | AAATTCTGACAAGATATTCTTTTG | TTTG | 184 |
| Human-Exon 46 | 19 | -1 | CTTTTAGTTGCTGCTCTTTTCCAG | TTTT | 185 |
| Human-Exon 46 | 20 | -1 | AGAAAATAAAATTACCTTGACTTG | TTTG | 186 |
| Human-Exon 46 | 21 | -1 | TGCAAGCAGGCCCTGGGGGATTTG | TTTA | 187 |
| Human-Exon 46 | 22 | 1 | ATTTTCTCAAATCCCCCAGGGCCT | TTTT | 188 |
| Human-Exon 46 | 23 | 1 | TTTTCTCAAATCCCCCAGGGCCTG | TTTA | 189 |
| Human-Exon 46 | 24 | 1 | CTCAAATCCCCCAGGGCCTGCTTG | TTTT | 190 |
| Human-Exon 46 | 25 | 1 | TCAAATCCCCCAGGGCCTGCTTGC | TTTC | 191 |
| Human-Exon 46 | 26 | 1 | TTAATTCAATCATTGGTTTTCTGC | TTTT | 192 |
| Human-Exon 46 | 27 | 1 | TAATTCAATCATTGGTTTTCTGCC | TTTT | 193 |
| Human-Exon 46 | 28 | 1 | AATTCAATCATTGGTTTTCTGCCC | TTTT | 194 |
| Human-Exon 46 | 29 | 1 | ATTCAATCATTGGTTTTCTGCCCA | TTTA | 195 |
| Human-Exon 46 | 30 | -1 | GCAAGGAACTATGAATAACCTAAT | TTTA | 196 |
| Human-Exon 46 | 31 | 1 | CTGCCCATTAGGTTATTCATAGTT | TTTT | 197 |
| Human-Exon 46 | 32 | 1 | TGCCCATTAGGTTATTCATAGTTC | TTTC | 198 |
| Human-Exon 52 | 1 | -1 | TAGAAAACAATTTAACAGGAAATA | TTTA | 199 |
| Human-Exon 52 | 2 | 1 | CTGTTAAATTGTTTTCTATAAACC | TTTC | 200 |
| Human-Exon 52 | 3 | -1 | GAAATAAAAAAGATGTTACTGTAT | TTTA | 201 |
| Human-Exon 52 | 4 | -1 | AGAAATAAAAAAGATGTTACTGTA | TTTT | 202 |
| Human-Exon 52 | 5 | 1 | CTATAAACCCTTATACAGTAACAT | TTTT | 203 |
| Human-Exon 52 | 6 | 1 | TATAAACCCTTATACAGTAACATC | TTTC | 204 |
| Human-Exon 52 | 7 | 1 | TTATTTCTAAAAGTGTTTTGGCTG | TTTT | 205 |
| Human-Exon 52 | 8 | 1 | TATTTCTAAAAGTGTTTTGGCTGG | TTTT | 206 |
| Human-Exon 52 | 9 | 1 | ATTTCTAAAAGTGTTTTGGCTGGT | TTTT | 207 |
| Human-Exon 52 | 10 | 1 | TTTCTAAAAGTGTTTTGGCTGGTC | TTTA | 208 |
| Human-Exon 52 | 11 | 1 | TAAAAGTGTTTTGGCTGGTCTCAC | TTTC | 209 |
| Human-Exon 52 | 12 | -1 | CATAATACAAAGTAAAGTACAATT | TTTA | 210 |
| Human-Exon 52 | 13 | -1 | ACATAATACAAAGTAAAGTACAAT | TTTT | 211 |
| Human-Exon 52 | 14 | 1 | GGCTGGTCTCACAATTGTACTTTA | TTTT | 212 |
| Human-Exon 52 | 15 | 1 | GCTGGTCTCACAATTGTACTTTAC | TTTG | 213 |
| Human-Exon 52 | 16 | 1 | CTTTGTATTATGTAAAAGGAATAC | TTTA | 214 |
| Human-Exon 52 | 17 | 1 | TATTATGTAAAAGGAATACACAAC | TTTG | 215 |
| Human-Exon 52 | 18 | 1 | TTCTTACAGGCAACAATGCAGGAT | TTTG | 216 |
| Human-Exon 52 | 19 | 1 | GAACAGAGGCGTCCCCAGTTGGAA | TTTG | 217 |
| Human-Exon 52 | 20 | -1 | GGCAGCGGTAATGAGTTCTTCCAA | TTTG | 218 |
| Human-Exon 52 | 21 | -1 | TCAAATTTTGGGCAGCGGTAATGA | TTTT | 219 |
| Human-Exon 52 | 22 | 1 | AAAAACAAGACCAGCAATCAAGAG | TTTG | 220 |
| Human-Exon 52 | 23 | -1 | TGTGTCCCATGCTTGTTAAAAAAC | TTTG | 221 |
| Human-Exon 52 | 24 | 1 | TTAACAAGCATGGGACACACAAAG | TTTT | 222 |
| Human-Exon 52 | 25 | 1 | TAACAAGCATGGGACACACAAAGC | TTTT | 223 |
| Human-Exon 52 | 26 | 1 | AACAAGCATGGGACACACAAAGCA | TTTT | 224 |
| Human-Exon 52 | 27 | 1 | ACAAGCATGGGACACACAAAGCAA | TTTA | 225 |
| Human-Exon 52 | 28 | -1 | TTGAAACTTGTCATGCATCTTGCT | TTTA | 226 |
| Human-Exon 52 | 29 | -1 | ATTGAAACTTGTCATGCATCTTGC | TTTT | 227 |
| Human-Exon 52 | 30 | -1 | TATTGAAACTTGTCATGCATCTTG | TTTT | 228 |
| Human-Exon 52 | 31 | 1 | AATAAAAACTTAAGTTCATATATC | TTTC | 229 |
| Human-Exon 50 | 1 | -1 | GTGAATATATTATTGGATTTCTAT | TTTG | 230 |
| Human-Exon 50 | 2 | -1 | AAGATAATTCATGAACATCTTAAT | TTTG | 231 |
| Human-Exon 50 | 3 | -1 | ACAGAAAAGCATACACATTACTTA | TTTA | 232 |
| Human-Exon 50 | 4 | 1 | CTGTTAAAGAGGAAGTTAGAAGAT | TTTT | 233 |
| Human-Exon 50 | 5 | 1 | TGTTAAAGAGGAAGTTAGAAGATC | TTTC | 234 |
| Human-Exon 50 | 6 | -1 | CCGCCTTCCACTCAGAGCTCAGAT | TTTA | 235 |
| Human-Exon 50 | 7 | -1 | CCCTCAGCTCTTGAAGTAAACGGT | TTTG | 236 |
| Human-Exon 50 | 8 | 1 | CTTCAAGAGCTGAGGGCAAAGCAG | TTTA | 237 |
| Human-Exon 50 | 9 | -1 | AACAAATAGCTAGAGCCAAAGAGA | TTTG | 238 |
| Human-Exon 50 | 10 | -1 | GAACAAATAGCTAGAGCCAAAGAG | TTTT | 239 |
| Human-Exon 50 | 11 | 1 | GCTCTAGCTATTTGTTCAAAAGTG | TTTG | 240 |
| Human-Exon 50 | 12 | 1 | TTCAAAAGTGCAACTATGAAGTGA | TTTG | 241 |
| Human-Exon 50 | 13 | -1 | TCTCTCACCCAGTCATCACTTCAT | TTTC | 242 |
| Human-Exon 50 | 14 | -1 | CTCTCTCACCCAGTCATCACTTCA | TTTT | 243 |
| Human-Exon 43 | 1 | 1 | tatatatatatatatTTTTCTCTT | TTTG | 244 |
| Human-Exon 43 | 2 | 1 | TCTCTTTCTATAGACAGCTAATTC | tTTT | 245 |
| Human-Exon 43 | 3 | 1 | CTCTTTCTATAGACAGCTAATTCA | TTTT | 246 |
| Human-Exon 43 | 4 | -1 | AAACAGTAAAAAAATGAATTAGCT | TTTA | 247 |
| Human-Exon 43 | 5 | 1 | TCTTTCTATAGACAGCTAATTCAT | TTTC | 248 |
| Human-Exon 43 | 6 | -1 | AAAACAGTAAAAAAATGAATTAGC | TTTT | 249 |
| Human-Exon 43 | 7 | 1 | TATAGACAGCTAATTCATTTTTTT | TTTC | 250 |
| Human-Exon 43 | 8 | -1 | TATTCTGTAATATAAAAATTTTAA | TTTA | 251 |
| Human-Exon 43 | 9 | -1 | ATATTCTGTAATATAAAAATTTTA | TTTT | 252 |
| Human-Exon 43 | 10 | 1 | TTTACTGTTTTAAAATTTTTATAT | TTTT | 253 |
| Human-Exon 43 | 11 | 1 | TTACTGTTTTAAAATTTTTATATT | TTTT | 254 |
| Human-Exon 43 | 12 | 1 | TACTGTTTTAAAATTTTTATATTA | TTTT | 255 |
| Human-Exon 43 | 13 | 1 | ACTGTTTTAAAATTTTTATATTAC | TTTT | 256 |
| Human-Exon 43 | 14 | 1 | CTGTTTTAAAATTTTTATATTACA | TTTA | 257 |
| Human-Exon 43 | 15 | 1 | AAAATTTTTATATTACAGAATATA | TTTT | 258 |
| Human-Exon 43 | 16 | 1 | AAATTTTTATATTACAGAATATAA | TTTA | 259 |
| Human-Exon 43 | 17 | -1 | TTGTAGACTATCTTTTATATTCTG | TTTG | 260 |
| Human-Exon 43 | 18 | 1 | TATATTACAGAATATAAAAGATAG | TTTT | 261 |
| Human-Exon 43 | 19 | 1 | ATATTACAGAATATAAAAGATAGT | TTTT | 262 |
| Human-Exon 43 | 20 | 1 | TATTACAGAATATAAAAGATAGTC | TTTA | 263 |
| Human-Exon 43 | 21 | -1 | CAATGCTGCTGTCTTCTTGCTATG | TTTG | 264 |
| Human-Exon 43 | 22 | 1 | CAATGGGAAAAAGTTAACAAAATG | TTTC | 265 |
| Human-Exon 43 | 23 | -1 | TGCAAGTATCAAGAAAAATATATG | TTTC | 266 |
| Human-Exon 43 | 24 | 1 | TCTTGATACTTGCAGAAATGATTT | TTTT | 267 |
| Human-Exon 43 | 25 | 1 | CTTGATACTTGCAGAAATGATTTG | TTTT | 268 |
| Human-Exon 43 | 26 | 1 | TTGATACTTGCAGAAATGATTTGT | TTTC | 269 |
| Human-Exon 43 | 27 | 1 | TTTTCAGGGAACTGTAGAATTTAT | TTTG | 270 |
| Human-Exon 43 | 28 | -1 | CATGGAGGGTACTGAAATAAATTC | TTTC | 271 |
| Human-Exon 43 | 29 | -1 | CCATGGAGGGTACTGAAATAAATT | TTTT | 272 |
| Human-Exon 43 | 30 | 1 | CAGGGAACTGTAGAATTTATTTCA | TTTT | 273 |
| Human-Exon 43 | 31 | -1 | TCCATGGAGGGTACTGAAATAAAT | TTTT | 274 |
| Human-Exon 43 | 32 | 1 | AGGGAACTGTAGAATTTATTTCAG | TTTC | 275 |
| Human-Exon 43 | 33 | -1 | TTCCATGGAGGGTACTGAAATAAA | TTTT | 276 |
| Human-Exon 43 | 34 | -1 | CCTGTCTTTTTTCCATGGAGGGTA | TTTC | 277 |
| Human-Exon 43 | 35 | -1 | CCCTGTCTTTTTTCCATGGAGGGT | TTTT | 278 |
| Human-Exon 43 | 36 | -1 | TCCCTGTCTTTTTTCCATGGAGGG | TTTT | 279 |
| Human-Exon 43 | 37 | 1 | TTTCAGTACCCTCCATGGAAAAAA | TTTA | 280 |
| Human-Exon 43 | 38 | 1 | AGTACCCTCCATGGAAAAAAGACA | TTTC | 281 |
| Human-Exon 6 | 1 | 1 | AGTTTGCATGGTTCTTGCTCAAGG | TTTA | 282 |
| Human-Exon 6 | 2 | -1 | ATAAGAAAATGCATTCCTTGAGCA | TTTC | 283 |
| Human-Exon 6 | 3 | -1 | CATAAGAAAATGCATTCCTTGAGC | TTTT | 284 |
| Human-Exon 6 | 4 | 1 | CATGGTTCTTGCTCAAGGAATGCA | TTTG | 285 |
| Human-Exon 6 | 5 | -1 | ACCTACATGTGGAAATAAATTTTC | TTTG | 286 |
| Human-Exon 6 | 6 | -1 | GACCTACATGTGGAAATAAATTTT | TTTT | 287 |
| Human-Exon 6 | 7 | -1 | TGACCTACATGTGGAAATAAATTT | TTTT | 288 |
| Human-Exon 6 | 8 | 1 | CTTATGAAAATTTATTTCCACATG | TTTT | 289 |
| Human-Exon 6 | 9 | 1 | TTATGAAAATTTATTTCCACATGT | TTTC | 290 |
| Human-Exon 6 | 10 | -1 | ATTACATTTTTGACCTACATGTGG | TTTC | 291 |
| Human-Exon 6 | 11 | -1 | CATTACATTTTTGACCTACATGTG | TTTT | 292 |
| Human-Exon 6 | 12 | -1 | TCATTACATTTTTGACCTACATGT | TTTT | 293 |
| Human-Exon 6 | 13 | 1 | TTTCCACATGTAGGTCAAAAATGT | TTTA | 294 |
| Human-Exon 6 | 14 | 1 | CACATGTAGGTCAAAAATGTAATG | TTTC | 295 |
| Human-Exon 6 | 15 | -1 | TTGCAATCCAGCCATGATATTTTT | TTTG | 296 |
| Human-Exon 6 | 16 | -1 | ACTGTTGGTTTGTTGCAATCCAGC | TTTC | 297 |
| Human-Exon 6 | 17 | -1 | CACTGTTGGTTTGTTGCAATCCAG | TTTT | 298 |
| Human-Exon 6 | 18 | 1 | AATGCTCTCATCCATAGTCATAGG | TTTG | 299 |
| Human-Exon 6 | 19 | -1 | ATGTCTCAGTAATCTTCTTACCTA | TTTA | 300 |
| Human-Exon 6 | 20 | -1 | CAAGTTATTTAATGTCTCAGTAAT | TTTA | 301 |
| Human-Exon 6 | 21 | -1 | ACAAGTTATTTAATGTCTCAGTAA | TTTT | 302 |
| Human-Exon 6 | 22 | 1 | GACTCTGATGACATATTTTTCCCC | TTTA | 303 |
| Human-Exon 6 | 23 | 1 | TCCCCAGTATGGTTCCAGATCATG | TTTT | 304 |
| Human-Exon 6 | 24 | 1 | CCCCAGTATGGTTCCAGATCATGT | TTTT | 305 |
| Human-Exon 6 | 25 | 1 | CCCAGTATGGTTCCAGATCATGTC | TTTC | 306 |
| Human-Exon 7 | 1 | 1 | TATTTGTCTTtgtgtatgtgtgta | TTTA | 307 |
| Human-Exon 7 | 2 | 1 | TCTTtgtgtatgtgtgtatgtgta | TTTG | 308 |
| Human-Exon 7 | 3 | 1 | tgtatgtgtgtatgtgtatgtgtt | TTtg | 309 |
| Human-Exon 7 | 4 | 1 | AGGCCAGACCTATTTGACTGGAAT | ttTT | 310 |
| Human-Exon 7 | 5 | 1 | GGCCAGACCTATTTGACTGGAATA | tTTA | 311 |
| Human-Exon 7 | 6 | 1 | ACTGGAATAGTGTGGTTTGCCAGC | TTTG | 312 |
| Human-Exon 7 | 7 | 1 | CCAGCAGTCAGCCACACAACGACT | TTTG | 313 |
| Human-Exon 7 | 8 | -1 | TCTATGCCTAATTGATATCTGGCG | TTTC | 314 |
| Human-Exon 7 | 9 | -1 | CCAACCTTCAGGATCGAGTAGTTT | TTTA | 315 |
| Human-Exon 7 | 10 | 1 | TGGACTACCACTGCTTTTAGTATG | TTTC | 316 |
| Human-Exon 7 | 11 | 1 | AGTATGGTAGAGTTTAATGTTTTC | TTTT | 317 |
| Human-Exon 7 | 12 | 1 | GTATGGTAGAGTTTAATGTTTTCA | TTTA | 318 |
| Human-Exon 8 | 1 | -1 | AGACTCTAAAAGGATAATGAACAA | TTTG | 319 |
| Human-Exon 8 | 2 | 1 | ACTTTGATTTGTTCATTATCCTTT | TTTA | 320 |
| Human-Exon 8 | 3 | -1 | TATATTTGAGACTCTAAAAGGATA | TTTC | 321 |
| Human-Exon 8 | 4 | 1 | ATTTGTTCATTATCCTTTTAGAGT | TTTG | 322 |
| Human-Exon 8 | 5 | -1 | GTTTCTATATTTGAGACTCTAAAA | TTTG | 323 |
| Human-Exon 8 | 6 | -1 | GGTTTCTATATTTGAGACTCTAAA | TTTT | 324 |
| Human-Exon 8 | 7 | -1 | TGGTTTCTATATTTGAGACTCTAA | TTTT | 325 |
| Human-Exon 8 | 8 | 1 | TTCATTATCCTTTTAGAGTCTCAA | TTTG | 326 |
| Human-Exon 8 | 9 | 1 | AGAGTCTCAAATATAGAAACCAAA | TTTT | 327 |
| Human-Exon 8 | 10 | 1 | GAGTCTCAAATATAGAAACCAAAA | TTTA | 328 |
| Human-Exon 8 | 11 | -1 | CACTTCCTGGATGGCTTCAATGCT | TTTC | 329 |
| Human-Exon 8 | 12 | 1 | GCCTCAACAAGTGAGCATTGAAGC | TTTT | 330 |
| Human-Exon 8 | 13 | 1 | CCTCAACAAGTGAGCATTGAAGCC | TTTG | 331 |
| Human-Exon 8 | 14 | -1 | GGTGGCCTTGGCAACATTTCCACT | TTTA | 332 |
| Human-Exon 8 | 15 | -1 | GTCACTTTAGGTGGCCTTGGCAAC | TTTA | 333 |
| Human-Exon 8 | 16 | -1 | ATGATGTAACTGAAAATGTTCTTC | TTTG | 334 |
| Human-Exon 8 | 17 | -1 | CCTGTTGAGAATAGTGCATTTGAT | TTTA | 335 |
| Human-Exon 8 | 18 | 1 | CAGTTACATCATCAAATGCACTAT | TTTT | 336 |
| Human-Exon 8 | 19 | 1 | AGTTACATCATCAAATGCACTATT | TTTC | 337 |
| Human-Exon 8 | 20 | -1 | CACACTTTACCTGTTGAGAATAGT | TTTA | 338 |
| Human-Exon 8 | 21 | 1 | CTGTTTTATATGCATTTTTAGGTA | TTTT | 339 |
| Human-Exon 8 | 22 | 1 | TGTTTTATATGCATTTTTAGGTAT | TTTC | 340 |
| Human-Exon 8 | 23 | 1 | ATATGCATTTTTAGGTATTACGTG | TTTT | 341 |
| Human-Exon 8 | 24 | 1 | TATGCATTTTTAGGTATTACGTGC | TTTA | 342 |
| Human-Exon 8 | 25 | 1 | TAGGTATTACGTGCACatatatat | TTTT | 343 |
| Human-Exon 8 | 26 | 1 | AGGTATTACGTGCACatatatata | TTTT | 344 |
| Human-Exon 8 | 27 | 1 | GGTATTACGTGCACatatatatat | TTTA | 345 |
| Human-Exon 55 | 1 | -1 | AGCAACAACTATAATATTGTGCAG | TTTA | 346 |
| Human-Exon 55 | 2 | 1 | GTTCCTCCATCTTTCTCTTTTTAT | TTTA | 347 |
| Human-Exon 55 | 3 | 1 | TCTTTTTATGGAGTTCACTAGGTG | TTTC | 348 |
| Human-Exon 55 | 4 | 1 | TATGGAGTTCACTAGGTGCACCAT | TTTT | 349 |
| Human-Exon 55 | 5 | 1 | ATGGAGTTCACTAGGTGCACCATT | TTTT | 350 |
| Human-Exon 55 | 6 | 1 | TGGAGTTCACTAGGTGCACCATTC | TTTA | 351 |
| Human-Exon 55 | 7 | 1 | ATAATTGCATCTGAACATTTGGTC | TTTA | 352 |
| Human-Exon 55 | 8 | 1 | GTCCTTTGCAGGGTGAGTGAGCGA | TTTG | 353 |
| Human-Exon 55 | 9 | -1 | TTCCAAAGCAGCCTCTCGCTCACT | TTTC | 354 |
| Human-Exon 55 | 10 | 1 | CAGGGTGAGTGAGCGAGAGGCTGC | TTTG | 355 |
| Human-Exon 55 | 11 | 1 | GAAGAAACTCATAGATTACTGCAA | TTTG | 356 |
| Human-Exon 55 | 12 | -1 | CAGGTCCAGGGGGAACTGTTGCAG | TTTC | 357 |
| Human-Exon 55 | 13 | -1 | CCAGGTCCAGGGGGAACTGTTGCA | TTTT | 358 |
| Human-Exon 55 | 14 | -1 | AGCTTCTGTAAGCCAGGCAAGAAA | TTTC | 359 |
| Human-Exon 55 | 15 | 1 | TTGCCTGGCTTACAGAAGCTGAAA | TTTC | 360 |
| Human-Exon 55 | 16 | -1 | CTTACGGGTAGCATCCTGTAGGAC | TTTC | 361 |
| Human-Exon 55 | 17 | -1 | CTCCCTTGGAGTCTTCTAGGAGCC | TTTA | 362 |
| Human-Exon 55 | 18 | -1 | ACTCCCTTGGAGTCTTCTAGGAGC | TTTT | 363 |
| Human-Exon 55 | 19 | -1 | ATCAGCTCTTTTACTCCCTTGGAG | TTTC | 364 |
| Human-Exon 55 | 20 | 1 | CGCTTTAGCACTCTTGTGGATCCA | TTTC | 365 |
| Human-Exon 55 | 21 | 1 | GCACTCTTGTGGATCCAATTGAAC | TTTA | 366 |
| Human-Exon 55 | 22 | -1 | TCCCTGGCTTGTCAGTTACAAGTA | TTTG | 367 |
| Human-Exon 55 | 23 | -1 | GTCCCTGGCTTGTCAGTTACAAGT | TTTT | 368 |
| Human-Exon 55 | 24 | -1 | TTTTGTCCCTGGCTTGTCAGTTAC | TTTG | 369 |
| Human-Exon 55 | 25 | -1 | GTTTTGTCCCTGGCTTGTCAGTTA | TTTT | 370 |
| Human-Exon 55 | 26 | 1 | TACTTGTAACTGACAAGCCAGGGA | TTTG | 371 |
| Human-G1-exon51 | | 1 | gCTCCTACTCAGACTGTTACTCTG | TTTA | 372 |
| Human-G2-exon51 | | 1 | taccatgtattgctaaacaaagta | TTTC | 373 |
| Human-G3-exon51 | | -1 | attgaagagtaacaatttgagcca | TTTA | 374 |
| mouse-Exon23-G1 | | 1 | aggctctgcaaagttctTTGAAAG | TTTG | 375 |
| mouse-Exon23-G2 | | 1 | AAAGAGCAACAAAATGGCttcaac | TTTG | 376 |
| mouse-Exon23-G3 | | 1 | AAAGAGCAATAAAATGGCttcaac | TTTG | 377 |
| mouse-Exon23-G4 | | -1 | AAAGAACTTTGCAGAGCctcaaaa | TTTC | 378 |
| mouse-Exon23-G5 | | -1 | ctgaatatctatgcattaataact | TTTA | 379 |
| mouse-Exon23-G6 | | -1 | tattatattacagggcatattata | TTTC | 380 |
| mouse-Exon23-G7 | | 1 | Aggtaagccgaggtttggccttta | TTTC | 381 |
| mouse-Exon23-G8 | | 1 | cccagagtccttcaaagatattga | TTTA | 382 |

*In this table, upper case letters represent nucleotides that align to the exon sequence of the gene. Lower case letters represent nucleotides that align to the intron sequence of the gene.

TABLE 7
gRNA sequences

| Targeted | | | | | SEQ ID |
|---|---|---|---|---|---|
| gRNA Exon | Guide # | Strand | gRNA sequence* | PAM | NO. |
| Human-Exon 51 | 4 | 1 | aaaaaggaaaaaagaagaaaaaga | tttt | 383 |
| Human-Exon 51 | 5 | 1 | Caaaaaggaaaaaagaagaaaaag | tttt | 384 |
| Human-Exon 51 | 6 | 1 | GCaaaaaggaaaaaagaagaaaaa | tttc | 385 |
| Human-Exon 51 | 7 | 1 | UUUUGCaaaaaggaaaaaagaaga | tttt | 386 |
| Human-Exon 51 | 8 | 1 | UUUUUGCaaaaaggaaaaaagaag | tttt | 387 |
| Human-Exon 51 | 9 | 1 | GUUUUUGCaaaaaggaaaaaagaa | tttc | 388 |
| Human-Exon 51 | 10 | 1 | AUUUUGGGUUUUUGCaaaaaggaa | tttt | 389 |
| Human-Exon 51 | 11 | 1 | UAUUUUGGGUUUUUGCaaaaagga | tttt | 390 |
| Human-Exon 51 | 12 | 1 | AUAUUUUGGGUUUUUGCaaaaagg | tttt | 391 |
| Human-Exon 51 | 13 | 1 | AAUAUUUUGGGUUUUUGCaaaaag | tttc | 392 |
| Human-Exon 51 | 14 | 1 | GCUAAAAUAUUUUGGGUUUUUGCa | tttt | 393 |
| Human-Exon 51 | 15 | 1 | AGCUAAAAUAUUUUGGGUUUUUGC | tttt | 394 |
| Human-Exon 51 | 16 | 1 | GAGCUAAAAUAUUUUGGGUUUUUG | tttG | 395 |
| Human-Exon 51 | 17 | 1 | AGAGUAACAGUCUGAGUAGGAGCU | TTTT | 396 |
| Human-Exon 51 | 18 | 1 | CAGAGUAACAGUCUGAGUAGGAGC | TTTA | 397 |
| Human-Exon 51 | 19 | -1 | GUGACACAACCUGUGGUUACUAAG | TTTC | 398 |
| Human-Exon 51 | 20 | -1 | GGUUACUAAGGAAACUGCCAUCU | TTTG | 399 |
| Human-Exon 51 | 21 | -1 | AAGGAAACUGCCAUCUCCAAACUA | TTTC | 400 |
| Human-Exon 51 | 22 | -1 | AUCAUCAAGCAGAAGGUAUGAGAA | TTTT | 401 |
| Human-Exon 51 | 23 | -1 | AGCAGAAGGUAUGAGAAAAAAUGA | TTTA | 402 |
| Human-Exon 51 | 24 | -1 | GCAGAAGGUAUGAGAAAAAAUGAU | TTTT | 403 |
| Human-Exon 51 | 25 | -1 | UAAAAGUUGGCAGAAGUUUUUCUU | TTTA | 404 |
| Human-Exon 51 | 26 | -1 | AAAAGUUGGCAGAAGUUUUUCUUU | TTTT | 405 |
| Human-Exon 51 | 27 | 1 | GGUGGAAAAUCUUCAUUUUAAAGA | TTTT | 406 |
| Human-Exon 51 | 28 | 1 | UGGUGGAAAAUCUUCAUUUUAAAG | TTTT | 407 |
| Human-Exon 51 | 29 | 1 | UUGGUGGAAAAUCUUCAUUUUAAA | TTTC | 408 |
| Human-Exon 51 | 30 | 1 | GUGAUUGGUGGAAAAUCUUCAUUU | TTTA | 409 |
| Human-Exon 51 | 31 | 1 | CUAGGAGAGUAAAGUGAUUGGUGG | TTTT | 410 |
| Human-Exon 51 | 32 | 1 | UCUAGGAGAGUAAAGUGAUUGGUG | TTTC | 411 |
| Human-Exon 51 | 33 | 1 | CUGGUGGGAAAUGGUCUAGGAGA | TTTA | 412 |
| Human-Exon 45 | 1 | -1 | guagcacacuguuuaaucuuuucu | tttg | 413 |
| Human-Exon 45 | 2 | -1 | cacacuguuuaaucuuuucucaaa | TTTa | 414 |
| Human-Exon 45 | 3 | -1 | acacuguuuaaucuuuucucaaau | TTTT | 415 |
| Human-Exon 45 | 4 | -1 | cacuguuuaaucuuuucucaaauA | TTTT | 416 |
| Human-Exon 45 | 5 | 1 | AUGUCUUUUUauuugagaaaagau | ttta | 417 |
| Human-Exon 45 | 6 | 1 | AAGCCCCAUGUCUUUUUauuugag | tttt | 418 |
| Human-Exon 45 | 7 | 1 | GAAGCCCCAUGUCUUUUUauuuga | tttc | 419 |
| Human-Exon 45 | 8 | 1 | GUAAGAUACCAAAAAGGCAAAACA | TTTT | 420 |
| Human-Exon 45 | 9 | 1 | UGUAAGAUACCAAAAAGGCAAAAC | TTTT | 421 |
| Human-Exon 45 | 10 | 1 | CUGUAAGAUACCAAAAAGGCAAAA | TTTG | 422 |
| Human-Exon 45 | 11 | 1 | GUUCCUGUAAGAUACCAAAAAGGC | TTTT | 423 |
| Human-Exon 45 | 12 | 1 | AGUUCCUGUAAGAUACCAAAAAGG | TTTG | 424 |
| Human-Exon 45 | 13 | 1 | UCCUGGAGUUCCUGUAAGAUACCA | TTTT | 425 |
| Human-Exon 45 | 14 | 1 | AUCCUGGAGUUCCUGUAAGAUACC | TTTT | 426 |
| Human-Exon 45 | 15 | -1 | GGGAAGAAAUAAUUCAGCAAUCCU | TTTG | 427 |
| Human-Exon 45 | 16 | -1 | GGAAGAAAUAAUUCAGCAAUCCUC | TTTT | 428 |
| Human-Exon 45 | 17 | -1 | GAAGAAAUAAUUCAGCAAUCCUCA | TTTT | 429 |
| Human-Exon 45 | 18 | -1 | AAAACAGAUGCCAGUAUUCUACAG | TTTC | 430 |
| Human-Exon 45 | 19 | -1 | AAACAGAUGCCAGUAUUCUACAGG | TTTT | 431 |
| Human-Exon 45 | 20 | -1 | AACAGAUGCCAGUAUUCUACAGGA | TTTT | 432 |
| Human-Exon 45 | 21 | -1 | GAAUCUGCGGUGGCAGGAGGUCUG | TTTG | 433 |
| Human-Exon 45 | 22 | -1 | AGGUCUGCAAACAGCUGUCAGACA | TTTC | 434 |
| Human-Exon 45 | 23 | -1 | GGUCUGCAAACAGCUGUCAGACAG | TTTT | 435 |
| Human-Exon 45 | 24 | -1 | GUCUGCAAACAGCUGUCAGACAGA | TTTT | 436 |
| Human-Exon 45 | 25 | -1 | UCUGCAAACAGCUGUCAGACAGAA | TTTT | 437 |
| Human-Exon 45 | 26 | -1 | UAGGGCGACAGAUCUAAUAGGAAU | TTTC | 438 |
| Human-Exon 45 | 27 | -1 | AGGGCGACAGAUCUAAUAGGAAUG | TTTT | 439 |
| Human-Exon 45 | 28 | 1 | UAAAGAAAGCUUAAAAAGUCUGCU | TTTT | 440 |
| Human-Exon 45 | 29 | 1 | CUAAAGAAAGCUUAAAAAGUCUGC | TTTA | 441 |
| Human-Exon 45 | 30 | 1 | AAAUAUUCUUCUAAAGAAAGCUUA | TTTT | 442 |
| Human-Exon 45 | 31 | 1 | GAAAUAUUCUUCUAAAGAAAGCUU | TTTT | 443 |
| Human-Exon 45 | 32 | 1 | UGAAAUAUUCUUCUAAAGAAAGCU | TTTA | 444 |
| Human-Exon 45 | 33 | 1 | UCUCUCAUGAAAUAUUCUUCUAAA | TTTC | 445 |
| Human-Exon 45 | 34 | 1 | AUAAUCUCUCAUGAAAUAUUCUUC | TTTA | 446 |
| Human-Exon 44 | 1 | 1 | GCGUAUAUUUUUUGGUUAUACUGA | TTTG | 447 |
| Human-Exon 44 | 2 | 1 | ucaagaaaaauagauggauuaugu | tttt | 448 |
| Human-Exon 44 | 3 | 1 | aucaagaaaaauagauggauuaug | ttta | 449 |
| Human-Exon 44 | 4 | 1 | CAGGUaaaagcauauggaucaaga | tttt | 450 |
| Human-Exon 44 | 5 | 1 | GCAGGUaaaagcauauggaucaag | tttt | 451 |
| Human-Exon 44 | 6 | 1 | UGCAGGUaaaagcauauggaucaa | tttc | 452 |
| Human-Exon 44 | 7 | -1 | CAGGCGAUUUGACAGAUCUGUUGA | TTTC | 453 |
| Human-Exon 44 | 8 | 1 | AGAUCUGUCAAAUCGCCUGCAGGU | tttt | 454 |
| Human-Exon 44 | 9 | 1 | CAGAUCUGUCAAAUCGCCUGCAGG | tttA | 455 |
| Human-Exon 44 | 10 | 1 | GCCGCCAUUUCUCAACAGAUCUGU | TTTG | 456 |
| Human-Exon 44 | 11 | -1 | AAUGGCGGCGUUUUCAUUAUGAUA | TTTA | 457 |
| Human-Exon 44 | 12 | 1 | AUUAAAUAUCUUUAUAUCAUAAUG | TTTT | 458 |
| Human-Exon 44 | 13 | -1 | UGAGAAUUGGGAACAUGCUAAAUA | TTTG | 459 |
| Human-Exon 44 | 14 | -1 | GGUAAGUCUUUGAUUUGUUUUUUC | TTTC | 460 |
| Human-Exon 44 | 15 | 1 | AAAUACAAUUUCGAAAAAACAAAU | TTTG | 461 |
| Human-Exon 44 | 16 | 1 | AAGAUAAAUACAAUUUCGAAAAAA | TTTG | 462 |
| Human-Exon 44 | 17 | 1 | GCUGAAGAUAAAUACAAUUUCGAA | TTTT | 463 |
| Human-Exon 44 | 18 | 1 | UGCUGAAGAUAAAUACAAUUUCGA | TTTT | 464 |
| Human-Exon 44 | 19 | 1 | GUGCUGAAGAUAAAUACAAUUUCG | TTTT | 465 |
| Human-Exon 44 | 20 | 1 | UGUGCUGAAGAUAAAUACAAUUUC | TTTC | 466 |
| Human-Exon 44 | 21 | -1 | GCACAUCUGGACUCUUUAACUUCU | TTTA | 467 |
| Human-Exon 44 | 22 | 1 | UAAAGAGUCCAGAUGUGCUGAAGA | TTTA | 468 |
| Human-Exon 44 | 23 | -1 | AAGAUCAGGUUCUGAAGGGUGAUG | TTTC | 469 |
| Human-Exon 44 | 24 | 1 | UUCAGAACCUGAUCUUUAAGAAGU | TTTA | 470 |
| Human-Exon 44 | 25 | 1 | AAUAUAAUGAUGACAACAACAGUC | TTTT | 471 |
| Human-Exon 44 | 26 | 1 | UAAUAUAAUGAUGACAACAACAGU | TTTG | 472 |
| Human-Exon 53 | 1 | -1 | UUUAUUUUUCCUUUUAUUCUAGUU | TTTC | 473 |
| Human-Exon 53 | 2 | 1 | AAAGGAAAAAUAAAUAUAUAGUAG | TTTA | 474 |
| Human-Exon 53 | 3 | 1 | UUUCAACUAGAAUAAAAGGAAAAA | TTTA | 475 |
| Human-Exon 53 | 4 | 1 | AUUCUUUCAACUAGAAUAAAAGGA | TTTT | 476 |
| Human-Exon 53 | 5 | 1 | AAUUCUUUCAACUAGAAUAAAAGG | TTTT | 477 |
| Human-Exon 53 | 6 | 1 | GAAUUCUUUCAACUAGAAUAAAAG | TTTC | 478 |
| Human-Exon 53 | 7 | 1 | AUUCUGAAUUCUUUCAACUAGAAU | TTTT | 479 |
| Human-Exon 53 | 8 | 1 | GAUUCUGAAUUCUUUCAACUAGAA | TTTA | 480 |
| Human-Exon 53 | 9 | -1 | CAGAACCGGAGGCAACAGUUGAAU | TTTC | 481 |
| Human-Exon 53 | 10 | -1 | GGAGGCAACAGUUGAAUGAAAUGU | TTTA | 482 |
| Human-Exon 53 | 11 | -1 | UAUACAGUAGAUGCAAUCCAAAAG | TTTT | 483 |
| Human-Exon 53 | 12 | -1 | GAUGCAAUCCAAAAGAAAAUCACA | TTTC | 484 |
| Human-Exon 53 | 13 | -1 | AAUCACAGAAACCAAGGUUAGUAU | TTTG | 485 |
| Human-Exon 53 | 14 | -1 | AGGUUAGUAUCAAAGAUACCUUU | TTTA | 486 |
| Human-Exon 53 | 15 | -1 | GGUUAGUAUCAAAGAUACCUUUUU | TTTT | 487 |
| Human-Exon 53 | 16 | -1 | AGUAUCAAAGAUACCUUUUUAAAA | TTTA | 488 |
| Human-Exon 53 | 17 | -1 | GUAUCAAAGAUACCUUUUUAAAAU | TTTT | 489 |
| Human-Exon 46 | 1 | -1 | UGUUUGUGUCCCAGUUUGCAUUAA | TTTG | 490 |
| Human-Exon 46 | 2 | 1 | CUGGGACACAAACAUGGCAAUUUA | TTTT | 491 |
| Human-Exon 46 | 3 | 1 | ACUGGGACACAAACAUGGCAAUUU | TTTT | 492 |
| Human-Exon 46 | 4 | 1 | AACUGGGACACAAACAUGGCAAUU | TTTA | 493 |
| Human-Exon 46 | 5 | 1 | UAUUUGUUAAUGCAAACUGGGACA | TTTG | 494 |
| Human-Exon 46 | 6 | -1 | ACAAAUAGUUUGAGAACUAUGUUG | tttC | 495 |
| Human-Exon 46 | 7 | -1 | CAAAUAGUUUGAGAACUAUGUUGG | tttt | 496 |
| Human-Exon 46 | 8 | -1 | AAAUAGUUUGAGAACUAUGUUGGa | tttt | 497 |
| Human-Exon 46 | 9 | -1 | AUAGUUUGAGAACUAUGUUGGaaa | tilt | 498 |
| Human-Exon 46 | 10 | -1 | UAGUUUGAGAACUAUGUUGGaaaa | tttt | 499 |
| Human-Exon 46 | 11 | -1 | AGUUUGAGAACUAUGUUGGaaaaa | tttt | 500 |
| Human-Exon 46 | 12 | 1 | UAGUUCUCAAACUAUUUGUUAAUG | TTTG | 501 |
| Human-Exon 46 | 13 | 1 | UAuuuuuuuuuCCAACAUAGUUCU | TTTG | 502 |
| Human-Exon 46 | 14 | -1 | CUUCUUUCUCCAGGCUAGAAGAAC | TTTT | 503 |
| Human-Exon 46 | 15 | 1 | CUUCUAGCCUGGAGAAAGAAGAAU | TTTT | 504 |
| Human-Exon 46 | 16 | 1 | UCUUCUAGCCUGGAGAAAGAAGAA | TTTA | 505 |
| Human-Exon 46 | 17 | 1 | AUUCUUUUGUUCUUCUAGCCUGGA | TTTC | 506 |
| Human-Exon 46 | 18 | -1 | CAAAAGAAUAUCUUGUCAGAAUUU | TTTG | 507 |
| Human-Exon 46 | 19 | -1 | CUGGAAAAGAGCAGCAACUAAAAG | TTTT | 508 |
| Human-Exon 46 | 20 | -1 | CAAGUCAAGGUAAUUUUAUUUUCU | TTTG | 509 |
| Human-Exon 46 | 21 | -1 | CAAAUCCCCCAGGGCCUGCUUGCA | TTTA | 510 |
| Human-Exon 46 | 22 | 1 | AGGCCCUGGGGGAUUUGAGAAAAU | TTTT | 511 |
| Human-Exon 46 | 23 | 1 | CAGGCCCUGGGGGAUUUGAGAAAA | TTTA | 512 |
| Human-Exon 46 | 24 | 1 | CAAGCAGGCCCUGGGGGAUUUGAG | TTTT | 513 |
| Human-Exon 46 | 25 | 1 | GCAAGCAGGCCCUGGGGGAUUUGA | TTTC | 514 |
| Human-Exon 46 | 26 | 1 | GCAGAAAACCAAUGAUUGAAUUAA | TTTT | 515 |
| Human-Exon 46 | 27 | 1 | GGCAGAAAACCAAUGAUUGAAUUA | TTTT | 516 |
| Human-Exon 46 | 28 | 1 | GGGCAGAAAACCAAUGAUUGAAUU | TTTT | 517 |
| Human-Exon 46 | 29 | 1 | UGGGCAGAAAACCAAUGAUUGAAU | TTTA | 518 |
| Human-Exon 46 | 30 | -1 | AUUAGGUUAUUCAUAGUUCCUUGC | TTTA | 519 |
| Human-Exon 46 | 31 | 1 | AACUAUGAAUAACCUAAUGGGCAG | TTTT | 520 |
| Human-Exon 46 | 32 | 1 | GAACUAUGAAUAACCUAAUGGGCA | TTTC | 521 |
| Human-Exon 52 | 1 | -1 | UAUUUCCUGUUAAAUUGUUUUCUA | TTTA | 522 |
| Human-Exon 52 | 2 | 1 | GGUUUAUAGAAAACAAUUUAACAG | TTTC | 523 |
| Human-Exon 52 | 3 | -1 | AUACAGUAACAUCUUUUUUAUUUC | TTTA | 524 |
| Human-Exon 52 | 4 | -1 | UACAGUAACAUCUUUUUUAUUUCU | TTTT | 525 |
| Human-Exon 52 | 5 | 1 | AUGUUACUGUAUAAGGGUUUAUAG | TTTT | 526 |
| Human-Exon 52 | 6 | 1 | GAUGUUACUGUAUAAGGGUUUAUA | TTTC | 527 |
| Human-Exon 52 | 7 | 1 | CAGCCAAAACACUUUUAGAAAUAA | TTTT | 528 |
| Human-Exon 52 | 8 | 1 | CCAGCCAAAACACUUUUAGAAAUA | TTTT | 529 |
| Human-Exon 52 | 9 | 1 | ACCAGCCAAAACACUUUUAGAAAU | TTTT | 530 |
| Human-Exon 52 | 10 | 1 | GACCAGCCAAAACACUUUUAGAAA | TTTA | 531 |
| Human-Exon 52 | 11 | 1 | GUGAGACCAGCCAAAACACUUUUA | TTTC | 532 |
| Human-Exon 52 | 12 | -1 | AAUUGUACUUUACUUUGUAUUAUG | TTTA | 533 |
| Human-Exon 52 | 13 | -1 | AUUGUACUUUACUUUGUAUUAUGU | TTTT | 534 |
| Human-Exon 52 | 14 | 1 | UAAAGUACAAUUGUGAGACCAGCC | TTTT | 535 |
| Human-Exon 52 | 15 | 1 | GUAAAGUACAAUUGUGAGACCAGC | TTTG | 536 |
| Human-Exon 52 | 16 | 1 | GUAUUCCUUUUACAUAAUACAAAG | TTTA | 537 |
| Human-Exon 52 | 17 | 1 | GUUGUGUAUUCCUUUUACAUAAUA | TTTG | 538 |
| Human-Exon 52 | 18 | 1 | AUCCUGCAUUGUUGCCUGUAAGAA | TTTG | 539 |
| Human-Exon 52 | 19 | 1 | UUCCAACUGGGGACGCCUCUGUUC | TTTG | 540 |
| Human-Exon 52 | 20 | -1 | UUGGAAGAACUCAUUACCGCUGCC | TTTG | 541 |
| Human-Exon 52 | 21 | -1 | UCAUUACCGCUGCCCAAAAUUUGA | TTTT | 542 |
| Human-Exon 52 | 22 | 1 | CUCUUGAUUGCUGGUCUUGUUUUU | TTTG | 543 |
| Human-Exon 52 | 23 | -1 | GUUUUUUAACAAGCAUGGGACACA | TTTG | 544 |
| Human-Exon 52 | 24 | 1 | CUUUGUGUGUCCCAUGCUUGUUAA | TTTT | 545 |
| Human-Exon 52 | 25 | 1 | GCUUUGUGUGUCCCAUGCUUGUUA | TTTT | 546 |
| Human-Exon 52 | 26 | 1 | UGCUUUGUGUGUCCCAUGCUUGUU | TTTT | 547 |
| Human-Exon 52 | 27 | 1 | UUGCUUUGUGUGUCCCAUGCUUGU | TTTA | 548 |
| Human-Exon 52 | 28 | -1 | AGCAAGAUGCAUGACAAGUUUCAA | TTTA | 549 |
| Human-Exon 52 | 29 | -1 | GCAAGAUGCAUGACAAGUUUCAAU | TTTT | 550 |
| Human-Exon 52 | 30 | -1 | CAAGAUGCAUGACAAGUUUCAAUA | TTTT | 551 |
| Human-Exon 52 | 31 | 1 | GAUAUAUGAACUUAAGUUUUUAUU | TTTC | 552 |
| Human-Exon 50 | 1 | -1 | AUAGAAAUCCAAUAAUAUAUUCAC | TTTG | 553 |
| Human-Exon 50 | 2 | -1 | AUUAAGAUGUUCAUGAAUUAUCUU | TTTG | 554 |
| Human-Exon 50 | 3 | -1 | UAAGUAAUGUGUAUGCUUUUCUGU | TTTA | 555 |
| Human-Exon 50 | 4 | 1 | AUCUUCUAACUUCCUCUUUAACAG | TTTT | 556 |
| Human-Exon 50 | 5 | 1 | GAUCUUCUAACUUCCUCUUUAACA | TTTC | 557 |
| Human-Exon 50 | 6 | -1 | AUCUGAGCUCUGAGUGGAAGGCGG | TTTA | 558 |
| Human-Exon 50 | 7 | -1 | ACCGUUUACUUCAAGAGCUGAGGG | TTTG | 559 |
| Human-Exon 50 | 8 | 1 | CUGCUUUGCCCUCAGCUCUUGAAG | TTTA | 560 |
| Human-Exon 50 | 9 | -1 | UCUCUUUGGCUCUAGCUAUUUGUU | TTTG | 561 |
| Human-Exon 50 | 10 | -1 | CUCUUUGGCUCUAGCUAUUUGUUC | TTTT | 562 |
| Human-Exon 50 | 11 | 1 | CACUUUUGAACAAAUAGCUAGAGC | TTTG | 563 |
| Human-Exon 50 | 12 | 1 | UCACUUCAUAGUUGCACUUUUGAA | TTTG | 564 |
| Human-Exon 50 | 13 | -1 | AUGAAGUGAUGACUGGGUGAGAGA | TTTC | 565 |
| Human-Exon 50 | 14 | -1 | UGAAGUGAUGACUGGGUGAGAGAG | TTTT | 566 |
| Human-Exon 43 | 1 | 1 | AAGAGAAAAauauauauauauaua | TTTG | 567 |
| Human-Exon 43 | 2 | 1 | GAAUUAGCUGUCUAUAGAAAGAGA | tTTT | 568 |
| Human-Exon 43 | 3 | 1 | UGAAUUAGCUGUCUAUAGAAAGAG | TTTT | 569 |
| Human-Exon 43 | 4 | -1 | AGCUAAUUCAUUUUUUUACUGUUU | TTTA | 570 |
| Human-Exon 43 | 5 | 1 | AUGAAUUAGCUGUCUAUAGAAAGA | TTTC | 571 |
| Human-Exon 43 | 6 | -1 | GCUAAUUCAUUUUUUUACUGUUUU | TTTT | 572 |
| Human-Exon 43 | 7 | 1 | AAAAAAAUGAAUUAGCUGUCUAUA | TTTC | 573 |
| Human-Exon 43 | 8 | -1 | UUAAAAUUUUUAUAUUACAGAAUA | TTTA | 574 |
| Human-Exon 43 | 9 | -1 | UAAAAUUUUUAUAUUACAGAAUAU | TTTT | 575 |
| Human-Exon 43 | 10 | 1 | AUAUAAAAAUUUUAAAACAGUAAA | TTTT | 576 |
| Human-Exon 43 | 11 | 1 | AAUAUAAAAAUUUUAAAACAGUAA | TTTT | 577 |
| Human-Exon 43 | 12 | 1 | UAAUAUAAAAAUUUUAAAACAGUA | TTTT | 578 |
| Human-Exon 43 | 13 | 1 | GUAAUAUAAAAAUUUUAAAACAGU | TTTT | 579 |
| Human-Exon 43 | 14 | 1 | UGUAAUAUAAAAAUUUUAAAACAG | TTTA | 580 |
| Human-Exon 43 | 15 | 1 | UAUAUUCUGUAAUAUAAAAAUUUU | TTTT | 581 |
| Human-Exon 43 | 16 | 1 | UUAUAUUCUGUAAUAUAAAAAUUU | TTTA | 582 |
| Human-Exon 43 | 17 | -1 | CAGAAUAUAAAAGAUAGUCUACAA | TTTG | 583 |
| Human-Exon 43 | 18 | 1 | CUAUCUUUUAUAUUCUGUAAUAUA | TTTT | 584 |
| Human-Exon 43 | 19 | 1 | ACUAUCUUUUAUAUUCUGUAAUAU | TTTT | 585 |
| Human-Exon 43 | 20 | 1 | GACUAUCUUUUAUAUUCUGUAAUA | TTTA | 586 |
| Human-Exon 43 | 21 | -1 | CAUAGCAAGAAGACAGCAGCAUUG | TTTG | 587 |
| Human-Exon 43 | 22 | 1 | CAUUUUGUUAACUUUUUCCCAUUG | TTTC | 588 |
| Human-Exon 43 | 23 | -1 | CAUAUAUUUUUCUUGAUACUUGCA | TTTC | 589 |
| Human-Exon 43 | 24 | 1 | AAAUCAUUUCUGCAAGUAUCAAGA | TTTT | 590 |
| Human-Exon 43 | 25 | 1 | CAAAUCAUUUCUGCAAGUAUCAAG | TTTT | 591 |
| Human-Exon 43 | 26 | 1 | ACAAAUCAUUUCUGCAAGUAUCAA | TTTC | 592 |
| Human-Exon 43 | 27 | 1 | AUAAAUUCUACAGUUCCCUGAAAA | TTTG | 593 |
| Human-Exon 43 | 28 | -1 | GAAUUUAUUUCAGUACCCUCCAUG | TTTC | 594 |
| Human-Exon 43 | 29 | -1 | AAUUUAUUUCAGUACCCUCCAUGG | TTTT | 595 |
| Human-Exon 43 | 30 | 1 | UGAAAUAAAUUCUACAGUUCCCUG | TTTT | 596 |
| Human-Exon 43 | 31 | -1 | AUUUAUUUCAGUACCCUCCAUGGA | TTTT | 597 |
| Human-Exon 43 | 32 | 1 | CUGAAAUAAAUUCUACAGUUCCCU | TTTC | 598 |
| Human-Exon 43 | 33 | -1 | UUUAUUUCAGUACCCUCCAUGGAA | TTTT | 599 |
| Human-Exon 43 | 34 | -1 | UACCCUCCAUGGAAAAAAGACAGG | TTTC | 600 |
| Human-Exon 43 | 35 | -1 | ACCCUCCAUGGAAAAAAGACAGGG | TTTT | 601 |
| Human-Exon 43 | 36 | -1 | CCCUCCAUGGAAAAAAGACAGGGA | TTTT | 602 |
| Human-Exon 43 | 37 | 1 | UUUUUUCCAUGGAGGGUACUGAAA | TTTA | 603 |
| Human-Exon 43 | 38 | 1 | UGUCUUUUUUCCAUGGAGGGUACU | TTTC | 604 |
| Human-Exon 6 | 1 | 1 | CCUUGAGCAAGAACCAUGCAAACU | TTTA | 605 |
| Human-Exon 6 | 2 | -1 | UGCUCAAGGAAUGCAUUUUCUUAU | TTTC | 606 |
| Human-Exon 6 | 3 | -1 | GCUCAAGGAAUGCAUUUUCUUAUG | TTTT | 607 |
| Human-Exon 6 | 4 | 1 | UGCAUUCCUUGAGCAAGAACCAUG | TTTG | 608 |
| Human-Exon 6 | 5 | -1 | GAAAAUUUAUUUCCACAUGUAGGU | TTTG | 609 |
| Human-Exon 6 | 6 | -1 | AAAAUUUAUUUCCACAUGUAGGUC | TTTT | 610 |
| Human-Exon 6 | 7 | -1 | AAAUUUAUUUCCACAUGUAGGUCA | TTTT | 611 |
| Human-Exon 6 | 8 | 1 | CAUGUGGAAAUAAAUUUUCAUAAG | TTTT | 612 |
| Human-Exon 6 | 9 | 1 | ACAUGUGGAAAUAAAUUUUCAUAA | TTTC | 613 |
| Human-Exon 6 | 10 | -1 | CCACAUGUAGGUCAAAAAUGUAAU | TTTC | 614 |
| Human-Exon 6 | 11 | -1 | CACAUGUAGGUCAAAAAUGUAAUG | TTTT | 615 |
| Human-Exon 6 | 12 | -1 | ACAUGUAGGUCAAAAAUGUAAUGA | TTTT | 616 |
| Human-Exon 6 | 13 | 1 | ACAUUUUUGACCUACAUGUGGAAA | TTTA | 617 |
| Human-Exon 6 | 14 | 1 | CAUUACAUUUUUGACCUACAUGUG | TTTC | 618 |
| Human-Exon 6 | 15 | -1 | AAAAAUAUCAUGGCUGGAUUGCAA | TTTG | 619 |
| Human-Exon 6 | 16 | -1 | GCUGGAUUGCAACAAACCAACAGU | TTTC | 620 |
| Human-Exon 6 | 17 | -1 | CUGGAUUGCAACAAACCAACAGUG | TTTT | 621 |
| Human-Exon 6 | 18 | 1 | CCUAUGACUAUGGAUGAGAGCAUU | TTTG | 622 |
| Human-Exon 6 | 19 | -1 | UAGGUAAGAAGAUUACUGAGACAU | TTTA | 623 |
| Human-Exon 6 | 20 | -1 | AUUACUGAGACAUUAAAUAACUUG | TTTA | 624 |
| Human-Exon 6 | 21 | -1 | UUACUGAGACAUUAAAUAACUUGU | TTTT | 625 |
| Human-Exon 6 | 22 | 1 | GGGGAAAAAUAUGUCAUCAGAGUC | TTTA | 626 |
| Human-Exon 6 | 23 | 1 | CAUGAUCUGGAACCAUACUGGGGA | TTTT | 627 |
| Human-Exon 6 | 24 | 1 | ACAUGAUCUGGAACCAUACUGGGG | TTTT | 628 |
| Human-Exon 6 | 25 | 1 | GACAUGAUCUGGAACCAUACUGGG | TTTC | 629 |
| Human-Exon 7 | 1 | 1 | uacacacauacacaAAGACAAAUA | TTTA | 630 |
| Human-Exon 7 | 2 | 1 | uacacauacacacauacacaAAGA | TTTG | 631 |
| Human-Exon 7 | 3 | 1 | aacacauacacauacacacauaca | TTtg | 632 |
| Human-Exon 7 | 4 | 1 | AUUCCAGUCAAAUAGGUCUGGCCU | ttTT | 633 |
| Human-Exon 7 | 5 | 1 | UAUUCCAGUCAAAUAGGUCUGGCC | tTTA | 634 |
| Human-Exon 7 | 6 | 1 | GCUGGCAAACCACACUAUUCCAGU | TTTG | 635 |
| Human-Exon 7 | 7 | 1 | AGUCGUUGUGUGGCUGACUGCUGG | TTTG | 636 |
| Human-Exon 7 | 8 | -1 | CGCCAGAUAUCAAUUAGGCAUAGA | TTTC | 637 |
| Human-Exon 7 | 9 | -1 | AAACUACUCGAUCCUGAAGGUUGG | TTTA | 638 |
| Human-Exon 7 | 10 | 1 | CAUACUAAAAGCAGUGGUAGUCCA | TTTC | 639 |
| Human-Exon 7 | 11 | 1 | GAAAACAUUAAACUCUACCAUACU | TTTT | 640 |
| Human-Exon 7 | 12 | 1 | UGAAAACAUUAAACUCUACCAUAC | TTTA | 641 |
| Human-Exon 8 | 1 | -1 | UUGUUCAUUAUCCUUUUAGAGUCU | TTTG | 642 |
| Human-Exon 8 | 2 | 1 | AAAGGAUAAUGAACAAAUCAAAGU | TTTA | 643 |
| Human-Exon 8 | 3 | -1 | UAUCCUUUUAGAGUCUCAAAUAUA | TTTC | 644 |
| Human-Exon 8 | 4 | 1 | ACUCUAAAAGGAUAAUGAACAAAU | TTTG | 645 |
| Human-Exon 8 | 5 | -1 | UUUUAGAGUCUCAAAUAUAGAAAC | TTTG | 646 |
| Human-Exon 8 | 6 | -1 | UUUAGAGUCUCAAAUAUAGAAACC | TTTT | 647 |
| Human-Exon 8 | 7 | -1 | UUAGAGUCUCAAAUAUAGAAACCA | TTTT | 648 |
| Human-Exon 8 | 8 | 1 | UUGAGACUCUAAAAGGAUAAUGAA | TTTG | 649 |
| Human-Exon 8 | 9 | 1 | UUUGGUUUCUAUAUUUGAGACUCU | TTTT | 650 |
| Human-Exon 8 | 10 | 1 | UUUUGGUUUCUAUAUUUGAGACUC | TTTA | 651 |
| Human-Exon 8 | 11 | -1 | AGCAUUGAAGCCAUCCAGGAAGUG | TTTC | 652 |
| Human-Exon 8 | 12 | 1 | GCUUCAAUGCUCACUUGUUGAGGC | TTTT | 653 |
| Human-Exon 8 | 13 | 1 | GGCUUCAAUGCUCACUUGUUGAGG | TTTG | 654 |
| Human-Exon 8 | 14 | -1 | AGUGGAAAUGUUGCCAAGGCCACC | TTTA | 655 |
| Human-Exon 8 | 15 | -1 | GUUGCCAAGGCCACCUAAAGUGAC | TTTA | 656 |
| Human-Exon 8 | 16 | -1 | GAAGAACAUUUUCAGUUACAUCAU | TTTG | 657 |
| Human-Exon 8 | 17 | -1 | AUCAAAUGCACUAUUCUCAACAGG | TTTA | 658 |
| Human-Exon 8 | 18 | 1 | AUAGUGCAUUUGAUGAUGUAACUG | TTTT | 659 |
| Human-Exon 8 | 19 | 1 | AAUAGUGCAUUUGAUGAUGUAACU | TTTC | 660 |
| Human-Exon 8 | 20 | -1 | ACUAUUCUCAACAGGUAAAGUGUG | TTTA | 661 |
| Human-Exon 8 | 21 | 1 | UACCUAAAAAUGCAUAUAAAACAG | TTTT | 662 |
| Human-Exon 8 | 22 | 1 | AUACCUAAAAAUGCAUAUAAAACA | TTTC | 663 |
| Human-Exon 8 | 23 | 1 | CACGUAAUACCUAAAAAUGCAUAU | TTTT | 664 |
| Human-Exon 8 | 24 | 1 | GCACGUAAUACCUAAAAAUGCAUA | TTTA | 665 |
| Human-Exon 8 | 25 | 1 | auauauauGUGCACGUAAUACCUA | TTTT | 666 |
| Human-Exon 8 | 26 | 1 | uauauauauGUGCACGUAAUACCU | TTTT | 667 |
| Human-Exon 8 | 27 | 1 | auauauauauGUGCACGUAAUACC | TTTA | 668 |
| Human-Exon 55 | 1 | -1 | CUGCACAAUAUUAUAGUUGUUGCU | TTTA | 669 |
| Human-Exon 55 | 2 | 1 | AUAAAAAGAGAAAGAUGGAGGAAC | TTTA | 670 |
| Human-Exon 55 | 3 | 1 | CACCUAGUGAACUCCAUAAAAAGA | TTTC | 671 |
| Human-Exon 55 | 4 | 1 | AUGGUGCACCUAGUGAACUCCAUA | TTTT | 672 |
| Human-Exon 55 | 5 | 1 | AAUGGUGCACCUAGUGAACUCCAU | TTTT | 673 |
| Human-Exon 55 | 6 | 1 | GAAUGGUGCACCUAGUGAACUCCA | TTTA | 674 |
| Human-Exon 55 | 7 | 1 | GACCAAAUGUUCAGAUGCAAUUAU | TTTA | 675 |
| Human-Exon 55 | 8 | 1 | UCGCUCACUCACCCUGCAAAGGAC | TTTG | 676 |
| Human-Exon 55 | 9 | -1 | AGUGAGCGAGAGGCUGCUUUGGAA | TTTC | 677 |
| Human-Exon 55 | 10 | 1 | GCAGCCUCUCGCUCACUCACCCUG | TTTG | 678 |
| Human-Exon 55 | 11 | 1 | UUGCAGUAAUCUAUGAGUUUCUUC | TTTG | 679 |
| Human-Exon 55 | 12 | -1 | CUGCAACAGUUCCCCCUGGACCUG | TTTC | 680 |
| Human-Exon 55 | 13 | -1 | UGCAACAGUUCCCCCUGGACCUGG | TTTT | 681 |
| Human-Exon 55 | 14 | -1 | UUUCUUGCCUGGCUUACAGAAGCU | TTTC | 682 |
| Human-Exon 55 | 15 | 1 | UUUCAGCUUCUGUAAGCCAGGCAA | TTTC | 683 |
| Human-Exon 55 | 16 | -1 | GUCCUACAGGAUGCUACCCGUAAG | TTTC | 684 |
| Human-Exon 55 | 17 | -1 | GGCUCCUAGAAGACUCCAAGGGAG | TTTA | 685 |
| Human-Exon 55 | 18 | -1 | GCUCCUAGAAGACUCCAAGGGAGU | TTTT | 686 |
| Human-Exon 55 | 19 | -1 | CUCCAAGGGAGUAAAAGAGCUGAU | TTTC | 687 |
| Human-Exon 55 | 20 | 1 | UGGAUCCACAAGAGUGCUAAAGCG | TTTC | 688 |
| Human-Exon 55 | 21 | 1 | GUUCAAUUGGAUCCACAAGAGUGC | TTTA | 689 |
| Human-Exon 55 | 22 | -1 | UACUUGUAACUGACAAGCCAGGGA | TTTG | 690 |
| Human-Exon 55 | 23 | -1 | ACUUGUAACUGACAAGCCAGGGAC | TTTT | 691 |
| Human-Exon 55 | 24 | -1 | GUAACUGACAAGCCAGGGACAAAA | TTTG | 692 |
| Human-Exon 55 | 25 | -1 | UAACUGACAAGCCAGGGACAAAAC | TTTT | 693 |
| Human-Exon 55 | 26 | 1 | UCCCUGGCUUGUCAGUUACAAGUA | TTTG | 694 |
| Human-G1-exon51 | | 1 | CAGAGUAACAGUCUGAGUAGGAGc | TTTA | 695 |
| Human-G2-exon51 | | 1 | uacuuuguuuagcaauacauggua | TTTC | 696 |
| Human-G3-exon51 | | -1 | uggcucaaauuguuacucuucaau | TTTA | 697 |
| mouse-Exon23-G1 | | 1 | CUUUCAAagaacuuugcagagccu | TTTG | 698 |
| mouse-Exon23-G2 | | 1 | guugaaGCCAUUUUGUUGCUCUUU | TTTG | 699 |
| mouse-Exon23-G3 | | 1 | guugaaGCCAUUUUAUUGCUCUUU | TTTG | 700 |
| mouse-Exon23-G4 | | -1 | uuuugagGCUCUGCAAAGUUCUUU | TTTC | 701 |
| mouse-Exon23-G5 | | -1 | aguuauuaaugcauagauauucag | TTTA | 702 |
| mouse-Exon23-G6 | | -1 | uauaauaugcccuguaauauaaua | TTTC | 703 |
| mouse-Exon23-G7 | | 1 | uaaaggccaaaccucggcuuaccU | TTTC | 704 |
| mouse-Exon23-G8 | | 1 | ucaauaucuuugaaggacucuggg | TTTA | 705 |

*In this table, upper case letters represent sgRNA nucleotides that align to the exon sequence of the gene. Lower case letters represent sgRNA nucleotides that align to the intron sequence of the gene.

TABLE 8
Genomic target sites for sgRNA in mouse Dmd
Exon 51

| | | | SEQ ID | |
|---|---|---|---|---|
| ID sgRNA | Strand | Target site | NO: | PAM |
| Ex51-SA1 | 3′ | AGAGTAACAGTCTGACTGG | 706 | CAG |
| Ex51-SD | 5′ | GAAATGATCATCAAACAGA | 707 | AGG |
| Ex51-SA-2 | 3′ | CACTAGAGTAACAGTCTGAC | 708 | TGG |

TABLE 9
gRNA sequences targeting mouse Dmd Exon 51

| | | | SEQ ID | |
|---|---|---|---|---|
| ID sgRNA | Strand | Target site | NO: | PAM |
| Ex51-SA1 | 3′ | CCAGUCAGACUGUUACUCU | 709 | CAG |
| Ex51-SD | 5′ | UCUGUUUGAUGAUCAUUUC | 710 | AGG |
| Ex51-SA-2 | 3′ | GUCAGACUGUUACUCUAGUG | 711 | TGG |

TABLE 10
Genomic target sequences for sgRNAs targeting
human Dmd Exon 51

| | | | SEQ ID | |
|---|---|---|---|---|
| ID sgRNA | Strand | Target site | NO: | PAM |
| Ex51-SA | 3 | AGAGTAACAGTCTGAGTAG | 712 | GAG |
| Ex51-SD | 5′ | GAGATGATCATCAAGCAGA | 713 | AGG |
| Ex51-SA-2 | 3′ | CACCAGAGTAACAGTCTGAG | 714 | TAG |

TABLE 11
sgRNA sequences targeting human Dmd Exon 51

| | | | SEQ ID | |
|---|---|---|---|---|
| ID sgRNA | Strand | Target site | NO: | PAM |
| Ex51-SA | 3′ | CUACUCAGACUGUUACUCU | 715 | GAG |
| Ex51-SD | 5′ | UCUGCUUGAUGAUCAUCUC | 716 | AGG |
| Ex51-SA-2 | 3′ | CUCAGACUGUUACUCUGGUG | 717 | TAG |

TABLE 12
Genomic target sequences for sgRNAs targeting
sites in various human Dmd Exons

| | | | SEQ | |
|---|---|---|---|---|
| | | | ID | |
| ID sgRNA | Strand | Target site | NO: | PAM |
| Exon51-#1 | 3′ | CAGAGTAACAGTCTGAGTAG | 947 | GAG |
| Exon51-#2 | 3′ | CACCAGAGTAACAGTCTGAG | 718 | TAG |
| Exon51-#3 | 3′ | TATTTTGGGTTTTTGCAAAA | 719 | AGG |
| Exon51-#4 | 3′ | AGTAGGAGCTAAAATATTTT | 720 | GGG |
| Exon51-#5 | 3′ | GAGTAGGAGCTAAAATATTT | 721 | TGG |
| Exon51-#6 | 3′ | ACCAGAGTAACAGTCTGAGT | 722 | AGG |
| Exon51-#7 | 5′ | TCCTACTCAGACTGTTACTC | 723 | TGG |
| Exon51-#8 | 5′ | TACTCTGGTGACACAACCTG | 724 | TGG |
| Exon51-#9 | 3′ | GCAGTTTCCTTAGTAACCAC | 725 | AGG |
| Exon51-#10 | 5′ | GACACAACCTGTGGTTACTA | 726 | AGG |
| Exon51-#11 | 3′ | TGTCACCAGAGTAACAGTCT | 727 | GAG |
| Exon51-#12 | 3′ | AGGTTGTGTCACCAGAGTAA | 728 | CAG |
| Exon51-#13 | 3′ | AACCACAGGTTGTGTCACCA | 729 | GAG |
| Exon51-#14 | 3′ | GTAACCACAGGTTGTGTCAC | 730 | CAG |
| Exon53-#1 | 5′ | ATTTATTTTTCCTTTTATTC | 731 | TAG |
| Exon53-#2 | 5′ | TTTCCTTTTATTCTAGTTGA | 732 | AAG |
| Exon53-#3 | 3′ | TGATTCTGAATTCTTTCAAC | 733 | TAG |
| Exon53-#4 | 3′ | AATTCTTTCAACTAGAATAA | 734 | AAG |
| Exon53-#6 | 5′ | TTATTCTAGTTGAAAGAATT | 735 | CAG |
| Exon53-#7 | 5′ | TAGTTGAAAGAATTCAGAAT | 736 | CAG |
| Exon53-#8 | 5′ | AATTCAGAATCAGTGGGATG | 737 | AAG |
| Exon53-#9 | 3′ | ATTCTTTCAACTAGAATAAA | 738 | AGG |
| Exon53-#10 | 5′ | TTGAAAGAATTCAGAATCAG | 739 | TGG |
| Exon53-#11 | 5′ | TGAAAGAATTCAGAATCAGT | 740 | GGG |
| Exon53-#12 | 3′ | ACTGTTGCCTCCGGTTCTGA | 741 | AGG |
| Exon44-#1 | 3′ | CAGATCTGTCAAATCGCCTG | 742 | CAG |
| Exon44-#2 | 3′ | AAAACGCCGCCATTTCTCAA | 743 | CAG |
| Exon44-#3 | 3′ | AGATCTGTCAAATCGCCTGC | 744 | AGG |
| Exon44-#4 | 3′ | TATGGATCAAGAAAAATAGA | 745 | TGG |
| Exon44-#5 | 3′ | CGCCTGCAGGTAAAAGCATA | 746 | TGG |
| Exon44-#6 | 5′ | ATCCATATGCTTTTACCTGC | 747 | AGG |
| Exon44-#8 | 5′ | TTGACAGATCTGTTGAGAAA | 748 | TGG |
| Exon44-#9 | 5′ | ACAGATCTGTTGAGAAATGG | 749 | CGG |
| Exon44-#11 | 5′ | GGCGATTTGACAGATCTGTT | 750 | GAG |
| Exon44-#13 | 5′ | GGCGTTTTCATTATGATATA | 751 | AAG |
| Exon44-#14 | 5′ | ATGATATAAAGATATTTAAT | 752 | CAG |
| Exon44-#15 | 5′ | GATATTTAATCAGTGGCTAA | 753 | CAG |
| Exon44-#16 | 5′ | ATTTAATCAGTGGCTAACAG | 754 | AAG |
| Exon44-#17 | 3′ | AGAAACTGTTCAGCTTCTGT | 755 | TAG |
| Exon43-#1 | 5′ | GTTTTAAAATTTTTATATTA | 756 | CAG |
| Exon43-#2 | 5′ | TTTTATATTACAGAATATAA | 757 | AAG |
| Exon43-#3 | 5′ | ATATTACAGAATATAAAAGA | 758 | TAG |
| Exon45-#1 | 3′ | GTTCCTGTAAGATACCAAAA | 759 | AGG |
| Exon45-#2 | 5′ | TTGCCTTTTTGGTATCTTAC | 760 | AGG |
| Exon45-#3 | 5′ | TGGTATCTTACAGGAACTCC | 761 | AGG |
| Exon45-#4 | 5′ | ATCTTACAGGAACTCCAGGA | 762 | TGG |
| Exon45-#5 | 3′ | GCCGCTGCCCAATGCCATCC | 763 | TGG |
| Exon45-#6 | 5′ | CAGGAACTCCAGGATGGCAT | 764 | TGG |
| Exon45-#7 | 5′ | AGGAACTCCAGGATGGCATT | 765 | GGG |
| Exon45-#8 | 5′ | TCCAGGATGGCATTGGGCAG | 766 | CGG |
| Exon45-#9 | 5′ | GTCAGAACATTGAATGCAAC | 767 | TGG |
| Exon45-#10 | 3′ | AGTTCCTGTAAGATACCAAA | 768 | AAG |
| Exon45-#11 | 3′ | TGCCATCCTGGAGTTCCTGT | 769 | AAG |
| Exon45-#12 | 5′ | TTGGTATCTTACAGGAACTC | 770 | CAG |
| Exon45-#13 | 3′ | CGCTGCCCAATGCCATCCTG | 771 | GAG |
| Exon45-#14 | 5′ | AACTCCAGGATGGCATTGGG | 772 | CAG |
| Exon45-#15 | 5′ | GGGCAGCGGCAAACTGTTGT | 773 | CAG |
| Exon52-#1 | 3′ | AGATCTGTCAAATCGCCTGC | 774 | AGG |
| Exon52-#2 | 3′ | AATCCTGCATTGTTGCCTGT | 775 | AAG |
| Exon52-#3 | 5′ | CGCTGAAGAACCCTGATACT | 776 | AAG |
| Exon52-#4 | 3′ | GAACAAATATCCCTTAGTAT | 777 | CAG |
| Exon52-#5 | 3′ | CTGTAAGAACAAATATCCCT | 778 | TAG |
| Exon52-#6 | 5′ | CTAAGGGATATTTGTTCTTA | 779 | CAG |
| Exon52-#8 | 5′ | TGTTCTTACAGGCAACAATG | 780 | CAG |
| Exon52-#9 | 5′ | CAACAATGCAGGATTTGGAA | 781 | CAG |
| Exon52-#10 | 5′ | ACAATGCAGGATTTGGAACA | 782 | GAG |
| Exon52-#11 | 5′ | ATTTGGAACAGAGGCGTCCC | 783 | CAG |
| Exon52-#12 | 5′ | ACAGAGGCGTCCCCAGTTGG | 784 | AAG |
| Exon2-#1 | 5′ | TATTTTTTTATTTTGCATTT | 785 | TAG |
| Exon2-#2 | 5′ | TTATTTTGCATTTTAGATGA | 786 | AAG |
| Exon2-#3 | 5′ | ATTTTGCATTTTAGATGAAA | 787 | GAG |
| Exon2-#4 | 5′ | TTGCATTTTAGATGAAAGAG | 788 | AAG |
| Exon2-#5 | 5′ | ATGAAAGAGAAGATGTTCAA | 789 | AAG |

TABLE 13
gRNA sequences for targeting sites in various
human Dmd Exons

| | | | SEQ | |
|---|---|---|---|---|
| | | | ID | |
| ID sgRNA | Strand | Target site | NO: | PAM |
| Exon51-#1 | 3′ | CUACUCAGACUGUUACUCUG | 790 | GAG |
| Exon51-#2 | 3′ | CUCAGACUGUUACUCUGGUG | 791 | TAG |
| Exon51-#3 | 3′ | UUUUGCAAAAACCCAAAAUA | 792 | AGG |
| Exon51-#4 | 3′ | AAAAUAUUUUAGCUCCUACU | 793 | GGG |
| Exon51-#5 | 3′ | AAAUAUUUUAGCUCCUACUC | 794 | TGG |
| Exon51-#6 | 3′ | ACUCAGACUGUUACUCUGGU | 795 | AGG |
| Exon51-#7 | 5′ | GAGUAACAGUCUGAGUAGGA | 796 | TGG |
| Exon51-#8 | 5′ | CAGGUUGUGUCACCAGAGUA | 797 | TGG |
| Exon51-#9 | 3′ | GUGGUUACUAAGGAAACUGC | 798 | AGG |
| Exon51-#10 | 5′ | UAGUAACCACAGGUUGUGUC | 799 | AGG |
| Exon51-#11 | 3′ | AGACUGUUACUCUGGUGACA | 800 | GAG |
| Exon51-#12 | 3′ | UUACUCUGGUGACACAACCU | 801 | CAG |
| Exon51-#13 | 3′ | UGGUGACACAACCUGUGGUU | 802 | GAG |
| Exon51-#14 | 3′ | GUGACACAACCUGUGGUUAC | 803 | CAG |
| Exon53-#1 | 5′ | GAAUAAAAGGAAAAAUAAAU | 804 | TAG |
| Exon53-#2 | 5′ | UCAACUAGAAUAAAAGGAAA | 805 | AAG |
| Exon53-#3 | 3′ | GUUGAAAGAAUUCAGAAUCA | 806 | TAG |
| Exon53-#4 | 3′ | UUAUUCUAGUUGAAAGAAUU | 807 | AAG |
| Exon53-#6 | 5′ | AAUUCUUUCAACUAGAAUAA | 808 | CAG |
| Exon53-#7 | 5′ | AUUCUGAAUUCUUUCAACUA | 809 | CAG |
| Exon53-#8 | 5′ | CAUCCCACUGAUUCUGAAUU | 810 | AAG |
| Exon53-#9 | 3′ | UUUAUUCUAGUUGAAAGAAU | 811 | AGG |
| Exon53-#10 | 5′ | CUGAUUCUGAAUUCUUUCAA | 812 | TGG |
| Exon53-#11 | 5′ | ACUGAUUCUGAAUUCUUUCA | 813 | GGG |
| Exon53-#12 | 3′ | UCAGAACCGGAGGCAACAGU | 814 | AGG |
| Exon44-#1 | 3′ | CAGGCGAUUUGACAGAUCUG | 815 | CAG |
| Exon44-#2 | 3′ | UUGAGAAAUGGCGGCGUUUU | 816 | CAG |
| Exon44-#3 | 3′ | GCAGGCGAUUUGACAGAUCU | 817 | AGG |
| Exon44-#4 | 3′ | UCUAUUUUUCUUGAUCCAUA | 818 | TGG |
| Exon44-#5 | 3′ | UAUGCUUUUACCUGCAGGCG | 819 | TGG |
| Exon44-#6 | 5′ | GCAGGUAAAAGCAUAUGGAU | 820 | AGG |
| Exon44-#8 | 5′ | UUUCUCAACAGAUCUGUCAA | 821 | TGG |
| Exon44-#9 | 5′ | CCAUUUCUCAACAGAUCUGU | 822 | CGG |
| Exon44-#11 | 5′ | AACAGAUCUGUCAAAUCGCC | 823 | GAG |
| Exon44-#13 | 5′ | UAUAUCAUAAUGAAAACGCC | 824 | AAG |
| Exon44-#14 | 5′ | AUUAAAUAUCUUUAUAUCAU | 825 | CAG |
| Exon44-#15 | 5′ | UUAGCCACUGAUUAAAUAUC | 826 | CAG |
| Exon44-#16 | 5′ | CUGUUAGCCACUGAUUAAAU | 827 | AAG |
| Exon44-#17 | 3′ | ACAGAAGCUGAACAGUUUCU | 828 | TAG |
| Exon43-#1 | 5′ | UAAUAUAAAAAUUUUAAAAC | 829 | CAG |
| Exon43-#2 | 5′ | UUAUAUUCUGUAAUAUAAAA | 830 | AAG |
| Exon43-#3 | 5′ | UCUUUUAUAUUCUGUAAUAU | 831 | TAG |
| Exon45-#1 | 3′ | UUUUGGUAUCUUACAGGAAC | 832 | AGG |
| Exon45-#2 | 5′ | GUAAGAUACCAAAAAGGCAA | 833 | AGG |
| Exon45-#3 | 5′ | GGAGUUCCUGUAAGAUACCA | 834 | AGG |
| Exon45-#4 | 5′ | UCCUGGAGUUCCUGUAAGAU | 835 | TGG |
| Exon45-#5 | 3′ | GGAUGGCAUUGGGCAGCGGC | 836 | TGG |
| Exon45-#6 | 5′ | AUGCCAUCCUGGAGUUCCUG | 837 | TGG |
| Exon45-#7 | 5′ | AAUGCCAUCCUGGAGUUCCU | 838 | GGG |
| Exon45-#8 | 5′ | CUGCCCAAUGCCAUCCUGGA | 839 | CGG |
| Exon45-#9 | 5′ | GUUGCAUUCAAUGUUCUGAC | 840 | TGG |
| Exon45-#10 | 3′ | UUUGGUAUCUUACAGGAACU | 841 | AAG |
| Exon45-#11 | 3′ | ACAGGAACUCCAGGAUGGCA | 842 | AAG |
| Exon45-#12 | 5′ | GAGUUCCUGUAAGAUACCAA | 843 | CAG |
| Exon45-#13 | 3′ | CAGGAUGGCAUUGGGCAGCG | 844 | GAG |
| Exon45-#14 | 5′ | CCCAAUGCCAUCCUGGAGUU | 845 | CAG |
| Exon45-#15 | 5′ | ACAACAGUUUGCCGCUGCCC | 846 | CAG |
| Exon52-#1 | 3′ | GCAGGCGAUUUGACAGAUCU | 847 | AGG |
| Exon52-#2 | 3′ | ACAGGCAACAAUGCAGGAUU | 848 | AAG |
| Exon52-#3 | 5′ | AGUAUCAGGGUUCUUCAGCG | 849 | AAG |
| Exon52-#4 | 3′ | AUACUAAGGGAUAUUUGUUC | 850 | CAG |
| Exon52-#5 | 3′ | AGGGAUAUUUGUUCUUACAG | 851 | TAG |
| Exon52-#6 | 5′ | UAAGAACAAAUAUCCCUUAG | 852 | CAG |
| Exon52-#8 | 5′ | CAUUGUUGCCUGUAAGAACA | 853 | CAG |
| Exon52-#9 | 5′ | UUCCAAAUCCUGCAUUGUUG | 854 | CAG |
| Exon52-#10 | 5′ | UGUUCCAAAUCCUGCAUUGU | 855 | GAG |
| Exon52-#11 | 5′ | GGGACGCCUCUGUUCCAAAU | 856 | CAG |
| Exon52-#12 | 5′ | CCAACUGGGGACGCCUCUGU | 857 | AAG |
| Exon2-#1 | 5′ | ACAGAGGCGUCCCCAGUUGG | 858 | TAG |
| Exon2-#2 | 5′ | UCAUCUAAAAUGCAAAAUAA | 859 | AAG |
| Exon2-#3 | 5′ | UUUCAUCUAAAAUGCAAAAU | 860 | GAG |
| Exon2-#4 | 5′ | CUCUUUCAUCUAAAAUGCAA | 861 | AAG |
| Exon2-#5 | 5′ | UUGAACAUCUUCUCUUUCAU | 862 | AAG |

TABLE 14
Genomic targeting sequence for sgRNAs targeting
dog Dmd Exon 51

| | | | SEQ ID | |
|---|---|---|---|---|
| ID sgRNA | Strand | Target site | NO: | PAM |
| Ex51-SA-2 | 3′ | CACCAGAGTAACAGTCTGAC | 863 | TGG |

TABLE 15
gRNA sequence for targeting dog Dmd Exon 51

| | | | SEQ ID | |
|---|---|---|---|---|
| ID sgRNA | Strand | Target site | NO: | PAM |
| Ex51-SA-2 | 3′ | GUCAGACUGUUACUCUGGUG | 864 | TGG |

TABLE 16
Exon 43 & 45 gRNA sequences

| sgRNA ID | Sequence (5′-3′) | SEQ ID NO. |
|---|---|---|
| Ex45-gRNA#3 | CGCTGCCCAATGCCATCCTG | 948 |
| Ex45-gRNA#4 | ATCTTACAGGAACTCCAGGA | 949 |
| Ex45-gRNA#5 | AGGAACTCCAGGATGGCATT | 950 |
| Ex45-gRNA#6 | CGCTGCCCAATGCCATCC | 951 |
| Ex43-gRNA#1 | GTTTTAAAATTTTTATATTA | 952 |
| Ex43-gRNA#2 | TTTTATATTACAGAATATAA | 953 |
| Ex43-gRNA#4 | TATGTGTTACCTACCCTTGT | 954 |
| Ex43-gRNA#6 | GTACAAGGACCGACAAGGGT | 955 |

TABLE 17
Exon 43 & 45 gRNA sequences

| sgRNA ID | Sequence (5′-3′) | SEQ ID NO. |
|---|---|---|
| Ex45-gRNA#3 | CAGGAUGGCAUUGGGCAGCG | 956 |
| Ex45-gRNA#4 | UCCUGGAGUUCCUGUAAGAU | 957 |
| Ex45-gRNA#5 | AAUGCCAUCCUGGAGUUCCU | 958 |
| Ex45-gRNA#6 | GGAUGGCAUUGGGCAGCG | 959 |
| Ex43-gRNA#1 | UAAUAUAAAAAUUUUAAAAC | 960 |
| Ex43-gRNA#2 | UUAUAUUCUGUAAUAUAAAA | 961 |
| Ex43-gRNA#4 | ACAAGGGUAGGUAACACAUA | 962 |
| Ex43-gRNA#6 | ACCCUUGUCGGUCCUUGUAC | 963 |
| Ex45-gRNA#3′ | CGCUGCCCAAUGCCAUCCUG | 964 |
| Ex45-gRNA#4′ | AUCUUACAGGAACUCCAGGA | 965 |
| Ex45-gRNA#5′ | AGGAACUCCAGGAUGGCAUU | 966 |
| Ex45-gRNA#6′ | CGCUGCCCAAUGCCAUCC | 967 |
| Ex43-gRNA#1′ | GUUUUAAAAUUUUUAUAUUA | 968 |
| Ex43-gRNA#2′ | UUUUAUAUUACAGAAUAUAA | 969 |
| Ex43-gRNA#4′ | UAUGUGUUACCUACCCUUGU | 970 |
| Ex43-gRNA#6′ | GUACAAGGACCGACAAGGGU | 971 |

TABLE 18
gRNA sequences

| Targeted | | | | | | | |
|---|---|---|---|---|---|---|---|
| gRNA | Guide | | | | SEQ ID | | SEQ ID |
| Exon | # | Strand | PAM | DNA sequence* | NO. | RNA sequence* | NO. |
| Human- | 4 | 1 | tttt | tctttttcttcttttttccttttt | 972 | ucuuuuucuucuuuuuuccuuuuu | 1305 |
| Exon 51 | | | | | | | |
| Human- | 5 | 1 | tttt | ctttttcttcttttttcctttttG | 973 | cuuuuucuucuuuuuuccuuuuuG | 1306 |
| Exon 51 | | | | | | | |
| Human- | 6 | 1 | tttc | tttttcttcttttttcctttttGC | 974 | uuuuucuucuuuuuuccuuuuuGC | 1307 |
| Exon 51 | | | | | | | |
| Human- | 7 | 1 | tttt | tcttcttttttcctttttGCAAAA | 975 | ucuucuuuuuuccuuuuuGCAAAA | 1308 |
| Exon 51 | | | | | | | |
| Human- | 8 | 1 | tttt | cttcttttttcctttttGCAAAAA | 976 | cuucuuuuuuccuuuuuGCAAAAA | 1309 |
| Exon 51 | | | | | | | |
| Human- | 9 | 1 | tttc | ttcttttttcctttttGCAAAAAC | 977 | uucuuuuuuccuuuuuGCAAAAAC | 1310 |
| Exon 51 | | | | | | | |
| Human- | 10 | 1 | tttt | ttcctttttGCAAAAACCCAAAAT | 978 | uuccuuuuuGCAAAAACCCAAAAU | 1311 |
| Exon 51 | | | | | | | |
| Human- | 11 | 1 | tttt | tcctttttGCAAAAACCCAAAATA | 979 | uccuuuuuGCAAAAACCCAAAAUA | 1312 |
| Exon 51 | | | | | | | |
| Human- | 12 | 1 | tttt | cctttttGCAAAAACCCAAAATAT | 980 | ccuuuuuGCAAAAACCCAAAAUAU | 1313 |
| Exon 51 | | | | | | | |
| Human- | 13 | 1 | tttc | ctttttGCAAAAACCCAAAATATT | 981 | cuuuuuGCAAAAACCCAAAAUAUU | 1314 |
| Exon 51 | | | | | | | |
| Human- | 14 | 1 | tttt | tGCAAAAACCCAAAATATTTTAGC | 982 | uGCAAAAACCCAAAAUAUUUUAGC | 1315 |
| Exon 51 | | | | | | | |
| Human- | 15 | 1 | tttt | GCAAAAACCCAAAATATTTTAGCT | 983 | GCAAAAACCCAAAAUAUUUUAGCU | 1316 |
| Exon 51 | | | | | | | |
| Human- | 16 | 1 | tttG | CAAAAACCCAAAATATTTTAGCTC | 984 | CAAAAACCCAAAAUAUUUUAGCUC | 1317 |
| Exon 51 | | | | | | | |
| Human- | 17 | 1 | TTTT | AGCTCCTACTCAGACTGTTACTCT | 985 | AGCUCCUACUCAGACUGUUACUCU | 1318 |
| Exon 51 | | | | | | | |
| Human- | 18 | 1 | TTTA | GCTCCTACTCAGACTGTTACTCTG | 986 | GCUCCUACUCAGACUGUUACUCUG | 1319 |
| Exon 51 | | | | | | | |
| Human- | 19 | -1 | TTTC | CTTAGTAACCACAGGTTGTGTCAC | 987 | CUUAGUAACCACAGGUUGUGUCAC | 1320 |
| Exon 51 | | | | | | | |
| Human- | 20 | -1 | TTTG | GAGATGGCAGTTTCCTTAGTAACC | 988 | GAGAUGGCAGUUUCCUUAGUAACC | 1321 |
| Exon 51 | | | | | | | |
| Human- | 21 | -1 | TTTC | TAGTTTGGAGATGGCAGTTTCCTT | 999 | UAGUUUGGAGAUGGCAGUUUCCUU | 1322 |
| Exon 51 | | | | | | | |
| Human- | 22 | -1 | TTTT | TTCTCATACCTTCTGCTTGATGAT | 1000 | UUCUCAUACCUUCUGCUUGAUGAU | 1323 |
| Exon 51 | | | | | | | |
| Human- | 23 | -1 | TTTA | TCATTTTTTCTCATACCTTCTGCT | 1001 | UCAUUUUUUCUCAUACCUUCUGCU | 1324 |
| Exon 51 | | | | | | | |
| Human- | 24 | -1 | TTTT | ATCATTTTTTCTCATACCTTCTGC | 1002 | AUCAUUUUUUCUCAUACCUUCUGC | 1325 |
| Exon 51 | | | | | | | |
| Human- | 25 | -1 | TTTA | AAGAAAAACTTCTGCCAACTTTTA | 1003 | AAGAAAAACUUCUGCCAACUUUUA | 1326 |
| Exon 51 | | | | | | | |
| Human- | 26 | -1 | TTTT | AAAGAAAAACTTCTGCCAACTTTT | 1004 | AAAGAAAAACUUCUGCCAACUUUU | 1327 |
| Exon 51 | | | | | | | |
| Human- | 27 | 1 | TTTT | TCTTTAAAATGAAGATTTTCCACC | 1005 | UCUUUAAAAUGAAGAUUUUCCACC | 1328 |
| Exon 51 | | | | | | | |
| Human- | 28 | 1 | TTTT | CTTTAAAATGAAGATTTTCCACCA | 1006 | CUUUAAAAUGAAGAUUUUCCACCA | 1329 |
| Exon 51 | | | | | | | |
| Human- | 29 | 1 | TTTC | TTTAAAATGAAGATTTTCCACCAA | 1007 | UUUAAAAUGAAGAUUUUCCACCAA | 1330 |
| Exon 51 | | | | | | | |
| Human- | 30 | 1 | TTTA | AAATGAAGATTTTCCACCAATCAC | 1008 | AAAUGAAGAUUUUCCACCAAUCAC | 1331 |
| Exon 51 | | | | | | | |
| Human- | 31 | 1 | TTTT | CCACCAATCACTTTACTCTCCTAG | 1009 | CCACCAAUCACUUUACUCUCCUAG | 1332 |
| Exon 51 | | | | | | | |
| Human- | 32 | 1 | TTTC | CACCAATCACTTTACTCTCCTAGA | 1010 | CACCAAUCACUUUACUCUCCUAGA | 1333 |
| Exon 51 | | | | | | | |
| Human- | 33 | 1 | TTTA | CTCTCCTAGACCATTTCCCACCAG | 1011 | CUCUCCUAGACCAUUUCCCACCAG | 1334 |
| Exon 51 | | | | | | | |
| Human- | 1 | -1 | tttg | agaaaagattaaacagtgtgctac | 1012 | agaaaagauuaaacagugugcuac | 1335 |
| Exon 45 | | | | | | | |
| Human- | 2 | -1 | TTTa | tttgagaaaagattaaacagtgtg | 1013 | uuugagaaaagauuaaacagugug | 1336 |
| Exon 45 | | | | | | | |
| Human- | 3 | -1 | TTTT | atttgagaaaagattaaacagtgt | 1014 | auuugagaaaagauuaaacagugu | 1337 |
| Exon 45 | | | | | | | |
| Human- | 4 | -1 | TTTT | Tatttgagaaaagattaaacagtg | 1015 | Uauuugagaaaagauuaaacagug | 1338 |
| Exon 45 | | | | | | | |
| Human- | 5 | 1 | ttta | atcttttctcaaatAAAAAGACAT | 1016 | aucuuuucucaaauAAAAAGACAU | 1339 |
| Exon 45 | | | | | | | |
| Human- | 6 | 1 | tttt | ctcaaatAAAAAGACATGGGGCTT | 1017 | cucaaauAAAAAGACAUGGGGCUU | 1340 |
| Exon 45 | | | | | | | |
| Human- | 7 | 1 | tttc | tcaaatAAAAAGACATGGGGCTTC | 1018 | ucaaauAAAAAGACAUGGGGCUUC | 1341 |
| Exon 45 | | | | | | | |
| Human- | 8 | 1 | TTTT | TGTTTTGCCTTTTTGGTATCTTAC | 1019 | UGUUUUGCCUUUUUGGUAUCUUAC | 1342 |
| Exon 45 | | | | | | | |
| Human- | 9 | 1 | TTTT | GTTTTGCCTTTTTGGTATCTTACA | 1020 | GUUUUGCCUUUUUGGUAUCUUACA | 1343 |
| Exon 45 | | | | | | | |
| Human- | 10 | 1 | TTTG | TTTTGCCTTTTTGGTATCTTACAG | 1021 | UUUUGCCUUUUUGGUAUCUUACAG | 1344 |
| Exon 45 | | | | | | | |
| Human- | 11 | 1 | TTTT | GCCTTTTTGGTATCTTACAGGAAC | 1022 | GCCUUUUUGGUAUCUUACAGGAAC | 1345 |
| Exon 45 | | | | | | | |
| Human- | 12 | 1 | TTTG | CCTTTTTGGTATCTTACAGGAACT | 1023 | CCUUUUUGGUAUCUUACAGGAACU | 1346 |
| Exon 45 | | | | | | | |
| Human- | 13 | 1 | TTTT | TGGTATCTTACAGGAACTCCAGGA | 1024 | UGGUAUCUUACAGGAACUCCAGGA | 1347 |
| Exon 45 | | | | | | | |
| Human- | 14 | 1 | TTTT | GGTATCTTACAGGAACTCCAGGAT | 1025 | GGUAUCUUACAGGAACUCCAGGAU | 1348 |
| Exon 45 | | | | | | | |
| Human- | 15 | -1 | TTTG | AGGATTGCTGAATTATTTCTTCCC | 1026 | AGGAUUGCUGAAUUAUUUCUUCCC | 1349 |
| Exon 45 | | | | | | | |
| Human- | 16 | -1 | TTTT | GAGGATTGCTGAATTATTTCTTCC | 1027 | GAGGAUUGCUGAAUUAUUUCUUCC | 1350 |
| Exon 45 | | | | | | | |
| Human- | 17 | -1 | TTTT | TGAGGATTGCTGAATTATTTCTTC | 1028 | UGAGGAUUGCUGAAUUAUUUCUUC | 1351 |
| Exon 45 | | | | | | | |
| Human- | 18 | -1 | TTTC | CTGTAGAATACTGGCATCTGTTTT | 1029 | CUGUAGAAUACUGGCAUCUGUUUU | 1352 |
| Exon 45 | | | | | | | |
| Human- | 19 | -1 | TTTT | CCTGTAGAATACTGGCATCTGTTT | 1030 | CCUGUAGAAUACUGGCAUCUGUUU | 1353 |
| Exon 45 | | | | | | | |
| Human- | 20 | -1 | TTTT | TCCTGTAGAATACTGGCATCTGTT | 1031 | UCCUGUAGAAUACUGGCAUCUGUU | 1354 |
| Exon 45 | | | | | | | |
| Human- | 21 | -1 | TTTG | CAGACCTCCTGCCACCGCAGATTC | 1032 | CAGACCUCCUGCCACCGCAGAUUC | 1355 |
| Exon 45 | | | | | | | |
| Human- | 22 | -1 | TTTC | TGTCTGACAGCTGTTTGCAGACCT | 1033 | UGUCUGACAGCUGUUUGCAGACCU | 1356 |
| Exon 45 | | | | | | | |
| Human- | 23 | -1 | TTTT | CTGTCTGACAGCTGTTTGCAGACC | 1034 | CUGUCUGACAGCUGUUUGCAGACC | 1357 |
| Exon 45 | | | | | | | |
| Human- | 24 | -1 | TTTT | TCTGTCTGACAGCTGTTTGCAGAC | 1035 | UCUGUCUGACAGCUGUUUGCAGAC | 1358 |
| Exon 45 | | | | | | | |
| Human- | 25 | -1 | TTTT | TTCTGTCTGACAGCTGTTTGCAGA | 1036 | UUCUGUCUGACAGCUGUUUGCAGA | 1359 |
| Exon 45 | | | | | | | |
| Human- | 26 | -1 | TTTC | ATTCCTATTAGATCTGTCGCCCTA | 1037 | AUUCCUAUUAGAUCUGUCGCCCUA | 1360 |
| Exon 45 | | | | | | | |
| Human- | 27 | -1 | TTTT | CATTCCTATTAGATCTGTCGCCCT | 1038 | CAUUCCUAUUAGAUCUGUCGCCCU | 1361 |
| Exon 45 | | | | | | | |
| Human- | 28 | 1 | TTTT | AGCAGACTTTTTAAGCTTTCTTTA | 1039 | AGCAGACUUUUUAAGCUUUCUUUA | 1362 |
| Exon 45 | | | | | | | |
| Human- | 29 | 1 | TTTA | GCAGACTTTTTAAGCTTTCTTTAG | 1040 | GCAGACUUUUUAAGCUUUCUUUAG | 1363 |
| Exon 45 | | | | | | | |
| Human- | 30 | 1 | TTTT | TAAGCTTTCTTTAGAAGAATATTT | 1041 | UAAGCUUUCUUUAGAAGAAUAUUU | 1364 |
| Exon 45 | | | | | | | |
| Human- | 31 | 1 | TTTT | AAGCTTTCTTTAGAAGAATATTTC | 1042 | AAGCUUUCUUUAGAAGAAUAUUUC | 1365 |
| Exon 45 | | | | | | | |
| Human- | 32 | 1 | TTTA | AGCTTTCTTTAGAAGAATATTTCA | 1043 | AGCUUUCUUUAGAAGAAUAUUUCA | 1366 |
| Exon 45 | | | | | | | |
| Human- | 33 | 1 | TTTC | TTTAGAAGAATATTTCATGAGAGA | 1044 | UUUAGAAGAAUAUUUCAUGAGAGA | 1367 |
| Exon 45 | | | | | | | |
| Human- | 34 | 1 | TTTA | GAAGAATATTTCATGAGAGATTAT | 1045 | GAAGAAUAUUUCAUGAGAGAUUAU | 1368 |
| Exon 45 | | | | | | | |
| Human- | 1 | 1 | TTTG | TCAGTATAACCAAAAAATATACGC | 1046 | UCAGUAUAACCAAAAAAUAUACGC | 1369 |
| Exon 44 | | | | | | | |
| Human- | 2 | 1 | tttt | acataatccatctatttttcttga | 1047 | acauaauccaucuauuuuucuuga | 1370 |
| Exon 44 | | | | | | | |
| Human- | 3 | 1 | ttta | cataatccatctatttttcttgat | 1048 | cauaauccaucuauuuuucuugau | 1371 |
| Exon 44 | | | | | | | |
| Human- | 4 | 1 | tttt | tcttgatccatatgcttttACCTG | 1049 | ucuugauccauaugcuuuuACCUG | 1372 |
| Exon 44 | | | | | | | |
| Human- | 5 | 1 | tttt | cttgatccatatgcttttACCTGC | 1050 | cuugauccauaugcuuuuACCUGC | 1373 |
| Exon 44 | | | | | | | |
| Human- | 6 | 1 | tttc | ttgatccatatgcttttACCTGCA | 1051 | uugauccauaugcuuuuACCUGCA | 1374 |
| Exon 44 | | | | | | | |
| Human- | 7 | -1 | TTTC | TCAACAGATCTGTCAAATCGCCTG | 1052 | UCAACAGAUCUGUCAAAUCGCCUG | 1375 |
| Exon 44 | | | | | | | |
| Human- | 8 | 1 | tttt | ACCTGCAGGCGATTTGACAGATCT | 1053 | ACCUGCAGGCGAUUUGACAGAUCU | 1376 |
| Exon 44 | | | | | | | |
| Human- | 9 | 1 | tttA | CCTGCAGGCGATTTGACAGATCTG | 1054 | CCUGCAGGCGAUUUGACAGAUCUG | 1377 |
| Exon 44 | | | | | | | |
| Human- | 10 | 1 | TTTG | ACAGATCTGTTGAGAAATGGCGGC | 1055 | ACAGAUCUGUUGAGAAAUGGCGGC | 1378 |
| Exon 44 | | | | | | | |
| Human- | 11 | -1 | TTTA | TATCATAATGAAAACGCCGCCATT | 1056 | UAUCAUAAUGAAAACGCCGCCAUU | 1379 |
| Exon 44 | | | | | | | |
| Human- | 12 | 1 | TTTT | CATTATGATATAAAGATATTTAAT | 1057 | CAUUAUGAUAUAAAGAUAUUUAAU | 1380 |
| Exon 44 | | | | | | | |
| Human- | 13 | -1 | TTTG | TATTTAGCATGTTCCCAATTCTCA | 1058 | UAUUUAGCAUGUUCCCAAUUCUCA | 1381 |
| Exon 44 | | | | | | | |
| Human- | 14 | -1 | TTTC | GAAAAAACAAATCAAAGACTTACC | 1059 | GAAAAAACAAAUCAAAGACUUACC | 1382 |
| Exon 44 | | | | | | | |
| Human- | 15 | 1 | TTTG | ATTTGTTTTTTCGAAATTGTATTT | 1060 | AUUUGUUUUUUCGAAAUUGUAUUU | 1383 |
| Exon 44 | | | | | | | |
| Human- | 16 | 1 | TTTG | TTTTTTCGAAATTGTATTTATCTT | 1061 | UUUUUUCGAAAUUGUAUUUAUCUU | 1384 |
| Exon 44 | | | | | | | |
| Human- | 17 | 1 | TTTT | TTCGAAATTGTATTTATCTTCAGC | 1062 | UUCGAAAUUGUAUUUAUCUUCAGC | 1385 |
| Exon 44 | | | | | | | |
| Human- | 18 | 1 | TTTT | TCGAAATTGTATTTATCTTCAGCA | 1063 | UCGAAAUUGUAUUUAUCUUCAGCA | 1386 |
| Exon 44 | | | | | | | |
| Human- | 19 | 1 | TTTT | CGAAATTGTATTTATCTTCAGCAC | 1064 | CGAAAUUGUAUUUAUCUUCAGCAC | 1387 |
| Exon 44 | | | | | | | |
| Human- | 20 | 1 | TTTC | GAAATTGTATTTATCTTCAGCACA | 1065 | GAAAUUGUAUUUAUCUUCAGCACA | 1388 |
| Exon 44 | | | | | | | |
| Human- | 21 | -1 | TTTA | AGAAGTTAAAGAGTCCAGATGTGC | 1066 | AGAAGUUAAAGAGUCCAGAUGUGC | 1389 |
| Exon 44 | | | | | | | |
| Human- | 22 | 1 | TTTA | TCTTCAGCACATCTGGACTCTTTA | 1067 | UCUUCAGCACAUCUGGACUCUUUA | 1390 |
| Exon 44 | | | | | | | |
| Human- | 23 | -1 | TTTC | CATCACCCTTCAGAACCTGATCTT | 1068 | CAUCACCCUUCAGAACCUGAUCUU | 1391 |
| Exon 44 | | | | | | | |
| Human- | 24 | 1 | TTTA | ACTTCTTAAAGATCAGGTTCTGAA | 1069 | ACUUCUUAAAGAUCAGGUUCUGAA | 1392 |
| Exon 44 | | | | | | | |
| Human- | 25 | 1 | TTTT | GACTGTTGTTGTCATCATTATATT | 1070 | GACUGUUGUUGUCAUCAUUAUAUU | 1393 |
| Exon 44 | | | | | | | |
| Human- | 26 | 1 | TTTG | ACTGTTGTTGTCATCATTATATTA | 1071 | ACUGUUGUUGUCAUCAUUAUAUUA | 1394 |
| Exon 44 | | | | | | | |
| Human- | 1 | -1 | TTTC | AACTAGAATAAAAGGAAAAATAAA | 1072 | AACUAGAAUAAAAGGAAAAAUAAA | 1395 |
| Exon 53 | | | | | | | |
| Human- | 2 | 1 | TTTA | CTACTATATATTTATTTTTCCTTT | 1073 | CUACUAUAUAUUUAUUUUUCCUUU | 1396 |
| Exon 53 | | | | | | | |
| Human- | 3 | 1 | TTTA | TTTTTCCTTTTATTCTAGTTGAAA | 1074 | UUUUUCCUUUUAUUCUAGUUGAAA | 1397 |
| Exon 53 | | | | | | | |
| Human- | 4 | 1 | TTTT | TCCTTTTATTCTAGTTGAAAGAAT | 1075 | UCCUUUUAUUCUAGUUGAAAGAAU | 1398 |
| Exon 53 | | | | | | | |
| Human- | 5 | 1 | TTTT | CCTTTTATTCTAGTTGAAAGAATT | 1076 | CCUUUUAUUCUAGUUGAAAGAAUU | 1399 |
| Exon 53 | | | | | | | |
| Human- | 6 | 1 | TTTC | CTTTTATTCTAGTTGAAAGAATTC | 1077 | CUUUUAUUCUAGUUGAAAGAAUUC | 1400 |
| Exon 53 | | | | | | | |
| Human- | 7 | 1 | TTTT | ATTCTAGTTGAAAGAATTCAGAAT | 1078 | AUUCUAGUUGAAAGAAUUCAGAAU | 1401 |
| Exon 53 | | | | | | | |
| Human- | 8 | 1 | TTTA | TTCTAGTTGAAAGAATTCAGAATC | 1079 | UUCUAGUUGAAAGAAUUCAGAAUC | 1402 |
| Exon 53 | | | | | | | |
| Human- | 9 | -1 | TTTC | ATTCAACTGTTGCCTCCGGTTCTG | 1080 | AUUCAACUGUUGCCUCCGGUUCUG | 1403 |
| Exon 53 | | | | | | | |
| Human- | 10 | -1 | TTTA | ACATTTCATTCAACTGTTGCCTCC | 1081 | ACAUUUCAUUCAACUGUUGCCUCC | 1404 |
| Exon 53 | | | | | | | |
| Human- | 11 | -1 | TTTT | CTTTTGGATTGCATCTACTGTATA | 1082 | CUUUUGGAUUGCAUCUACUGUAUA | 1405 |
| Exon 53 | | | | | | | |
| Human- | 12 | -1 | TTTC | TGTGATTTTCTTTTGGATTGCATC | 1083 | UGUGAUUUUCUUUUGGAUUGCAUC | 1406 |
| Exon 53 | | | | | | | |
| Human- | 13 | -1 | TTTG | ATACTAACCTTGGTTTCTGTGATT | 1084 | AUACUAACCUUGGUUUCUGUGAUU | 1407 |
| Exon 53 | | | | | | | |
| Human- | 14 | -1 | TTTA | AAAAGGTATCTTTGATACTAACCT | 1085 | AAAAGGUAUCUUUGAUACUAACCU | 1408 |
| Exon 53 | | | | | | | |
| Human- | 15 | -1 | TTTT | AAAAAGGTATCTTTGATACTAACC | 1086 | AAAAAGGUAUCUUUGAUACUAACC | 1409 |
| Exon 53 | | | | | | | |
| Human- | 16 | -1 | TTTA | TTTTAAAAAGGTATCTTTGATACT | 1087 | UUUUAAAAAGGUAUCUUUGAUACU | 1410 |
| Exon 53 | | | | | | | |
| Human- | 17 | -1 | TTTT | ATTTTAAAAAGGTATCTTTGATAC | 1088 | AUUUUAAAAAGGUAUCUUUGAUAC | 1411 |
| Exon 53 | | | | | | | |
| Human- | 1 | -1 | TTTG | TTAATGCAAACTGGGACACAAACA | 1089 | UUAAUGCAAACUGGGACACAAACA | 1412 |
| Exon 46 | | | | | | | |
| Human- | 2 | 1 | TTTT | TAAATTGCCATGTTTGTGTCCCAG | 1090 | UAAAUUGCCAUGUUUGUGUCCCAG | 1413 |
| Exon 46 | | | | | | | |
| Human- | 3 | 1 | TTTT | AAATTGCCATGTTTGTGTCCCAGT | 1091 | AAAUUGCCAUGUUUGUGUCCCAGU | 1414 |
| Exon 46 | | | | | | | |
| Human- | 4 | 1 | TTTA | AATTGCCATGTTTGTGTCCCAGTT | 1092 | AAUUGCCAUGUUUGUGUCCCAGUU | 1415 |
| Exon 46 | | | | | | | |
| Human- | 5 | 1 | TTTG | TGTCCCAGTTTGCATTAACAAATA | 1093 | UGUCCCAGUUUGCAUUAACAAAUA | 1416 |
| Exon 46 | | | | | | | |
| Human- | 6 | -1 | tttC | CAACATAGTTCTCAAACTATTTGT | 1094 | CAACAUAGUUCUCAAACUAUUUGU | 1417 |
| Exon 46 | | | | | | | |
| Human- | 7 | -1 | tttt | CCAACATAGTTCTCAAACTATTTG | 1095 | CCAACAUAGUUCUCAAACUAUUUG | 1418 |
| Exon 46 | | | | | | | |
| Human- | 8 | -1 | tttt | tCCAACATAGTTCTCAAACTATTT | 1096 | uCCAACAUAGUUCUCAAACUAUUU | 1419 |
| Exon 46 | | | | | | | |
| Human- | 9 | -1 | tttt | tttCCAACATAGTTCTCAAACTAT | 1097 | uuuCCAACAUAGUUCUCAAACUAU | 1420 |
| Exon 46 | | | | | | | |
| Human- | 10 | -1 | tttt | ttttCCAACATAGTTCTCAAACTA | 1098 | uuuuCCAACAUAGUUCUCAAACUA | 1421 |
| Exon 46 | | | | | | | |
| Human- | 11 | -1 | tttt | tttttCCAACATAGTTCTCAAACT | 1099 | uuuuuCCAACAUAGUUCUCAAACU | 1422 |
| Exon 46 | | | | | | | |
| Human- | 12 | 1 | TTTG | CATTAACAAATAGTTTGAGAACTA | 1100 | CAUUAACAAAUAGUUUGAGAACUA | 1423 |
| Exon 46 | | | | | | | |
| Human- | 13 | 1 | TTTG | AGAACTATGTTGGaaaaaaaaaTA | 1101 | AGAACUAUGUUGGaaaaaaaaaUA | 1424 |
| Exon 46 | | | | | | | |
| Human- | 14 | -1 | TTTT | GTTCTTCTAGCCTGGAGAAAGAAG | 1102 | GUUCUUCUAGCCUGGAGAAAGAAG | 1425 |
| Exon 46 | | | | | | | |
| Human- | 15 | 1 | TTTT | ATTCTTCTTTCTCCAGGCTAGAAG | 1103 | AUUCUUCUUUCUCCAGGCUAGAAG | 1426 |
| Exon 46 | | | | | | | |
| Human- | 16 | 1 | TTTA | TTCTTCTTTCTCCAGGCTAGAAGA | 1104 | UUCUUCUUUCUCCAGGCUAGAAGA | 1427 |
| Exon 46 | | | | | | | |
| Human- | 17 | 1 | TTTC | TCCAGGCTAGAAGAACAAAAGAAT | 1105 | UCCAGGCUAGAAGAACAAAAGAAU | 1428 |
| Exon 46 | | | | | | | |
| Human- | 18 | -1 | TTTG | AAATTCTGACAAGATATTCTTTTG | 1106 | AAAUUCUGACAAGAUAUUCUUUUG | 1429 |
| Exon 46 | | | | | | | |
| Human- | 19 | -1 | TTTT | CTTTTAGTTGCTGCTCTTTTCCAG | 1107 | CUUUUAGUUGCUGCUCUUUUCCAG | 1430 |
| Exon 46 | | | | | | | |
| Human- | 20 | -1 | TTTG | AGAAAATAAAATTACCTTGACTTG | 1108 | AGAAAAUAAAAUUACCUUGACUUG | 1431 |
| Exon 46 | | | | | | | |
| Human- | 21 | -1 | TTTA | TGCAAGCAGGCCCTGGGGGATTTG | 1109 | UGCAAGCAGGCCCUGGGGGAUUUG | 1432 |
| Exon 46 | | | | | | | |
| Human- | 22 | 1 | TTTT | ATTTTCTCAAATCCCCCAGGGCCT | 1110 | AUUUUCUCAAAUCCCCCAGGGCCU | 1433 |
| Exon 46 | | | | | | | |
| Human- | 23 | 1 | TTTA | TTTTCTCAAATCCCCCAGGGCCTG | 1111 | UUUUCUCAAAUCCCCCAGGGCCUG | 1434 |
| Exon 46 | | | | | | | |
| Human- | 24 | 1 | TTTT | CTCAAATCCCCCAGGGCCTGCTTG | 1112 | CUCAAAUCCCCCAGGGCCUGCUUG | 1435 |
| Exon 46 | | | | | | | |
| Human- | 25 | 1 | TTTC | TCAAATCCCCCAGGGCCTGCTTGC | 1113 | UCAAAUCCCCCAGGGCCUGCUUGC | 1436 |
| Exon 46 | | | | | | | |
| Human- | 26 | 1 | TTTT | TTAATTCAATCATTGGTTTTCTGC | 1114 | UUAAUUCAAUCAUUGGUUUUCUGC | 1437 |
| Exon 46 | | | | | | | |
| Human- | 27 | 1 | TTTT | TAATTCAATCATTGGTTTTCTGCC | 1115 | UAAUUCAAUCAUUGGUUUUCUGCC | 1438 |
| Exon 46 | | | | | | | |
| Human- | 28 | 1 | TTTT | AATTCAATCATTGGTTTTCTGCCC | 1116 | AAUUCAAUCAUUGGUUUUCUGCCC | 1439 |
| Exon 46 | | | | | | | |
| Human- | 29 | 1 | TTTA | ATTCAATCATTGGTTTTCTGCCCA | 1117 | AUUCAAUCAUUGGUUUUCUGCCCA | 1440 |
| Exon 46 | | | | | | | |
| Human- | 30 | -1 | TTTA | GCAAGGAACTATGAATAACCTAAT | 1118 | GCAAGGAACUAUGAAUAACCUAAU | 1441 |
| Exon 46 | | | | | | | |
| Human- | 31 | 1 | TTTT | CTGCCCATTAGGTTATTCATAGTT | 1119 | CUGCCCAUUAGGUUAUUCAUAGUU | 1442 |
| Exon 46 | | | | | | | |
| Human- | 32 | 1 | TTTC | TGCCCATTAGGTTATTCATAGTTC | 1120 | UGCCCAUUAGGUUAUUCAUAGUUC | 1443 |
| Exon 46 | | | | | | | |
| Human- | 1 | -1 | TTTA | TAGAAAACAATTTAACAGGAAATA | 1121 | UAGAAAACAAUUUAACAGGAAAUA | 1444 |
| Exon 52 | | | | | | | |
| Human- | 2 | 1 | TTTC | CTGTTAAATTGTTTTCTATAAACC | 1122 | CUGUUAAAUUGUUUUCUAUAAACC | 1445 |
| Exon 52 | | | | | | | |
| Human- | 3 | -1 | TTTA | GAAATAAAAAAGATGTTACTGTAT | 1123 | GAAAUAAAAAAGAUGUUACUGUAU | 1446 |
| Exon 52 | | | | | | | |
| Human- | 4 | -1 | TTTT | AGAAATAAAAAAGATGTTACTGTA | 1124 | AGAAAUAAAAAAGAUGUUACUGUA | 1447 |
| Exon 52 | | | | | | | |
| Human- | 5 | 1 | TTTT | CTATAAACCCTTATACAGTAACAT | 1125 | CUAUAAACCCUUAUACAGUAACAU | 1448 |
| Exon 52 | | | | | | | |
| Human- | 6 | 1 | TTTC | TATAAACCCTTATACAGTAACATC | 1126 | UAUAAACCCUUAUACAGUAACAUC | 1449 |
| Exon 52 | | | | | | | |
| Human- | 7 | 1 | TTTT | TTATTTCTAAAAGTGTTTTGGCTG | 1127 | UUAUUUCUAAAAGUGUUUUGGCUG | 1450 |
| Exon 52 | | | | | | | |
| Human- | 8 | 1 | TTTT | TATTTCTAAAAGTGTTTTGGCTGG | 1128 | UAUUUCUAAAAGUGUUUUGGCUGG | 1451 |
| Exon 52 | | | | | | | |
| Human- | 9 | 1 | TTTT | ATTTCTAAAAGTGTTTTGGCTGGT | 1129 | AUUUCUAAAAGUGUUUUGGCUGGU | 1452 |
| Exon 52 | | | | | | | |
| Human- | 10 | 1 | TTTA | TTTCTAAAAGTGTTTTGGCTGGTC | 1130 | UUUCUAAAAGUGUUUUGGCUGGUC | 1453 |
| Exon 52 | | | | | | | |
| Human- | 11 | 1 | TTTC | TAAAAGTGTTTTGGCTGGTCTCAC | 1131 | UAAAAGUGUUUUGGCUGGUCUCAC | 1454 |
| Exon 52 | | | | | | | |
| Human- | 12 | -1 | TTTA | CATAATACAAAGTAAAGTACAATT | 1132 | CAUAAUACAAAGUAAAGUACAAUU | 1455 |
| Exon 52 | | | | | | | |
| Human- | 13 | -1 | TTTT | ACATAATACAAAGTAAAGTACAAT | 1133 | ACAUAAUACAAAGUAAAGUACAAU | 1456 |
| Exon 52 | | | | | | | |
| Human- | 14 | 1 | TTTT | GGCTGGTCTCACAATTGTACTTTA | 1134 | GGCUGGUCUCACAAUUGUACUUUA | 1457 |
| Exon 52 | | | | | | | |
| Human- | 15 | 1 | TTTG | GCTGGTCTCACAATTGTACTTTAC | 1135 | GCUGGUCUCACAAUUGUACUUUAC | 1458 |
| Exon 52 | | | | | | | |
| Human- | 16 | 1 | TTTA | CTTTGTATTATGTAAAAGGAATAC | 1136 | CUUUGUAUUAUGUAAAAGGAAUAC | 1459 |
| Exon 52 | | | | | | | |
| Human- | 17 | 1 | TTTG | TATTATGTAAAAGGAATACACAAC | 1137 | UAUUAUGUAAAAGGAAUACACAAC | 1460 |
| Exon 52 | | | | | | | |
| Human- | 18 | 1 | TTTG | TTCTTACAGGCAACAATGCAGGAT | 1138 | UUCUUACAGGCAACAAUGCAGGAU | 1461 |
| Exon 52 | | | | | | | |
| Human- | 19 | 1 | TTTG | GAACAGAGGCGTCCCCAGTTGGAA | 1139 | GAACAGAGGCGUCCCCAGUUGGAA | 1462 |
| Exon 52 | | | | | | | |
| Human- | 20 | -1 | TTTG | GGCAGCGGTAATGAGTTCTTCCAA | 1140 | GGCAGCGGUAAUGAGUUCUUCCAA | 1463 |
| Exon 52 | | | | | | | |
| Human- | 21 | -1 | TTTT | TCAAATTTTGGGCAGCGGTAATGA | 1141 | UCAAAUUUUGGGCAGCGGUAAUGA | 1464 |
| Exon 52 | | | | | | | |
| Human- | 22 | 1 | TTTG | AAAAACAAGACCAGCAATCAAGAG | 1142 | AAAAACAAGACCAGCAAUCAAGAG | 1465 |
| Exon 52 | | | | | | | |
| Human- | 23 | -1 | TTTG | TGTGTCCCATGCTTGTTAAAAAAC | 1143 | UGUGUCCCAUGCUUGUUAAAAAAC | 1466 |
| Exon 52 | | | | | | | |
| Human- | 24 | 1 | TTTT | TTAACAAGCATGGGACACACAAAG | 1144 | UUAACAAGCAUGGGACACACAAAG | 1467 |
| Exon 52 | | | | | | | |
| Human- | 25 | 1 | TTTT | TAACAAGCATGGGACACACAAAGC | 1145 | UAACAAGCAUGGGACACACAAAGC | 1468 |
| Exon 52 | | | | | | | |
| Human- | 26 | 1 | TTTT | AACAAGCATGGGACACACAAAGCA | 1146 | AACAAGCAUGGGACACACAAAGCA | 1469 |
| Exon 52 | | | | | | | |
| Human- | 27 | 1 | TTTA | ACAAGCATGGGACACACAAAGCAA | 1147 | ACAAGCAUGGGACACACAAAGCAA | 1470 |
| Exon 52 | | | | | | | |
| Human- | 28 | -1 | TTTA | TTGAAACTTGTCATGCATCTTGCT | 1148 | UUGAAACUUGUCAUGCAUCUUGCU | 1471 |
| Exon 52 | | | | | | | |
| Human- | 29 | -1 | TTTT | ATTGAAACTTGTCATGCATCTTGC | 1149 | AUUGAAACUUGUCAUGCAUCUUGC | 1472 |
| Exon 52 | | | | | | | |
| Human- | 30 | -1 | TTTT | TATTGAAACTTGTCATGCATCTTG | 1150 | UAUUGAAACUUGUCAUGCAUCUUG | 1473 |
| Exon 52 | | | | | | | |
| Human- | 31 | 1 | TTTC | AATAAAAACTTAAGTTCATATATC | 1151 | AAUAAAAACUUAAGUUCAUAUAUC | 1474 |
| Exon 52 | | | | | | | |
| Human- | 1 | -1 | TTTG | GTGAATATATTATTGGATTTCTAT | 1152 | GUGAAUAUAUUAUUGGAUUUCUAU | 1475 |
| Exon 50 | | | | | | | |
| Human- | 2 | -1 | TTTG | AAGATAATTCATGAACATCTTAAT | 1153 | AAGAUAAUUCAUGAACAUCUUAAU | 1476 |
| Exon 50 | | | | | | | |
| Human- | 3 | -1 | TTTA | ACAGAAAAGCATACACATTACTTA | 1154 | ACAGAAAAGCAUACACAUUACUUA | 1477 |
| Exon 50 | | | | | | | |
| Human- | 4 | 1 | TTTT | CTGTTAAAGAGGAAGTTAGAAGAT | 1155 | CUGUUAAAGAGGAAGUUAGAAGAU | 1478 |
| Exon 50 | | | | | | | |
| Human- | 5 | 1 | TTTC | TGTTAAAGAGGAAGTTAGAAGATC | 1156 | UGUUAAAGAGGAAGUUAGAAGAUC | 1479 |
| Exon 50 | | | | | | | |
| Human- | 6 | -1 | TTTA | CCGCCTTCCACTCAGAGCTCAGAT | 1157 | CCGCCUUCCACUCAGAGCUCAGAU | 1480 |
| Exon 50 | | | | | | | |
| Human- | 7 | -1 | TTTG | CCCTCAGCTCTTGAAGTAAACGGT | 1158 | CCCUCAGCUCUUGAAGUAAACGGU | 1481 |
| Exon 50 | | | | | | | |
| Human- | 8 | 1 | TTTA | CTTCAAGAGCTGAGGGCAAAGCAG | 1159 | CUUCAAGAGCUGAGGGCAAAGCAG | 1482 |
| Exon 50 | | | | | | | |
| Human- | 9 | -1 | TTTG | AACAAATAGCTAGAGCCAAAGAGA | 1160 | AACAAAUAGCUAGAGCCAAAGAGA | 1483 |
| Exon 50 | | | | | | | |
| Human- | 10 | -1 | TTTT | GAACAAATAGCTAGAGCCAAAGAG | 1161 | GAACAAAUAGCUAGAGCCAAAGAG | 1484 |
| Exon 50 | | | | | | | |
| Human- | 11 | 1 | TTTG | GCTCTAGCTATTTGTTCAAAAGTG | 1162 | GCUCUAGCUAUUUGUUCAAAAGUG | 1485 |
| Exon 50 | | | | | | | |
| Human- | 12 | 1 | TTTG | TTCAAAAGTGCAACTATGAAGTGA | 1163 | UUCAAAAGUGCAACUAUGAAGUGA | 1486 |
| Exon 50 | | | | | | | |
| Human- | 13 | -1 | TTTC | TCTCTCACCCAGTCATCACTTCAT | 1164 | UCUCUCACCCAGUCAUCACUUCAU | 1487 |
| Exon 50 | | | | | | | |
| Human- | 14 | -1 | TTTT | CTCTCTCACCCAGTCATCACTTCA | 1165 | CUCUCUCACCCAGUCAUCACUUCA | 1488 |
| Exon 50 | | | | | | | |
| Human- | 1 | 1 | TTTG | tatatatatatatatTTTTCTCTT | 1166 | uauauauauauauauUUUUCUCUU | 1489 |
| Exon 43 | | | | | | | |
| Human- | 2 | 1 | tTTT | TCTCTTTCTATAGACAGCTAATTC | 1167 | UCUCUUUCUAUAGACAGCUAAUUC | 1490 |
| Exon 43 | | | | | | | |
| Human- | 3 | 1 | TTTT | CTCTTTCTATAGACAGCTAATTCA | 1168 | CUCUUUCUAUAGACAGCUAAUUCA | 1491 |
| Exon 43 | | | | | | | |
| Human- | 4 | -1 | TTTA | AAACAGTAAAAAAATGAATTAGCT | 1169 | AAACAGUAAAAAAAUGAAUUAGCU | 1492 |
| Exon 43 | | | | | | | |
| Human- | 5 | 1 | TTTC | TCTTTCTATAGACAGCTAATTCAT | 1170 | UCUUUCUAUAGACAGCUAAUUCAU | 1493 |
| Exon 43 | | | | | | | |
| Human- | 6 | -1 | TTTT | AAAACAGTAAAAAAATGAATTAGC | 1171 | AAAACAGUAAAAAAAUGAAUUAGC | 1494 |
| Exon 43 | | | | | | | |
| Human- | 7 | 1 | TTTC | TATAGACAGCTAATTCATTTTTTT | 1172 | UAUAGACAGCUAAUUCAUUUUUUU | 1495 |
| Exon 43 | | | | | | | |
| Human- | 8 | -1 | TTTA | TATTCTGTAATATAAAAATTTTAA | 1173 | UAUUCUGUAAUAUAAAAAUUUUAA | 1496 |
| Exon 43 | | | | | | | |
| Human- | 9 | -1 | TTTT | ATATTCTGTAATATAAAAATTTTA | 1174 | AUAUUCUGUAAUAUAAAAAUUUUA | 1497 |
| Exon 43 | | | | | | | |
| Human- | 10 | 1 | TTTT | TTTACTGTTTTAAAATTTTTATAT | 1175 | UUUACUGUUUUAAAAUUUUUAUAU | 1498 |
| Exon 43 | | | | | | | |
| Human- | 11 | 1 | TTTT | TTACTGTTTTAAAATTTTTATATT | 1176 | UUACUGUUUUAAAAUUUUUAUAUU | 1499 |
| Exon 43 | | | | | | | |
| Human- | 12 | 1 | TTTT | TACTGTTTTAAAATTTTTATATTA | 1177 | UACUGUUUUAAAAUUUUUAUAUUA | 1500 |
| Exon 43 | | | | | | | |
| Human- | 13 | 1 | TTTT | ACTGTTTTAAAATTTTTATATTAC | 1178 | ACUGUUUUAAAAUUUUUAUAUUAC | 1501 |
| Exon 43 | | | | | | | |
| Human- | 14 | 1 | TTTA | CTGTTTTAAAATTTTTATATTACA | 1179 | CUGUUUUAAAAUUUUUAUAUUACA | 1502 |
| Exon 43 | | | | | | | |
| Human- | 15 | 1 | TTTT | AAAATTTTTATATTACAGAATATA | 1180 | AAAAUUUUUAUAUUACAGAAUAUA | 1503 |
| Exon 43 | | | | | | | |
| Human- | 16 | 1 | TTTA | AAATTTTTATATTACAGAATATAA | 1181 | AAAUUUUUAUAUUACAGAAUAUAA | 1504 |
| Exon 43 | | | | | | | |
| Human- | 17 | -1 | TTTG | TTGTAGACTATCTTTTATATTCTG | 1182 | UUGUAGACUAUCUUUUAUAUUCUG | 1505 |
| Exon 43 | | | | | | | |
| Human- | 18 | 1 | TTTT | TATATTACAGAATATAAAAGATAG | 1183 | UAUAUUACAGAAUAUAAAAGAUAG | 1506 |
| Exon 43 | | | | | | | |
| Human- | 19 | 1 | TTTT | ATATTACAGAATATAAAAGATAGT | 1184 | AUAUUACAGAAUAUAAAAGAUAGU | 1507 |
| Exon 43 | | | | | | | |
| Human- | 20 | 1 | TTTA | TATTACAGAATATAAAAGATAGTC | 1185 | UAUUACAGAAUAUAAAAGAUAGUC | 1508 |
| Exon 43 | | | | | | | |
| Human- | 21 | -1 | TTTG | CAATGCTGCTGTCTTCTTGCTATG | 1186 | CAAUGCUGCUGUCUUCUUGCUAUG | 1509 |
| Exon 43 | | | | | | | |
| Human- | 22 | 1 | TTTC | CAATGGGAAAAAGTTAACAAAATG | 1187 | CAAUGGGAAAAAGUUAACAAAAUG | 1510 |
| Exon 43 | | | | | | | |
| Human- | 23 | -1 | TTTC | TGCAAGTATCAAGAAAAATATATG | 1188 | UGCAAGUAUCAAGAAAAAUAUAUG | 1511 |
| Exon 43 | | | | | | | |
| Human- | 24 | 1 | TTTT | TCTTGATACTTGCAGAAATGATTT | 1189 | UCUUGAUACUUGCAGAAAUGAUUU | 1512 |
| Exon 43 | | | | | | | |
| Human- | 25 | 1 | TTTT | CTTGATACTTGCAGAAATGATTTG | 1190 | CUUGAUACUUGCAGAAAUGAUUUG | 1513 |
| Exon 43 | | | | | | | |
| Human- | 26 | 1 | TTTC | TTGATACTTGCAGAAATGATTTGT | 1191 | UUGAUACUUGCAGAAAUGAUUUGU | 1514 |
| Exon 43 | | | | | | | |
| Human- | 27 | 1 | TTTG | TTTTCAGGGAACTGTAGAATTTAT | 1192 | UUUUCAGGGAACUGUAGAAUUUAU | 1515 |
| Exon 43 | | | | | | | |
| Human- | 28 | -1 | TTTC | CATGGAGGGTACTGAAATAAATTC | 1193 | CAUGGAGGGUACUGAAAUAAAUUC | 1516 |
| Exon 43 | | | | | | | |
| Human- | 29 | -1 | TTTT | CCATGGAGGGTACTGAAATAAATT | 1194 | CCAUGGAGGGUACUGAAAUAAAUU | 1517 |
| Exon 43 | | | | | | | |
| Human- | 30 | 1 | TTTT | CAGGGAACTGTAGAATTTATTTCA | 1195 | CAGGGAACUGUAGAAUUUAUUUCA | 1518 |
| Exon 43 | | | | | | | |
| Human- | 31 | -1 | TTTT | TCCATGGAGGGTACTGAAATAAAT | 1196 | UCCAUGGAGGGUACUGAAAUAAAU | 1519 |
| Exon 43 | | | | | | | |
| Human- | 32 | 1 | TTTC | AGGGAACTGTAGAATTTATTTCAG | 1197 | AGGGAACUGUAGAAUUUAUUUCAG | 1520 |
| Exon 43 | | | | | | | |
| Human- | 33 | -1 | TTTT | TTCCATGGAGGGTACTGAAATAAA | 1198 | UUCCAUGGAGGGUACUGAAAUAAA | 1521 |
| Exon 43 | | | | | | | |
| Human- | 34 | -1 | TTTC | CCTGTCTTTTTTCCATGGAGGGTA | 1199 | CCUGUCUUUUUUCCAUGGAGGGUA | 1522 |
| Exon 43 | | | | | | | |
| Human- | 35 | -1 | TTTT | CCCTGTCTTTTTTCCATGGAGGGT | 1200 | CCCUGUCUUUUUUCCAUGGAGGGU | 1523 |
| Exon 43 | | | | | | | |
| Human- | 36 | -1 | TTTT | TCCCTGTCTTTTTTCCATGGAGGG | 1201 | UCCCUGUCUUUUUUCCAUGGAGGG | 1524 |
| Exon 43 | | | | | | | |
| Human- | 37 | 1 | TTTA | TTTCAGTACCCTCCATGGAAAAAA | 1202 | UUUCAGUACCCUCCAUGGAAAAAA | 1525 |
| Exon 43 | | | | | | | |
| Human- | 38 | 1 | TTTC | AGTACCCTCCATGGAAAAAAGACA | 1203 | AGUACCCUCCAUGGAAAAAAGACA | 1526 |
| Exon 43 | | | | | | | |
| Human- | 1 | 1 | TTTA | AGTTTGCATGGTTCTTGCTCAAGG | 1204 | AGUUUGCAUGGUUCUUGCUCAAGG | 1527 |
| Exon 6 | | | | | | | |
| Human- | 2 | -1 | TTTC | ATAAGAAAATGCATTCCTTGAGCA | 1205 | AUAAGAAAAUGCAUUCCUUGAGCA | 1528 |
| Exon 6 | | | | | | | |
| Human- | 3 | -1 | TTTT | CATAAGAAAATGCATTCCTTGAGC | 1206 | CAUAAGAAAAUGCAUUCCUUGAGC | 1529 |
| Exon 6 | | | | | | | |
| Human- | 4 | 1 | TTTG | CATGGTTCTTGCTCAAGGAATGCA | 1207 | CAUGGUUCUUGCUCAAGGAAUGCA | 1530 |
| Exon 6 | | | | | | | |
| Human- | 5 | -1 | TTTG | ACCTACATGTGGAAATAAATTTTC | 1208 | ACCUACAUGUGGAAAUAAAUUUUC | 1531 |
| Exon 6 | | | | | | | |
| Human- | 6 | -1 | TTTT | GACCTACATGTGGAAATAAATTTT | 1209 | GACCUACAUGUGGAAAUAAAUUUU | 1532 |
| Exon 6 | | | | | | | |
| Human- | 7 | -1 | TTTT | TGACCTACATGTGGAAATAAATTT | 1210 | UGACCUACAUGUGGAAAUAAAUUU | 1533 |
| Exon 6 | | | | | | | |
| Human- | 8 | 1 | TTTT | CTTATGAAAATTTATTTCCACATG | 1211 | CUUAUGAAAAUUUAUUUCCACAUG | 1534 |
| Exon 6 | | | | | | | |
| Human- | 9 | 1 | TTTC | TTATGAAAATTTATTTCCACATGT | 1212 | UUAUGAAAAUUUAUUUCCACAUGU | 1535 |
| Exon 6 | | | | | | | |
| Human- | 10 | -1 | TTTC | ATTACATTTTTGACCTACATGTGG | 1213 | AUUACAUUUUUGACCUACAUGUGG | 1536 |
| Exon 6 | | | | | | | |
| Human- | 11 | -1 | TTTT | CATTACATTTTTGACCTACATGTG | 1214 | CAUUACAUUUUUGACCUACAUGUG | 1537 |
| Exon 6 | | | | | | | |
| Human- | 12 | -1 | TTTT | TCATTACATTTTTGACCTACATGT | 1215 | UCAUUACAUUUUUGACCUACAUGU | 1538 |
| Exon 6 | | | | | | | |
| Human- | 13 | 1 | TTTA | TTTCCACATGTAGGTCAAAAATGT | 1216 | UUUCCACAUGUAGGUCAAAAAUGU | 1539 |
| Exon 6 | | | | | | | |
| Human- | 14 | 1 | TTTC | CACATGTAGGTCAAAAATGTAATG | 1217 | CACAUGUAGGUCAAAAAUGUAAUG | 1540 |
| Exon 6 | | | | | | | |
| Human- | 15 | -1 | TTTG | TTGCAATCCAGCCATGATATTTTT | 1218 | UUGCAAUCCAGCCAUGAUAUUUUU | 1541 |
| Exon 6 | | | | | | | |
| Human- | 16 | -1 | TTTC | ACTGTTGGTTTGTTGCAATCCAGC | 1219 | ACUGUUGGUUUGUUGCAAUCCAGC | 1542 |
| Exon 6 | | | | | | | |
| Human- | 17 | -1 | TTTT | CACTGTTGGTTTGTTGCAATCCAG | 1220 | CACUGUUGGUUUGUUGCAAUCCAG | 1543 |
| Exon 6 | | | | | | | |
| Human- | 18 | 1 | TTTG | AATGCTCTCATCCATAGTCATAGG | 1221 | AAUGCUCUCAUCCAUAGUCAUAGG | 1544 |
| Exon 6 | | | | | | | |
| Human- | 19 | -1 | TTTA | ATGTCTCAGTAATCTTCTTACCTA | 1222 | AUGUCUCAGUAAUCUUCUUACCUA | 1545 |
| Exon 6 | | | | | | | |
| Human- | 20 | -1 | TTTA | CAAGTTATTTAATGTCTCAGTAAT | 1223 | CAAGUUAUUUAAUGUCUCAGUAAU | 1546 |
| Exon 6 | | | | | | | |
| Human- | 21 | -1 | TTTT | ACAAGTTATTTAATGTCTCAGTAA | 1224 | ACAAGUUAUUUAAUGUCUCAGUAA | 1547 |
| Exon 6 | | | | | | | |
| Human- | 22 | 1 | TTTA | GACTCTGATGACATATTTTTCCCC | 1225 | GACUCUGAUGACAUAUUUUUCCCC | 1548 |
| Exon 6 | | | | | | | |
| Human- | 23 | 1 | TTTT | TCCCCAGTATGGTTCCAGATCATG | 1226 | UCCCCAGUAUGGUUCCAGAUCAUG | 1549 |
| Exon 6 | | | | | | | |
| Human- | 24 | 1 | TTTT | CCCCAGTATGGTTCCAGATCATGT | 1227 | CCCCAGUAUGGUUCCAGAUCAUGU | 1550 |
| Exon 6 | | | | | | | |
| Human- | 25 | 1 | TTTC | CCCAGTATGGTTCCAGATCATGTC | 1228 | CCCAGUAUGGUUCCAGAUCAUGUC | 1551 |
| Exon 6 | | | | | | | |
| Human- | 1 | 1 | TTTA | TATTTGTCTTtgtgtatgtgtgta | 1229 | UAUUUGUCUUuguguaugugugua | 1552 |
| Exon 7 | | | | | | | |
| Human- | 2 | 1 | TTTG | TCTTtgtgtatgtgtgtatgtgta | 1230 | UCUUuguguauguguguaugugua | 1553 |
| Exon 7 | | | | | | | |
| Human- | 3 | 1 | TTtg | tgtatgtgtgtatgtgtatgtgtt | 1231 | uguauguguguauguguauguguu | 1554 |
| Exon 7 | | | | | | | |
| Human- | 4 | 1 | ttTT | AGGCCAGACCTATTTGACTGGAAT | 1232 | AGGCCAGACCUAUUUGACUGGAAU | 1555 |
| Exon 7 | | | | | | | |
| Human- | 5 | 1 | tTTA | GGCCAGACCTATTTGACTGGAATA | 1233 | GGCCAGACCUAUUUGACUGGAAUA | 1556 |
| Exon 7 | | | | | | | |
| Human- | 6 | 1 | TTTG | ACTGGAATAGTGTGGTTTGCCAGC | 1234 | ACUGGAAUAGUGUGGUUUGCCAGC | 1557 |
| Exon 7 | | | | | | | |
| Human- | 7 | 1 | TTTG | CCAGCAGTCAGCCACACAACGACT | 1235 | CCAGCAGUCAGCCACACAACGACU | 1558 |
| Exon 7 | | | | | | | |
| Human- | 8 | -1 | TTTC | TCTATGCCTAATTGATATCTGGCG | 1236 | UCUAUGCCUAAUUGAUAUCUGGCG | 1559 |
| Exon 7 | | | | | | | |
| Human- | 9 | -1 | TTTA | CCAACCTTCAGGATCGAGTAGTTT | 1237 | CCAACCUUCAGGAUCGAGUAGUUU | 1560 |
| Exon 7 | | | | | | | |
| Human- | 10 | 1 | TTTC | TGGACTACCACTGCTTTTAGTATG | 1238 | UGGACUACCACUGCUUUUAGUAUG | 1561 |
| Exon 7 | | | | | | | |
| Human- | 11 | 1 | TTTT | AGTATGGTAGAGTTTAATGTTTTC | 1239 | AGUAUGGUAGAGUUUAAUGUUUUC | 1562 |
| Exon 7 | | | | | | | |
| Human- | 12 | 1 | TTTA | GTATGGTAGAGTTTAATGTTTTCA | 1240 | GUAUGGUAGAGUUUAAUGUUUUCA | 1563 |
| Exon 7 | | | | | | | |
| Human- | 1 | -1 | TTTG | AGACTCTAAAAGGATAATGAACAA | 1241 | AGACUCUAAAAGGAUAAUGAACAA | 1564 |
| Exon 8 | | | | | | | |
| Human- | 2 | 1 | TTTA | ACTTTGATTTGTTCATTATCCTTT | 1242 | ACUUUGAUUUGUUCAUUAUCCUUU | 1565 |
| Exon 8 | | | | | | | |
| Human- | 3 | -1 | TTTC | TATATTTGAGACTCTAAAAGGATA | 1243 | UAUAUUUGAGACUCUAAAAGGAUA | 1566 |
| Exon 8 | | | | | | | |
| Human- | 4 | 1 | TTTG | ATTTGTTCATTATCCTTTTAGAGT | 1244 | AUUUGUUCAUUAUCCUUUUAGAGU | 1567 |
| Exon 8 | | | | | | | |
| Human- | 5 | -1 | TTTG | GTTTCTATATTTGAGACTCTAAAA | 1245 | GUUUCUAUAUUUGAGACUCUAAAA | 1568 |
| Exon 8 | | | | | | | |
| Human- | 6 | -1 | TTTT | GGTTTCTATATTTGAGACTCTAAA | 1246 | GGUUUCUAUAUUUGAGACUCUAAA | 1569 |
| Exon 8 | | | | | | | |
| Human- | 7 | -1 | TTTT | TGGTTTCTATATTTGAGACTCTAA | 1247 | UGGUUUCUAUAUUUGAGACUCUAA | 1570 |
| Exon 8 | | | | | | | |
| Human- | 8 | 1 | TTTG | TTCATTATCCTTTTAGAGTCTCAA | 1248 | UUCAUUAUCCUUUUAGAGUCUCAA | 1571 |
| Exon 8 | | | | | | | |
| Human- | 9 | 1 | TTTT | AGAGTCTCAAATATAGAAACCAAA | 1249 | AGAGUCUCAAAUAUAGAAACCAAA | 1572 |
| Exon 8 | | | | | | | |
| Human- | 10 | 1 | TTTA | GAGTCTCAAATATAGAAACCAAAA | 1250 | GAGUCUCAAAUAUAGAAACCAAAA | 1573 |
| Exon 8 | | | | | | | |
| Human- | 11 | -1 | TTTC | CACTTCCTGGATGGCTTCAATGCT | 1251 | CACUUCCUGGAUGGCUUCAAUGCU | 1574 |
| Exon 8 | | | | | | | |
| Human- | 12 | 1 | TTTT | GCCTCAACAAGTGAGCATTGAAGC | 1252 | GCCUCAACAAGUGAGCAUUGAAGC | 1575 |
| Exon 8 | | | | | | | |
| Human- | 13 | 1 | TTTG | CCTCAACAAGTGAGCATTGAAGCC | 1253 | CCUCAACAAGUGAGCAUUGAAGCC | 1576 |
| Exon 8 | | | | | | | |
| Human- | 14 | -1 | TTTA | GGTGGCCTTGGCAACATTTCCACT | 1254 | GGUGGCCUUGGCAACAUUUCCACU | 1577 |
| Exon 8 | | | | | | | |
| Human- | 15 | -1 | TTTA | GTCACTTTAGGTGGCCTTGGCAAC | 1255 | GUCACUUUAGGUGGCCUUGGCAAC | 1578 |
| Exon 8 | | | | | | | |
| Human- | 16 | -1 | TTTG | ATGATGTAACTGAAAATGTTCTTC | 1256 | AUGAUGUAACUGAAAAUGUUCUUC | 1579 |
| Exon 8 | | | | | | | |
| Human- | 17 | -1 | TTTA | CCTGTTGAGAATAGTGCATTTGAT | 1257 | CCUGUUGAGAAUAGUGCAUUUGAU | 1580 |
| Exon 8 | | | | | | | |
| Human- | 18 | 1 | TTTT | CAGTTACATCATCAAATGCACTAT | 1258 | CAGUUACAUCAUCAAAUGCACUAU | 1581 |
| Exon 8 | | | | | | | |
| Human- | 19 | 1 | TTTC | AGTTACATCATCAAATGCACTATT | 1259 | AGUUACAUCAUCAAAUGCACUAUU | 1582 |
| Exon 8 | | | | | | | |
| Human- | 20 | -1 | TTTA | CACACTTTACCTGTTGAGAATAGT | 1260 | CACACUUUACCUGUUGAGAAUAGU | 1583 |
| Exon 8 | | | | | | | |
| Human- | 21 | 1 | TTTT | CTGTTTTATATGCATTTTTAGGTA | 1261 | CUGUUUUAUAUGCAUUUUUAGGUA | 1584 |
| Exon 8 | | | | | | | |
| Human- | 22 | 1 | TTTC | TGTTTTATATGCATTTTTAGGTAT | 1262 | UGUUUUAUAUGCAUUUUUAGGUAU | 1585 |
| Exon 8 | | | | | | | |
| Human- | 23 | 1 | TTTT | ATATGCATTTTTAGGTATTACGTG | 1263 | AUAUGCAUUUUUAGGUAUUACGUG | 1586 |
| Exon 8 | | | | | | | |
| Human- | 24 | 1 | TTTA | TATGCATTTTTAGGTATTACGTGC | 1264 | UAUGCAUUUUUAGGUAUUACGUGC | 1587 |
| Exon 8 | | | | | | | |
| Human- | 25 | 1 | TTTT | TAGGTATTACGTGCACatatatat | 1265 | UAGGUAUUACGUGCACauauauau | 1588 |
| Exon 8 | | | | | | | |
| Human- | 26 | 1 | TTTT | AGGTATTACGTGCACatatatata | 1266 | AGGUAUUACGUGCACauauauaua | 1589 |
| Exon 8 | | | | | | | |
| Human- | 27 | 1 | TTTA | GGTATTACGTGCACatatatatat | 1267 | GGUAUUACGUGCACauauauauau | 1590 |
| Exon 8 | | | | | | | |
| Human- | 1 | -1 | TTTA | AGCAACAACTATAATATTGTGCAG | 1268 | AGCAACAACUAUAAUAUUGUGCAG | 1591 |
| Exon 55 | | | | | | | |
| Human- | 2 | 1 | TTTA | GTTCCTCCATCTTTCTCTTTTTAT | 1269 | GUUCCUCCAUCUUUCUCUUUUUAU | 1592 |
| Exon 55 | | | | | | | |
| Human- | 3 | 1 | TTTC | TCTTTTTATGGAGTTCACTAGGTG | 1270 | UCUUUUUAUGGAGUUCACUAGGUG | 1593 |
| Exon 55 | | | | | | | |
| Human- | 4 | 1 | TTTT | TATGGAGTTCACTAGGTGCACCAT | 1271 | UAUGGAGUUCACUAGGUGCACCAU | 1594 |
| Exon 55 | | | | | | | |
| Human- | 5 | 1 | TTTT | ATGGAGTTCACTAGGTGCACCATT | 1272 | AUGGAGUUCACUAGGUGCACCAUU | 1595 |
| Exon 55 | | | | | | | |
| Human- | 6 | 1 | TTTA | TGGAGTTCACTAGGTGCACCATTC | 1273 | UGGAGUUCACUAGGUGCACCAUUC | 1596 |
| Exon 55 | | | | | | | |
| Human- | 7 | 1 | TTTA | ATAATTGCATCTGAACATTTGGTC | 1274 | AUAAUUGCAUCUGAACAUUUGGUC | 1597 |
| Exon 55 | | | | | | | |
| Human- | 8 | 1 | TTTG | GTCCTTTGCAGGGTGAGTGAGCGA | 1275 | GUCCUUUGCAGGGUGAGUGAGCGA | 1598 |
| Exon 55 | | | | | | | |
| Human- | 9 | -1 | TTTC | TTCCAAAGCAGCCTCTCGCTCACT | 1276 | UUCCAAAGCAGCCUCUCGCUCACU | 1599 |
| Exon 55 | | | | | | | |
| Human- | 10 | 1 | TTTG | CAGGGTGAGTGAGCGAGAGGCTGC | 1277 | CAGGGUGAGUGAGCGAGAGGCUGC | 1600 |
| Exon 55 | | | | | | | |
| Human- | 11 | 1 | TTTG | GAAGAAACTCATAGATTACTGCAA | 1278 | GAAGAAACUCAUAGAUUACUGCAA | 1601 |
| Exon 55 | | | | | | | |
| Human- | 12 | -1 | TTTC | CAGGTCCAGGGGGAACTGTTGCAG | 1279 | CAGGUCCAGGGGGAACUGUUGCAG | 1602 |
| Exon 55 | | | | | | | |
| Human- | 13 | -1 | TTTT | CCAGGTCCAGGGGGAACTGTTGCA | 1280 | CCAGGUCCAGGGGGAACUGUUGCA | 1603 |
| Exon 55 | | | | | | | |
| Human- | 14 | -1 | TTTC | AGCTTCTGTAAGCCAGGCAAGAAA | 1281 | AGCUUCUGUAAGCCAGGCAAGAAA | 1604 |
| Exon 55 | | | | | | | |
| Human- | 15 | 1 | TTTC | TTGCCTGGCTTACAGAAGCTGAAA | 1282 | UUGCCUGGCUUACAGAAGCUGAAA | 1605 |
| Exon 55 | | | | | | | |
| Human- | 16 | -1 | TTTC | CTTACGGGTAGCATCCTGTAGGAC | 1283 | CUUACGGGUAGCAUCCUGUAGGAC | 1606 |
| Exon 55 | | | | | | | |
| Human- | 17 | -1 | TTTA | CTCCCTTGGAGTCTTCTAGGAGCC | 1284 | CUCCCUUGGAGUCUUCUAGGAGCC | 1607 |
| Exon 55 | | | | | | | |
| Human- | 18 | -1 | TTTT | ACTCCCTTGGAGTCTTCTAGGAGC | 1285 | ACUCCCUUGGAGUCUUCUAGGAGC | 1608 |
| Exon 55 | | | | | | | |
| Human- | 19 | -1 | TTTC | ATCAGCTCTTTTACTCCCTTGGAG | 1286 | AUCAGCUCUUUUACUCCCUUGGAG | 1609 |
| Exon 55 | | | | | | | |
| Human- | 20 | 1 | TTTC | CGCTTTAGCACTCTTGTGGATCCA | 1287 | CGCUUUAGCACUCUUGUGGAUCCA | 1610 |
| Exon 55 | | | | | | | |
| Human- | 21 | 1 | TTTA | GCACTCTTGTGGATCCAATTGAAC | 1288 | GCACUCUUGUGGAUCCAAUUGAAC | 1611 |
| Exon 55 | | | | | | | |
| Human- | 22 | -1 | TTTG | TCCCTGGCTTGTCAGTTACAAGTA | 1289 | UCCCUGGCUUGUCAGUUACAAGUA | 1612 |
| Exon 55 | | | | | | | |
| Human- | 23 | -1 | TTTT | GTCCCTGGCTTGTCAGTTACAAGT | 1290 | GUCCCUGGCUUGUCAGUUACAAGU | 1613 |
| Exon 55 | | | | | | | |
| Human- | 24 | -1 | TTTG | TTTTGTCCCTGGCTTGTCAGTTAC | 1291 | UUUUGUCCCUGGCUUGUCAGUUAC | 1614 |
| Exon 55 | | | | | | | |
| Human- | 25 | -1 | TTTT | GTTTTGTCCCTGGCTTGTCAGTTA | 1292 | GUUUUGUCCCUGGCUUGUCAGUUA | 1615 |
| Exon 55 | | | | | | | |
| Human- | 26 | 1 | TTTG | TACTTGTAACTGACAAGCCAGGGA | 1293 | UACUUGUAACUGACAAGCCAGGGA | 1616 |
| Exon 55 | | | | | | | |
| Human- | | 1 | TTTA | gCTCCTACTCAGACTGTTACTCTG | 1294 | gCUCCUACUCAGACUGUUACUCUG | 1617 |
| G1-exon51 | | | | | | | |
| Human- | | 1 | TTTC | taccatgtattgctaaacaaagta | 1295 | uaccauguauugcuaaacaaagua | 1618 |
| G2-exon51 | | | | | | | |
| Human- | | -1 | TTTA | attgaagagtaacaatttgagcca | 1296 | auugaagaguaacaauuugagcca | 1619 |
| G3-exon51 | | | | | | | |
| mouse- | | 1 | TTTG | aggctctgcaaagttctTTGAAAG | 1297 | aggcucugcaaaguucuUUGAAAG | 1620 |
| Exon23- | | | | | | | |
| G1 | | | | | | | |
| mouse- | | 1 | TTTG | AAAGAGCAACAAAATGGCttcaac | 1298 | AAAGAGCAACAAAAUGGCuucaac | 1621 |
| Exon23- | | | | | | | |
| G2 | | | | | | | |
| mouse- | | 1 | TTTG | AAAGAGCAATAAAATGGCttcaac | 1299 | AAAGAGCAAUAAAAUGGCuucaac | 1622 |
| Exon23- | | | | | | | |
| G3 | | | | | | | |
| mouse- | | -1 | TTTC | AAAGAACTTTGCAGAGCctcaaaa | 1300 | AAAGAACUUUGCAGAGCcucaaaa | 1623 |
| Exon23- | | | | | | | |
| G4 | | | | | | | |
| mouse- | | -1 | TTTA | ctgaatatctatgcattaataact | 1301 | cugaauaucuaugcauuaauaacu | 1624 |
| Exon23- | | | | | | | |
| G5 | | | | | | | |
| mouse- | | -1 | TTTC | tattatattacagggcatattata | 1302 | ummaummacagggcaummaua | 1625 |
| Exon23- | | | | | | | |
| G6 | | | | | | | |
| mouse- | | 1 | TTTC | Aggtaagccgaggtttggccttta | 1303 | Agguaagccgagguuuggccuuua | 1626 |
| Exon23- | | | | | | | |
| G7 | | | | | | | |
| mouse- | | 1 | TTTA | cccagagtccttcaaagatattga | 1304 | cccagaguccuucaaagauauuga | 1627 |
| Exon23- | | | | | | | |
| G8 | | | | | | | |

*In this table, upper case letters represent sgRNA nucleotides that align to the exon sequence of the gene. Lower case letters represent sgRNA nucleotides that align to the intron sequence of the gene

TABLE 19
Additional gRNA targeting sequences

| | | | | | | SEQ | |
|---|---|---|---|---|---|---|---|
| Name | Species | Gene | Target | Strand | Sequence | ID NO | PAM |
| DCR1 | Human | DMD | Intron | + | attggctttgatttcccta | 1628 | GGG |
| | | | 50 | | | | |
| DCR2 | Human | DMD | Intron | - | tgtagagtaagtcagccta | 1629 | TGG |
| | | | 50 | | | | |
| DCR3 | Human | DMD | Exon | + | cctactcagactgttactc | 1630 | TGG |
| | | | 51-55′ | | | | |
| DCR4 | Human | DMD | Exon | + | ttggacagaacttaccgac | 1631 | TGG |
| | | | 51-53′ | | | | |
| DCR5 | Human | DMD | Intron | - | cagttgcctaagaactggt | 1632 | GGG |
| | | | 51 | | | | |
| DCR6 | Human | DMD | Intron | - | GGGCTCCACCCTCACGAGT | 1633 | GGG |
| | | | 44 | | | | |
| DCR7 | Human | DMD | Intron | + | TTTGCTTCGCTATAAAACG | 1634 | AGG |
| | | | 55 | | | | |
| DCR8 | Human | DMD | Exon 41 | + | TCTGAGGATGGGGCCGCAA | 1635 | TGG |
| DCR9 | Human | DMD | Exon 44 | - | GATCTGTCAAATCGCCTGC | 1636 | AGG |
| DCR10 | Human | DMD | Exon 45 | + | CCAGGATGGCATTGGGCAG | 1637 | CGG |
| DCR11 | Human | DMD | Exon 45 | + | CTGAATCTGCGGTGGCAGG | 1638 | AGG |
| DCR12 | Human | DMD | Exon 46 | - | TTCTTTTGTTCTTCTAGCc | 1639 | TGG |
| DCR13 | Human | DMD | Exon 46 | + | GAAAAGCTTGAGCAAGTCA | 1640 | AGG |
| DCR14 | Human | DMD | Exon 47 | + | GAAGAGTTGCCCCTGCGCC | 1641 | AGG |
| DCR15 | Human | DMD | Exon 47 | + | ACAAATCTCCAGTGGATAA | 1642 | AGG |
| DCR16 | Human | DMD | Exon 48 | - | TGTTTCTCAGGTAAAGCTC | 1643 | TGG |
| DCR17 | Human | DMD | Exon 48 | + | GAAGGACCATTTGACGTTa | 1644 | AGG |
| DCR18 | Human | DMD | Exon 49 | - | AACTGCTATTTCAGTTTCc | 1645 | TGG |
| DCR19 | Human | DMD | Exon 49 | + | CCAGCCACTCAGCCAGTGA | 1646 | AGG |
| DCR20 | Human | DMD | Exon 50 | + | gtatgcttttctgttaaag | 1647 | AGG |
| DCR21 | Human | DMD | Exon 50 | + | CTCCTGGACTGACCACTAT | 1648 | TGG |
| DCR22 | Human | DMD | Exon 52 | + | GAACAGAGGCGTCCCCAGT | 1649 | TGG |
| DCR23 | Human | DMD | Exon 52 | + | GAGGCTAGAACAATCATTA | 1650 | CGG |
| DCR24 | Human | DMD | Exon 53 | + | ACAAGAACACCTTCAGAAC | 1651 | CGG |
| DCR25 | Human | DMD | Exon 53 | - | GGTTTCTGTGATTTTCTTT | 1652 | TGG |
| DCR26 | Human | DMD | Exon 54 | + | GGCCAAAGACCTCCGCCAG | 1653 | TGG |
| DCR27 | Human | DMD | Exon 54 | + | TTGGAGAAGCATTCATAAA | 1654 | AGG |
| DCR28 | Human | DMD | Exon 55 | - | TCGCTCACTCACCctgcaa | 1655 | AGG |
| DCR29 | Human | DMD | Exon 55 | + | AAAAGAGCTGATGAAACAA | 1656 | TGG |
| DCR30 | Human | DMD | 5′UTR/ | + | TAcACTTTTCaAAATGCTT | 1657 | TGG |
| | | | Exon 1 | | | | |
| DCR31 | Human | DMD | Exon 51 | + | gagatgatcatcaagcaga | 1658 | AGG |
| DCR32 | Mouse | DMD | mdx | + | ctttgaaagagcaaTaaaa | 1659 | TGG |
| DCR33 | Human | DMD | Intron | - | CACAAAAGTCAAATCGGAA | 1660 | TGG |
| | | | 44 | | | | |
| DCR34 | Human | DMD | Intron | - | ATTTCAATATAAGATTCGG | 1661 | AGG |
| | | | 44 | | | | |
| DCR35 | Human | DMD | Intron | - | CTTAAGCAATCCCGAACTC | 1662 | TGG |
| | | | 55 | | | | |
| DCR36 | Human | DMD | Intron | - | CCTTCTTTATCCCCTATCG | 1663 | AGG |
| | | | 55 | | | | |
| DCR40 | Mouse | DMD | Exon 23 | - | aggccaaacctcggcttac | 1664 | NNGRR |
| DCR41 | Mouse | DMD | Exon 23 | + | TTCGAAAATTTCAGgtaag | 1665 | NNGRR |
| DCR42 | Mouse | DMD | Exon 23 | + | gcagaacaggagataacag | 1666 | NNGRRT |
| DCR43 | Mouse | ACV | Exon 1 | + | gcggccctcgcccttctct | 1667 | ggggat |
| | | R2B | | | | | |
| DCR48 | Human | DMD | Intron | - | TAGTGATCGTGGATACGAG | 1668 | AGG |
| | | | 45 | | | | |
| DCR49 | Human | DMD | Intron | - | TACAGCCCTCGGTGTATAT | 1669 | TGG |
| | | | 45 | | | | |
| DCR50 | Human | DMD | Intron | - | GGAAGGAATTAAGCCCGAA | 1670 | TGG |
| | | | 52 | | | | |
| DCR51 | Human | DMD | Intron | - | GGAACAGCTTTCGTAGTTG | 1671 | AGG |
| | | | 53 | | | | |
| DCR52 | Human | DMD | Intron | + | ATAAAGTCCAGTGTCGATC | 1672 | AGG |
| | | | 54 | | | | |
| DCR53 | | | Intron | + | AAAACCAGAGCTTCGGTCA | 1673 | AGG |
| | | | 54 | | | | |
| DCR54 | Mouse | Rosa26 | ZFN | + | GAGTCTTCTGGGCAGGCTTAA | 1674 | TGG |
| | | | region | | | | |
| DCR55 | Mouse | Rosa26 | mRNA | - | TCGGGTGAGCATGTCTTTAAT | 1675 | TGG |
| DCR49 | Human | DMD | Ex 51 | - | gtgtcaccagagtaacagt | 1676 | ctgagt |
| DCR50 | Human | DMD | Ex 51 | + | tgatcatcaagcagaaggt | 1677 | atgag |
| DCR60 | Mouse | DMD | Exon 23 | + | AACTTCGAAAATTTCAGgta | 1678 | agccgagg |
| DCR61 | Mouse | DMD | Intron | + | gaaactcatcaaatatgcgt | 1679 | gttagtgt |
| | | | 22 | | | | |
| DCR62 | Mouse | DMD | Intron | - | tcatttacactaacacgcat | 1680 | atttgatg |
| | | | 22 | | | | |
| DCR63 | Mouse | DMD | Intron | + | gaatgaaactcatcaaatat | 1681 | gcgtgtta |
| | | | 22 | | | | |
| DCR64 | Mouse | DMD | Intron | - | tcatcaatatctttgaagga | 1682 | ctctgggt |
| | | | 23 | | | | |
| DCR65 | Mouse | DMD | Intron | - | tgttttcataggaaaaatag | 1683 | gcaagttg |
| | | | 23 | | | | |
| DCR66 | Mouse | DMD | Intron | + | aattggaaaatgtgatggga | 1684 | aacagata |
| | | | 23 | | | | |
| DCR67 | Human | DMD | Exon 51 | + | atgatcatcaagcagaaggt | 1685 | atgagaaa |
| DCR68 | Human | DMD | Exon 51 | + | agatgatcatcaagcagaag | 1686 | gtatgaga |
| DCR69 | Human | DMD | Exon 51 | - | cattttttctcataccttct | 1687 | gcttgatg |
| DCR70 | Human | DMD | Exon 51 | + | tcctactcagactgttactc | 1688 | tggtgaca |
| DCR71 | Human | DMD | Exon 51 | - | acaggttgtgtcaccagagt | 1689 | aacagtct |
| DCR72 | Human | DMD | Exon 51 | - | ttatcattttttctcatacc | 1690 | ttctgctt |
| DCR73 | Human | DMD | Intron | - | ttgcctaagaactggtggga | 1691 | aatggtct |
| | | | 51 | | | | |
| DCR74 | Human | DMD | Intron | - | aaacagttgcctaagaactg | 1692 | gtgggaaa |
| | | | 51 | | | | |
| DCR75 | Human | DMD | Intron | + | tttcccaccagttcttaggc | 1693 | aactgttt |
| | | | 51 | | | | |
| DCR76 | Human | DMD | Intron | + | tggctttgatttccctaggg | 1694 | tccagctt |
| | | | 50 | | | | |
| DCR77 | Human | DMD | Intron | - | tagggaaatcaaagccaatg | 1695 | aaacgttc |
| | | | 50 | | | | |
| DCR78 | Human | DMD | Intron | - | gaccctagggaaatcaaagc | 1696 | caatgaaa |
| | | | 50 | | | | |
| DCR79 | Human | DMD | Intron | - | TGAGGGCTCCACCCTCACGA | 1697 | GTGGGT |
| | | | 44 | | | | TT |
| DCR80 | Human | DMD | Intron | - | AAGGATTGAGGGCTCCACCC | 1698 | TCACGA |
| | | | 44 | | | | GT |
| DCR81 | Human | DMD | Intron | - | GCTCCACCCTCACGAGTGGG | 1699 | TTTGGT |
| | | | 44 | | | | TC |
| DCR82 | Human | DMD | Intron | - | TATCCCCTATCGAGGAAACC | 1700 | ACGAGT |
| | | | 55 | | | | TT |
| DCR83 | Human | DMD | Intron | + | GATAAAGAAGGCCTATTTCA | 1701 | TAGAGT |
| | | | 55 | | | | TG |
| DCR84 | Human | DMD | Intron | - | AGGCCTTCTTTATCCCCTAT | 1702 | CGAGG |
| | | | 55 | | | | AAA |
| DCR85 | Human | DMD | Intron | - | TGAGGGCTCCACCCTCACGA | 1703 | GTGGGT |
| | | | 44 | | | | |
| DCR86 | Human | DMD | Intron | + | GATAAAGAAGGCCTATTTCA | 1704 | TAGAGT |
| | | | 55 | | | | |
| DCR1 | Human | DMD | Intron | + | attggctttgatttcccta | 1705 | GGG |
| | | | 50 | | | | |
| DCR2 | Human | DMD | Intron | - | tgtagagtaagtcagccta | 1706 | TGG |
| | | | 50 | | | | |
| DCR3 | Human | DMD | Exon | + | cctactcagactgttactc | 1707 | TGG |
| | | | 51-5′ | | | | |
| DCR4 | Human | DMD | Exon | + | ttggacagaacttaccgac | 1708 | TGG |
| | | | 51-3′ | | | | |
| DCR5 | Human | DMD | Intron | - | cagttgcctaagaactggt | 1709 | GGG |
| | | | 51 | | | | |
| DCR6 | Human | DMD | Intron | - | GGGCTCCACCCTCACGAGT | 1710 | GGG |
| | | | 44 | | | | |
| DCR7 | Human | DMD | Intron | + | TTTGCTTCGCTATAAAACG | 1711 | AGG |
| | | | 55 | | | | |
| DCR8 | Human | DMD | Exon 41 | + | TCTGAGGATGGGGCCGCAA | 1712 | TGG |
| DCR9 | Human | DMD | Exon 44 | - | GATCTGTCAAATCGCCTGC | 1713 | AGG |
| DCR10 | Human | DMD | Exon 45 | + | CCAGGATGGCATTGGGCAG | 1714 | CGG |
| DCR11 | Human | DMD | Exon 45 | + | CTGAATCTGCGGTGGCAGG | 1715 | AGG |
| DCR12 | Human | DMD | Exon 46 | - | TTCTTTTGTTCTTCTAGCc | 1716 | TGG |
| DCR13 | Human | DMD | Exon 46 | + | GAAAAGCTTGAGCAAGTCA | 1717 | AGG |
| DCR14 | Human | DMD | Exon 47 | + | GAAGAGTTGCCCCTGCGCC | 1718 | AGG |
| DCR15 | Human | DMD | Exon 47 | + | ACAAATCTCCAGTGGATAA | 1719 | AGG |
| DCR16 | Human | DMD | Exon 48 | - | TGTTTCTCAGGTAAAGCTC | 1720 | TGG |
| DCR17 | Human | DMD | Exon 48 | + | GAAGGACCATTTGACGTTa | 1721 | AGG |
| DCR18 | Human | DMD | Exon 49 | - | AACTGCTATTTCAGTTTCc | 1722 | TGG |
| DCR19 | Human | DMD | Exon 49 | + | CCAGCCACTCAGCCAGTGA | 1723 | AGG |
| DCR20 | Human | DMD | Exon 50 | + | gtatgcttttctgttaaag | 1724 | AGG |
| DCR21 | Human | DMD | Exon 50 | + | CTCCTGGACTGACCACTAT | 1725 | TGG |
| DCR22 | Human | DMD | Exon 52 | + | GAACAGAGGCGTCCCCAGT | 1726 | TGG |
| DCR23 | Human | DMD | Exon 52 | + | GAGGCTAGAACAATCATTA | 1727 | CGG |
| DCR24 | Human | DMD | Exon 53 | + | ACAAGAACACCTTCAGAAC | 1728 | CGG |
| DCR25 | Human | DMD | Exon 53 | - | GGTTTCTGTGATTTTCTTT | 1729 | TGG |
| DCR26 | Human | DMD | Exon 54 | + | GGCCAAAGACCTCCGCCAG | 1730 | TGG |
| DCR27 | Human | DMD | Exon 54 | + | TTGGAGAAGCATTCATAAA | 1731 | AGG |
| DCR28 | Human | DMD | Exon 55 | - | TCGCTCACTCACCctgcaa | 1732 | AGG |
| DCR29 | Human | DMD | Exon 55 | + | AAAAGAGCTGATGAAACAA | 1733 | TGG |
| DCR30 | Human | DMD | 5′UTR/ | + | TAcACTTTTCaAAATGCTT | 1734 | TGG |
| | | | Exon 1 | | | | |
| DCR31 | Human | DMD | Exon 51 | + | gagatgatcatcaagcaga | 1735 | AGG |
| DCR32 | Mouse | DMD | mdx | + | ctttgaaagagcaaTaaaa | 1736 | TGG |
| DCR33 | Human | DMD | Intron | - | CACAAAAGTCAAATCGGAA | 1737 | TGG |
| | | | 44 | | | | |
| DCR34 | Human | DMD | Intron | - | ATTTCAATATAAGATTCGG | 1738 | AGG |
| | | | 44 | | | | |
| DCR35 | Human | DMD | Intron | - | CTTAAGCAATCCCGAACTC | 1739 | TGG |
| | | | 55 | | | | |
| DCR36 | Human | DMD | Intron | - | CCTTCTTTATCCCCTATCG | 1740 | AGG |
| | | | 55 | | | | |
| DCR40 | Mouse | DMD | Exon 23 | - | aggccaaacctcggcttac | 1741 | NNGRR |
| DCR41 | Mouse | DMD | Exon 23 | + | TTCGAAAATTTCAGgtaag | 1742 | NNGRR |
| DCR42 | Mouse | DMD | Exon 23 | + | gcagaacaggagataacag | 1743 | NNGRRT |
| DCR43 | Mouse | ACV | Exon 1 | + | gcggccctcgcccttctct | 1744 | ggggat |
| | | R2B | | | | | |
| DCR48 | Human | DMD | Intron | - | TAGTGATCGTGGATACGAG | 1745 | AGG |
| | | | 45 | | | | |
| DCR49 | Human | DMD | Intron | - | TACAGCCCTCGGTGTATAT | 1746 | TGG |
| | | | 45 | | | | |
| DCR50 | Human | DMD | Intron | - | GGAAGGAATTAAGCCCGAA | 1747 | TGG |
| | | | 52 | | | | |
| DCR51 | Human | DMD | Intron | - | GGAACAGCTTTCGTAGTTG | 1748 | AGG |
| | | | 53 | | | | |
| DCR52 | Human | DMD | Intron | + | ATAAAGTCCAGTGTCGATC | 1749 | AGG |
| | | | 54 | | | | |
| DCR53 | | | Intron | + | AAAACCAGAGCTTCGGTCA | 1750 | AGG |
| | | | 54 | | | | |
| DCR54 | Mouse | Rosa26 | ZFN | + | GAGTCTTCTGGGCAGGCTTAA | 1751 | TGG |
| | | | region | | | | |
| DCR55 | Mouse | Rosa26 | mRNA | - | TCGGGTGAGCATGTCTTTAAT | 1752 | TGG |
| DCR49 | Human | DMD | Ex 51 | - | gtgtcaccagagtaacagt | 1753 | ctgagt |
| DCR50 | Human | DMD | Ex 51 | + | tgatcatcaagcagaaggt | 1754 | atgag |
| DCR60 | Mouse | DMD | Exon 23 | + | AACTTCGAAAATTTCAGgta | 1755 | agccgagg |
| DCR61 | Mouse | DMD | Intron | + | gaaactcatcaaatatgcgt | 1756 | gttagtgt |
| | | | 22 | | | | |
| DCR62 | Mouse | DMD | Intron | - | tcatttacactaacacgcat | 1757 | atttgatg |
| | | | 22 | | | | |
| DCR63 | Mouse | DMD | Intron | + | gaatgaaactcatcaaatat | 1758 | gcgtgtta |
| | | | 22 | | | | |
| DCR64 | Mouse | DMD | Intron | - | tcatcaatatctttgaagga | 1759 | ctctgggt |
| | | | 23 | | | | |
| DCR65 | Mouse | DMD | Intron | - | tgttttcataggaaaaatag | 1760 | gcaagttg |
| | | | 23 | | | | |
| DCR66 | Mouse | DMD | Intron | + | aattggaaaatgtgatggga | 1761 | aacagata |
| | | 23 | | | | | |
| DCR67 | Human | DMD | Exon 51 | + | atgatcatcaagcagaaggt | 1762 | atgagaaa |
| DCR68 | Human | DMD | Exon 51 | + | agatgatcatcaagcagaag | 1763 | gtatgaga |
| DCR69 | Human | DMD | Exon 51 | - | cattttttctcataccttct | 1764 | gcttgatg |
| DCR70 | Human | DMD | Exon 51 | + | tcctactcagactgttactc | 1765 | tggtgaca |
| DCR71 | Human | DMD | Exon 51 | - | acaggttgtgtcaccagagt | 1766 | aacagtct |
| DCR72 | Human | DMD | Exon 51 | - | ttatcattttttctcatacc | 1767 | ttctgctt |
| DCR73 | Human | DMD | Intron | - | ttgcctaagaactggtggga | 1768 | aatggtct |
| | | | 51 | | | | |
| DCR74 | Human | DMD | Intron | - | aaacagttgcctaagaactg | 1769 | gtgggaaa |
| | | | 51 | | | | |
| DCR75 | Human | DMD | Intron | + | tttcccaccagttcttaggc | 1770 | aactgttt |
| | | | 51 | | | | |
| DCR76 | Human | DMD | Intron | + | tggctttgatttccctaggg | 1771 | tccagctt |
| | | | 50 | | | | |
| DCR77 | Human | DMD | Intron | - | tagggaaatcaaagccaatg | 1772 | aaacgttc |
| | | | 50 | | | | |
| DCR78 | Human | DMD | Intron | - | gaccctagggaaatcaaagc | 1773 | caatgaaa |
| | | | 50 | | | | |
| DCR79 | Human | DMD | Intron | - | TGAGGGCTCCACCCTCACGA | 1774 | GTGGGT |
| | | | 44 | | | | TT |
| DCR80 | Human | DMD | Intron | - | AAGGATTGAGGGCTCCACCC | 1775 | TCACGA |
| | | | 44 | | | | GT |
| DCR81 | Human | DMD | Intron | - | GCTCCACCCTCACGAGTGGG | 1776 | TTTGGT |
| | | | 44 | | | | TC |
| DCR82 | Human | DMD | Intron | - | TATCCCCTATCGAGGAAACC | 1777 | ACGAGT |
| | | | 55 | | | | TT |
| DCR83 | Human | DMD | Intron | + | GATAAAGAAGGCCTATTTCA | 1778 | TAGAGT |
| | | | 55 | | | | TG |
| DCR84 | Human | DMD | Intron | - | AGGCCTTCTTTATCCCCTAT | 1779 | CGAGG |
| | | | 55 | | | | AAA |
| DCR85 | Human | DMD | Intron | - | TGAGGGCTCCACCCTCACGA | 1780 | GTGGGT |
| | | | 44 | | | | |
| DCR86 | Human | DMD | Intron | + | GATAAAGAAGGCCTATTTCA | 1781 | TAGAGT |
| | | | 55 | | | | |
| | | DMD | | | UAGAAGAUCUGAGCUCUGAG | 1782 | |
| | | DMD | | | AGAUCUGAGCUCUGAGUGGA | 1783 | |
| | | DMD | | | UCUGAGCUCUGAGUGGAAGG | 1784 | |
| | | DMD | | | CCGUUUACUUCAAGAGCUGA | 1785 | |
| | | DMD | | | AAGCAGCCUGACCUAGCUCC | 1786 | |
| | | DMD | | | GCUCCUGGACUGACCACUAU | 1787 | |
| | | DMD | | | CCCUCAGCUCUUGAAGUAAA | 1788 | |
| | | DMD | | | GUCAGUCCAGGAGCUAGGUC | 1789 | |
| | | DMD | | | UAGUGGUCAGUCCAGGAGCU | 1790 | |
| | | DMD | | | GCUCCAAUAGUGGUCAGUCC | 1791 | |
| | | DMD | | | UGGCCAAAGACCUCCGCCAG | 1792 | |
| | | DMD | | | GUGGCAGACAAAUGUAGAUG | 1793 | |
| | | DMD | | | UGUAGAUGUGGCAAAUGACU | 1794 | |
| | | DMD | | | CUUGGCCCUGAAACUUCUCC | 1795 | |
| | | DMD | | | CAGAGAAUAUCAAUGCCUCU | 1796 | |
| | | DMD | | | CAGAGAAUAUCAAUGCCUCU | 1797 | |
| | | DMD | | | CAUUUGUCUGCCACUGGCGG | 1798 | |
| | | DMD | | | CUACAUUUGUCUGCCACUGG | 1799 | |
| | | DMD | | | CAUCUACAUUUGUCUGCCAC | 1800 | |
| | | DMD | | | AUAAUCCCGGAGAAGUUUCA | 1801 | |
| | | DMD | | | UAUCAUCUGCAGAAUAAUCC | 1802 | |
| | | DMD | | | UGUUAUCAUGUGGACUUUUC | 1803 | |
| | | DMD | | | UGAUAUAUCAUUUCUCUGUG | 1804 | |
| | | DMD | | | UUUAUGAAUGCUUCUCCAAG | 1805 | |
| | | DMD | | | UUCUCCAGGCUAGAAGAACAA | 1806 | |
| | | DMD | | | CUGCUCUUUUCCAGGUUCAAG | 1807 | |
| | | DMD | | | GUCUGUUUCAGUUACUGGUGG | 1808 | |
| | | DMD | | | UCCAGUUUCAUUUAAUUGUUU | 1809 | |
| | | DMD | | | CUUAUGGGAGCACUUACAAGC | 1810 | |
| | | DMD | | | UUGCUUCAUUACCUUCACUGG | 1811 | |
| | | DMD | | | UUGUGUCACCAGAGUAACAGU | 1812 | |
| | | DMD | | | AGUAACCACAGGUUGUGUCAC | 1813 | |
| | | DMD | | | UUCAAAUUUUGGGCAGCGGUA | 1814 | |
| | | DMD | | | CAAGAGGCUAGAACAAUCAUU | 1815 | |
| | | DMD | | | UUGUACUUCAUCCCACUGAUU | 1816 | |
| | | DMD | | | CUUCAGAACCGGAGGCAACAG | 1817 | |
| | | DMD | | | CAACAGUUGAAUGAAAUGUUA | 1818 | |
| | | DMD | | | GCCAAGCUUGAGUCAUGGAAG | 1819 | |
| | | DMD | | | CUUGGUUUCUGUGAUUUUCUU | 1820 | |
| | | DMD | | | UCAUUUCACAGGCCUUCAAGA | 1821 | |
| | | DMD | | | CAGAAAUAUUCGUACAGUCUC | 1822 | |
| | | DMD | | | CAAUUACCUCUGGGCUCCUGG | 1823 | |
| | | DMD | | | GATACTAGGGTGGCAAATAG | 1824 | |
| | | DMD | | | GTGTTCTTAAAAGAATGGTG | 1825 | |
| | | DMD | | | GTCAAGAACAGCTGCAGAAC | 1826 | |
| | | DMD | | | GCAGTTGAATGAAATGTTAA | 1827 | |
| | | DMD | | | GATACTAGTGTGGCTCATAG | 1828 | |
| | | DMD | | | GATACGATGGTGGCAAATCG | 1829 | |
| | | DMD | | | GATACTAGGGTGGGGAATAA | 1830 | |
| | | DMD | | | TTTTTCTTAAAAGAATGGTA | 1831 | |
| | | DMD | | | TTGATCTTAGAAGAATGGTG | 1832 | |
| | | DMD | | | GTTTTCTTGAAAAAATGGTG | 1833 | |
| | | DMD | | | CTGTTCTTAAAAGGTTGGTG | 1834 | |
| | | DMD | | | GAGTTCTTCAAAGAATAGTG | 1835 | |
| | | DMD | | | TCTAGGGCAGCTGCAGAAC | 1836 | |
| | | DMD | | | TCATTCACAGCTGCAGAAC | 1837 | |
| | | DMD | | | CAAAGAATAGCTGCAGAAC | 1838 | |
| | | DMD | | | TCAAGAACAGCTGCAGCAG | 1839 | |
| | | DMD | | | TCAAGAACAGCTGCATCAC | 1840 | |
| | | DMD | | | CAGTTACATGAAATGTTAA | 1841 | |
| | | DMD | | | CATTTTAATGAAATGTTAA | 1842 | |
| | | DMD | | | AAGTTGAATGAAATTTTAA | 1843 | |
| | | DMD | | | CAGTGGAATAAAATGTTAA | 1844 | |
| | | DMD | | | AAAGATATATAATGTCATGAAT | 1845 | |
| | | DMD | | | GCAGAATCAAATATAATAGTCT | 1846 | |
| | | DMD | | | AACAAATATCCCTTAGTATC | 1847 | |
| | | DMD | | | AATGTATTTCTTCTATTCAA | 1848 | |
| | | DMD | | | AACAATAAGTCAAATTTAATTG | 1849 | |
| | | DMD | | | GAACTGGTGGGAAATGGTCTA | 1850 | |
| | | | | | G | | |
| | | DMD | | | TCCTTTGGTAAATAAAAGTCCT | 1851 | |
| | | DMD | | | TAGGAATCAAATGGACTTGGAT | 1852 | |
| | | DMD | | | TAATTCTTTCTAGAAAGAGCCT | 1853 | |
| | | DMD | | | CTCTTGCATCTTGCACATGTCC | 1854 | |
| | | DMD | | | ACTTAGAGGTCTTCTACATACA | 1855 | |
| | | DMD | | | TCAGAGGTGAGTGGTGAGGGG | 1856 | |
| | | | | | A | | |
| | | DMD | | | ACACACAGCTGGGTTATCAGA | 1857 | |
| | | | | | G | | |
| | | DMD | | | CACAGCTGGGTTATCAGAG | 1858 | |
| | | DMD | | | ACACAGCTGGGTTATCAGAG | 1859 | |
| | | DMD | | | CACACAGCTGGGTTATCAGAG | 1860 | |
| | | DMD | | | AACACACAGCTGGGTTATCAG | 1861 | |
| | | | | | AG | | |
| | | DMD | | | CTGSTGGGARATGGTCTAG | 1862 | |
| | | DMD | | | ACTGGTGGGAAATGGTCTAG | 1863 | |
| | | DMD | | | AACTGGTGGGAAATGGTCTAG | 1864 | |
| | | DMD | | | AGAACTGGTGGGAAATGGTCT | 1865 | |
| | | | | | AG | | |
| | | DMD | | | ATATCTTCTTAAATACCCGA | 1866 | |
| | | DMD | | | AGTCTCACAAAACTGCAGAG | 1867 | |
| | | DMD | | | TACTTATGTATTTTAAAAAC | 1868 | |
| | | DMD | | | GAATAATTTCTATTATATTACA | 1869 | |
| | | DMD | | | TTCGAAAATTTCAGGTAAGCCG | 1870 | |
| | | DMD | | | TCATTTCTAAAAGTCTTTTGCC | 1871 | |
| | | DMD | | | TTTGAGACACAGTATAGGTTAT | 1872 | |
| | | DMD | | | ATATAATAGAAATTATTCAT | 1873 | |
| | | DMD | | | TAATATGCCCTGTAATATAA | 1874 | |
| | | DMD | | | TGATATCATCAATATCTTTG | 1875 | |
| | | DMD | | | GCAATTAATTGGAAAATGTG | 1876 | |
| | | DMD | | | CTTTAAGCTTAGGTAAAATCA | 1877 | |
| | | DMD | | | CAGTAATGTGTCATACCTTC | 1878 | |
| | | DMD | | | CAGGGCATATTATATTTAGA | 1879 | |
| | | DMD | | | CAAAAGCCAAATCTATTTCA | 1880 | |
| | | DMD | | | ATGCTTTGGTGGGAAGAAGTA | 1881 | |
| | | | | | GAGGA | | |
| | | DMD | | | ATGCTTTGGTGGGAAGAATAG | 1882 | |
| | | | | | AGGAC | | |
| | | DMD | | | TTGTGACAAGCTCACTAATTAG | 1883 | |
| | | | | | G | | |
| | | DMD | | | AAGTTTGAAGAACTTTTACCAG | 1884 | |
| | | | | | G | | |
| | | DMD | | | AGGCAGCGATAAAAAAAACCT | 1885 | |
| | | | | | GG | | |
| | | DMD | | | GCTTTGGTGGGAAGAAGTAGA | 1886 | |
| | | | | | GG | | |
| | | DMD | | | GCTGGGTGTCCCATTGAAA | 1887 | |
| | | DMD | | | CAGCCGCTCGCTGCAGCAG | 1888 | |
| | | DMD | | | TGGAGAGTTTGCAAGGAGC | 1889 | |
| | | DMD | | | GTTTATTCAGCCGGGAGTC | 1890 | |
| | | DMD | | | CGCCAGGAGGGGTGGGTCTA | 1891 | |
| | | DMD | | | CCTTGGTGAGACTGGTAGA | 1892 | |
| | | DMD | | | GTCTTCAGGTTCTGTTGCT | 1893 | |
| | | DMD | | | ATATTCCTGATTTAAAAGT | 1894 | |
| | | DMD | | | TTAAAAGTCGGCTGGTAGC | 1895 | |
| | | DMD | | | CGGGCCGGGGGCGGGGTCC | 1896 | |
| | | DMD | | | GCCCGAGCCGCGTGTGGAA | 1897 | |
| | | DMD | | | CCTTCATTGCGGCGGGCTG | 1898 | |
| | | DMD | | | CCGACCCCTCCCGGGTCCC | 1899 | |
| | | DMD | | | CAGGACCGCGCTTCCCACG | 1900 | |
| | | DMD | | | TGCACCCTGGGAGCGCGAG | 1901 | |
| | | DMD | | | CCGCACGCACCTGTTCCCA | 1902 | |
| | | DMD | | | AAAACAGCGAGGGAGAAAC | 1903 | |
| | | DMD | | | TTAACTTGATTGTGAAATC | 1904 | |
| | | DMD | | | AAAACAATGCATATTTGCA | 1905 | |
| | | DMD | | | AAAATCCAGTATTTTAATG | 1906 | |
| | | DMD | | | ACCCAGCACTGCAGCCTGG | 1907 | |
| | | DMD | | | AACTTATGCGGCGTTTCCT | 1908 | |
| | | DMD | | | TCACTTTAAAACCACCTCT | 1909 | |
| | | DMD | | | GCATCTTTTTCTCTTTAAT | 1910 | |
| | | DMD | | | TGTACTCTCTGAGGTGCTC | 1911 | |
| | | DMD | | | ACGCAGATAAGAACCAGTT | 1912 | |
| | | DMD | | | CATCAAGTCAGCCATCAGC | 1913 | |
| | | DMD | | | GAGTCACCCTCCTGGAAAC | 1914 | |
| | | DMD | | | GCTAGGGATGAAGAATAAA | 1915 | |
| | | DMD | | | TTGACCAATAGCCTTGACA | 1916 | |
| | | DMD | | | TGCAAATATCTGTCTGAAA | 1917 | |
| | | DMD | | | AAATTAGCAGTATCCTCTT | 1918 | |
| | | DMD | | | CCTGGGCTCCGGGGCGTTT | 1919 | |
| | | DMD | | | GGCCCCTGCGGCCACCCCG | 1920 | |
| | | DMD | | | CTCCCTCCCTGCCCGGTAG | 1921 | |
| | | DMD | | | AGGTTTGGAAAGGGCGTGC | 1922 | |
| | | DMD | | | GATTGGCTTTGATTTCCCTA | 1923 | |
| | | DMD | | | GTGTAGAGTAAGTCAGCCTATG | 1924 | |
| | | | | | G | | |
| | | DMD | | | GCCTACTCAGACTGTTACTC | 1925 | |
| | | DMD | | | GTTGGACAGAACTTACCGACTG | 1926 | |
| | | | | | G | | |
| | | DMD | | | GCAGTTGCCTAAGAACTGGT | 1927 | |
| | | DMD | | | GGGGCTCCACCCTCACGAGT | 1928 | |
| | | DMD | | | GTTTGCTTCGCTATAAAACGAG | 1929 | |
| | | | | | G | | |
| | | DMD | | | GTCTGAGGATGGGGCCGCAAT | 1930 | |
| | | | | | GG | | |
| | | DMD | | | GGATCTGTCAAATCGCCTGCAG | 1931 | |
| | | | | | G | | |
| | | DMD | | | GCCAGGATGGCATTGGGCAGC | 1932 | |
| | | | | | GG | | |
| | | DMD | | | GCTGAATCTGCGGTGGCAGGA | 1933 | |
| | | | | | GG | | |
| | | DMD | | | GTTCTTTTGTTCTTCTAGCCTGG | 1934 | |
| | | DMD | | | GGAAAAGCTTGAGCAAGTCAA | 1935 | |
| | | | | | GG | | |
| | | DMD | | | GGAAGAGTTGCCCCTGCGCCA | 1936 | |
| | | | | | GG | | |
| | | DMD | | | GACAAATCTCCAGTGGATAAA | 1937 | |
| | | | | | GG | | |
| | | DMD | | | GTGTTTCTCAGGTAAAGCTCTG | 1938 | |
| | | | | | G | | |
| | | DMD | | | GGAAGGACCATTTGACGTTAA | 1939 | |
| | | | | | GG | | |
| | | DMD | | | GAACTGCTATTTCAGTTTCCTG | 1940 | |
| | | | | | G | | |
| | | DMD | | | GCCAGCCACTCAGCCAGTGAA | 1941 | |
| | | | | | GG | | |
| | | DMD | | | GGTATGCTTTTCTGTTAAAGAG | 1942 | |
| | | | | | G | | |
| | | DMD | | | GCTCCTGGACTGACCACTATTG | 1943 | |
| | | | | | G | | |
| | | DMD | | | GGAACAGAGGCGTCCCCAGTT | 1944 | |
| | | | | | GG | | |
| | | DMD | | | GGAGGCTAGAACAATCATTAC | 1945 | |
| | | | | | GG | | |
| | | DMD | | | GACAAGAACACCTTCAGAACC | 1946 | |
| | | | | | GG | | |
| | | DMD | | | GGGTTTCTGTGATTTTCTTTTGG | 1947 | |
| | | DMD | | | GGGCCAAAGACCTCCGCCAGT | 1948 | |
| | | | | | GG | | |
| | | DMD | | | GTTGGAGAAGCATTCATAAAA | 1949 | |
| | | | | | GG | | |
| | | DMD | | | GTCGCTCACTCACCCTGCAAAG | 1950 | |
| | | | | | G | | |
| | | DMD | | | GAAAAGAGCTGATGAAACAAT | 1951 | |
| | | | | | GG | | |
| | | DMD | | | GTACACTTTTCAAAATGCTTTG | 1952 | |
| | | | | | G | | |
| | | DMD | | | GGAGATGATCATCAAGCAGAA | 1953 | |
| | | | | | GG | | |
| | | DMD | | | GCTTTGAAAGAGCAATAAAAT | 1954 | |
| | | | | | GG | | |
| | | DMD | | | GCACAAAAGTCAAATCGGAAT | 1955 | |
| | | | | | GG | | |
| | | DMD | | | GATTTCAATATAAGATTCGGAG | 1956 | |
| | | | | | G | | |
| | | DMD | | | GCTTAAGCAATCCCGAACTCTG | 1957 | |
| | | | | | G | | |
| | | DMD | | | GCCTTCTTTATCCCCTATCG | 1958 | |
| | | DMD | | | GAGGCCAAACCTCGGCTTACN | 1959 | |
| | | | | | NGRR | | |
| | | DMD | | | GTTCGAAAATTTCAGGTAAGNN | 1960 | |
| | | | | | GRR | | |
| | | DMD | | | GGCAGAACAGGAGATAACAGN | 1961 | |
| | | | | | NGRRT | | |
| | | DMD | | | GGCGGCCCTCGCCCTTCTCTGG | 1962 | |
| | | | | | GGAT | | |
| | | DMD | | | GTAGTGATCGTGGATACGAGA | 1963 | |
| | | | | | GG | | |
| | | DMD | | | GTACAGCCCTCGGTGTATATTG | 1964 | |
| | | | | | G | | |
| | | DMD | | | GGGAAGGAATTAAGCCCGAAT | 1965 | |
| | | | | | GG | | |
| | | DMD | | | GGGAACAGCTTTCGTAGTTGAG | 1966 | |
| | | | | | G | | |
| | | DMD | | | GATAAAGTCCAGTGTCGATCAG | 1967 | |
| | | | | | G | | |
| | | DMD | | | GAAAACCAGAGCTTCGGTCAA | 1968 | |
| | | | | | GG | | |
| | | DMD | | | GGAGTCTTCTGGGCAGGCTTAA | 1969 | |
| | | | | | AGGCTAACCTGG | | |
| | | DMD | | | GTCGGGTGAGCATGTCTTTAAT | 1970 | |
| | | | | | CTACCTCGATGG | | |
| | | DMD | | | GGTGTCACCAGAGTAACAGTCT | 1971 | |
| | | | | | GAGT | | |
| | | DMD | | | GTGATCATCAAGCAGAAGGTA | 1972 | |
| | | | | | TGAG | | |
| | | DMD | | | GAACTTCGAAAATTTCAGGTAA | 1973 | |
| | | | | | GCCGAGG | | |
| | | DMD | | | GGAAACTCATCAAATATGCGTG | 1974 | |
| | | | | | TTAGTGT | | |
| | | DMD | | | GTCATTTACACTAACACGCATA | 1975 | |
| | | | | | TTTGATG | | |
| | | DMD | | | GGAATGAAACTCATCAAATAT | 1976 | |
| | | | | | GCGTGTTA | | |
| | | DMD | | | GTCATCAATATCTTTGAAGGAC | 1977 | |
| | | | | | TCTGGGT | | |
| | | DMD | | | GTGTTTTCATAGGAAAAATAGG | 1978 | |
| | | | | | CAAGTTG | | |
| | | DMD | | | GAATTGGAAAATGTGATGGGA | 1979 | |
| | | | | | AACAGATA | | |
| | | DMD | | | GATGATCATCAAGCAGAAGGT | 1980 | |
| | | | | | ATGAGAAA | | |
| | | DMD | | | GAGATGATCATCAAGCAGAAG | 1981 | |
| | | | | | GTATGAGA | | |
| | | DMD | | | GCATTTTTTCTCATACCTTCTGC | 1982 | |
| | | | | | TTGATG | | |
| | | DMD | | | GTCCTACTCAGACTGTTACTCT | 1983 | |
| | | | | | GGTGACA | | |
| | | DMD | | | GACAGGTTGTGTCACCAGAGTA | 1984 | |
| | | | | | ACAGTCT | | |
| | | DMD | | | GTTATCATTTTTTCTCATACCTT | 1985 | |
| | | | | | CTGCTT | | |
| | | DMD | | | GTTGCCTAAGAACTGGTGGGA | 1986 | |
| | | | | | AATGGTCT | | |
| | | DMD | | | GAAACAGTTGCCTAAGAACTG | 1987 | |
| | | | | | GTGGGAAA | | |
| | | DMD | | | GTTTCCCACCAGTTCTTAGGCA | 1988 | |
| | | | | | ACTGTTT | | |
| | | DMD | | | GTGGCTTTGATTTCCCTAGGGT | 1989 | |
| | | | | | CCAGCTT | | |
| | | DMD | | | GTAGGGAAATCAAAGCCAATG | 1990 | |
| | | | | | AAACGTTC | | |
| | | DMD | | | GGACCCTAGGGAAATCAAAGC | 1991 | |
| | | | | | CAATGAAA | | |
| | | DMD | | | GTGAGGGCTCCACCCTCACGAG | 1992 | |
| | | | | | TGGGTTT | | |
| | | DMD | | | GAAGGATTGAGGGCTCCACCCT | 1993 | |
| | | | | | CACGAGT | | |
| | | DMD | | | GGCTCCACCCTCACGAGTGGGT | 1994 | |
| | | | | | TTGGTTC | | |
| | | DMD | | | GTATCCCCTATCGAGGAAACCA | 1995 | |
| | | | | | CGAGTTT | | |
| | | DMD | | | GGATAAAGAAGGCCTATTTCAT | 1996 | |
| | | | | | AGAGTTG | | |
| | | DMD | | | GAGGCCTTCTTTATCCCCTATC | 1997 | |
| | | | | | GAGGAAA | | |
| | | DMD | | | GTGAGGGCTCCACCCTCACGAG | 1998 | |
| | | | | | TGGGT | | |
| | | DMD | | | GGATAAAGAAGGCCTATTTCAT | 1999 | |
| | | | | | AGAGT | | |
| | | DMD | | | CACCGCAGCCGCTCGCTGCAGC | 2000 | |
| | | | | | AG | | |
| | | DMD | | | AAACCTGCTGCAGCGAGCGGC | 2001 | |
| | | | | | TGC | | |
| | | DMD | | | CACCGGCTGGGTGTCCCATTGA | 2002 | |
| | | | | | AA | | |
| | | DMD | | | AAACTTTCAATGGGACACCCAG | 2003 | |
| | | | | | CC | | |
| | | DMD | | | CACCGGTTTATTCAGCCGGGAG | 2004 | |
| | | | | | TC | | |
| | | DMD | | | AAACGACTCCCGGCTGAATAA | 2005 | |
| | | | | | ACC | | |
| | | DMD | | | CACCGTGGAGAGTTTGCAAGG | 2006 | |
| | | | | | AGC | | |
| | | DMD | | | AAACGCTCCTTGCAAACTCTCC | 2007 | |
| | | | | | AC | | |
| | | DMD | | | CACCGCCCTCCAGACTTTCCAC | 2008 | |
| | | | | | CT | | |
| | | DMD | | | AAACAGGTGGAAAGTCTGGAG | 2009 | |
| | | | | | GGC | | |
| | | DMD | | | CACCGAATTTTCTTCCAAGTTC | 2010 | |
| | | | | | TC | | |
| | | DMD | | | AAACGAGAACTTGGAAGAAAA | 2011 | |
| | | | | | TTC | | |
| | | DMD | | | CACCGCTGCGGAGAGAAGAAA | 2012 | |
| | | | | | GGG | | |
| | | DMD | | | AAACCCCTTTCTTCTCTCCGCA | 2013 | |
| | | | | | GC | | |
| | | DMD | | | CACCGAGAGCCACCCCCTGGCT | 2014 | |
| | | | | | CC | | |
| | | DMD | | | AAACGGAGCCAGGGGGTGGCT | 2015 | |
| | | | | | CTC | | |
| | | DMD | | | CACCGCGAAGCCAACCGCGGC | 2016 | |
| | | | | | GGG | | |
| | | DMD | | | AAACCCCGCCGCGGTTGGCTTC | 2017 | |
| | | | | | GC | | |
| | | DMD | | | CACCGAGAGGGAAGACGATCG | 2018 | |
| | | | | | CCC | | |
| | | DMD | | | AAACGGGCGATCGTCTTCCCTC | 2019 | |
| | | | | | TC | | |
| | | DMD | | | CACCGCCCCTTTAACTTTCCTC | 2020 | |
| | | | | | CG | | |
| | | DMD | | | AAACCGGAGGAAAGTTAAAGG | 2021 | |
| | | | | | GGC | | |
| | | DMD | | | CACCGGCAGCCCCGCTTCCTTC | 2022 | |
| | | | | | AA | | |
| | | DMD | | | AAACTTGAAGGAAGCGGGGCT | 2023 | |
| | | | | | GCC | | |
| | | DMD | | | CACCGCGAGAGCGAGAGGAGG | 2024 | |
| | | | | | GAG | | |
| | | DMD | | | AAACCTCCCTCCTCTCGCTCTC | 2025 | |
| | | | | | GC | | |
| | | DMD | | | CACCGGAGAGAGCTTGAGAGC | 2026 | |
| | | | | | GCG | | |
| | | DMD | | | AAACCGCGCTCTCAAGCTCTCT | 2027 | |
| | | | | | CC | | |
| | | DMD | | | CACCGGGTGGAGGGGGCGGGG | 2028 | |
| | | | | | CCC | | |
| | | DMD | | | AAACGGGCCCCGCCCCCTCCAC | 2029 | |
| | | | | | CC | | |
| | | DMD | | | CACCGGGTATCCACGTAAATCA | 2030 | |
| | | | | | AA | | |
| | | DMD | | | AAACTTTGATTTACGTGGATAC | 2031 | |
| | | | | | CC | | |
| | | DMD | | | CACCGCCAATCACTGGCTCCGG | 2032 | |
| | | | | | TC | | |
| | | DMD | | | AAACGACCGGAGCCAGTGATT | 2033 | |
| | | | | | GGC | | |
| | | DMD | | | CACCGGGCGCCCGAGGGAAGA | 2034 | |
| | | | | | AGA | | |
| | | DMD | | | AAACTCTTCTTCCCTCGGGCGC | 2035 | |
| | | | | | CC | | |
| | | DMD | | | CACCGGGGTGGGGGTACCAGA | 2036 | |
| | | | | | GGA | | |
| | | DMD | | | AAACTCCTCTGGTACCCCCACC | 2037 | |
| | | | | | CC | | |
| | | DMD | | | CACCGCCGGGGACAGAAGAGA | 2038 | |
| | | | | | GGG | | |
| | | DMD | | | AAACCCCTCTCTTCTGTCCCCG | 2039 | |
| | | | | | GC | | |
| | | DMD | | | CACCGGAGAGAGAGTGGGAGA | 2040 | |
| | | | | | AGC | | |
| | | DMD | | | AAACGCTTCTCCCACTCTCTCT | 2041 | |
| | | | | | CC | | |
| | | DMD | | | CACCGAAAGTAACTGTCAAAT | 2042 | |
| | | | | | GCG | | |
| | | DMD | | | AAACCGCATTTGACAGTTACTT | 2043 | |
| | | | | | TC | | |
| | | DMD | | | CACCGTTAACCAGAGCGCCCA | 2044 | |
| | | | | | GTC | | |
| | | DMD | | | AAACGACTGGGCGCTCTGGTTA | 2045 | |
| | | | | | AC | | |
| | | DMD | | | CACCGCGTCGGAGCTGCCCGCT | 2046 | |
| | | | | | AG | | |
| | | DMD | | | AAACCTAGCGGGCAGCTCCGA | 2047 | |
| | | | | | CGC | | |
| | | DMD | | | TGTACTCTCTGAGGTGCTC | 2048 | |
| | | DMD | | | ACGCAGATAAGAACCAGTT | 2049 | |
| | | DMD | | | CATCAAGTCAGCCATCAGC | 2050 | |
| | | DMD | | | GAGTCACCCTCCTGGAAAC | 2051 | |
| | | DMD | | | CCTGGGCTCCGGGGCGTTT | 2052 | |
| | | DMD | | | GGCCCCTGCGGCCACCCCG | 2053 | |
| | | DMD | | | CTCCCTCCCTGCCCGGTAG | 2054 | |
| | | DMD | | | AGGTTTGGAAAGGGCGTGC | 2055 | |
| | | DMD | | | ACTCCACTGCACTCCAGTCT | 2056 | |
| | | DMD | | | TCTGTGGGGGACCTGCACTG | 2057 | |
| | | DMD | | | GGGGCGCCAGTTGTGTCTCC | 2058 | |
| | | DMD | | | ACACCATTGCCACCACCATT | 2059 | |
| | | DMD | | | CAATGACCCCTTCATTGACC | 2060 | |
| | | DMD | | | TTGATTTTGGAGGGATCTCG | 2061 | |
| | | DMD | | | GGAATCCATGGAGGGAAGAT | 2062 | |
| | | DMD | | | TGTTCTCGCTCAGGTCAGTG | 2063 | |
| | | DMD | | | CTCTCTGCTCCTTTGCCACA | 2064 | |
| | | DMD | | | GTGCTCTTCGGGTTTCAGGA | 2065 | |
| | | DMD | | | CGAAAGAGAAAGCGAACCAGT | 2066 | |
| | | | | | ATCGAGAAC | | |
| | | DMD | | | CGTTGTGCATAGTCGCTGCTTG | 2067 | |
| | | | | | ATCGC | | |
| | | DMD | | | UAGAAGAUCUGAGCUCUGAG | 2068 | |
| | | DMD | | | AGAUCUGAGCUCUGAGUGGA | 2069 | |
| | | DMD | | | UCUGAGCUCUGAGUGGAAGG | 2070 | |
| | | DMD | | | CCGUUUACUUCAAGAGCUGA | 2071 | |
| | | DMD | | | AAGCAGCCUGACCUAGCUCC | 2072 | |
| | | DMD | | | GCUCCUGGACUGACCACUAU | 2073 | |
| | | DMD | | | CCCUCAGCUCUUGAAGUAAA | 2074 | |
| | | DMD | | | GUCAGUCCAGGAGCUAGGUC | 2075 | |
| | | DMD | | | UAGUGGUCAGUCCAGGAGCU | 2076 | |
| | | DMD | | | GCUCCAAUAGUGGUCAGUCC | 2077 | |
| | | DMD | | | UGGCCAAAGACCUCCGCCAG | 2078 | |
| | | DMD | | | GUGGCAGACAAAUGUAGAUG | 2079 | |
| | | DMD | | | UGUAGAUGUGGCAAAUGACU | 2080 | |
| | | DMD | | | CUUGGCCCUGAAACUUCUCC | 2081 | |
| | | DMD | | | CAGAGAAUAUCAAUGCCUCU | 2082 | |
| | | DMD | | | CAGAGAAUAUCAAUGCCUCU | 2083 | |
| | | DMD | | | CAUUUGUCUGCCACUGGCGG | 2084 | |
| | | DMD | | | CUACAUUUGUCUGCCACUGG | 2085 | |
| | | DMD | | | CAUCUACAUUUGUCUGCCAC | 2086 | |
| | | DMD | | | AUAAUCCCGGAGAAGUUUCA | 2087 | |
| | | DMD | | | UAUCAUCUGCAGAAUAAUCC | 2088 | |
| | | DMD | | | UGUUAUCAUGUGGACUUUUC | 2089 | |
| | | DMD | | | UGAUAUAUCAUUUCUCUGUG | 2090 | |
| | | DMD | | | UUUAUGAAUGCUUCUCCAAG | 2091 | |
| | | DMD | | | UUCUCCAGGCUAGAAGAACAA | 2092 | |
| | | DMD | | | CUGCUCUUUUCCAGGUUCAAG | 2093 | |
| | | DMD | | | GUCUGUUUCAGUUACUGGUGG | 2094 | |
| | | DMD | | | UCCAGUUUCAUUUAAUUGUUU | 2095 | |
| | | DMD | | | CUUAUGGGAGCACUUACAAGC | 2096 | |
| | | DMD | | | UUGCUUCAUUACCUUCACUGG | 2097 | |
| | | DMD | | | UUGUGUCACCAGAGUAACAGU | 2098 | |
| | | DMD | | | AGUAACCACAGGUUGUGUCAC | 2099 | |
| | | DMD | | | UUCAAAUUUUGGGCAGCGGUA | 2100 | |
| | | DMD | | | CAAGAGGCUAGAACAAUCAUU | 2101 | |
| | | DMD | | | UUGUACUUCAUCCCACUGAUU | 2102 | |
| | | DMD | | | CUUCAGAACCGGAGGCAACAG | 2103 | |
| | | DMD | | | CAACAGUUGAAUGAAAUGUUA | 2104 | |
| | | DMD | | | GCCAAGCUUGAGUCAUGGAAG | 2105 | |
| | | DMD | | | CUUGGUUUCUGUGAUUUUCUU | 2106 | |
| | | DMD | | | UCAUUUCACAGGCCUUCAAGA | 2107 | |
| | | DMD | | | CAGAAAUAUUCGUACAGUCUC | 2108 | |
| | | DMD | | | CAAUUACCUCUGGGCUCCUGG | 2109 | |
| | | DMD | | | GAACUUCUAUUUAAUUUUG | 2110 | |
| | | DMD | | | AUUUCAGGUAAGCCGAGGUU | 2111 | |
| | | DMD | | | UCUUAAUAAUGUUUCACUGU | 2112 | |
| | | DMD | | | AUAAUUUCUAUUAUAUUACA | 2113 | |
| | | DMD | | | UUUCAUUCAUAUCAAGAAGA | 2114 | |
| | | DMD | | | AUAGUUUAAAGGCCAAACCU | 2115 | |
| | | DMD | | | UGUGAAAAAAUAUAGUUUAA | 2116 | |
| | | DMD | | | CGAAAAUUUCAGGUAAGCCG | 2117 | |
| | | DMD | | | CAAAAACCCAAAATATTTTAGC | 2118 | |
| | | | | | T | | |
| | | DMD | | | CCTTTTTGGTATCTTACAGGAA | 2119 | |
| | | | | | C | | |
| | | DMD | | | CCGCTGCCCAATGCCATCCTGG | 2120 | |
| | | | | | A | | |
| | | DMD | | | TTTTTCCTTTTATTCTAGTTGAA | 2121 | |
| | | DMD | | | TTGATCCATATGCTTTTACCTG | 2122 | |
| | | | | | C | | |
| | | DMD | | | TCAACAGATCTGTCAAATCGCC | 2123 | |
| | | | | | T | | |
| | | DMD | | | TTCTTCTTTCTCCAGGCTAGAA | 2124 | |
| | | | | | G | | |
| | | DMD | | | GTTCTTCTAGCCTGGAGAAAGA | 2125 | |
| | | | | | A | | |
| | | DMD | | | CAAATCCTGCATTGTTGCCTGT | 2126 | |
| | | | | | A | | |
| | | DMD | | | CTGTTAAAGAGGAAGTTAGAA | 2127 | |
| | | | | | GA | | |
| | | DMD | | | AAAATTTTTATATTACAGAATA | 2128 | |
| | | | | | T | | |
| | | DMD | | | TTGTAGACTATCTTTTATATTCT | 2129 | |
| | | DMD | | | TTTTGCATTTTAGATGAAAGAG | 2130 | |
| | | | | | A | | |
| | | DMD | | | AACATCTTCTCTTTCATCTAAA | 2131 | |
| | | | | | A | | |
| | | DMD | | | TTTTGAACATCTTCTCTTTCATC | 2132 | |
| | | DMD | | | CAAAAACCCAAAATATTTTAGC | 2133 | |
| | | | | | T | | |
| | | DMD | | | GCTTGTGTTTCTAATTTTTCTTT | 2134 | |
| | | DMD | | | ACTTATTGTTATTGAAATTGGC | 2135 | |
| | | | | | T | | |
| | | DMD | | | TACCATGTATTGCTAAACAAAG | 2136 | |
| | | | | | T | | |
| | | DMD | | | GTATCAATTCACACCAGCAAGT | 2137 | |
| | | | | | T | | |
| | | DMD | | | CTCCTCTGTAAAGTGGCGATTA | 2138 | |
| | | | | | T | | |
| | | DMD | | | TTTAAAATGAAGATTTTCCACC | 2139 | |
| | | | | | A | | |
| | | DMD | | | AAATGAAGATTTTCCACCAATC | 2140 | |
| | | | | | A | | |
| | | DMD | | | CCACCAATCACTTTACTCTCCT | 2141 | |
| | | | | | A | | |
| | | DMD | | | CCACCAGTTCTTAGGCAACTGT | 2142 | |
| | | | | | T | | |
| | | DMD | | | CATTAATTTATATCCTTGATTAT | 2143 | |
| | | DMD | | | GTTGTTGTTGTTAAGGTCAAAG | 2144 | |
| | | | | | T | | |
| | | DMD | | | AAATTACCCTAGATCTTAAAGT | 2145 | |
| | | | | | T | | |
| | | DMD | | | GCCTCTGATTAGGGTGGGGGCG | 2146 | |
| | | | | | TG | | |
| | | DMD | | | TCACAGGCTCCAGGAAGGGTTT | 2147 | |
| | | | | | GG | | |
| | | DMD | | | CCCAGGGGGGCCTCTTTCGGAA | 2148 | |
| | | | | | GG | | |
| | | DMD | | | GGAAGGCTCTCTTGGTGATGGA | 2149 | |
| | | | | | GA | | |
| | | DMD | | | AAGCTAGTCTAGTGCAAGCTAA | 2150 | |
| | | | | | CA | | |
| | | DMD | | | CTGGCCTATGTTATTACCTGTA | 2151 | |
| | | | | | TG | | |
| | | DMD | | | TGGCCTATGTTATTACCTGTAT | 2152 | |
| | | | | | GG | | |
| | | DMD | | | TTCCATTCTAATGGGTGGCTGT | 2153 | |
| | | | | | T | | |
| | | DMD | | | CTCCTCTGTAAAGTGGCGAT | 2154 | |
| | | DMD | | | TTCCATTCTAATGGGTGGCT | 2155 | |
| | | DMD | | | GTATCAATTCACACCAGCAA | 2156 | |
| | | DMD | | | TACCATGTATTGCTAAACAA | 2157 | |
| | | DMD | | | ACTTATTGTTATTGAAATTG | 2158 | |
| | | DMD | | | GCTTGTGTTTCTAATTTTTC | 2159 | |
| | | DMD | | | CAAAAACCCAAAATATTTTA | 2160 | |
| | | DMD | | | TTTAAAATGAAGATTTTCCA | 2161 | |
| | | DMD | | | AAATGAAGATTTTCCACCAA | 2162 | |
| | | DMD | | | CCACCAATCACTTTACTCTC | 2163 | |
| | | DMD | | | CCACCAGTTCTTAGGCAACT | 2164 | |
| | | DMD | | | CATTAATTTATATCCTTGAT | 2165 | |
| | | DMD | | | AGTTATAGCTCTCTTTCAAT | 2166 | |
| | | DMD | | | ATGTATAACAATTCCAACAT | 2167 | |
| | | DMD | | | AAATTACCCTAGATCTTAAA | 2168 | |
| | | DMD | | | GTTGTTGTTGTTAAGGTCAA | 2169 | |
| | | DMD | | | GCTTGTGTTTCTAATTTTTC | 2170 | |
| | | DMD | | | TAATTTTTCTTTTTCTTCTT | 2171 | |
| | | DMD | | | GCAAAAAGGAAAAAAGAAGA | 2172 | |
| | | DMD | | | GGGTTTTTGCAAAAAGGAAA | 2173 | |
| | | DMD | | | AGCTCCTACTCAGACTGTTA | 2174 | |
| | | DMD | | | TGCAAAAACCCAAAATATTT | 2175 | |
| | | DMD | | | TGTCACCAGAGTAACAGTCT | 2176 | |
| | | DMD | | | CTTAGTAACCACAGGTTGTG | 2177 | |
| | | DMD | | | TAGTTTGGAGATGGCAGTTT | 2178 | |
| | | DMD | | | GAGATGGCAGTTTCCTTAGT | 2179 | |
| | | DMD | | | CTTGATGTTGGAGGTACCTG | 2180 | |
| | | DMD | | | ATGTTGGAGGTACCTGCTCT | 2181 | |
| | | DMD | | | TAACTTGATCAAGCAGAGAA | 2182 | |
| | | DMD | | | TCTGCTTGATCAAGTTATAA | 2183 | |
| | | DMD | | | TAAAATCACAGAGGGTGATG | 2184 | |
| | | DMD | | | ATATCCTCAAGGTCACCCAC | 2185 | |
| | | DMD | | | ATGATCATCTCGTTGATATC | 2186 | |
| | | DMD | | | TCATACCTTCTGCTTGATGA | 2187 | |
| | | DMD | | | TCATTTTTTCTCATACCTTC | 2188 | |
| | | DMD | | | TGCCAACTTTTATCATTTTT | 2189 | |
| | | DMD | | | AATCAGAAAGAAGATCTTAT | 2190 | |
| | | DMD | | | ATTTCCCTAGGGTCCAGCTT | 2191 | |
| | | DMD | | | GCTCAAATTGTTACTCTTCA | 2192 | |
| | | DMD | | | AGCTCCTACTCAGACTGTTA | 2193 | |
| | | DMD | | | ATTCTAGTACTATGCATCTT | 2194 | |
| | | DMD | | | ACTTAAGTTACTTGTCCAGG | 2195 | |
| | | DMD | | | CCAAGGTCCCAGAGTTCCTA | 2196 | |
| | | DMD | | | TTTCCCTGGCAAGGTCTGAA | 2197 | |
| | | DMD | | | GCTCATTCTCATGCCTGGAC | 2198 | |
| | | DMD | | | TTTAGCAATACATGGTAGAA | 2199 | |
| | | DMD | | | AGCCAAACTCTTATTCATGA | 2200 | |
| | | DMD | | | TAACAATGTGGATACTTTGT | 2201 | |
| | | DMD | | | GUGUUAUUACUUGCUACUGCA | 2202 | |
| | | DMD | | | GUGUAUUGCUUGUACUACUCA | 2203 | |
| | | DMD | | | GUUUAAAUGUAAAUAGCUCAG | 2204 | |
| | | DMD | | | GAAUUUUCAAUGAUGUUCUGG | 2205 | |
| | | | | | G | | |
| | | DMD | | | GAACUGGUGGGAAAUGGUCUA | 2206 | |
| | | | | | G | | |
| | | DMD | | | GUUUCAUUGGCUUUGAUUUCC | 2207 | |
| | | | | | C | | |
| | | DMD | | | GGCAAUUCUCCUGAAUAGAAA | 2208 | |
| | | DMD | | | GAUUAUACUUAGGCUGAAUAG | 2209 | |
| | | | | | U | | |
| | | DMD | | | GACUUCCAGAAUUAUGUGUUC | 2210 | |
| | | DMD | | | GUGAGGGCCUGACACAUGGUA | 2211 | |
| | | DMD | | | GUGAAGAUCAUUUCUUGGUAG | 2212 | |
| | | DMD | | | GCACAGUCAGAACUAGUGUGC | 2213 | |
| | | DMD | | | GAGUAAGCCCGAUCAUUAUUG | 2214 | |
| | | DMD | | | GGAAGGGACAUAUUCUAUGGG | 2215 | |
| | | DMD | | | GACCACAAGCUGACUUGGGGG | 2216 | |
| | | DMD | | | GGAUUUGUAUCCAUUAUCUGG | 2217 | |
| | | DMD | | | CUCUGCAUUGUUUUGGCCUC | 2218 | |
| | | DMD | | | UCCUCCAAAGAGUAGAAUGG | 2219 | |
| | | DMD | | | GCCCUAAACUUACACUGUUC | 2220 | |
| | | DMD | | | AAAGAUAGAUUAGAUUGUCC | 2221 | |
| | | DMD | | | GUUGCUAAAUUACAUAGUUU | 2222 | |
| | | DMD | | | UGUUGCAAUAGUCAAUCAAG | 2223 | |
| | | DMD | | | AUACUGAUUAAGACAGAUGA | 2224 | |
| | | DMD | | | AAUACUGAUUAAGACAGAUG | 2225 | |
| | | DMD | | | CUCUAUACAAAUGCCAACGC | 2226 | |
| | | DMD | | | ACUUGCAUGCACACCAGCGU | 2227 | |
| | | DMD | | | UUGGGCUAAUGUAGCAUAAU | 2228 | |
| | | DMD | | | GCGUUGGCAUUUGUAUAGAG | 2229 | |
| | | DMD | | | UGGGCUAAGUAGCAUAAUG | 2230 | |
| | | DMD | | | UUUGGGCUAAUGUAGCAUAA | 2231 | |
| | | DMD | | | GCUUAACUCCUUAAUAUUAA | 2232 | |
| | | DMD | | | UCUUCUAUAUUAAAGCAGAU | 2233 | |
| | | DMD | | | CUUCUAUAUUAAAGCAGAUU | 2234 | |
| | | DMD | | | AAUAUAUAACUACCUUGGGU | 2235 | |
| | | DMD | | | ACCUCCAUUCUACUCUUUGG | 2236 | |
| | | DMD | | | UUUCAAUGAUAUCCAACCCA | 2237 | |
| | | DMD | | | AGUACCUCCAUUCUACUCUU | 2238 | |
| | | DMD | | | CUAUCCUCCAAAGAGUAGAA | 2239 | |
| | | DMD | | | UUUUGCUACAUAUUUCAGGC | 2240 | |
| | | DMD | | | UUUGCUACAUAUUUCAGGCU | 2241 | |
| | | DMD | | | GGGUUGGAUAUCAUUGAAAA | 2242 | |
| | | DMD | | | AUAUUUCAGGCUGGGUUCU | 2243 | |
| | | DMD | | | UUGAAAUAUAUAACUACCUU | 2244 | |
| | | DMD | | | AUUGAAAUAUAUAACUACCU | 2245 | |
| | | DMD | | | GUGAGUAGUGGGGCACUUUA | 2246 | |
| | | DMD | | | UGUAUGUAGAAGGUUAACUA | 2247 | |
| | | DMD | | | GAGCCUAAUAAAUGUACAAU | 2248 | |
| | | DMD | | | UUGUAUGUAGAAGGUUAACU | 2249 | |
| | | DMD | | | CAAUUUGUUUUGAGUAACU | 2250 | |
| | | DMD | | | UGCCUUCUGAAAUAGUCCAG | 2251 | |
| | | DMD | | | GUUAAUAGGGAAACAGCAUA | 2252 | |
| | | DMD | | | AACAAUGCAGAGUUAAUUGU | 2253 | |
| | | DMD | | | GAACAUGUUGAGUAGACACA | 2254 | |
| | | DMD | | | UUUAUCAUCUGUGUCUAUUC | 2255 | |
| | | DMD | | | UCUUUACUUUCUUGACUAUA | 2256 | |
| | | DMD | | | AAUAUUCUCAAACCUCGUUC | 2257 | |
| | | DMD | | | AUUAACUGUGUUCCAGAACG | 2258 | |
| | | DMD | | | UAACUGCUUCUUUGGAUGAC | 2259 | |
| | | DMD | | | GACCAGAACAGUGUAAGUUU | 2260 | |
| | | DMD | | | ACCAGAACAGUGUAAGUUUA | 2261 | |
| | | DMD | | | CUACUUUUUCCCCACUACUG | 2262 | |
| | | DMD | | | UGGAACACAGUUAAUUCACU | 2263 | |
| | | DMD | | | GUGUUGUUUAACUGCUUCUU | 2264 | |
| | | DMD | | | AACUGUCAGUUGCAUAUUCC | 2265 | |
| | | DMD | | | CAGAAAGGAAUGCUGGUACC | 2266 | |
| | | DMD | | | UCUGCCUACACAAUGAAUGG | 2267 | |
| | | DMD | | | CACAGAUCAAUCCAAUUGUU | 2268 | |
| | | DMD | | | UUGACAGGUGGAAAGUACAU | 2269 | |
| | | DMD | | | ACAUUUUUAGGCUUGACAGG | 2270 | |
| | | DMD | | | CUCUCCCAUGACAGACUCCC | 2271 | |
| | | DMD | | | UUGGUAAGAGUUAUGAUAAG | 2272 | |
| | | DMD | | | AACACAAAUUAAGUUCACCU | 2273 | |
| | | DMD | | | AGGAUCAGUGCUGUAGUGCC | 2274 | |
| | | DMD | | | GGCCGUUUAUUAUUAUUGAC | 2275 | |
| | | DMD | | | UCUCAGGAUUGCUAUGCAAC | 2276 | |
| | | DMD | | | CAGGAAGACAUACCAUGUAA | 2277 | |
| | | DMD | | | AGCAGGGCUCUUUCAGUUUC | 2278 | |
| | | DMD | | | UAACAUUUUCAGCUUGAACC | 2279 | |
| | | DMD | | | UCAAGCUGAAAAUGUUACAC | 2280 | |
| | | DMD | | | GUAACAUUUUCAGCUUGAAC | 2281 | |
| | | DMD | | | CAGAAUGAAUUUUGGAGCAC | 2282 | |
| | | DMD | | | UUUAUUAUUAUUGACUGGUG | 2283 | |
| | | DMD | | | AGAAGAAUCUGACCUUUACA | 2284 | |
| | | DMD | | | GCAGGGCUCUUUCAGUUUCU | 2285 | |
| | | DMD | | | CUAAACAGUAGCCAGGCGUG | 2286 | |
| | | DMD | | | CGCCUGGCUACUGUUUAGUG | 2287 | |
| | | DMD | | | CUCCGCACUAAACAGUAGCC | 2288 | |
| | | DMD | | | GUAGCCAGGCGUGUGGAUGU | 2289 | |
| | | DMD | | | CUUGGCUUUGACUAUUCUGC | 2290 | |
| | | DMD | | | AGUAGCCAGGCGUGUGGAUG | 2291 | |
| | | DMD | | | UCCUCCCACAUCCACACGCC | 2292 | |
| | | DMD | | | UUGGCUUUGACUAUUCUGCU | 2293 | |
| | | DMD | | | AUAAUGUCUCUGGCUUGUAA | 2294 | |
| | | DMD | | | UGGUACCCGGCAGCUCUCUG | 2295 | |
| | | DMD | | | GUGGGAGGAACCUCAAAGAG | 2296 | |
| | | DMD | | | UGACUAUUCUGCUGGGAACA | 2297 | |
| | | DMD | | | CUCUCUGAGGAAUGUUCCCU | 2298 | |
| | | DMD | | | AACAUUCCUCAGAGAGCUGC | 2299 | |
| | | DMD | | | AUUCUGAAGCUCCAAACAAU | 2300 | |
| | | DMD | | | UAAAUUACUCUGCUAAAGUA | 2301 | |
| | | DMD | | | AGUACAAACCAGGUUUGUAC | 2302 | |
| | | DMD | | | AUAUCCUUCCAGUACAAACC | 2303 | |
| | | DMD | | | CAAACCAGGUUUGUACUGGA | 2304 | |
| | | DMD | | | GGCAGCUAAAGCAUCACUGA | 2305 | |
| | | DMD | | | AUCUCUGAGUAGUACAAACC | 2306 | |
| | | DMD | | | GUGUCCCAUUCUCUUUGACU | 2307 | |
| | | DMD | | | UGUGUCCCAUUCUCUUUGAC | 2308 | |
| | | DMD | | | UUCUGAAUGUUGAACAAGUA | 2309 | |
| | | DMD | | | GUCUCCCAGUCAAAGAGAAU | 2310 | |
| | | DMD | | | AUUCUCUUUGACUGGGAGAC | 2311 | |
| | | DMD | | | UCUUUGACUGGGAGACAGGC | 2312 | |
| | | DMD | | | GUGGUGUCCUUUGAAUAUGC | 2313 | |
| | | DMD | | | AGAUUGUCCAGGAUAUAAUU | 2314 | |
| | | DMD | | | UUAGCAACCAAAUUAUAUCC | 2315 | |
| | | DMD | | | GUUGAAAUUAAACUACACAC | 2316 | |
| | | DMD | | | AUCUUUACCUGCAUAUUCAA | 2317 | |
| | | DMD | | | GUGUCCUUUGAAUAUGC | 2318 | |
| | | DMD | | | UUGUCCAGGAUAUAAUU | 2319 | |
| | | DMD | | | GCAACCAAAUUAUAUCC | 2320 | |
| | | DMD | | | GAAAUUAAACUACACAC | 2321 | |
| | | DMD | | | UUUACCUGCAUAUUCAA | 2322 | |
| | | DMD | | | UACACAUUUUUAGGCUUGAC | 2323 | |
| | | DMD | | | CAUUCCUGGGAGUCUGUCAU | 2324 | |
| | | DMD | | | UGUAUGAUGCUAUAAUACCA | 2325 | |
| | | DMD | | | GUGGAAAGUACAUAGGACCU | 2326 | |
| | | DMD | | | UCUUAUCAUAACUCUUACCA | 2327 | |
| | | DMD | | | ACAUUUUUAGGCUUGAC | 2328 | |
| | | DMD | | | UCCUGGGAGUCUGUCAU | 2329 | |
| | | DMD | | | AUGAUGCUAUAAUACCA | 2330 | |
| | | DMD | | | GAAAGUACAUAGGACCU | 2331 | |
| | | DMD | | | UAUCAUAACUCUUACCA | 2332 | |
| | | DMD | | | GAGTTCCTACTCAGACTGTTAC | 2333 | |
| | | | | | TC | | |
| | | DMD | | | GTGAGTTCCTACTCAGACTGTT | 2334 | |
| | | | | | ACTC | | |
| | | DMD | | | GTCTGAGTTCCTACTCAGACTG | 2335 | |
| | | | | | TTACTC | | |
| | | DMD | | | AAAGATATATAATGTCATGAAT | 2336 | |
| | | DMD | | | GCAGAATCAAATATAATAGTCT | 2337 | |
| | | DMD | | | AACAAATATCCCTTAGTATC | 2338 | |
| | | DMD | | | AATGTATTTCTTCTATTCAA | 2339 | |
| | | DMD | | | AACAATAAGTCAAATTTAATTG | 2340 | |
| | | DMD | | | GAACTGGTGGGAAATGGTCTA | 2341 | |
| | | | | | G | | |
| | | DMD | | | TCCTTTGGTAAATAAAAGTCCT | 2342 | |
| | | DMD | | | TAGGAATCAAATGGACTTGGAT | 2343 | |
| | | DMD | | | TAATTCTTTCTAGAAAGAGCCT | 2344 | |
| | | DMD | | | CTCTTGCATCTTGCACATGTCC | 2345 | |
| | | DMD | | | ACTTAGAGGTCTTCTACATACA | 2346 | |
| | | DMD | | | TCAGAGGTGAGTGGTGAGGGG | 2347 | |
| | | | | | A | | |
| | | DMD | | | ACACACAGCTGGGTTATCAGA | 2348 | |
| | | | | | G | | |
| | | DMD | | | CACAGCTGGGTTATCAGAG | 2349 | |
| | | DMD | | | ACACAGCTGGGTTATCAGAG | 2350 | |
| | | DMD | | | CACACAGCTGGGTTATCAGAG | 2351 | |
| | | DMD | | | AACACACAGCTGGGTTATCAG | 2352 | |
| | | | | | AG | | |
| | | DMD | | | CTGSTGGGARATGGTCTAG | 2353 | |
| | | DMD | | | ACTGGTGGGAAATGGTCTAG | 2354 | |
| | | DMD | | | AACTGGTGGGAAATGGTCTAG | 2355 | |
| | | DMD | | | AGAACTGGTGGGAAATGGTCT | 2356 | |
| | | | | | AG | | |
| | | DMD | | | ATATCTTCTTAAATACCCGA | 2357 | |
| | | DMD | | | AGTCTCACAAAACTGCAGAG | 2358 | |
| | | DMD | | | TACTTATGTATTTTAAAAAC | 2359 | |
| | | DMD | | | GAATAATTTCTATTATATTACA | 2360 | |
| | | DMD | | | TTCGAAAATTTCAGGTAAGCCG | 2361 | |
| | | DMD | | | TCATTTCTAAAAGTCTTTTGCC | 2362 | |
| | | DMD | | | TTTGAGACACAGTATAGGTTAT | 2363 | |
| | | DMD | | | ATATAATAGAAATTATTCAT | 2364 | |
| | | DMD | | | TAATATGCCCTGTAATATAA | 2365 | |
| | | DMD | | | TGATATCATCAATATCTTTG | 2366 | |
| | | DMD | | | GCAATTAATTGGAAAATGTG | 2367 | |
| | | DMD | | | CTTTAAGCTTAGGTAAAATCA | 2368 | |
| | | DMD | | | CAGTAATGTGTCATACCTTC | 2369 | |
| | | DMD | | | CAGGGCATATTATATTTAGA | 2370 | |
| | | DMD | | | CAAAAGCCAAATCTATTTCA | 2371 | |
| | | DMD | | | ATGCTTTGGTGGGAAGAAGTA | 2372 | |
| | | | | | GAGGA | | |
| | | DMD | | | ATGCTTTGGTGGGAAGAATAG | 2373 | |
| | | | | | AGGAC | | |
| | | DMD | | | TTGTGACAAGCTCACTAATTAG | 2374 | |
| | | | | | G | | |
| | | DMD | | | AAGTTTGAAGAACTTTTACCAG | 2375 | |
| | | | | | G | | |
| | | DMD | | | AGGCAGCGATAAAAAAAACCT | 2376 | |
| | | | | | GG | | |
| | | DMD | | | GCTTTGGTGGGAAGAAGTAGA | 2377 | |
| | | | | | GG | | |

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Genomic editing with CRISPR/Cas9 is a promising new approach for correcting or mitigating disease-causing mutations. Duchenne muscular dystrophy (DMD) is associated with lethal degeneration of cardiac and skeletal muscle caused by more than 3000 different mutations in the X-linked dystrophin gene (DMD). Most of these mutations are clustered in “hotspots.” There is a fortuitous correspondence between the eukaryotic splice acceptor and splice donor sequences and the protospacer adjacent motif sequences that govern prokaryotic CRISPR/Cas9 target gene recognition and cleavage. Taking advantage of this correspondence, optimal guide RNAs capable of introducing insertion/deletion (indel) mutations by nonhomologous end joining that abolish conserved RNA splice sites in 12 exons that potentially allow skipping of the most common mutant or out-of-frame DMD exons within or nearby mutational hotspots were screened. Correction of DMD mutations by exon skipping is referred to herein as “myoediting.” In proof-of-concept studies, myoediting was performed in representative induced pluripotent stem cells from multiple patients with large deletions, point mutations, or duplications within the DMD gene and efficiently restored dystrophin protein expression in derivative cardiomyocytes. In three-dimensional engineered heart muscle (EHM), myoediting of DMD mutations restored dystrophin expression and the corresponding mechanical force of contraction. Correcting only a subset of cardiomyocytes (30 to 50%) was sufficient to rescue the mutant EHM phenotype to near-normal control levels. Thus, abolishing conserved RNA splicing acceptor/donor sites and directing the splicing machinery to skip mutant or out-of-frame exons through myoediting allow correction of the cardiac abnormalities associated with DMD by eliminating the underlying genetic basis of the disease.

Identification of Optimal Guide RNAs to Target 12 Different Exons Associated with Hotspot Regions of DMD Mutations

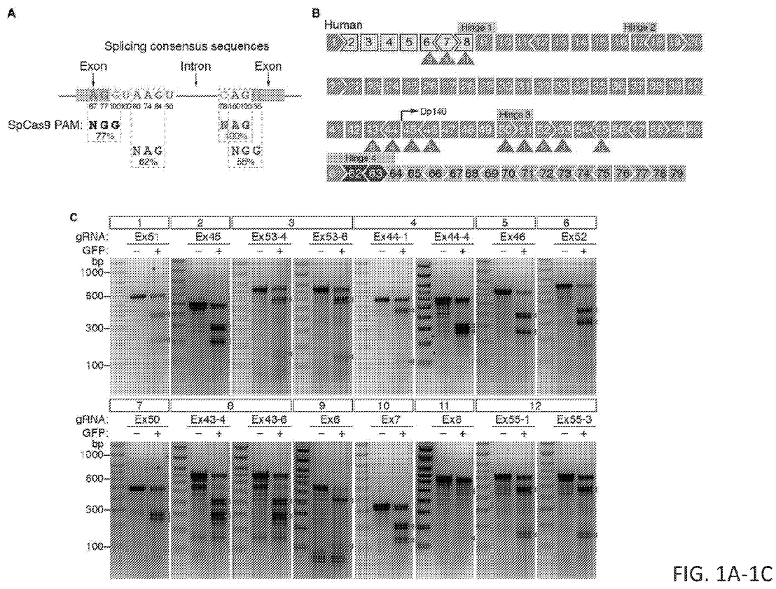

A list of the top 12 exons that, when skipped, can potentially restore the dystrophin open reading frame in most of the hotspot regions of DMD mutations is shown in Table 5. As an initial step toward correcting a majority of human DMD mutations by exon skipping, pools of guide RNAs were screened to target the top 12 exons of the human DMD gene (FIGS. 1A and 1B). Three to six PAM sequences (NAG or NGG) were selected to target the 3′ or 5′ splice sites, respectively, of each exon (FIG. 1A and Table 5). These guide RNAs were cloned in plasmid SpCas9-2A-GFP. Indels that remove essential splice donor or acceptor sequences allow for skipping of the corresponding target exon. On the basis of the frequency of known DMD mutations, these guide RNAs would be predicted to be capable of rescuing dystrophin function in up to 60% of DMD patients.

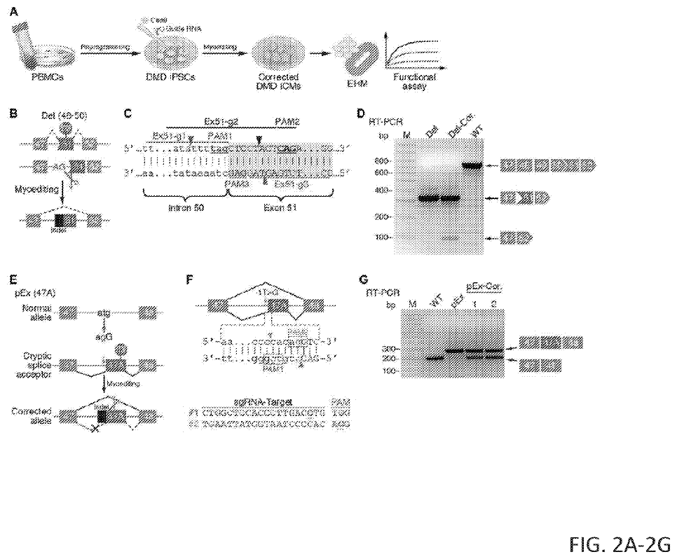

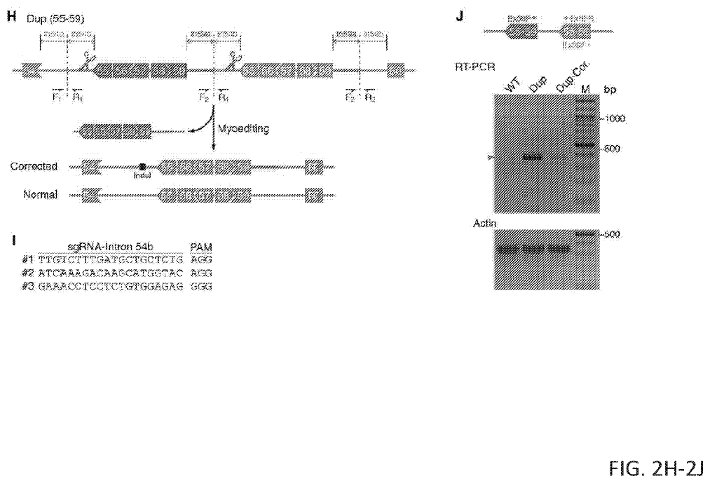

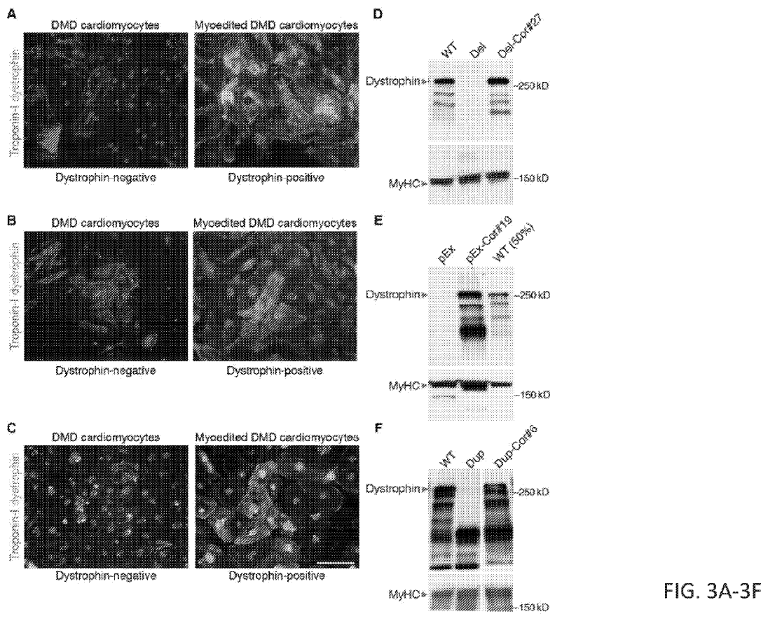

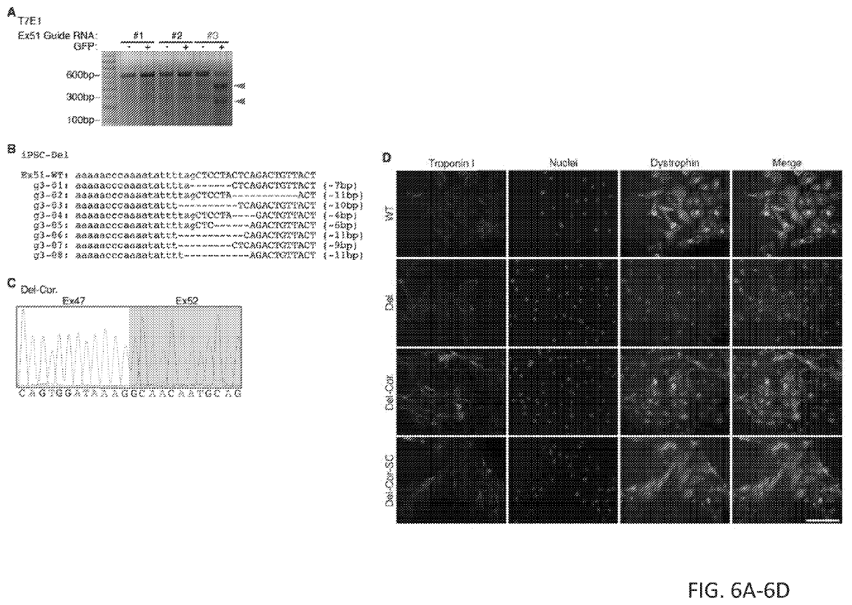

To test the feasibility and efficacy of this strategy in the human genome, human embryonic kidney 293 cells (239 cells) were used to target the splice acceptor site of exon 51 (FIG. 1C). Transfected 293 cells were sorted by green fluorescent protein (GFP) expression, and gene editing efficiency was detected by the mismatch-specific T7E1 endo-nuclease assay (FIG. 6A). The ability of three guide RNAs (Ex51-g1, Ex51-g2, and Ex51-g3) to target the splice acceptor site of exon 51 is shown in Table 5 and FIG. 2B. In GFP-positive sorted 293 cells, Ex51-g3 showed high editing activity, whereas Ex51-g1 and Ex51-g2 had no detectable activity. Next, cleavage efficiency of guide RNAs, which target the top 12 exons, including exons 51, 45, 53, 44, 46, 52, 50, 43, 6, 7, 8, and 55, was evaluated. One or two guide RNAs with the highest efficiency of editing of each exon are shown in FIG. 1C. The selected guide RNAs for exons 51, 45, and 55 use NAG as the PAM (Table 5). Genomic polymerase chain reaction (PCR) products from the myoedited top 12 exons were cloned and sequenced (FIG. 5A and Table 20). Indels were observed that removed essential splice sites or shifted the open reading frame (FIG. 5A). In brain and kidney tissues, an N-terminally truncated form of dystrophin (Dp140) is transcribed from an alternative promoter in intron 44. Skipping of six targeted exons (exons 51, 53, 46, 52, 50, and 55) in Dp140 mRNA was confirmed in 293 cells by sequencing of reverse transcription PCR (RT-PCR) products (FIG. 5B).

TABLE 20
Sequence of primers for top 12 exons.
PCR/T7E1 and RT-PCR primers

| Exon | | SEQ ID | | SEQ ID |
|---|---|---|---|---|
| # | PCR/T7E1 | NO: | RT-PCR | NO: |
| 51 | F: TTCCCTGGCAAGGTCTGA | 2427 | F-E47: CCCAGAAGAGCAAGATAAACTTGAA | 2451 |
| | R: ATCCTCAAGGTCACCCACC | 2428 | R-E52: CTCTGTTCCAAATCCTGCATTGT | 2452 |
| 45 | F: GTCTTTCTGTCTTGTATCCTTTGG | 2429 | | |
| | R: AATGTTAGTGCCTTTCACCC | 2430 | | |
| 53 | F: GGGAAATCAGGCTGATGGGT | 2431 | F-E52: CAAGACCAGCAATCAAGAGGCTAG | 2453 |
| | R: GTCTACTGTTCATTTCAGC | 2432 | R-E54: TCATGTGGACTTTTCTGGTATCATC | 2454 |
| 44 | F: GCAGGAAACTATCAGAGTG | 2433 | | |
| | R: ACACCTTGCTGTTACGAT | 2434 | | |
| 46 | F: CCACCAAACCTGGCAAAT | 2435 | F-E45: GAACTCCAGGATGGCATTGG | 2455 |
| | R: | 2436 | R-E52: CTCTGTTCCAAATCCTGCATTGT | 2456 |
| | GAACTATGAATAACCTAATGGGC | | | |
| | AG | | | |
| 52 | F: TTCTTACTCAAGGCATTCAGAC | 2437 | F-E51: GAAACTGCCATCTCCAAACTAGAAA | 2457 |
| | R: GGTCACCACACCCATCAAT | 2438 | R-E54: TTCTCCAAGAGGCATTGATATTCTC | 2458 |
| 50 | F: TGCCTGGAGAAAGGGTTT | 2439 | R-E47: CCCAGAAGAGCAAGATAAACTTGAA | 2459 |
| | R: GCACAGTCAATAACACAAAGGT | 2440 | R-E52: CTCTGTTCCAAATCCTGCATTGT | 2460 |
| 43 | F: AGCGATCCACTCTCTCAGGATG | 2441 | | |
| | R: | 2442 | | |
| | GCACCTCAATGCCCCAATCTGATT | | | |
| | TACG | | | |
| 6 | F: GGGTCTAATATGGCAGAATCCA | 2443 | | |
| | R: | 2444 | | |
| | GTTGTAAAGTAGGACATGATCTG | | | |
| | G | | | |
| 7 | F: AGGACTATGGGCATTGGTT | 2445 | | |
| | R: | 2446 | | |
| | GTGTAGAAATGACAAGTCTCAGA | | | |
| | TG | | | |
| 8 | F: | 2447 | | |
| | GAAAGCTACTCTGTTAGATGGGCT | | | |
| | AG | | | |
| | R: GGCTTTGTATATATACACGTG | 2448 | | |
| 55 | F: GCAGCATCAAAGACAAGCA | 2449 | F-E52: CAAGACCAGCAATCAAGAGGCTAG | 2461 |
| | R: TCCTTACGGGTAGCATCC | 2450 | R-E56: GAGAGACTTTTTCCGAAGTTCAC | 2462 |

### Correction of Diverse DMD Patient Mutations by Myoediting

To evaluate the effectiveness of a single-guide RNA to correct different types of human DMD mutations by exon skipping, three DMD iPSC lines with representative types of DMD mutations were obtained: a large deletion (termed Del; lacking exons 48 to 50), a pseudo-exon mutation (termed pEx; caused by an intronic point mutation), and a duplication mutation (termed Dup). Briefly, peripheral blood mono-nuclear cells (PBMCs) obtained from whole blood were cultured and then reprogrammed into iPSCs using recombinant Sendai viral vectors expressing reprogramming factors. Cas9 and guide RNAs for correction or bypass of the mutations in iPSC myoediting on an iPSC line (also known as Del) from a DMD patient with a large deletion of exons 48 to lines were introduced into cells by nucleofection. Pools of treated cells or single clones were then differentiated into induced cardiomyocytes (iCMs) using standardized conditions. Purified iCMs were used to generate 3D-EHM and to perform functional assays (FIG. 2A).

### Correction of a Large Deletion Mutation

It is estimated that ˜60 to 70% of DMD cases are caused by large deletions of one or more exons. Myoediting was performed on an iPSC line from a DMD patient with a large deletion of exons 48 to 50 in a hotspot. The large deletion creates a frameshift mutation and introduces a premature stop codon in exon 51, as shown in FIG. 2B. Destruction of the splice acceptor in exon 51 will, in principle, allow for splicing of exons 47 to 52, thereby reconstituting the open reading frame (FIG. 2B and FIG. 6B). Theoretically, skipping exon 51 can potentially correct ˜13% of DMD patients. Optimized guide RNA Ex51-g3 and Cas9 (FIG. 2C) were nucleofected into this iPSC line, resulting in successful destruction of the splice acceptor or reframing of exon 51 by NHEJ, as demonstrated by genomic sequencing, and restoration of the open reading frame (FIG. 6B). The pool of myoedited and DMD iPSCs (Del-Cor.) was differentiated into iCMs and rescue of in-frame dystrophin mRNA expression was confirmed by sequencing of RT-PCR products from amplification of exons 47 to 52 (FIG. 2D and FIG. 6C).

### Correction of a Pseudo-Exon Mutation

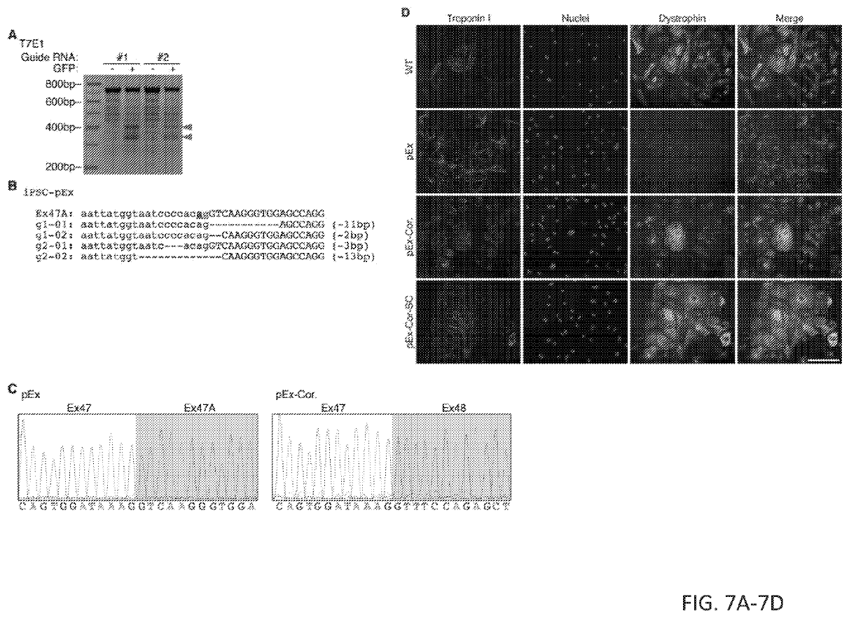

To further extend this approach to rare mutations, attempts were made to correct a point mutation within iPSCs from a DMD patient (also known as pEx), who has a spontaneous point mutation in intron 47 (c.6913-4037T>G). This point mutation generates a novel RNA splicing acceptor site (YnNYAG) and results in a pseudo-exon of exon 47A (FIG. 2E), which encodes a premature stop signal. Two guide RNAs (Ex47A-g1 and Ex47A-g2) were designed to precisely target the mutation (FIG. 2F and FIG. 7A and 7B). As shown in FIG. 2G, myoediting abolished the cryptic splice acceptor site and permanently skipped the pseudo-exon, restoring full-length dystrophin protein in the corrected cells (pEx-Cor.). The efficacy of exon skipping was tested by RT-PCR in these DMD iCMs (FIG. 2G). Sequencing of the RT-PCR products confirmed that exon 47 was spliced to exon 48 (FIG. 7C).

It is noteworthy that Ex47A-g2 targets only the mutant allele because the wild-type intron lacks the PAM sequence (NAG) for SpCas9. Moreover, the T>G mutation in this patient creates a disease-specific PAM sequence (AG) for Cas9. It is also noteworthy that this type of correction restores the normal dystrophin protein without any internal deletions (FIGS. 7B and 7C).

### Correction of a Large Duplication Mutation

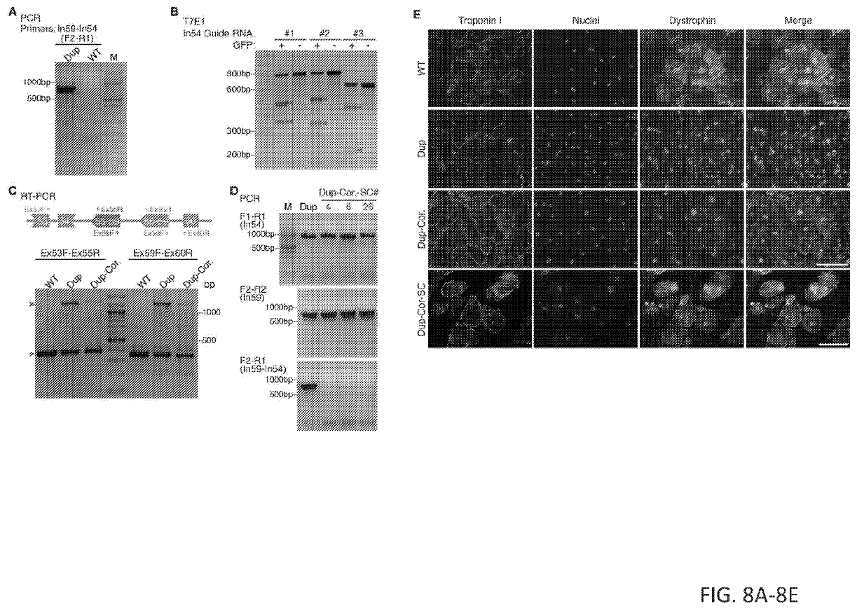

Exon duplications account for ˜10 to 15% of identified DMD-causing mutations. Myoediting was tested on an iPSC line (also known as Dup) from a DMD patient with a large duplication (exons 55 to 59), which disrupts the dystrophin open reading frame (FIG. 2H). Whole-genome sequencing and analysis the copy number variation profile in cells from this patient was performed and identified the precise insertion site in intron 54 (FIG. 2H). This insertion site (In59-In54 junction) was confirmed by PCR (FIG. 8A and Table 4).

It was hypothesized that the 5′ flanking sequence of the duplicated exon 55 is identical such that one guide RNA targeting this region should be able to make two DSBs and delete the entire duplicated region (exons 55 to 59; ˜150 kb). To test this hypothesis, three guide RNAs (In54-g1, In54-g2, and In54-g3) were designed to target sequences near the junction of intron 54 and exon 55 (FIG. 2I). The efficiency of DNA cutting with these guide RNAs was evaluated in 293 cells by T7E1 (FIG. 8B). Guide RNA In54-g1 was selected for subsequent experiments on Dup iPSCs. Genomic PCR products from the myoedited Dup iPSC mixture were cloned and sequenced (FIG. 8C).

To confirm the correction of the duplication mutation, the pool of treated DMD iPSCs (also known as Dup-Cor.) was differentiated into cardiomyocytes. mRNA with duplicated exons was semiquantified by RT-PCR using the duplication-specific primers (Ex59F, a forward primer in exon 59, and Ex55R, a reverse primer in exon 55) and normalized to expression of the b-actin gene (FIG. 2J and Table 4). As expected, the duplication-specific RT-PCR band was absent in wild-type (WT) cells and was decreased dramatically in Dup-Cor. cells. To confirm this result, RT-PCR on the duplication borders of exon 53 to Ex55 and Ex59 to exon 60 (FIG. 8D) was performed. The intensity of duplication-specific upper bands was decreased in corrected iCMs. Single colonies were picked from the treated mixture of cells. Duplication-specific PCR primers (F2-R1) were used to screen the corrected colonies (FIG. 8E). PCR results of three representative corrected colonies (Dup-Cor. #4, #6, and #26) and the uncorrected control (Dup) are shown in FIG. 8E. The absence of a duplication-specific PCR band in colonies 4, 6, and 26 confirmed the deletion of the duplicated DNA region.

Restoration of Dystrophin Protein in Patient-Derived iCMs by Myoediting

Next, the restoration and stable expression of dystrophin protein in single clones and pools of treated iCMs was confirmed by immunocytochemistry (FIG. 3A to 3C, and FIGS. 6D, 7D, and 8F) and Western blot analysis (FIG. 24, D to F). Even without clonal selection and expansion, most of the iCMs in Del-Cor., pEx-Cor., and Dup-Cor. were dystrophin-positive (FIG. 3A to 3C, and FIGS. 6D, 7D, and 8F). From mixtures of myoedited Del iPSCs, two clones (#16 and #27) were picked and differentiated into cardiomyocytes. Clone #27, which has a higher dystrophin expression level, was selected for subsequent experiments (also known as Del-Cor-SC). One selected clone for corrected pEx (#19) was used for further studies (also known as pEx-Cor-SC). Two selected clones for corrected Dup (#26 and #6) were differentiated into iCMs. Clone #6 was used for functional assay experiments (also known as Dup-Cor-SC). Dystrophin protein expression levels of the corrected iCMs were estimated to be comparable to WT cardiomyocytes (50 to 100%) by immunocytochemistry and Western blot analysis (FIG. 3).

Restoration of Function of Patient-Derived iCMs by Myoediting

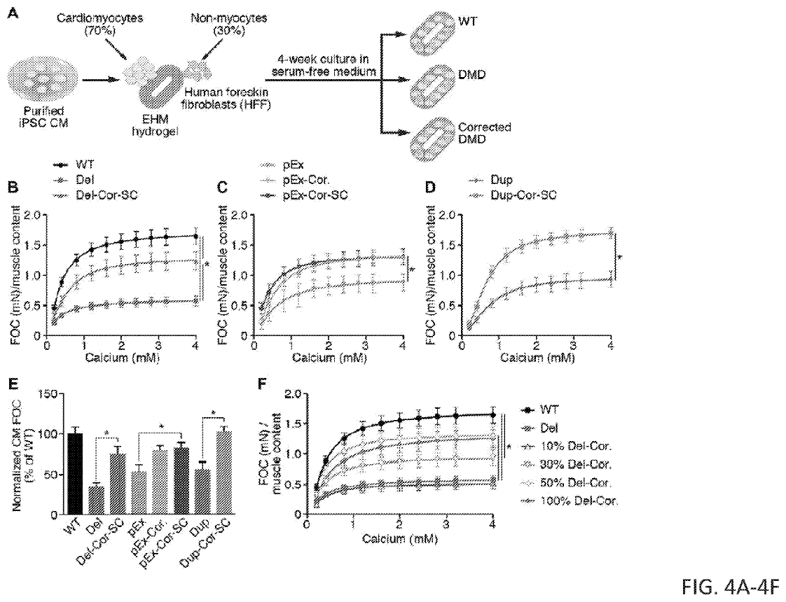

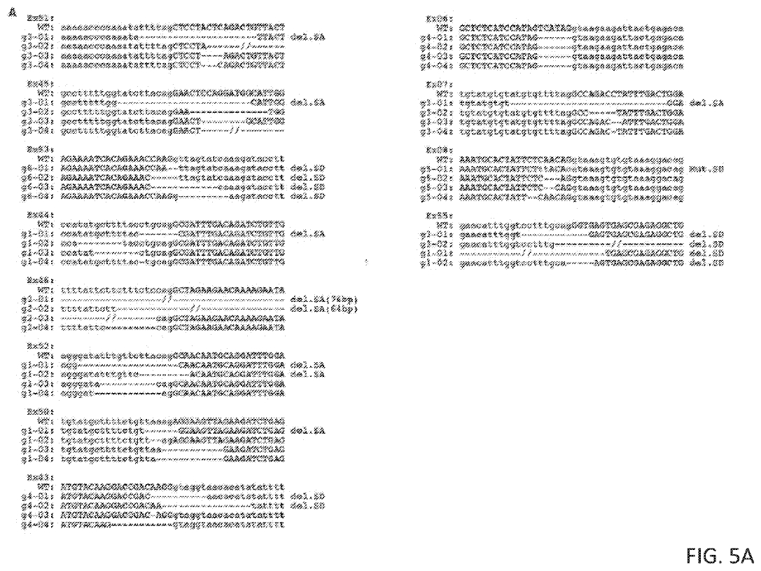

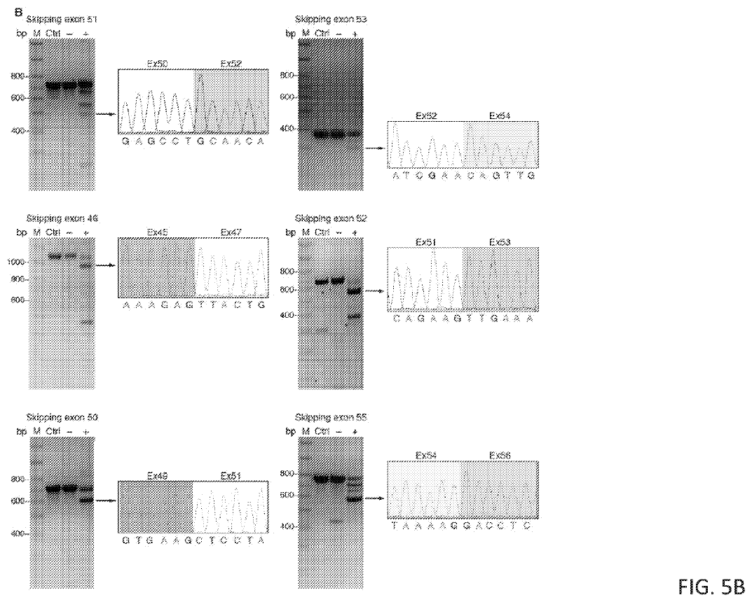

In addition to measuring dystrophin mRNA and protein expression by biochemical methods, functional analysis to the macroscale was used, using 3D-EHM derived from normal, DMD, and corrected DMD iCMs. Briefly, iPSCs-derived cardiomyocytes were metabolically purified by glucose deprivation. Purified cardiomyocytes were mixed with human foreskin fibroblasts (HFFs) at a 70%:30% ratio. The cell mixture was reconstituted in a mixture of bovine collagen and serum-free medium. After 4 weeks in culture, contraction experiments were performed (FIG. 4A).

EHMs from eight iPSC lines were tested: (i) WT, (ii) uncorrected Del, (iii) Del-Cor-SC, (iv) uncorrected pEx, (v) pEx-Cor., (vi) pEx-Cor-SC, (vii) uncorrected Dup, and (viii) Dup-Cor-SC. Functional phenotyping of DMD and corrected DMD cardiomyocytes in EHM revealed a contractile dysfunction in all DMD EHMs (Del, pEx, and Dup) compared to WT EHMs (FIG. 4B to 4E). A more pronounced contractile dysfunction was seen in Del compared with pEx and Dup EHM. Force of contraction (FOC) was markedly reduced in DMD EHMs and was significantly improved in corrected DMD EHMs (Del-Cor-SC, pEx-Cor-SC, and Dup-Cor-SC) (FIG. 4B to 4E) with completely restored cardiomyocyte maximal inotropic capacity in Dup-Cor-SC (FIGS. 4D and 4E).

Because current gene therapy delivery methods are only able to affect a portion of the heart muscle, an obvious question is what percentage of corrected cardiomyocytes is needed to rescue the phenotype of DCM. To address this question, DMD cells (Del) and corrected DMD cells (Del-Cor-SC) were precisely mixed to simulate a wide range of “therapeutic efficiency” (10 to 100%) in EHM (FIG. 4F). This revealed that 30 to 50% of cardiomyocytes need to be repaired for partial (30%) or maximal (50%) rescue of the contractile phenotype (FIG. 4F). These findings are consistent with previous in vivo studies showing that mosaic dystrophin expression in 50% cardiomyocytes in carrier mice resulted in a near-normal cardiac phenotype. Our findings show that contractile dysfunction was efficiently restored in corrected DMD EHM to a comparable level of WT EHM. Myoediting is thus a highly specific and efficient approach to rescue clinical phenotypes of DMD in EHM.

### Discussion

The DMD gene is the largest known gene in the human genome, encompassing 2.6 million base pairs and encoding 79 exons. The large size and complicated structure of the DMD gene contribute to its high rate of spontaneous mutation. There are ˜3000 documented mutations in humans, which include large deletions or duplications (˜77%), small indels (˜12%), and point mutations (˜9%). These mutations mainly affect exons; however, intronic mutations can alter the splicing pattern and cause the disease, as shown here for the pEx mutation.

To potentially simplify the correction of diverse DMD mutations by CRISPR/Cas9 gene editing, guide RNAs were identified that are capable of skipping the top 12 exons, which account for ˜60% of DMD patients. Thus, it is not necessary to design individual guides for each DMD mutation or excise large genomic regions with pairs of guide RNAs.

Rather, patient mutations can be grouped such that skipping of individual exons can restore dystrophin expression in large numbers of patients. In the proof-of-concept study described in Example 1, the optimized myoediting approach using only one guide RNA efficiently restored the DMD open reading frame in a wide spectrum of mutation types, including large deletions, point mutations, and duplications, which cover most of the DMD population. Even relatively large and complex deletions can be corrected by a single cut in the DNA sequence that eliminates a splice acceptor or donor site without the requirement for multiple guide RNAs to direct simultaneous cutting at distant sites with ligation of DNA ends. Although exon-skipping mainly converts DMD to milder BMD, for a subset of patients with duplication or pseudo-exon mutations, myoediting can eliminate the mutations and restore the production of normal dystrophin protein, as we have shown in this study for pEx and Dup mutations.

Dilated cardiomyopathy, characterized by contractile dysfunction and ventricular chamber enlargement, is one of the main causes of death in DMD patients. However, because of the marked interspecies differences in cardiac physiology and anatomy, as well as the natural history of the disease, the shortened longevity of these animals (˜2 years), and the small size of their hearts ( 1/3000 the size of the human heart), cardiomyopathy is not generally observed in mouse models of DMD at the young age. To overcome limitations and shortcomings of 2D cell culture systems and small animal models, human iPSC-derived 3D-EHM was used to show that dystrophin mutations impaired cardiac contractility and sensitivity to calcium concentration. Contractile dysfunction was observed in DMD EHM, resembling the DCM clinical phenotype of DMD patients. Contractile dysfunction was partially-to-fully restored in corrected DMD EHM by myoediting. Thus, genome editing represents an effective means of eliminating the genetic cause and correcting the muscle and cardiac abnormalities associated with DMD. The data presented herein further demonstrate that EHM serves as a suitable preclinical tool to approximate therapeutic efficiency of myoediting.

Human CRISPR clinical trials received approval in China and the United States. One key concern for the CRISPR/Cas9 system is specificity because off-target effects may cause unexpected mutations in the genome. Multiple approaches have been developed to evaluate possible off-target effects, including (i) in silico prediction of target sites and testing them by deep sequencing and (ii) unbiased whole-genome sequencing. In addition, several new approaches have been reported to minimize potential off-target effects and/or to improve the specificity of the CRISPR/Cas9 system, including titration of dosage of Cas9 and guide RNA, paired Cas9 nickases, truncated guide RNAs, and high-fidelity or enhanced Cas9. Although most studies have used in vitro cell culture systems, we and others did not observe off-target effects in our previous studies of germline editing and post-natal editing in mice. According to a recent study of gene editing in human preimplantation embryos, off-target mutations were also not detected in the edited genome. Although comprehensive and extensive analysis of off-target effects is beyond the scope of this study, we are aware that it will eventually be important to thoroughly evaluate possible off-target effects of individual guide RNAs before potential therapeutic application.

### Materials and Methods

Plasmids.

The pSpCas9(BB)-2A-GFP (PX458) plasmid containing the human codon-optimized SpCas9 gene with 2A-EGFP and the backbone of guide RNA was a gift from F. Zhang (plasmid #48138, Addgene). Cloning of guide RNA was carried out according to the Feng Zhang Lab CRISPR plasmid instructions (addgene.org/crispr/zhang/).

Transfection and Cell Sorting of Human 293 Cells.

Cells were transfected by Lipofectamine 2000 Transfection Reagent (Thermo Fisher Scientific) according to the manufacturer's instructions, and the cells were incubated for a total of 48 to 72 hours. Cell sorting was performed by the Flow Cytometry Core Facility at University of Texas (UT) Southwestern Medical Center. Transfected cells were dissociated using trypsin-EDTA solution. The mixture was incubated for 5 min at 37° C., and 2 ml of warm Dulbecco's modified Eagle's medium supplemented with 10% fetal bovine serum was added. The resuspended cells were transferred into a 15-ml Falcon tube and gently triturated 20 times. The cells were centrifuged at 1300 rpm for 5 min at room temperature. The medium was removed, and the cells were resuspended in 500 ml of phosphate-buffered saline (PBS) supplemented with 2% bovine serum albumin (BSA). Cells were filtered into a cell strainer tube through its mesh cap. Sorted single cells were separated into microfuge tubes into GFP+ and GFP-cell populations.

Human iPSC Maintenance, Nucleofection, and Differentiation.

The DMD iPSC line Del was purchased from Cell Bank RIKEN BioResource Center (cell no. HPS0164). The WT iPSC line was a gift from D. Garry (University of Minnesota). Other iPSC lines (pEx and Dup) were generated and maintained by UT Southwestern Wellstone Myoediting Core. Briefly, PBMCs obtained from DMD patients' whole blood were cultured and then reprogrammed into iPSCs using recombinant Sendai viral vectors expressing reprogramming factors (Cytotune 2.0, Life Technologies). iPSC colonies were validated by immuno-cytochemistry, mycoplasma testing, and teratoma formation. Human iPSCs were cultured in mTeSRTM1 medium (STEMCELL Technologies) and passaged approximately every 4 days (1:18 split ratio). One hour before nucleofection, iPSCs were treated with 10 mM ROCK inhibitor (Y-27632) and dissociated into single cells using Accutase (Innovative Cell Technologies Inc.). Cells (1×10⁶) were mixed with 5 mg of SpCas9-2A-GFP plasmid and nucleofected using the P3 Primary Cell 4D-Nucleofector X kit (Lonza) according to manufacturer's protocol. After nucleofection, iPSCs were cultured in mTeSRTM1 medium supplemented with 10 mM ROCK inhibitor, penicillin-streptomycin (1:100) (Thermo Fisher Scientific), and primosin (100 mg/ml; InvivoGen). Three days after nucleofection, GFP+ and GFP− were sorted by fluorescence-activated cell sorting, as described above, and subjected to PCR and T7E1 assay.

Isolation of Genomic DNA from Sorted Cells.

Protease K (20 mg/ml) was added to DirectPCR Lysis Reagent (Viagen Biotech Inc.) to a final concentration of 1 mg/ml. Cells were centrifuged at 4° C. at 6000 rpm for 10 min, and the supernatant was discarded. Cell pellets kept on ice were resuspended in 50 to 100 ml of DirectPCR/protease K solution and incubated at 55° C. for >2 hours or until no clumps were observed. Crude lysates were incubated at 85° C. for 30 min and then spun for 10 s. NaCl was added to a final concentration of 250 mM, followed by the addition of 0.7 volumes of isopropanol to precipitate DNA. The DNA was centrifuged at 4° C. at 13,000 rpm for 5 min, and the supernatant was discarded. The DNA pellet was washed with 1 ml of 70% EtOH and dissolved in water. The DNA concentration was measured using a NanoDrop instrument (Thermo Fisher Scientific).

Amplifying Targeted Genomic Regions by PCR.

PCR assays contained 2 ml of GoTaq polymerase (Promega), 20 ml of 5× green GoTaq reaction buffer, 8 ml of 25 mM MgCl₂, 2 ml of 10 mM primer, 2 ml of 10 mM deoxynucleotide triphosphate, 8 ml of genomic DNA, and double-distilled H₂O (ddH₂O) to 100 ml. PCR conditions were as follows: 94° C. for 2 min, 32× (94° C. for 15 s, 59° C. for 30 s, and 72° C. for 1 min), 72° C. for 7 min, and then held at 4° C. PCR products were analyzed by 2% agarose gel electrophoresis and purified from the gel using the QIAquick PCR Purification kit (Qiagen) for direct sequencing. These PCR products were subcloned into pCRII-TOPO vector (Invitrogen) according to the manufacturer's instructions. Individual clones were picked, and the DNA was sequenced.

T7E1 Analysis of PCR Products.

Mismatched duplex DNA was obtained by denaturation/renaturation of 25 ml of the genomic PCR samples using the following conditions: 95° C. for 10 min, 95° to 85° C. (−2.0° C./s), 85° C. for 1 min, 85° to 75° C. (−0.3° C./s), 75° C. for 1 min, 75° to 65° C. (−0.3° C./s), 65° C. for 1 min, 65° to 55° C. (−0.3° C./s), 55° C. for 1 min, 55° to 45° C. (−0.3° C./s), 45° C. for 1 min, 45° to 35° C. (−0.3° C./s), 35° C. for 1 min, 35° to 25° C. (−0.3° C./s), 25° C. for 1 min, and then held at 4° C.

Following denaturation/renaturation, the following was added to the samples: 3 ml of 10×NEBuffer 2, 0.3 ml of T7E1 (New England Biolabs), and ddH₂O to 30 ml. Digested reactions were incubated for 1 hour at 37° C. Undigested PCR samples and T7E1-digested PCR products were analyzed by 2% agarose gel electrophoresis.

Whole-Genome Sequencing.

Whole-genome sequencing was performed by submitting the blood samples to Novogene Corporation. Purified genomic DNA (1.0 mg) was used as input material for the DNA sample preparation. Sequencing libraries were generated using TruSeq Nano DNA HT Sample Preparation kit (Illumina) following the manufacturer's instructions. Briefly, the DNA sample was fragmented by sonication to a size of 350 bp. The DNA fragments were end-polished, A-tailed, and ligated with the full-length adapter for Illumina sequencing with further PCR amplification. The libraries were sequenced on an Illumina sequencing platform, and paired-end reads were generated.

Isolation of RNA.

RNA was isolated from cells using TRIzol RNA isolation reagent (Thermo Fisher Scientific) according to the manufacturer's instructions.

Cardiomyocyte Differentiation and Purification.

iPSCs were adapted and maintained in TESR-E8 (STEMCELL Technologies) on 1:120 Matrigel in PBS-coated plates and passaged using EDTA solution (Versene, Thermo Fisher Scientific) twice weekly. For cardiac differentiation, iPSCs were plated at 5×10⁴ to 1×10⁵ cells/cm² and induced with RPMI, 2% B27, 200 mM L-ascorbic acid-2-phosphate sesquimagnesium salt hydrate (Asc; Sigma-Aldrich), activin A (9 ng/ml; R&D Systems), BMP4 (5 ng/ml; R&D Systems), 1 mM CHIR99021 (Stemgent), and FGF-2 (5 ng/ml; Miltenyi Biotec) for 3 days; following another wash with RPMI medium, cells were cultured from days 4 to 13 with 5 mM IWP4 (Stemgent) in RPMI supplemented with 2% B27 and 200 mM Asc. Cardiomyocytes were metabolically purified by glucose deprivation from days 13 to 17 in glucose-free RPMI (Thermo Fisher Scientific) with 2.2 mM sodium lactate (Sigma-Aldrich), 100 mM b-mercaptoethanol (Sigma-Aldrich), penicillin (100 U/ml), and streptomycin (100 mg/ml). Cardiomyocyte purity was 92±2% from 15 independent differentiation runs (one to three for each cell line).

EHM Generation.

To generate defined, serum-free EHM, purified cardiomyocytes were mixed with HFFs (American Type Culture Collection) at a 70%:30% ratio. The cell mixture was reconstituted in a mixture of pH-neutralized medical-grade bovine collagen (0.4 mg per EHM; LLC Collagen Solutions) and concentrated serum-free medium [2×RPMI, 8% B27 without insulin, penicillin (200 U/ml), and streptomycin (200 mg/ml)] and cultured for 3 days in Iscove medium with 4% B27 without insulin, 1% nonessential amino acids, 2 mM glutamine, 300 mM ascorbic acid, IGF1 (100 ng/ml; AF-100-11), FGF-2 (10 ng/ml; AF-100-18B), VEGF165 (5 ng/ml; AF-100-20), TGF-b1 (5 ng/ml; AF-100-21C; all growth factors are from PeproTech), penicillin (100 U/ml), and streptomycin (100 mg/ml). After a 3-day condensation period, EHM were transferred to flexible holders to support auxotonic contractions. Analysis was carried out after a total EHM culture period of 4 weeks.

Analysis of Contractile Function.

Contraction experiments were performed under isometric conditions in organ baths at 37° C. in gassed (5% CO₂/95% 02) Tyrode's solution (containing 120 mM NaCl, 1 mM MgCl₂, 0.2 mM CaCl₂, 5.4 mM KCl, 22.6 mM NaHCO₃, 4.2 mM NaH₂PO₄, 5.6 mM glucose, and 0.56 mM ascorbate). EHM were electrically stimulated at 1.5 Hz with 5-ms square pulses of 200 mA. EHMs were mechanically stretched at intervals of 125 mm until the maximum systolic force amplitude (FOC) was observed according to the Frank-Starling law. Responses to increasing extracellular calcium (0.2 to 4 mM) were investigated to determine maximal inotropic capacity. Where indicated, forces were normalized to muscle content (sarcomeric α-actinin-positive cell content, as determined by flow cytometry).

Flow Cytometry of EHM-Derived Cells.

Single-cell suspensions of EHM were prepared as described previously and fixed in 70% ice-cold ethanol. Fixed cells were stained with Hoechst 3342 (10 mg/ml; Life Technologies) to exclude cell doublets. Cardiomyocytes were identified by sarcomeric a-actinin staining (clone EA-53, Sigma-Aldrich). Cells were run on a LSRII SORP cytometer (BD Biosciences) and analyzed using the DIVA software. At least 10,000 events were analyzed per sample.

Immunostaining.

iPSC-derived cardiomyocytes were fixed with acetone and subjected to immunostaining. Fixed cardiomyocytes were blocked with serum cocktail (2% normal horse serum/2% normal donkey serum/0.2% BSA/PBS), and incubated with dystrophin antibody (1:800; MANDYS8, Sigma-Aldrich) and troponin-I antibody (1:200; H170, Santa Cruz Biotechnology) in 0.2% BSA/PBS. Following overnight incubation at 4° C., they were incubated with secondary antibodies [biotinylated horse anti-mouse immunoglobulin G (IgG) (1:200; Vector Laboratories) and fluorescein-conjugated donkey anti-rabbit IgG (1:50; Jackson ImmunoResearch)] for 1 hour. Nuclei were counter-stained with Hoechst 33342 (Molecular Probes).

EHM cryosections to be immunostained were thawed, further air-dried, and fixed in cold acetone (10 min at −20° C.). Sections were briefly equilibrated in PBS (pH 7.3) and then blocked for 1 hour with serum cocktail (2% normal horse serum/2% normal donkey serum/0.2% BSA/PBS). Blocking cocktail was decanted, and dystrophin/troponin primary antibody cocktail [mouse anti-dystrophin, MANDYS8 (1:800; Sigma-Aldrich) and rabbit anti-troponin-I (1:200; H170, Santa Cruz Bio-technology)] in 0.2% BSA/PBS was applied without intervening wash. Following overnight incubation at 4° C., unbound primary antibodies were removed with PBS washes, and sections were probed for 1 hour with secondary antibodies [biotinylated horse anti-mouse IgG (1:200; Vector Laboratories) and rhodamine donkey anti-rabbit IgG (1:50; Jackson ImmunoResearch)] diluted in 0.2% BSA/PBS. Unbound secondary antibodies were removed with PBS washes, and final dystrophin labeling was carried out with a 10-min incubation of the sections with fluorescein-avidin-DCS (1:60; Vector Laboratories) diluted in PBS. Unbound rhodamine was removed with PBS washes, nuclei were counterstained with Hoechst 33342 (2 mg/ml; Molecular Probes), and slides were coverslipped with Vectashield (Vector Laboratories).

Western Blot Analysis.

Western blot analysis for human iPSC-derived cardiomyocytes was performed, using antibodies to dystrophin (ab15277, Abcam; D8168, Sigma-Aldrich), glyceraldehyde-3-phosphate dehydrogenase (MAB374, Millipore), and cardiac myosin heavy chain (ab50967, Abcam). Goat anti-mouse and goat anti-rabbit horseradish peroxidase-conjugated secondary antibodies (Bio-Rad) were used for described experiments.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
Angel et al., Mol. Cell. Biol., 7:2256, 1987a.
Angel et al., Cell, 49:729, 1987b.
Aartsma-Rus et al., Hum. Mutat. 30, 293-299, 2009.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Baskin et al., EMBO Mol Med 6, 1610-1621, 2014.
Benvenisty and Neshif, Proc. Natl. Acad. Sci. USA, 83:9551-9555, 1986.
Berkhout et al., Cell, 59:273-282, 1989.
Bhaysar et al., Genomics, 35(1):11-23, 1996.
Bikard et al., Nucleic Acids Res. 41(15): 7429-7437, 2013.
Blanar et al., EMBO J., 8:1139, 1989.
Bodine and Ley, EMBO J., 6:2997, 1987.
Boshart et al., Cell, 41:521, 1985.
Bostick et al., Mol Ther 19, 1826-1832, 2011.
Bosze et al., EMBO J., 5(7):1615-1623, 1986.
Braddock et al., Cell, 58:269, 1989.
Brinster et al., Proc. Natl. Acad. Sci. USA, 82(13):4438-4442, 1985.
Bulla and Siddiqui, J. Virol., 62:1437, 1986.
Burridge et al., Nat. Methods 11, 855-860, 2014.
Bushby et al., Lancet Neurol., 9(1): 77-93 (2010).
Bushby et al., Lancet Neurol., 9(2): 177-198 (2010).
Campbell & Kahl, Nature 338, 259-262, 1989.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chang et al., Stem Cells., 27:1042-1049, 2009.
Chatterjee et al., Proc. Natl. Acad. Sci. USA, 86:9114, 1989.
Chen and Okayama, Mol. Cell Biol., 7:2745-2752, 1987.
Cho et al., Nat. Biotechnol. 31(3): 230-232, 2013.
Choi et al., Cell, 53:519, 1988.
Cirak et al., The Lancet 378, 595-605, 2011.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
DeWitt et al., Sci Transl Med 8, 360ra134-360ra134, 2016.
Donnelly et al., J. Gen. Virol. 82, 1027-1041, 2001.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
EP 0273085
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Firak et al., Mol. Cell. Biol., 6:3667, 1986.
Foecking et al., Gene, 45(1):101-105, 1986.
Fonfara et al., Nature 532, 517-521, 2016.
Fraley et al., Proc Natl. Acad. Sci. USA, 76:3348-3352, 1979
Franz et al., Cardoscience, 5(4):235-43, 1994.
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goncalves et al., Mol Ther, 19(7): 1331-1341 (2011).
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101:1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hirochika et al., J. Virol., 61:2599, 1987.
Holbrook et al., Virology, 157:211, 1987.
Hollinger & Chamberlain, Current Opinion in Neurology 28, 522-527, 2015.
Horlick and Benfield, Mol. Cell. Biol., 9:2396, 1989.
Horwich et al., J. Virol., 64:642-650, 1990.
Hsu et al., Natl Biotechnol. 31:827-832, 2013
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jinek et al., Science 337, 816-821, 2012.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al., J Biol Chem., 266(6):3361-3364, 1991.
Kawamoto et al., Mol. Cell. Biol., 8:267, 1988.
Kelly et al., J. Cell Biol., 129(2):383-96, 1995.
Kiledjian et al., Mol. Cell. Biol., 8:145, 1988.
Kim et al., Nature Biotechnology, 1-2, 2016.
Kim et al., Nature Biotechnology 34, 876-881, 2016.
Kimura et al., Dev. Growth Differ., 39(3):257-65, 1997.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaPointe et al., Hypertension, 27(3):715-22, 1996
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Levinson et al., Nature, 295:79, 1982.
Levrero et al., Gene, 101:195-202, 1991.
Lin et al., Mol. Cell. Biol., 10:850, 1990.
Long et al., Science 345: 1184-1188, 2014.
Long et al., Science 351, 400-403, 2016.
Pinello et al., Nature Biotechnol. 34, 695-697, 2016.
Long et al., JAMA Neurol., 3388, 2016.
Long et al., Science 351, 400-403, 2016.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Mali et al., Science 339, 823-826, 2013a.
Mali et al., Nat Methods 10, 957-963, 2013b.
Mali et al., Nat. Biotechnol. 31:833-838, 2013c.
Mann et al., Cell, 33:153-159, 1983.
Maresca et al., Genome Research 23, 539-546, 2013.
Markowitz et al., J Virol., 62:1120-1124, 1988.
McNeall et al., Gene, 76:81, 1989.
Miksicek et al., Cell, 46:203, 1986.
Millay et al., Nat. Med. 14, 442-447, 2008.
Mojica et al., J Mol. Evol. 60, 174-182, 2005.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Nelson et al., Science 351, 403-407, 2016.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Ornitz et al., Mol. Cell. Biol., 7:3466, 1987.
Padgett, R. A., Trends Genet. 28, 147-154, 2012.
Palmiter et al., Cell, 29:701, 1982a.
Palmiter et al., Nature, 300:611, 1982b.
Paskind et al., Virology, 67:242-248, 1975.
Pech et al., Mol. Cell. Biol., 9:396, 1989.
Perales et al., Proc. Natl. Acad. Sci. USA, 91(9):4086-4090, 1994.
Perez-Stable and Constantin, Mol. Cell. Biol., 10:1116, 1990.
Picard et al., Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotech. Techniques, 9:169-174, 1995.
Ragot et al., Nature, 361:647-650, 1993.
Ran et al., Nature 520, 186-191, 2015.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Riffling et al., Nuc. Acids Res., 17:1619, 1989.
Rosen et al., Cell, 41:813, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sambrook and Russell, Molecular Cloning: A Laboratory Manual 3^{rd} Ed., Cold Spring Harbor Laboratory Press, 2001.
Sakai et al., Genes and Dev., 2:1144, 1988.
Satake et al., J Virology, 62:970, 1988.
Schaffner et al., J. Mol. Biol., 201:81, 1988.
Searle et al., Mol. Cell. Biol., 5:1480, 1985.
Sharp et al., Cell, 59:229, 1989.
Shaul and Ben-Levy, J. EMBO, 6:1913, 1987.
Sherman et al., Mol. Cell. Biol., 9:50, 1989.
Sleigh et al., J. EMBO, 4:3831, 1985.
Shimizu-Motohashi et al., Am J Transl Res 8, 2471-89, 2016.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. 1, 248:1, 1987.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Tabebordbar et al., Science 351, 407-411, 2016.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Top et al., J. Infect. Dis., 124:155-160, 1971.
Tóth et al., Biology Direct, 1-14, 2016.
Tronche et al., Mol. Biol. Med., 7:173, 1990.
Trudel and Constantini, Genes and Dev. 6:954, 1987.
Tsai et al., Nature Biotechnology 34, 882-887, 2016.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasseur et al., Proc Natl. Acad. Sci. USA., 77:1068, 1980.
Wagner et al., Proc. Natl. Acad. Sci. USA, 87(9):3410-3414, 1990.
Wang and Calame, Cell, 47:241, 1986.
Wang et al., Cell, 153:910-910, 2013.
Weber et al., Cell, 36:983, 1984.
Weinberger et al. Mol. Cell. Biol., 8:988, 1984.
Winoto et al., Cell, 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Wu et al., Cell Stem Cell 13, 659-662, 2013.
Wu et al., Nat Biotechnol 32, 670-676, 2014.
Xu et al., Mol Ther 24, 564-569, 2016.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yin et al., Nat Biotechnol 32, 551-553, 2014.
Yin et al., Physiol Rev 93, 23-67, 2013.
Young et al., Cell Stem Cell 18, 533-540, 2016.
Yutzey et al. Mol. Cell. Biol., 9:1397, 1989.
Zechner et al., Cell Metabolism 12, 633-642, 2010.
Zelenin et al., FEBS Lett., 280:94-96, 1991.
Zetsche et al., Cell 163, 759-771, 2015.
Ziober and Kramer, J. Bio. Chem., 271(37):22915-22922, 1996.

1. A method for editing a mutant dystrophin gene in a cardiomyocyte, the method comprising contacting the cardiomyocyte with:
a Cas9 nuclease, or a sequence encoding a Cas9 nuclease, and
a gRNA, or a sequence encoding a gRNA, wherein the gRNA targets a splice donor or splice acceptor site of the dystrophin gene.

2. The method of claim 1, wherein the gRNA targets a splice donor or splice acceptor site of exon 51, 45, 53, 44, 46, 52, 50, 43, 6, 7, 8, or 55.

3. The method of claim 1, wherein the gRNA comprises or targets a sequence of any one of SEQ ID NOs. 60-705, 712-862, 947-2377.

4. The method of claim 1, wherein a vector comprises the gRNA, or a sequence encoding the gRNA.

5. The method of claim 4, wherein the vector is a viral vector or a non-viral vector.

6. The method of claim 5, wherein the viral vector is an adeno-associated viral (AAV) vector.

7. The method of claim 6, wherein the AAV vector is selected from AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

8. The method of claim 5, wherein the non-viral vector is a plasmid.

9. The method of claim 5, wherein the non-viral vector is a nanoparticle.

10. The method of claim 1, wherein a first vector comprises the gRNA, or a sequence comprising the gRNA, and a second vector comprises the Cas9, or a sequence comprising the Cas9.

11. The method of claim 10, wherein the first vector and the second vector are AAVs.

12. The method of claim 1, wherein the mutant dystrophin gene comprises a point mutation.

13. The method of claim 12, wherein the point mutation is a pseudo-exon mutation.

14. The method of claim 1, wherein the mutant dystrophin gene comprises a deletion.

15. The method of claim 1, wherein the mutant dystrophin gene comprises a duplication mutation.

16. The method of claim 1, wherein the Cas9 nuclease is isolated or derived from a Streptococcus pyogenes (spCas9).

17. The method of claim 1, wherein the Cas9 nuclease is isolated or derived from a Staphylococcus aureus (saCas9).

18. A cardiomyocyte produced according to the method of claim 1, wherein the cardiomyocyte expresses a dystrophin protein.

19. The cardiomyocyte of claim 18, wherein the cardiomyocyte is derived from an induced pluripotent stem cell (iPSC).

20. A composition comprising a therapeutically effective amount of the cardiomyocyte of claim 18.

21. A method for treating or preventing Duchene Muscular Dystrophy (DMD) in a subject in need thereof, the method comprising administering to the subject a therapeutically effective amount of a composition of claim 20.

22. The method of claim 21, wherein the therapeutically effective amount at least partially or completely restores cardiac contractility in the patient.

23. An induced pluripotent stem cell (iPSC) comprising:
a Cas9 nuclease, or a sequence encoding a Cas9 nuclease, and
a gRNA, or a sequence encoding a gRNA,
wherein the gRNA targets a splice donor or splice acceptor site of the dystrophin gene.

24. A composition comprising a cardiomyocyte derived from the iPSC of claim 23.

25. A method for treating or preventing Duchene Muscular Dystrophy (DMD) in a subject in need thereof, the method comprising administering to the subject a therapeutically effective amount of the composition of claim 24.

26. The method of claim 25, wherein the therapeutically effective amount at least partially or completely restores cardiac contractility in the patient.

27. A method for treating or preventing Duchene Muscular Dystrophy (DMD) in a subject in need thereof, the method comprising administering to the subject:
a Cas9 nuclease, or a sequence encoding a Cas9 nuclease, and
a gRNA, or a sequence encoding a gRNA, wherein the gRNA targets a splice donor or splice acceptor site of the dystrophin gene;
wherein the administering restores dystrophin expression in at least 10% of the subject's cardiomyocytes.

28. The method of claim 27, wherein the gRNA targets a splice donor or splice acceptor site of exon 51, 45, 53, 44, 46, 52, 50, 43, 6, 7, 8, or 55.

29. The method of claim 27, wherein the gRNA comprises or targets a sequence of any one of SEQ ID NOs. 60-705, 712-862, or 947-2377.

30. The method of claim 27, wherein a vector comprises the gRNA, or a sequence encoding the gRNA.

31. The method of claim 30, wherein the vector is a viral vector or a non-viral vector.

32. The method of claim 31, wherein the viral vector is an adeno-associated viral (AAV) vector.

33. The method of claim 32, wherein the AAV vector is selected from an AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

34. The method of claim 31, wherein the non-viral vector is a plasmid.

35. The method of claim 31, wherein the non-viral vector is a nanoparticle.

36. The method of claim 27, wherein a first vector comprises the gRNA, or a sequence encoding the gRNA, and a second vector comprises the Cas9, or a sequence encoding the Cas9.

37. The method of claim 36, wherein the first vector and the second vector are AAVs.

38. The method of claim 27, wherein the mutant dystrophin gene comprises a point mutation.

39. The method of claim 38, wherein the point mutation is a pseudo-exon mutation.

40. The method of claim 27, wherein the mutant dystrophin gene comprises a deletion.

41. The method of claim 27, wherein the mutant dystrophin gene comprises a duplication mutation.

42. The method of claim 27, wherein the Cas9 nuclease is isolated or derived from a Streptococcus pyogenes (spCas9).

43. The method of claim 27, wherein the Cas9 nuclease is isolated or derived from a Staphylococcus aureus Cas9 (saCas9).

44. The method of claim 27, wherein the subject suffers from dilated cardiomyopathy.

45. The method of claim 27, wherein the administering restores dystrophin expression in at least 30% of the subject's cardiomyocytes.

46. The method of claim 27, wherein the administering at least partially rescues cardiac contractility.

47. The method of claim 27, wherein the administering restores dystrophin expression in at least 50% of the subject's cardiomyocytes.

48. The method of claim 27, wherein the administering completely rescues cardiac contractility.

49. A method for treating or preventing Duchene Muscular Dystrophy (DMD) in a subject in need thereof, the method comprising:
contacting an induced pluripotent stem cell (iPSC) with
a Cas9 nuclease, or a sequence encoding a Cas9 nuclease, and
a gRNA, or a sequence encoding a gRNA,
wherein the gRNA targets a splice donor or splice acceptor site of the dystrophin gene;
differentiating the iPSC into a cardiomyocyte; and
administering the cardiomyocyte to the subject.
